(19) United States
(12) Patent Application Publication
(10) Pub. No.: US 2004/0126389 A1
(43) Pub. Date: Jul. 1, 2004

(54) Vaccine composition

(76) Inventors: Francois-Xavier Jacques Berthet, Rixensart (BE); Wilfried L J Dalemans, Rixensart (BE); Philippe Denoel, Rixensart (BE); Guy Dequesne, Rixensart (BE); Chriatiane Feron, Rixensart (BE); Nathalie Garcon, Rixensart (BE); Yves Lobet, Rixensart (BE); Jan Poolman, Rixensart (BE); Georges Thiry, Rixensart (BE); Joelle Thonnard, Rixensart (BE); Pierre Voet, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/343,561

(22) PCT Filed: Jul. 31, 2001
(86) PCT No.: PCT/EP01/08857

(30) Foreign Application Priority Data
Feb. 8, 2001 (GB) 0103170.7

(51) Int. Cl. A61K 39/09; A61K 39/102; A61K 31/739

(52) U.S. Cl. 424/190.1; 514/54; 424/236.1; 424/256.1; 424/244.1

(57) ABSTRACT

The present invention relates to the field of vaccine formulation, particularly the field of novel adjuvant compositions comprising outer membrane vesicles (or blebs), and advantageous methods of detoxifying these compositions, and advantageous methods of use of such adjuvants.

### FIELD OF THE INVENTION

The present invention relates to the field of Gram-negative bacterial vaccine compositions, their manufacture, and the use of such compositions in medicine. More particularly it relates to the field of novel adjuvant compositions comprising outer-membrane vesicles (or bleb), and advantageous methods of use of such adjuvants.

BACKGROUND OF THE INVENTION

Adjuvants are important components in vaccines. Molecules that act as adjuvants may impact on innate immunity, antigen presenting cells (APC) and T lymphocytes. Indeed, by triggering the production of cytokines by macrophages, dendritic cells or natural killer cells, adjuvants will impact on innate immunity. Adjuvants may also stimulate antigen uptake and migration of dendritic cells and macrophages. Finally, adjuvants may also impact on the T-cells cytokine production profile and activate CD4 and/or CD8 T-cells. By impacting on immunity, adjuvants may modify the intrinsic immunogenic properties of an antigen and make this antigen more immunogenic and/or protective.

Although many adjuvant systems are known, there is need to define further, more advantageous adjuvant systems for the production of better vaccines. The present inventors have found bleb preparations in general (and in particular the genetically-modified bleb preparations described herein) are particularly suitable for formulating with other antigens, due to the adjuvant effect they confer on the antigens they are mixed with.

Blebs

The outer membrane (OM) of Gram-negative, bacteria is dynamic and, depending on the environmental conditions, can undergo drastic morphological transformations. Among these manifestations, the formation of outer-membrane vesicles or “blebs” has been studied and documented in many Gram-negative bacteria (Zhou, L et al. 1998. FEMS Microbiol. Lett. 163: 223-228). Among these, a non-exhaustive list of bacterial pathogens reported to produce blebs include: Bordetella pertussis, Borrelia burgdorferi, Brucella melitensis, Brucella ovis, Chlamydia psittaci, Chlamydia trachonmatis, Esherichia coli, Haemophilus influenzae, Legionella pneumophila, Neisseria gonorrhoeae, Neisseria meningitidis, Pseudomonas aeruginosa and Yersinia enterocolitica. Although the biochemical mechanism responsible for the production of OM blebs is not fully understood, these outer membrane vesicles have been extensively studied as they represent a powerful methodology in order to isolate outer-membrane protein preparations in their native conformation. In that context, the use of outer-membrane preparations is of particular interest to develop vaccines against Neisseria, Moraxella catarrhalis, Haemophilus influenzae, Pseudomonas aeruginosa and Chlamydia. Moreover, outer membrane blebs combine multiple proteinaceaous and non-proteinaceous antigens that are likely to confer extended protection against intra-species variants.

N. meningitidis serogroup B (menB) excretes outer membrane blebs in sufficient quantities to allow their manufacture on an industrial scale. Such multicomponent outer-membrane protein vaccines from naturally-occurring menB strains have been found to be efficacious in protecting teenagers from menB disease and have become registered in Latin America. An alternative method of preparing outer-membrane vesicles is via the process of detergent extraction of the bacterial cells (EP 11243).

Examples of bacterial species from which blebs can be made are the following.

Neisseria meningitidis:

Neisseria meningitidis (meningococcus) is a Gram-negative bacterium frequently isolated from the human upper respiratory tract. It occasionally causes invasive bacterial diseases such as bacteremia and meningitis.

For some years effort have been focused on developing meningococcal outer membrane based vaccines (de Moraes, J. C., Perkins, B., Camargo, M. C. et al. Lancet 340: 1074-1078, 1992; Bjune, G., Hoiby, E. A. Gronnesby, J. K. et al. 338: 1093-1096, 1991). Such vaccines have demonstrated efficacies from 57%-85% in older children (>4 years) and adolescents. Most of these efficacy trials were performed with OMV (outer membrane vesicles, derived by LPS depletion from blebs) vaccines derived from wild-type N. meningitidis B strains.

Many bacterial outer membrane components are present in these vaccines, such as PorA, PorB, Rmp, Opc, Opa, FrpB and the contribution of these components to the observed protection still needs further definition. Other bacterial outer membrane components have been defined (using animal or human antibodies) as potentially being relevant to the induction of protective immunity, such as TbpB, NspA (Martin, D., Cadieux, N., Hamel, J., Brodeux, B. R., J. Exp. Med. 185: 1173-1183, 1997; Lissolo, L., Maître-Wilmotte, C., Dumas, p. et al., Inf. Immun. 63: 884-890, 1995). The mechanism of protective immunity will involve antibody mediated bactericidal activity and opsonophagocytosis.

### Moraxella catarrhalis

Moraxella catarrhalis (also named Branhamella catarrhalis) is a Gram-negative bacterium frequently isolated from the human upper respiratory tract. It is responsible for several pathologies, the main ones being otitis media in infants and children, and pneumonia the elderly. It is also responsible for sinusitis, nosocomial infections and, less frequently, for invasive diseases.

M. catarrhalis produces outer membrane vesicles (Blebs). These Blebs have been isolated or extracted by using different methods (Murphy T. F., Loeb M. R. 1989. Microb. Pathog. 6: 159-174; Unhanand M., Maciver, I., Ramillo O., Arencibia-Mireles O., Argyle J. C., McCracken G. H. Jr., Hansen E. J. 1992. J. Infect. Dis. 165:644-650). The protective capacity of such Bleb preparations has been tested in a murine model for pulmonary clearance of M. catarrhalis. It has been shown that active immunization with Bleb vaccine or passive transfer of anti-Blebs antibody induces significant protection in this model (Maciver I., Unhanand M., McCracken G. H. Jr., Hansen, E. J. 1993. J. Infect. Dis. 168: 469-472). Proteins present on the surface of M. catarrhalis have been characterized using biochemical methods for their potential implication in the induction of a protective immunity (for review, see Murphy, T F (1996) Microbiol.Rev. 60:267) e.g. OMP B1, a 84 kDa protein, the expression of which is regulated by iron, and that is recognized by the sera of patients with pneumonia (Sethi, S, et al. (1995) Infect.Immun. 63:1516), and of UspA1 and UspA2 (Chen D. et al.(l999), Infect.Immun. 67:1310). In a mouse pneumonia model, the presence of antibodies raised against some of them (UspA, CopB) favors a faster clearance of the pulmonary infection. Another polypeptide (OMP CD) is highly conserved among M. catarrhalis strains, and presents homologies with a porin of Pseudomonas aeruginosa, which has been demonstrated to be efficacious against this bacterium in animal models.

Haemophilus influenzae

Haemophilus influenzae is a non-motile Gram-negative bacterium. Man is its only natural host. H. influenzae isolates are usually classified according to their polysaccharide capsule. Six different capsular types designated ‘a’ through ‘f’ have been identified. Isolates that fail to agglutinate with antisera raised against one of these six serotypes are classified as nontypeable, and do not express a capsule.

H. influenzae type b (Hib) is clearly different from the other types in that it is a major cause of bacterial meningitis and systemic diseases. Nontypeable strains of H. influenzae (NTHi) are only occasionally isolated from the blood of patients with systemic disease. NTHi is a common cause of pneumonia, exacerbation of chronic bronchitis, sinusitis and otitis media. NTHi strains demonstrate a large variability as identified by clonal analysis, whilst Hib strains as a whole are more homogeneous.

Outer membrane vesicles (or blebs) have been isolated from H. influenzae (Loeb M. R., Zachary A. L., Smith D. H. 1981. J. Bacteriol. 145:569-604; Stull T. L., Mack K., Haas J. E., Smit J., Smith A. L. 1985. Anal. Biochem. 150: 471-480). The vesicles have been associated with the induction of blood-brain barrier permeability (Wiwpelwey B., Hansen E. J., Scheld W. M. 1989 Infect. Immun. 57: 2559-2560), the induction of meningeal inflammation (Mustafa M. M., Ramilo O., Syrogiannopoulos G. A., Olsen K. D., McCraken G. H. Jr., Hansen, E. J. 1989. J. Infect. Dis. 159: 917-922) and to DNA uptake (Concino M. F., Goodgal S. H. 1982 J. Bacteriol. 152: 441-450). These vesicles are able to bind and be absorbed by the nasal mucosal epithelium (Harada T., Shimuzu T., Nishimoto K., Sakakura Y. 1989. Acta Otorhinolarygol. 246: 218-221) showing that adhesins and/or colonization factors could be present in Blebs. Immune response to proteins present in outer membrane vesicles has been observed in patients with various H. influenzae diseases (Sakakura Y., Harada T., Hamaguchi Y., Jin C. S. 1988. Acta Otorhinolarygol. Suppl. (Stockh.) 454: 222-226; Harada T., Sakakura Y., Miyoshi Y. 1986. Rhinology 24: 61-66).

Various surface-exposed proteins of H. influenzae have been shown to be involved in pathogenesis or have been shown to confer protection upon vaccination in animal models.

For instance various adhesins have been found (fimbriae and pili [Brinton C C. et al. 1989. Pediatr. Infect. Dis. J. 8:S54; Kar S. et al. 1990. Infect. Immun. 58:903; Gildorf J R. et al. 1992. Infect. Immun. 60:374; St. Geme J W et al. 1991. Infect. Immun. 59:3366; St. Geme J W et al. 1993. Infect. Immun. 61: 2233], HMW1 and HMW2 [St. Geme J W. et al. 1993. Proc. Natl. Acad. Sci. USA 90:2875], NTHi 115-kDa Hia protein [Barenkamp S J., St Geme S. W. 1996. Mol. Microbiol.] which is highly similar to H. influenzae type b Hsf [St. Geme J W. et al. 1996. J. Bact. 178:6281], and Hap [St. Geme J W. et al. 1994. Mol. Microbiol. 14:217].

Five major outer membrane proteins (OMP) have also been identified: P1, 2, 3, 4 and 5 (Loeb M R. et al. 1987. Infect. Immun. 55:2612; Musson R S. Jr. et al. 1983. J. Clin. Invest. 72:677; Haase E M. et al. 1994 Infect. Immun. 62:3712; Troelstra A. et al. 1994 Infect. Immun. 62:779; Green B A. et al. 1991. Infect.Immun.59:3191). OMP P6 is a conserved peptidoglycan associated lipoprotein making up 1-5% of the outer membrane Nelson M B. et al. 1991. Infect. Immun. 59:2658; Demaria T F. et al. 1996. Infect. Immun. 64:5187).

In line with the observations made with gonococci and meningococci, NTHi expresses on its surface a dual human transferrin receptor composed of ThpA and TbpB when grown under iron limitation (Loosmore S M. et al. 1996. Mol. Microbiol. 19:575). Hemoglobin/haptoglobin receptor also have been described for NTHi (Maciver I. et al. 1996. Infect. Immun. 64:3703). A receptor for Haem:Hemopexin has also been identified (Cope L D. et al. 1994. Mol.Microbiol. 13:868). A lactoferrin receptor is also present amongst NTHi (Schryvers A B. et al. 1989. J. Med. Microbiol. 29:121).

Other interesting antigens on the surface of the bacterium include an 80 kDa OMP, the D15 surface antigen (Flack F S. et al. 1995. Gene 156:97); a 42 kDa outer membrane lipoprotein, LPD (Akkoyunlu M. et al. 1996. Infect. Immun. 64:4586); a minor 98 kDa high molecular weight adhesin OMP (Kimura A. et al. 1985. Infect. Immun. 47:253); IgA1-protease (Mulks M H., Shoberg R J. 1994. Meth. Enzymol. 235:543); OMP26 (Kyd, J. M., Cripps, A. W. 1998. Infect. Immun. 66:2272); and NTHi HtrA protein (Loosmore S. M. et al. 1998. Infect. Immun. 66:899).

Pseudomonas aeruginosa:

The genus Pseudomonas consists of Gram-negative, polarly flagellated, straight and slightly curved rods that grow aerobically and do not forms spores. Because of their limited metabolic requirements, Pseudomonas spp. are ubiquitous and are widely distributed in the soil, the air, sewage water and in plants. Numerous species of Pseudomonas such as P. aeruginosa, P. pseudomallei, P. mallei P. maltophilia and P. cepacia have also been shown to be pathogenic for humans. Among this list P. aeruginosa is considered as an important human pathogen since it is associated with opportunistic infection of immuno-compromised host and is responsible for high morbidity in hospitalized patients. Nosocomial infection with P. aeruginosa afflicts primarily patients submitted for prolonged treatment and receiving immuno-suppressive agents, corticosteroids, antimetabolites antibiotics or radiation.

To examine the protective properties of OM proteins, a vaccine containing P. aeruginosa OM proteins of molecular masses ranging from 20 to 100 kDa has been used in pre-clinical and clinical trials. This vaccine was efficacious in animal models against P. aeruginosa challenge and induced high levels of specific antibodies in human volunteers. Plasma from human volunteers containing anti-P. aeruginosa antibodies provided passive protection and helped the recovery of 87% of patients with severe forms of P. aeruginosa infection. More recently, a hybrid protein containing parts of the outer membrane proteins OprF (amino acids 190-342) and OprI (amino acids 21-83) from Pseudomonas aeruginosa fused to the glutathione-S-transferase was shown to protect mice against a 975-fold 50% lethal dose of P. aeruginosa (Knapp et al. 1999. Vaccine. 17:1663-1669).

The present inventors have realised that blebs may be used as an effective adjuvant in conjunction with antigens.

Although wild-type blebs may be used, the inventors have realised a number of drawbacks associated with the use of wild-type blebs (either naturally occurring or chemically made).

Examples of such problems are the following:

the toxicity of the LPS remaining on the surface of the bleb

the potential induction of an autoimmune response because of host-identical structures (for example the capsular polysaccharide in Neisseria meningitidis serogroup B, the lacto-N-neotetraose in Neisseria LPS, saccharide structure within ntHi LPS, saccharide structures within Pili).

the presence of immunodominant but variable proteins on the bleb (PorA; TbpB, Opa [N. meningitidis B]; P2, P5 [non-typeable H. influenzae])—such blebs being effective only against a restricted selection of bacterial species. Type-specificity of the bactericidal antibody response may preclude the use of such vaccines in infancy.

the presence of unprotective (non relevant) antigens (Rmp, H8, . . . ) on the bleb—antigens that are decoys for the immune system

the lack of presence of important molecules which are produced conditionally (for instance iron-regulated outer membrane proteins, IROMP's, in vivo regulated expression mechanisms)—such conditions are hard to control in bleb production in order to optimise the amount of antigen on the surface

the low level of expression of protective, (particularly conserved) antigens (NspA, P6)

Although the first 2 problems are troublesome to use certain bleb preparations as adjuvants, the latter 4 problems are troublesome if the bleb is also to be included in a vaccine in its own right as an immunogenic component against the bacteria from which it is derived.

Such problems may prevent the use of bleb components in human vaccine reagents. This is particularly so for paediatric use (<4 years) where reactogenicity against bleb components is particularly important, and where bleb vaccines (for instance the previously mentioned marketed MenB bleb vaccine) have been shown to be ineffective at immuno-protecting.

Accordingly, the present invention provides methods of alleviating the above problems using genetically engineered bacterial strains, which result in improved bleb adjuvants. Such methods will be especially useful in the generation of new vaccines against bacterial pathogens such as Neisseiria meningitidis, Moraxella catarrhalis, Haemophilus influenzae, Pseudomonas aeruginosa, and others.

Each of these methods of improvement individually improve the bleb adjuvant, however a combination of one or more of these methods work in conjunction so as to produce an optimised engineered bleb vaccine component which is non-toxic, with a strong adjuvant activity, suitable for paediatric use, and which may be immuno-protective in its own right against the organism from which it is derived.

### SUMMARY OF THE INVENTION

The present invention provides various uses of Gram-negative bacterial blebs as an effective adjuvant in immunogenic compositions.

In one embodiment there is provided an immunogenic composition comprising an antigen derived from a pathogen which is capable of protecting a host against said pathogen, mixed with an adjuvant comprising a bleb preparation derived from a Gram-negative bacterial strain.

Preferably the bacterial source of the bleb adjuvant is from a difference strain or species than the source of the antigen (they are heterologous). Most preferably they are from different pathogens. Preferred compositions are made by adding blebs and antigen to the formulation separately.

The antigen may be a polysaccharide or polysaccharide conjugate antigen. In such case a composition consisting of N. meningitidis B bleb and N. meningitidis C polysaccharide (as described in WO 99/61053) is not included in the invention.

Alternatively, the antigen may be a peptide or protein antigen.

The inventors have realised that bleb adjuvants are particular useful where a fast-acting protective immune response against the antigen is required. Blebs can be particularly useful in this regard over other adjuvants. A method of inducing a fast-acting protective immune response against the antigen contained in the immunogenic compositions of the invention is also provided, comprising the step of administering to a host an effective amount of the immunogenic composition of the invention. This is particularly useful in vaccines for the elderly, thus a method of protecting an elderly patient against a pathogen by administering to said patient an effective amount of the immunogenic composition of the invention in which the antigen is derived from said pathogen, and the use of the adjuvant in this regard are further provided.

The blebs of the invention may be a wild-type preparation (collected from the bacterial culture or extracted with detergent such as deoxycholate), or may be a genetically-engineered bleb preparation from a Gram-negative bacterial strain characterized in that said preparation is obtainable by employing one or more processes selected from the following group:

a) a process of reducing immunodominant variable or non-protective antigens within the bleb preparation comprising the steps of determining the identity of such antigen, engineering a bacterial strain to produce less or none of said antigen, and making blebs from said strain;

b) a process of upregulating expression of protective, endogenous (and preferably conserved) OMP antigens within the bleb preparation comprising the steps of identifying such antigen, engineering a bacterial strain so as to introduce a stronger promoter sequence upstream of a gene encoding said antigen such that said gene is expressed at a level higher than in the non-modified bleb, and making blebs from said strain;

c) a process of upregulating expression of conditionally-expressed, protective (and preferably conserved) OMP antigens within the bleb preparation comprising the steps of identifying such an antigen, engineering a bacterial strain so as to remove the repressive control mechanisms of its expression (such as iron restriction), and making blebs from said strain;

d) a process of modifying lipid A portion of bacterial LPS within the bleb preparation, comprising the steps of identifying a gene involved in rendering the lipid A portion of LPS toxic, engineering a bacterial strain so as to reduce or switch off expression of said gene, and making blebs from said strain;

e) a process of modifying lipid A portion of bacterial LPS within the bleb preparation, comprising the steps of identifying a gene involved in rendering the lipid A portion of LPS less toxic, engineering a bacterial strain so as to introduce a stronger promoter sequence upstream of said gene such that said gene is expressed at a level higher than in the non-modified bleb, and making blebs from said strain;

f) a process of reducing lipid A toxicity within the bleb preparation and increasing the levels of protective antigens, comprising the steps of engineering the chromosome of a bacterial strain to incorporate a gene encoding a Polymyxin A peptide, or a derivative or analogue thereof, fused to a protective antigen, and making blebs from said strain;

g) a process of creating conserved OMP antigens on the bleb preparation comprising the steps of identifying such antigen, engineering a bacterial strain so as to delete variable regions of a gene encoding said antigen, and making blebs from said strain;

h) a process of reducing expression within the bleb preparation of an antigen which shares a structural similarity with a human structure and may be capable of inducing an auto-immune response in humans (such as the capsular polysaccharide of N. meningitidis B), comprising the steps of identifying a gene involved in the biosynthesis of the antigen, engineering a bacterial strain so as to reduce or switch off expression of said gene, and making blebs from said strain; or

i) a process of upregulating expression of protective, endogenous (and preferably conserved) OMP antigens within the bleb preparation comprising the steps of identifying such antigen, engineering a bacterial strain so as to introduce into the chromosome one or more further copies of a gene encoding said antigen controlled by a heterologous, stronger promoter sequence, and making blebs from said strain.

Processes d), e), f) and h) are particularly advantageous in the manufacture of bleb adjuvants of the invention that are safe in humans. One or more (2, 3, or 4) of these processes are preferably used to manufacture bleb adjuvant.

In a specific embodiment the immunogenic composition of the invention may thus comprise a bleb adjuvant made by process d) wherein the bleb preparation is derived from a strain which has a detoxified lipid A portion of bacterial LPS, due to the strain having been engineered to reduce or switch off expression of one or more genes selected from the group consisting of: htrB, msbB and lpxK (or homologues thereof).

In a further embodiment the immunogenic composition of the invention may comprise a bleb adjuvant made by process e) wherein the bleb preparation is derived from a strain which has a detoxified lipid A portion of bacterial LPS, due to the strain having been engineered to express at a higher level one or more genes selected from the group consisting of: pmrA, pmrB, pmrE and pmrF (or homologues thereof).

In a still further embodiment the immunogenic composition of the invention may comprise a bleb adjuvant made by process h) wherein the bleb preparation is derived from a strain engineered not produce a capsular polysaccharide, lipopolysaccharide or lipooligosaccharide comprising an antigen similar to a human structure by reducing or switching off expression of one or more genes selected from the group consisting of: galE, siaA, siaB, siaC, siaD, ctrA, ctrB, ctrC and ctrD (or homologues thereof).

### BRIEF DESCRIPTION OF THE DRAWINGS

FIG. 1: Reactivity of the 735 mAb on different colonies.

FIG. 2: Reactivities of specific monoclonal antibodies by whole cell Elisa.

FIG. 3: Schematic representation of the pCMK vectors used to deliver genes, operons and/or expression cassettes in the genome of Neisseria meningitidis.

FIG. 4: Analysis of PorA expression in total protein extracts of recombinant N. meningitidis serogroupB (H44/76 derivatives). Total proteins were recovered from cps− (lanes 3 and 4), cps− porA::pCMK+ (lanes 2 and 5) and cps− porA::nspA (lanes 1 and 6) recombinant N. meningitidis serogroupB strains and were analyzed under SDS-PAGE conditions in a 12% polyacrylamide gel. Gels were stained with Coomassie blue (lanes 1 to 3) or transferred to a nitrocellulose membrane and immuno-stained with an anti-PorA monoclonal antibody.

FIG. 5: Analysis of NspA expression in protein extracts of recombinant N. meningitidis serogroupB strains (H44/76 derivatives). Proteins were extracted from whole bacteria (lanes 1 to 3) or outer-membrane blebs preparations (lanes 4 to 6) separated by SDS-PAGE on a 12% acrylamide gel and analyzed by immuno-blotting using an anti-NspA polyclonal serum. Samples corresponding to cps− (lanes 1 and 6), cps− pora::pCMK+ (lanes 3 and 4) and cps− porA::nspA (lanes 2 and 5) were analyzed. Two forms of NspA were detected: a mature form (18 kDa) co-migrating with the recombinant purified NspA, and a shorter form (15 kDa).

FIG. 6: Analysis of D15/omp85 expression in protein extracts of recombinant N. meningitidis serogroupB strains (H44/76 derivatives). Proteins were extracted from outer-membrane blebs preparations and were separated by SDS-PAGE on a 12% acrylamide gel and analyzed by immuno-blotting using an anti-omp85 polyclonal serum. Samples corresponding to cps− (lane 2), and cps−, PorA+, pCMK+Omp85/D15 (lane 1) recombinant N. meningitidis serogroupB strains were analyzed.

FIG. 7: General strategy for modulating gene expression by promoter delivery (RS stands for restriction site).

FIG. 8: Analysis of outer-membrane blebs produced by recombinant N. meningitidis serogroupB cps− strains (H44/76 derivatives). Proteins were extracted from outer-membrane bleb preparations and were separated by SDS-PAGE under reducing conditions on a 4-20% gradient polyacrylamide gel. The gel was stained with Coomassie brilliant blue R250. Lanes 2, 4, 6 corresponded to 5 μg of total proteins whereas lanes 3, 5 and 7 were loaded with 10 μg proteins.

FIG. 9: Construction of a promoter replacement plasmid used to up-regulate the expression/production of Omp85/D15 in Neisseria meningitidis H44/76.

FIG. 10: Analysis of OMP85 expression in total protein extracts of recombinant NmB (H44/76 derivatives). Gels were stained with Coomassie blue (A) or transferred to nitrocellulose membrane and immuno-stained with rabbit anti-OMP85 (N.gono) monoclonal antibody (B).

FIG. 11: Analysis of OMP85 expression in OMV preparations from recombinant NmB (H44/76 derivatives). Gels were stained with Coomassie blue (A) or transferred to nitrocellulose membrane and immuno-stained with rabbit anti-OMP85 polyclonal antibody (B).

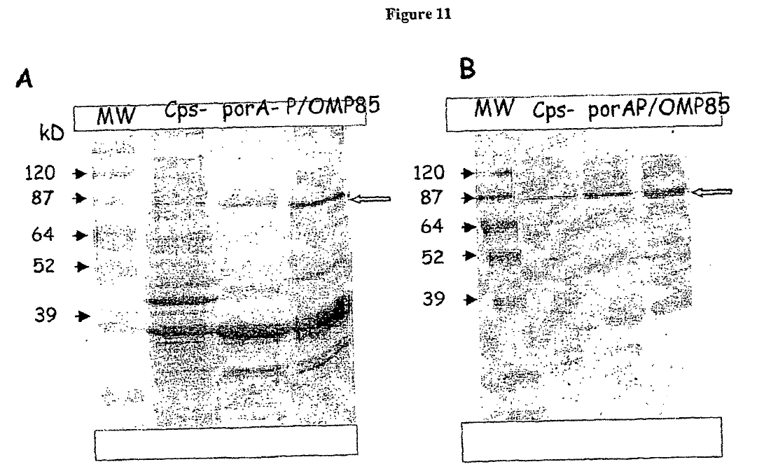

FIG. 12: Schematic representation of the recombinant PCR strategy used to delete the lacO in the chimeric porA/lacO promoter.

FIG. 13: Analysis of Hsf expression in total protein extracts of recombinant N. meningitidis serogroup B (H44/76 derivatives). Total proteins were recovered from Cps−PorA+(lanes 1), and Cps−PorA+/Hsf (lanes 2) recombinant N. meningitidis serogroup B strains and were analyzed under SDS-PAGE conditions in a 12% polyacrylamide gel. Gels were stained with Coomassie blue.

FIG. 14: Analysis of GFP expression in total protein extracts of recombinant N. meningitidis (H44/76 derivative). Total protein were recovered from Cps−, PorA+ (lanel), Cps−, PorA− GFP+ (lane2 & 3) recombinant strains. Proteins were separated by PAGE-SDS in a 12% polyacrylamide gel and then stained with Coomassie blue.

FIG. 15: Illustration of the pattern of major proteins on the surface of various recominant bleb preparations as analysed by SDS-PAGE (Coomassie staining).

FIG. 16: Specific anti-Hsf response for various bleb and recombinant bleb preparations using purified recombinant Hsf protein.

FIG. 17: Analysis of NspA expression in total protein extracts of recombinant NmB (serogroup B derivatives). Gels were stained with Coomassie blue (A) or transferred to nitrocellulose membrane and immuno-stained with mouse anti-PorA monoclonal antibody (B) or mouse anti-NspA polyclonal antibody (C).

### DESCRIPTION OF THE INVENTION

Vaccine Combinations & Advantageous Uses of Blebs as Adjuvants

Immunogenic compositions of the invention (preferably vaccine combinations) may comprise wild-type Gram-negative bacterial bleb preparations (isolated from the culture medium, or from cells by detergent [e.g. deoxycholate] extraction) or the genetically-modified bleb preparations described later. The antigen against a disease state is preferably from a heterologous source from the source of the blebs, and is preferably mixed with the bleb in the composition rather than having been expressed on its surface.

It has also been found that when antigens are formulated with a bleb adjuvant in a vaccine in this way, this vaccine may induce a faster immune response against the antigen (as well as a larger response). The adjuvant may therefore be particularly suitable for vaccines for the elderly (over 55 years of age).

The present invention provides an immunogenic composition comprising an antigen derived from a pathogen which is capable of protecting a host against said pathogen, mixed with an adjuvant comprising a bleb preparation derived from a Gram-negative bacterial strain. Although th source of the antigen and the bleb are preferably heterologous, they may still be derived from the same class of pathogen: for instance the antigen may be 1 or more (2 or 3) meningococcal capsular polysaccharides (plain or preferably conjugated) selected from a group comprising: A, Y or W (optionally also comprising group C conjugate), and the bleb preparation may be from a meningoccocus B strain. Such a vaccine may be advantageously used as a global meningococcus vaccine.

By conjugated it is meant that the antigen is covalently linked to a protein which is a source of T-helper epitopes such as tetanus toxoid, diphtheria toxoid, CRM197, pneumococcal pneumolysin, protein D from H. influenzae, or OmpC from meningococcus. When an antigen is conjugated the immunogenicity and the protective capacity of either or both the antigen and the carrier (against the organisms from which they are derived) may be significantly enhanced.

In a further embodiment, the antigen and the Gram-negative bacterial bleb preparation may be from different pathogens. For instance, the antigen may be a H. influenzae antigen (either a protein [as described below] or preferably a conjugated capsular polysaccharide from H. influenzae b), and the bleb preparation from meningoccocus B. If both a conjugated capsular polysaccharide from H. influenzae b and two or more conjugated meningococcal capsular polysaccharides (selected from A, C, Y and W) are included, such a vaccine may advantageously constitute a global meningitis vaccine (particularly if pneumococcal antigens are also included as described below).

Alternatively, the antigen is one or more capsular polysaccharide(s) from Streptococcus pneumoniae (plain or preferably conjugated), and/or one or more protein antigens that is capable of protecting a host against Streptococcus pneumoniae infection, and the bleb preparation is from meningococcus B.

The pneumococcal capsular polysaccharide antigens are preferably selected from serotypes 1, 2, 3, 4, 5, 6B, 7F, 8, 9N, 9V, 10A, 11A, 12F, 14, 15B, 17F, 18C, 19A, 19F, 20, 22F, 23F and 33F (most preferably from serotypes 1, 3, 4, 5, 6B, 7F, 9V, 14, 18C, 19F and 23F).

Preferred pneumococcal proteins antigens are those pneumococcal proteins which are exposed on the outer surface of the pneumococcus (capable of being recognised by a host's immune system during at least part of the life cycle of the pneumococcus), or are proteins which are secreted or released by the pneumococcus. Most preferably, the protein is a toxin, adhesin, 2-component signal tranducer, or lipoprotein of Streptococcus pneumoniae, or fragments thereof. Particularly preferred proteins include, but are not limited to: pneumolysin (preferably detoxified by chemical treatment or mutation) [Mitchell et al. Nucleic Acids Res. Jul. 11, 1990; 18(13): 4010 “Comparison of pneumolysin genes and proteins from Streptococcus pneumoniae types 1 and 2.”, Mitchell et al. Biochim Biophys Acta Jan. 23, 1989; 1007(1): 67-72 “Expression of the pneumolysin gene in Escherichia coli: rapid purification and biological properties.”, WO 96/05859 (A. Cyanamid), WO 90/06951 (Paton et al), WO 99/03884 (NAVA)]; PspA and transmembrane deletion variants thereof (U.S. Pat. No. 5,804,193—Briles et al.); PspC and transmembrane deletion variants thereof (WO 97/09994—Briles et al); PsaA and transmembrane deletion variants thereof (Berry & Paton, Infect Imun December 1996;64(12):5255-62 “Sequence heterogeneity of PsaA, a 37-kilodalton putative adhesin essential for virulence of Streptococcus pneumoniae”); pneumococcal choline binding proteins and transmembrane deletion variants thereof; CbpA and transmembrane deletion variants thereof (WO 97/41151; WO 99/51266); Glyceraldehyde-3-phosphate-dehydrogenase (Infect. Immun. 1996 64:3544); HSP70 (WO 96/40928); PcpA (Sanchez-Beato et al. FEMS Microbiol Lett 1998, 164:207-14); M like protein, SB patent application No. EP 0837130; and adhesin 18627, SB Patent application No. EP 0834568. Further preferred pneumococcal protein antigens are those disclosed in WO 98/18931, particularly those selected in WO 98/18930 and PCT/US99/30390 (incorporated by reference herein).

The above mentioned meingococcal blebs may be from a wild-type strain, or might be a mixture from 2 or more (preferably several) wild-type strains belonging to several subtype/serotypes (for instance chosen from P1.15, P1.7,16, P1.4, and P1.2).

The above mentioned meningococcal blebs may also be genetically engineered to improve them in a way discussed below. Preferably, the meningococcus B bleb preparation is derived from a strain which has a detoxified lipid A portion of bacterial LPS, due to the strain having been engineered to reduce or switch off expression of one or more genes selected from the group consisting of: htrB, msbB and lpxK (or homologues thereof).

By ‘reduce’ it is meant that expression from a gene has been decreased by 10, 20, 30, 40, 50 ,60, 70, 80, or 90%. By ‘switch off’ it is meant the gene is deleted from the genome or in some other way produces no active gene product.

Alternatively, or in combination, the meningococcal B bleb preparation is derived from a strain which has a detoxified lipid A portion of bacterial LPS, due to the strain having been engineered to express at a higher level one or more genes selected from the group consisting of: pmrA, pmrB, pmrE and pmrF.

By ‘express at a higher level’ it is meant that more than 10, 30, 50, 70, 90, 150, 300% additional gene product is made by the recombinant bacterium than in the wild-type strain.

A further improvement which may be an alternative or in combination with either or both of the previous improvements is that the meningococcal B bleb preparation is derived from a strain which does not produce B capsular polysaccharide, due to the strain having been engineered to reduce or switch off expression of one or more genes selected from the group consisting of: galE, siaA, siaB, siaC, siaD, ctrA, ctrB, ctrC and ctrD (or homologues thereof). These mutations may also remove human-like epitopes from the LOS of the bleb.

Compositions Useful for the Treatment of Otitis Media

In a further embodiment the antigen in the immunogenic composition is from H. influenzae, and the bleb preparation is from Moraxella catarrhalis. The antigen may be a conjugated capsular polysaccharide from H. influenzae b, or may be one or more protein antigens that can protect a host against non-typeable H. influenzae infection.

Preferred non-typeable H. influenzae protein antigens include Fimbrin protein (U.S. Pat. No. 5,766,608) and fusions comprising peptides therefrom (eg LB1 Fusion) (U.S. Pat. No. 5,843,464—Ohio State Research Foundation), OMP26, P6, protein D, TbpA, TbpB, Hia, Hmw1, Hmw2, Hap, and D15.

Alternatively, the antigen may be from Streptococcus pneumoniae, and the bleb preparation from Moraxella catarrhalis. The pneumococcal antigen may be one or more capsular polysaccharide(s) (preferably conjugated) from Streptococcus pneumoniae (as described above), and/or one or more proteins from Streptococcus pneumoniae capable of protecting a host against pneumococcal disease (as described above).

The above immunogenic compositions comprising a Moraxella catarrhalis bleb preparation adjuvant may also optionally comprise one or more antigens that can protect a host against RSV and/or one or more antigens that can protect a host against influenza virus.

Preferred influenza virus antigens include whole, live or inactivated virus, split influenza virus, grown in eggs or MDCK cells, or Vero cells or whole flu virosomes (as described by R. Gluck, Vaccine, 1992, 10, 915-920) or purified or recombinant proteins thereof, such as HA, NP, NA, or M proteins, or combinations thereof.

Preferred RSV (Respiratory Syncytial Virus) antigens include the F glycoprotein, the G glycoprotein, the HN protein, or derivatives thereof.

In a preferred embodiment, the Moraxella catarrhalis bleb adjuvant is formulated with one or more plain or conjugated pneumococcal capsular polysaccharides, and one or more antigens that can protect a host against non-typeable H. influenzae infection (as defined above). Optionally, the vaccine may also comprise one or more protein antigens that can protect a host against Streptococcus pneumoniae infection (as defined above). The vaccine may also optionally comprise one or more antigens that can protect a host against RSV and/or one or more antigens that can protect a host against influenza virus (as defined above). Such a vaccine may be advantageously used as a global otitis media vaccine.

The Moraxella catarrhalis bleb adjuvant mentioned above may be derived from a wild-type strain, or might be a mixture from 2 or more (preferably several) wild-type strains belonging to several subtype/serotypes.

The above mentioned Moraxella catarrhalis bleb adjuvant may also be genetically engineered to improve the blebs in a way discussed below. Preferably, the Moraxella catarrhalis bleb preparation is derived from a strain which has a detoxified lipid A portion of bacterial LPS, due to the strain having been engineered to reduce or switch off expression of one or more genes selected from the group consisting of: htrB, msbB and lpxK (or homologues thereof).

Alternatively, or in combination with the above improvement, the Moraxella catarrhalis bleb adjuvant is derived from a strain which has a detoxified lipid A portion of bacterial LPS, due to the strain having been engineered to express at a higher level one or more genes selected from the group consisting of: pmrA, pmrB, pmrE and pmrF.

A further improvement which may be an alternative or in combination with either or both of the previous improvements is that the Moraxella catarrhalis bleb adjuvant is derived from a strain which has been engineered to remove human-like epitopes from the LPS of the bleb. This could be done, for instance, by the strain having been engineered to reduce or switch off expression of one or more genes selected from the group consisting of: galE, siaA, siaB, siaC, siaD, ctrA, ctrB, ctrC and ctrD (or homologues thereof).

In a still further embodiment the antigen in the immunogenic composition is a conjugated capsular polysaccharide from H. influenzae b, and the bleb preparation is from non-typeable H. influenzae.

Alternatively, the antigen may be from Streptococcus pneumoniae, and the bleb preparation from non-typeable H. influenzae. The pneumococcal antigen may be one or more capsular polysaccharide(s) (preferably conjugated) from Streptococcus pneumoniae (as described above), and/or one or more proteins from Streptococcus pneumoniae capable of protecting a host against pneumococcal disease (as described above).

Alternatively, the antigen may be from Moraxella catarrhalis (preferably one or more proteins from M. catarrhalis capable of protecting a host against disease caused by this organism [most preferably one of the protective antigens mentioned above or mentioned below as being usefully upregulated in a Moraxella catarrhalis bleb vaccine]) and the bleb preparation from non-typeable H. influenzae.

The above immunogenic compositions comprising a non-typeable H. influenzae bleb preparation adjuvant may also optionally comprise one or more antigens that can protect a host against RSV (as described above) and/or one or more antigens that can protect a host against influenza virus (as described above).

In a preferred embodiment, the non-typeable H. influenzae bleb adjuvant is formulated with one or more plain or conjugated pneumococcal capsular polysaccharides, and one or more antigens that can protect a host against M. catarrhalis infection (as defined above). Optionally, the vaccine may also comprise one or more protein antigens that can protect a host against Streptococcus pneumoniae infection (as defined above). The vaccine may also optionally comprise one or more antigens that can protect a host against RSV and/or one or more antigens that can protect a host against influenza virus (as defined above). Such a vaccine may be advantageously used as a global otitis media vaccine.

The non-typeable H. influenzae bleb adjuvant mentioned above may be derived from a wild-type strain, or might be a mixture from 2 or more (preferably several) wild-type strains belonging to several subtype/serotypes.

The above mentioned non-typeable H. influenzae bleb adjuvant may also be genetically engineered to improve the blebs in a way discussed below. Preferably, the non-typeable H. influenzae bleb preparation is derived from a strain which has a detoxified lipid A portion of bacterial LPS, due to the strain having been engineered to reduce or switch off expression of one or more genes selected from the group consisting of: htrB, msbB and lpxK.

Alternatively, or in combination with the above improvement, the non-typeable H. influenzae bleb adjuvant is derived from a strain which has a detoxified lipid A portion of bacterial LPS, due to the strain having been engineered to express at a higher level one or more genes selected from the group consisting of: pmrA, pmrB, pmrE and pmrF.

A further improvement which may be an alternative or in combination with either or both of the previous improvements is that the H. influenzae bleb adjuvant is derived from a strain which has been engineered to remove human-like epitopes from the LPS of the bleb. This could be done, for instance, by the strain having been engineered to reduce or switch off expression of one or more genes selected from the group consisting of: galE, siaA, siaB, siaC, siaD, ctrA, ctrB, ctrC and ctrD (or homologues thereof).

A further aspect of the invention is a vaccine composition comprising the above immunogenic compositions of the invention, and a pharmaceutically acceptable excipient or carrier. Preferable such vaccines should be formulated as described below in “vaccine formulations”.

The amount of polysaccharide antigen (plain or in a conjugate) in each vaccine dose is selected as an amount which induces an immunoprotective response without significant, adverse side effects in typical vaccines. Such amount will vary depending upon which specific immunogen is employed and how it is presented. Generally, it is expected that each dose will comprise 0.1-100 μg of polysaccharide, preferably 0.1-50 μg, preferably 0.1-10 μg, of which 1 to 5 μg is the most preferable range.

The content of protein antigens in the vaccine will typically be in the range 1-100 kg, preferably 5-50 μg, most typically in the range 5-25 μg. The amount of bleb adjuvant present in the formulations should be present in a similar range of quantity.

Optimal amounts of components for a particular vaccine can be ascertained by standard studies involving observation of appropriate immune responses in subjects. Following an initial vaccination, subjects may receive one or several booster immunisations adequately spaced.

The immunogenic compositions or vaccines of this aspect of the invention may have one or more of the following advantages: i) higher immune response against the antigen; ii) higher protective capacity of the antigen; iii) faster immune response against the antigen; iv) faster protection by the antigen; v) where the antigen is a conjugated polysaccharide antigen, i) ii), iii) or iv) may apply to both the polysaccharide and the carrier; vi) the antigen may enhance the immune response or protective capacity of a protective antigen present on the surface of the bleb preparation.

A further embodiment of this aspect of the invention is a method of inducing a faster immune response against the antigen contained in the immunogenic composition of the invention, comprising the step of administering to a host an effective amount of the above mentioned immunogenic compositions. Preferably this is also a method of inducing faster protection against the pathogen from which the antigen is derived.

Such a method would be extremely valuable for treating patients with compromised or weakened immune systems, such as the elderly (people over 55 years). Thus another embodiment is a method of protecting an elderly patient against a pathogen by administering to said patient an effective amount of the immunogenic composition mentioned above in which the antigen is derived from said pathogen.

Further aspects include a use of the above mentioned immunogenic preparations in the manufacture of a medicament for the treatment of a disease caused by the pathogen from which the antigen present within is derived. A use of blebs derived from Moraxella catarrhalis as an adjuvant in an immunogenic composition comprising one or more pneumococcal capsular polysaccharides, a use of blebs derived from Moraxella catarrhalis as an adjuvant in an immunogenic composition comprising one or more pneumococcal (or H. influenzae) protein antigens, a use of blebs derived from non-typeable H. influenzae as an adjuvant in an immunogenic composition comprising one or more pneumococcal capsular polysaccharides, and a use of blebs derived from non-typeable H. influenzae as an adjuvant in an immunogenic composition comprising one or more pneumococcal (or M. catarrhalis) protein antigens, are further envisioned embodiments.

Genetically-Engineered Bleb Adjuvants

The bleb adjuvant of the present invention may be improved using a general set of tools and methods for making genetically engineered blebs from Gram-negative bacterial strains. The invention includes methods used to make recombinant bleb adjuvants more immunogenic, less toxic and safer for their use in a human and/or animal vaccine. Moreover, the present invention also describes specific methods necessary for constructing, producing, obtaining and using recombinant, engineered blebs from various Gram-negative bacteria, for vaccine/adjuvant purposes. By the methods of the invention, the biochemical composition of bacterial blebs can be manipulated by acting upon/altering the expression of bacterial genes encoding products present in or associated with bacterial outer-membrane blebs (outer membrane proteins or OMPs). The production of blebs using a method of genetic modification to increase, decrease or render conditional the expression of one or more genes encoding outer-membrane components are also included in the scope of this invention.

For clarity, the term “expression cassette” will refer herein to all the genetic elements necessary to express a gene or an operon and to produce and target the corresponding protein(s) of interest to outer-membrane blebs, derived from a given bacterial host. A non-exhaustive list of these features includes control elements (transcriptional and/or translational), protein coding regions and targeting signals, with appropriate spacing between them. Reference to the insertion of promoter sequences means, for the purposes of this invention, the insertion of a sequence with at least a promoter function, and preferably one or more other genetic regulatory elements comprised within an expression cassette. Moreover, the term “integrative cassette” will refer herein to all the genetic elements required to integrate a DNA segment in given bacterial host. A non-exhaustive list of these features includes a delivery vehicle (or vector), with recombinogenic regions, and selectable and counter selectable markers.

Again for the purpose of clarity, the terms ‘engineering a bacterial strain to produce less of said antigen’ refers to any means to reduce the expression of an antigen of interest, relative to that of the non-modified (i.e., naturally occurring) bleb such that expression is at least 10% lower than that of the non-modified bleb. Preferably it is at least 50% lower. “Stronger promoter sequence” refers to a regulatory control element that increases transcription for a gene encoding antigen of interest. “Upregulating expression” refers to any means to enhance the expression of an antigen of interest, relative to that of the non-modified (i.e., naturally occurring) bleb. It is understood that the amount of ‘upregulation’ will vary depending on the particular antigen of interest but will not exceed an amount that will disrupt the membrane integrity of the bleb. Upregulation of an antigen refers to expression that is at least 10% higher than that of the non-modified bleb. Preferably it is at least 50% higher. More preferably it is at least 100% (2 fold) higher.

Aspects of the invention relate to individual methods for making improved engineered bleb adjuvants, to a combination of such methods, and to the bleb compositions made as a result of these methods. Another aspect of the invention relates to the genetic tools used in order to genetically modify a chosen bacterial strain in order to extract improved engineered blebs from said strain.

The engineering steps of the processes of the invention can be carried out in a variety of ways known to the skilled person. For instance, sequences (e.g. promoters or open reading frames) can be inserted, and promoters/genes can be disrupted by the technique of transposon insertion. For instance, for upregulating a gene's expression, a strong promoter could be inserted via a transposon up to 2 kb upstream of the gene's initiation codon (more preferably 200-600 bp upstream, most preferably approximately 400 bp upstream). Point mutation or deletion may also be used (particularly for down-regulating expression of a gene).

Such methods, however, may be quite unstable or uncertain, and therefore it is preferred that the engineering step [particularly for processes a), b), c), d), e), h) and i) as described below] is performed via a homologous recombination event. Preferably, the event takes place between a sequence (a recombinogenic region) of at least 30 nucleotides on the bacterial chromosome, and a sequence (a second recombinogenic region) of at least 30 nucleotides on a vector transformed within the strain. Preferably the regions are 40-1000 nucleotides, more preferably 100-800 nucleotides, most preferably 500 nucleotides). These recombinogenic regions should be sufficiently similar that they are capable of hybridising to one another under highly stringent conditions (as defined later).

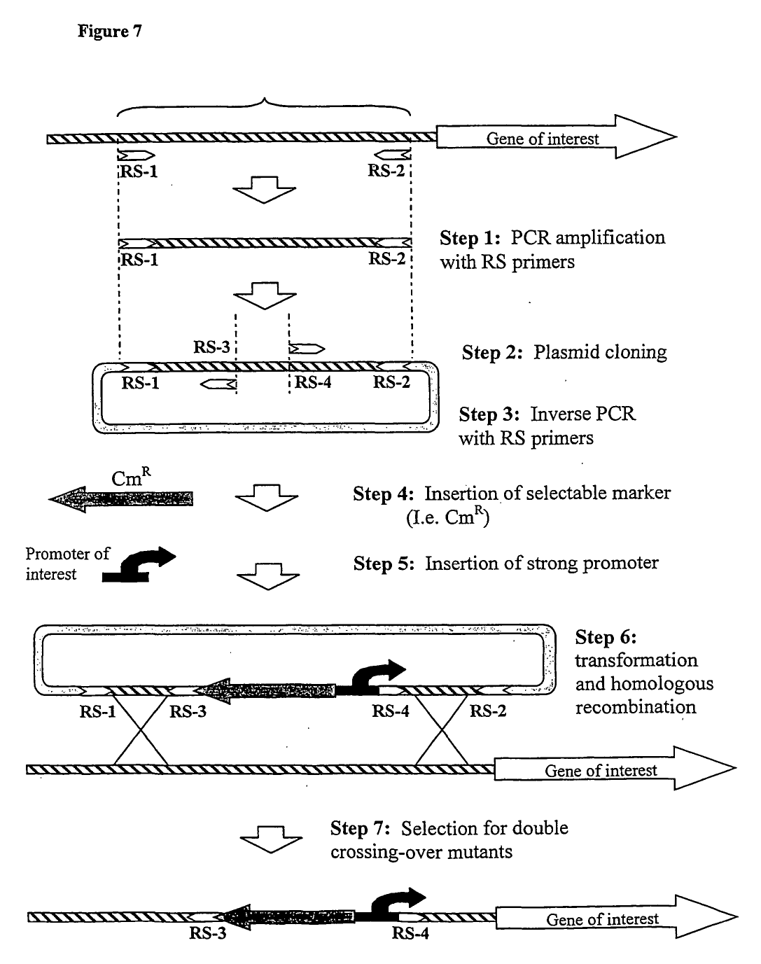

Recombination events may take place using a single recombinogenic region on chromosome and vector, or via a double cross-over event (with 2 regions on chromosome and vector). In order to perform a single recombination event, the vector should be a circular DNA molecule. In order to perform a double recombination event, the vector could be a circular or linear DNA molecule (see FIG. 7). It is preferable that a double recombination event is employed and that the vector used is linear, as the modified bacterium so produced will be more stable in terms of reversion events. Preferably the two recombinogenic regions on the chromosome (and on the vector) are of similar (most preferably the same) length so as to promote double cross-overs. The double cross-over functions such that the two recombinogenic regions on the chromosome (separated by nucleotide sequence ‘X’) and the two recombinogenic regions on the vector (separated by nucleotide sequence ‘Y’) recombine to leave a chromosome unaltered except that X and Y have interchanged. The position of the recombinogenic regions can both be positioned upstream or down stream of, or may flank, an open reading frame of interest. These regions can consist of coding, non-coding, or a mixture of coding and non-coding sequence. The identity of X and Y will depend on the effect desired. X may be all or part of an open reading frame, and Y no nucleotides at all, which would result in sequence X being deleted from the chromosome. Alternatively Y may be a strong promoter region for insertion upstream of an open reading frame, and therefore X may be no nucleotides at all.

Suitable vectors will vary in composition depending what type of recombination event is to be performed, and what the ultimate purpose of the recombination event is. Integrative vectors used to deliver region Y can be conditionally replicative or suicide plasmids, bacteriophages, transposons or linear DNA fragments obtained by restriction hydrolysis or PCR amplification. Selection of the recombination event is selected by means of selectable genetic marker such as genes conferring resistance to antibiotics (for instance kanamycin, erythromycin, chloramphenicol, or gentamycin), genes conferring resistance to heavy metals and/or toxic compounds or genes complementing auxotrophic mutations (for instance pur, leu, met, aro).

Process a) and f)—Down Regulation/Removal of Variable and Non-Protective Immunodominant Antigens in Bleb Adjuvants

Many surface antigens are variable among bacterial strains and as a consequence are protective only against a limited set of closely related strains. An aspect of this invention covers the reduction in expression, or, preferably, the deletion of the gene(s) encoding variable surface protein(s) which results in a bacterial strain producing blebs which, when administered in a vaccine, have a stronger potential for cross-reactivity against various strains due to a higher influence exerted by conserved proteins (retained on the outer membranes) on the vaccinee's immune system. Examples of such variable antigens include: for Neisseria—pili (PilC) which undergoes antigenic variations, PorA, Opa, TbpB, FrpB; for H. influenzae—P2, P5, pilin, IgA1-protease; and for Moracella—CopB, OMP106.

Other types of gene that could be down-regulated or switched off are genes which, in vivo, can easily be switched on (expressed) or off by the bacterium. As outer membrane proteins encoded by such genes are not always present on the bacteria, the presence of such proteins in the bleb preparations can also be detrimental to the effectiveness of the vaccine for the reasons stated above. A preferred example to down-regulate or delete is Neisseria Opc protein. Anti-Opc immunity induced by an Opc containing bleb vaccine would only have limited protective capacity as the infecting organism could easily become Opc⁻. H. influenzae HgpA and HgpB are other examples of such proteins.

In process a), these variable or non-protective genes are down-regulated in expression, or terminally switched off. This has the above-mentioned surprising advantage of concentrating the immune system on better antigens that are present in low amounts on the outer surface of blebs.

The strain can be engineered in this way by a number of strategies including transposon insertion to disrupt the coding region or promoter region of the gene, or point mutations or deletions to achieve a similar result. Homologous recombination may also be used to delete a gene from a chromosome (where sequence X comprises part (preferably all) of the coding sequence of the gene of interest). It may additionally be used to change its strong promoter for a weaker (or no) promoter (where nucleotide sequence X comprises part (preferably all) of the promoter region of the gene, and nucleotide sequence Y comprises either a weaker promoter region [resulting in a decreased expression of the gene(s)/operon(s) of interest], or no promoter region). In this case it is preferable for the recombination event to occur within the region of the chromosome 1000 bp upstream of the gene of interest.

Alternatively, Y may confer a conditional transcriptional activity, resulting in a conditional expression of the gene(s)/operon(s) of interest (down-regulation). This is useful in the expression of molecules that are toxic to or not well supported by the bacterial host.

Most of the above-exemplified proteins are integral OMPs and their variability may be limited only to one or few of their surface exposed loops. Another aspect of this invention [process g)] covers the deletion of DNA regions coding for these surface exposed loops which leads to the expression of an integral OMP containing conserved surface exposed loops. Surface exposed loops of H. influenzae P2 and P5 are preferred examples of proteins that could be transformed into cross-reactive antigens by using such a method. Again, homologous recombination is a preferred method of performing this engineering process.

Process b)—Promoter Delivery and Modulation:

A further aspect of the invention relates to modifying the composition of bleb adjuvants by altering in situ the regulatory region controlling the expression of gene(s) and/or operon(s) of interest. This alteration may include partial or total replacement of the endogenous promoter controlling the expression of a gene of interest, with one conferring a distinct transcriptional activity. This distinct transcriptional activity may be conferred by variants (point mutations, deletions and/or insertions) of the endogenous control regions, by naturally occurring or modified heterologous promoters or by a combination of both. Such alterations will preferably confer a transcriptional activity stronger than the endogenous one (introduction of a strong promoter), resulting in an enhanced expression of the gene(s)/operon(s) of interest (up-regulation). Such a method is particularly useful for enhancing the production of immunologically relevant Bleb components such as outer-membrane proteins and lipoproteins (preferably conserved OMPs, usually present in blebs at low concentrations).

Typical strong promoters that may be integrated in Neisseria are porA [SEQ ID NO: 24], porB [SEQ ID NO:26], lgtF, Opa, p110, 1st, and hpuAB. PorA and PorB are preferred as constitutive, strong promoters. It has been established (Example 9) that the PorB promoter activity is contained in a fragment corresponding to nucleotides −1 to −250 upstream of the initation codon of porB. In Moraxella, it is preferred to use the ompH, ompG, ompE, OmpB1, ompB2, ompA, OMPCD and Omp106 promoters, and in H. influenzae, it is preferred to integrate the P2, P4, P1, P5 and P6 promoters.

Using the preferred double cross-over homologous recombination technology to introduce the promoter in the 1000 bp upstream region, promoters can be placed anywhere from 30-970 bp upstream of the initiation codon of the gene to be up-regulated. Although conventionally it is thought the promoter region should be relatively close to the open reading frame in order to obtain optimal expression of the gene, the present inventors have surprisingly found that placement of the promoter further away from the initiation codon results in large increases in expression levels. Thus it is preferred if the promoter is inserted 200-600 bp from the initiation codon of the gene, more preferably 300-500 bp, and most preferably approximately 400 bp from the initiation ATG.

Process c)—Bleb Components Produced Conditionally

The expression of some genes coding for certain bleb components is carefully regulated. The production of the components is conditionally modulated and depends upon various metabolic and/or environmental signals. Such signals include, for example, iron-limitation, modulation of the redox potential, pH and temperature variations, nutritional changes. Some examples of bleb components known to be produced conditionally include iron-regulated outer-membrane proteins from Neisseiria and Moraxella (for instance TbpB, LbpB), and substrate-inducible outer-membrane porins. The present invention covers the use of the genetic methods described previously (process a) or b)) to render constitutive the expression of such molecules. In this way, the influence of environmental signal upon the expression of gene(s) of interest can be overcome by modifying/replacing the gene's corresponding control region so that it becomes constitutively active (for instance by deleting part [preferably all] or the repressive control sequence—e.g. the operator region), or inserting a constitutive strong promoter. For iron regulated genes the fur operator may be removed. Alternatively, process i) may be used to deliver an additional copy of the gene/operon of interest in the chromosome which is placed artificially under the control of a constitutive promoter.

Processes d), and e)—Detoxification of LPS

The toxicity of bleb adjuvant preparations presents one of the largest problems in the use of blebs in vaccines. A further aspect of the invention relates to methods of genetically detoxifying the LPS present in Blebs. Lipid A is the primary component of LPS responsible for cell activation. Many mutations in genes involved in this pathway lead to essential phenotypes. However, mutations in the genes responsible for the terminal modifications steps lead to temperature-sensitive (htrB) or permissive (msbB) phenotypes. Mutations resulting in a decreased (or no) expression of these genes (or decreased or no activity of the product of these genes) result in altered toxic activity of lipid A. Indeed, the non-lauroylated (htrB mutant) or non-myristoylated (msbB mutant) lipid A are less toxic than the wild-type lipid A. Mutations in the lipid A 4′-kinase encoding gene (lpxK) also decreases the toxic activity of lipid A.

Process d) thus involves either the deletion of part (or preferably all) of one or more of the above open reading frames or promoters. Alternatively, the promoters could be replaced with weaker promoters, or the enzyme activity of the gene product may be significantly reduced by site specific mutagenesis. Preferably the homologous recombination techniques described above are used to carry out the process.

The sequences of the htrB and msbB genes from Neisseria meningitidis B, Moraxella catarrhalis, and Haemophilus influenzae are additionally provided for this purpose.

LPS toxic activity could also be altered by introducing mutations in genes/loci involved in polymyxin B resistance (such resistance has been correlated with addition of aminoarabinose on the 4′ phosphate of lipid A). These genes/loci could be pmrE that encodes a UDP-glucose dehydrogenase, or a region of antimicrobial peptide-resistance genes common to many enterobacteriaciae which could be involved in aminoarabinose synthesis and transfer. The gene pmrF that is present in this region encodes a dolicol-phosphate manosyl transferase (Gunn J. S., Kheng, B. L., Krueger J., Kim K., Guo L., Hackett M., Miller S. I. 1998. Mol. Microbiol. 27: 1171-1182).

Mutations in the PhoP-PhoQ regulatory system, which is a phospho-relay two component regulatory system (f. i. PhoP constitutive phenotype, PhoP^{c}), or low Mg⁺⁺ environmental or culture conditions (that activate the PhoP-PhoQ regulatory system) lead to the addition of aminoarabinose on the 4′-phosphate and 2-hydroxymyristate replacing myristate (hydroxylation of myristate). This modified lipid A displays reduced ability to stimulate E-selectin expression by human endothelial cells and TNF-α secretion from human monocytes.

Process e) involves the upregulation of these genes using a strategy as described above (strong promoters being incorporated, preferably using homologous recombination techniques to carry out the process).

Alternatively, rather than performing any such mutation, a polymyxin B resistant strain could be used as a bleb adjuvant production strain (in conjunction with one or more of the other processes of the invention), as blebs from such strains also have reduced LPS toxicity (for instance as shown for meningococcus—van der Ley, P, Hamstra, H J, Kramer, M, Steeghs, L, Petrov, A and Poolman, J T. 1994. In: Proceedings of the ninth international pathogenic Neisseria conference. The Guildhall, Winchester, England).

As a further alternative (and further aspect of the invention) the inventors provide a method of detoxifying a Gram-negative bacterial strain comprising the step of culturing the strain in a growth medium containing 0.1 mg-100 g of aminoarabinose per litre medium, and the bleb adjuvant derived from such a strain.

As a further still alternative, synthetic peptides that mimic the binding activity of polymyxin B (described below) may be added to the Bleb preparation in order to reduce LPS toxic activity (Rustici, A, Velucchi, M, Faggioni, R, Sironi, M, Ghezzi, P, Quataert, S, Green, B and Porro M 1993. Science 259: 361-365; Velucchi, M, Rustici, A, Meazza, C, Villa, P, Ghezzi, P and Porro, M. 1997. J Endotox. Res. 4:).

Process f)—Anchoring Homologous or Heterologous Proteins to Outer-Membrane Bleb Adjuvants Whilst Reducing the Toxicity of LPS

A further aspect of this invention covers the use of genetic sequences encoding polymyxin B peptides (or analogues thereof) as a means to target fusion proteins to the outer-membrane. Polymyxin B is a cyclic peptide composed of non tRNA-encoded amino acids (produced by Gram-positive actinomycetal organisms) that binds very strongly to the Lipid A part of LPS present in the outer-membrane. This binding decreases the intrinsic toxicity of LPS (endotoxin activity). Peptides mimicking the structure of Polymyxin B and composed of canonical (tRNA encoded) amino acids have been developed and also bind lipid A with a strong affnity. These peptides have been used for detoxifying LPS. One of these peptides known as SAEP-2 (Nterminus-Lys-Thr-Lys-Cys-Lys-Phe-Leu-Lys-Lys-Cys-Cterminus) was shown to be very promising in that respect (Molecular Mapping and detoxifying of the Lipid A binding site by synthetic peptides (1993). Rustici, A., Velucchi, M., Faggioni, R., Sironi, M., Ghezzi, P., Quataert, S., Green, B. and M. Porro. Science 259, 361-365).

The present process f) of the invention provides an improvement of this use. It has been found that the use of DNA sequences coding for the SEAP-2 peptide (or derivatives thereof), fused genetically to a gene of interest (encoding for instance a T cell antigen or a protective antigen that is usually secreted such as a toxin, or a cytosolic or periplasmic protein) is a means for targeting the corresponding recombinant protein to the outer-membrane of a preferred bacterial host (whilst at the same time reducing the toxicity of the LPS).

This system is suitable for labile proteins which would not be directly exposed to the outside of the bleb. The bleb would therefore act as a delivery vehicle which would expose the protein to the immune system once the blebs had been engulfed by T-cells. Alternatively, the genetic fusion should also comprise a signal peptide or transmembrane domain such that the recombinant protein may cross the outer membrane for exposure to the host's immune system.

This targeting strategy might be of particular interest in the case of genes encoding proteins that are not normally targeted to the outer-membrane. This methodology also allows the isolation of recombinant blebs enriched in the protein of interest. Preferably, such a peptide targeting signal allows the enrichment of outer membrane blebs in one or several proteins of interest, which are naturally not found in that given subcellular localization. A non exhaustive list of bacteria that can be used as a recipient host for such a production of recombinant blebs includes Neisseria meningitidis, Neisseiria gonorrhoeae Moraxella catarrhalis, Haemophilus influenzae, Pseudomonas aeruginosa, Chlamydia trachomatis, and Chlamydia pneumoniae.

Although it is preferred that the gene for the construct is engineered into the chromosome of the bacterium [using process i)], an alternative preferred embodiment is for SAEP-2-tagged recombinant proteins to be made independently, and attached at a later stage to a bleb preparation.

A further embodiment is the use of such constructs in a method of protein purification. The system could be used as part of an expression system for producing recombinant proteins in general. The SAEP-2 peptide tag can be used for affinity purification of the protein to which it is attached using a column containing immobilised lipid A molecules.

Process h)—Cross-Reactive Polysaccharides on Bleb Adjuvant

The isolation of bacterial outer-membrane blebs from encapsulated Gram-negative bacteria often results in the co-purification of capsular polysaccharide. In some cases, this “contaminant” material may prove useful since polysaccharide may enhance the immune response conferred by other bleb components. In other cases however, the presence of contaminating polysaccharide material in bacterial bleb preparations may prove detrimental to the use of the blebs in a vaccine. For instance, it has been shown at least in the case of N. meningitidis that the serogroup B capsular polysaccharide does not confer protective immunity and is susceptible to induce an adverse auto-immune response in humans. Such human-like epitopes may also be present on LPS/LOS within the blebs. Consequently, process h) of the invention is the engineering of the bacterial strain for bleb production such that it is free of human-like epitopes, particularly capsular polysaccharide. The blebs will then be suitable for use in humans. A particularly preferred example of such a bleb preparation is one from N. meningitidis serogroup B devoid of capsular polysaccharide.

This may be achieved by using modified bleb production strains in which the genes necessary for polysaccharide biosynthesis and/or export have been impaired. Inactivation of the gene coding for polysaccharide biosynthesis or export can be achieved by mutating (point mutation, deletion or insertion) either the control region, the coding region or both (preferably using the homologous recombination techniques described above). Moreover, inactivation of capsular biosynthesis genes may also be achieved by antisense over-expression or transposon mutagenesis. A preferred method is the deletion of some or all of the Neisseria meningitidis cps genes required for polysaccharide biosynthesis and export. For this purpose, the replacement plasmid pMF121 (described in Frosh et al.1990, Mol. Microbiol 4:1215-1218) can be used to deliver a mutation deleting the cpsCAD (+galE) gene cluster. Alternatively the siaD gene could be deleted, or down-regulated in expression (the meningococcal siaD gene encodes alpha-2,3-sialyltransferase, an enzyme required for capsular polysaccharide and LOS synthesis). Such mutations may also remove host-similar structures on the saccharide portion of the LPS of the bacteria.

Process i)—Delivery of One or More Further Copies of a Gene and/or Operon in a Host Chromosome, or Delivery of a Heterologous Gene and/or Operon in a Host Chromosome.

An efficient strategy to modulate the composition of a Bleb preparation is to deliver one or more copies of a DNA segment containing an expression cassette into the genome of a Gram-negative bacterium. A non exhaustive list of preferred bacterial species that could be used as a recipient for such a cassette includes Neisseria meningitidis, Neisseiria gonorrhoeae, Moraxella catarrhalis, Haemophilus influenzae, Pseudonmonas aeruginosa, Chlamydia trachomatis, Chlamydia pneumoniae. The gene(s) contained in the expression cassette may be homologous (or endogenous) (i.e. exist naturally in the genome of the manipulated bacterium) or heterologous (i.e. do not exist naturally in the genome of the manipulated bacterium). The reintroduced expression cassette may consist of unmodified, “natural” promoter/gene/operon sequences or engineered expression cassettes in which the promoter region and/or the coding region or both have been altered. A non-exhaustive list of preferred promoters that could be used for expression includes the promoters porA, porB, lbpB, tbpB, p110, 1st, hpuAB from N. meningitidis or N. gonorroheae, the promoters p2, p5, p4, ompF, p1, ompH, p6, hin47 from H. influenzae, the promoters ompH, ompG, ompCD, ompE, ompB1, ompB2, ompA of M. catarrhalis, the promoter λpL, lac, tac, araB of Escherichia coli or promoters recognized specifically by bacteriophage RNA polymerase such as the E. coli bacteriophage T7. A non-exhaustive list of preferred genes that could be expressed in such a system includes Neisseria NspA, Omp85, PilQ, ThpA/B complex, Hsf, PlDA, HasR; Chlamydia MOMP, HMWP; Moraxella OMP106, HasR, PilQ, OMP85, PlDA; Bordetella pertussis FHA, PRN, PT.

In a preferred embodiment of the invention the expression cassette is delivered and integrated in the bacterial chromosome by means of homologous and/or site specific recombination. Integrative vectors used to deliver such genes and/or operons can be conditionally replicative or suicide plasmids, bacteriophages, transposons or linear DNA fragments obtained by restriction hydrolysis or PCR amplification. Integration is preferably targeted to chromosomal regions dispensable for growth in vitro. A non exhaustive list of preferred loci that can be used to target DNA integration includes the porA, porB, opa, opc, rmp, omp26, lecA, cps, lgtB genes of Neisseiria meningitidis and Neisseria gonorrhoeae, the P1, P5, hmw1/2, IgA-protease, fimE genes of NTHi; the lecA1, lecA2, omp106, uspA1, uspA2 genes of Moraxella catarrhalis. Alternatively, the expression cassette used to modulate the expression of bleb component(s) can be delivered into a bacterium of choice by means of episomal vectors such as circular/linear replicative plasmids, cosmids, phasmids, lysogenic bacteriophages or bacterial artificial chromosomes. Selection of the recombination event can be selected by means of selectable genetic marker such as genes conferring resistance to antibiotics (for instance kanamycin, erythromycin, chloramphenicol, or gentamycin), genes conferring resistance to heavy metals and/or toxic compounds or genes complementing auxotrophic mutations (for instance pur, leu, met, aro).

Heterologous Genes—Expression of Foreign Proteins in Outer-Membrane Blebs

Outer-membrane bacterial blebs represent a very attractive system to produce, isolate and deliver recombinant proteins. A further aspect of this invention is in respect of the expression, production and targeting of foreign, heterologous proteins to the outer-membrane, and the use of the bacteria to produce recombinant blebs.

A preferred method of achieving this is via a process comprising the steps of: introducing a heterologous gene, optionally controlled by a strong promoter sequence, into the chromosome of a Gram-negative strain by homologous recombination. Blebs may be made from the resulting modified strain.

A non-exhaustive list of bacteria that can be used as a recipient host for production of recombinant blebs includes Neisseria meningitidis, Neisseiria gonorrhoeae Moraxella catarrhalis, Haemophilus influenzae, Pseudomonas aeruginosa, Chlamydia trachomatis, Chlamydia pneumoniae. The gene expressed in such a system can be of viral, bacterial, fungal, parasitic or higher eukaryotic origin.

A preferred application of the invention includes a process for the expression of Moraxella, Haemophilus and/or Pseudomonas outer-membrane proteins (integral, polytopic and/or lipoproteins) in Neisseria meningitidis recombinant blebs. The preferable integration loci are stated above, and genes that are preferably introduced are those that provide protection against the bacterium from which they were isolated. Preferred protective genes for each bacterium are described below.

Further preferred applications are: blebs produced from a modified Haemophilus influenzae strain where the heterologous gene is a protective OMP from Moraxella catarrhalis; and blebs produced from a modified Moraxella catarrhalis strain where the heterologous gene is a protective OMP from Haemophilus influenzae (preferred loci for gene insertion are given above, and preferred protective antigens are described below).

A particularly preferred application of this aspect is in the field of the prophylaxis or treatment of sexually-transmitted diseaseses (STDs). It is often difficult for practitioners to determine whether the principal cause of a STD is due to gonococcus or Chlamydia trachomatis infection. These two organisms are the main causes of salpingitis—a disease which can lead to sterility in the host. It would therefore be useful if a STD could be vaccinated against or treated with a combined vaccine effective against disease caused by both organisms. The Major Outer Membrane Protein (MOMP) of C. trachomatis has been shown to be the target of protective antibodies. However, the structural integrity of this integral membrane protein is important for inducing such antibodies. In addition, the epitopes recognised by these antibodies are variable and define more than 10 serovars. The previously described aspect of this invention allows the proper folding of one or more membrane proteins within a bleb outer membrane preparation. The engineering of a gonococcal strain expressing multiple C. trachomatis MOMP serovars in the outer membrane, and the production of blebs therefrom, produces a single solution to the multiple problems of correctly folded membrane proteins, the presentation of sufficient MOMP serovars to protect against a wide spectrum of serovars, and the simultaneous prophylaxis/treatment of gonococcal infection (and consequently the non-requirement of practitioners to initially decide which organism is causing particular clinical symptoms—both organisms can be vaccinated against simultaneously thus allowing the treatment of the STD at a very early stage). Preferred loci for gene insertion in the gonoccocal chromosome are give above. Other preferred, protective C. trachoinatis genes that could be incorporated are HMWP, PmpG and those OMPs disclosed in WO 99/28475.

Targeting of Heterologous Proteins to Outer-Membrane Blebs:

The expression of some heterologous proteins in bacterial blebs may require the addition of outer-membrane targeting signal(s). The preferred method to solve this problem is by creating a genetic fusion between a heterologous gene and a gene coding for a resident OMP as a specific approach to target recombinant proteins to blebs. Most preferably, the heterologous gene is fused to the signal peptides sequences of such an OMP.

Neisserial Bleb Preparations

One or more of the following genes (encoding protective antigens) are preferred for upregulation via processes b) and/or i) when carried out on a Neisserial strain, including gonococcus, and meningococcus (particularly N. meningitidis B): NspA (WO 96/29412), Hsf-like (WO 99/31132), Hap (PCT/EP99/02766), PorA, PorB, OMP85 (WO 00/23595), PilQ (PCT/EP99/03603), PlDA (PCT/EP99/06718), FrpB (WO 96/31618), ThpA (U.S. Pat. No. 5,912,336), TbpB, FrpA/FrpC (WO 92/01460), LbpA/LbpB (PCT/EP98/05117), FhaB (WO 98/02547), HasR (PCT/EP99/05989), lipo02 (PCT/EP99/08315), Thp2 (WO 99/57280), MltA (WO 99/57280), and ctrA (PCT/EP00/00135). They are also preferred as genes which may be heterologously introduced into other Gram-negative bacteria.

One or more of the following genes are preferred for downregulation via process a): PorA, PorB, PilC, ThpA, TbpB, LbpA, LbpB, Opa, and Opc.

One or more of the following genes are preferred for downregulation via process d): htrB, msbB and lpxK (or homologues thereof).

One or more of the following genes are preferred for upregulation via process e): pmrA, pmrB, pmrE, and pmrF (or homologues thereof).

Preferred repressive control sequences for process c) are: the fur operator region (particularly for either or both of the TbpB or LbpB genes); and the DtxR operator region.

One or more of the following genes are preferred for downregulation via process h): galE, siaA, siaB, siaC, siaD, ctrA, ctrB, ctrC, and ctrD (or homologues thereof).

Pseudomonas aeruginosa Bleb Preparations

One or more of the following genes (encoding protective antigens) are preferred for upregulation via processes b) and/or i): PcrV, OprF, OprI. They are also preferred as genes which may be heterologously introduced into other Gram-negative bacteria.

Moraxella catarrhalis Bleb Preparations

One or more of the following genes (encoding protective antigens) are preferred for upregulation via processes b) and/or i): OMP106 (WO 97/41731 & WO 96/34960), HasR (PCT/EP99/03824), PilQ (PCT/EP99/03823), OMP85 (PCT/EP00/01468), lipo06 (GB 9917977.2), lipo10 (GB 9918208.1), lipo11 (GB 9918302.2), lipo18 (GB 9918038.2), P6 (PCT/EP99/03038), ompCD, CopB (Helminen M E, et al (1993) Infect. Immun. 61:2003-2010), D15 (PCT/EP99/03822), OmplA1 (PCT/EP99/06781), Hly3 (PCT/EP99/03257), LbpA and LbpB (WO 98/55606), ThpA and TbpB (WO 97/13785 & WO 97/32980), OmpE, UspA1 and UspA2 (WO 93/03761), and Omp21. They are also preferred as genes which may be heterologously introduced into other Gram-negative bacteria.

One or more of the following genes are preferred for downregulation via process a): CopB, OMP106, OmpB1, ThpA, TbpB, LbpA, and LbpB.

One or more of the following genes are preferred for downregulation via process d): htrB, msbB and lpxK (or homologues thereof).

One or more of the following genes are preferred for upregulation via process e): pmrA, pmrB, pmrE, and pmrF (or homologues thereof).

One or more of the following genes are preferred for downregulation via process h) to remove any human-like epitopes from LPS: galE, siaA, siaB, siaC, siaD, ctrA, ctrB, ctrC, and ctrD (or homologues thereof).

Haemophilus influenzae Bleb Preparations

One or more of the following genes (encoding protective antigens) are preferred for upregulation via processes b) and/or i): D15 (WO 94/12641), P6 (EP 281673), TbpA, TbpB, P2, P5 (WO 94/26304), OMP26 (WO 97/01638), HMW1, HMW2, HMW3, HMW4, Hia, Hsf, Hap, Hin47, and Hif (all genes in this operon should be upregulated in order to upregulate pilin). They are also preferred as genes which may be heterologously introduced into other Gram-negative bacteria.

One or more of the following genes are preferred for downregulation via process a): P2, P5, Hif, IgA1-protease, HgpA, HgpB, HMW1, HMW2, Hxu, TbpA, and TbpB.

One or more of the following genes are preferred for downregulation via process d): htrB, msbB and lpxK (or homologues thereof).

One or more of the following genes are preferred for upregulation via process e): pmrA, pmrB, pmrE, and pmrF (or homologues thereof).

One or more of the following genes are preferred for downregulation via process h) to remove any human-like epitopes from LPS: galE, siaA, siaB, siaC, siaD, ctrA, ctrB, ctrC, and ctrD (or homologues thereof).

Vaccine Formulations

A preferred embodiment of the invention is the formulation of the bleb adjuvant preparations of the invention in a vaccine which may also comprise a pharmaceutically acceptable excipient.

The manufacture of bleb preparations from any of the aforementioned modified strains may be achieved by any of the methods well known to a skilled person. Preferably the methods disclosed in EP 301992, U.S. Pat. No. 5,597,572, EP 11243 or U.S. Pat. No. 4,271,147 are used. Most preferably, the method described in Example 8 is used.

Vaccine preparation is generally described in Vaccine Design (“The subunit and adjuvant approach” (eds Powell M. F. & Newman M. J.) (1995) Plenum Press New York).

The bleb adjuvants of the present invention may be advantageously combined with further adjuvants in the vaccine formulation of the invention. Suitable further adjuvants include an aluminium salt such as aluminum hydroxide gel (alum) or aluminium phosphate, but may also be a salt of calcium (particularly calcium carbonate), iron or zinc, or may be an insoluble suspension of acylated tyrosine, or acylated sugars, cationically or anionically derivatised polysaccharides, or polyphosphazenes.

Suitable Th1 adjuvant systems that may be used in combination with bleb adjuvant include, Monophosphoryl lipid A, particularly 3-de-O-acylated monophosphoryl lipid A, and a combination of monophosphoryl lipid A, preferably 3-de-O-acylated monophosphoryl lipid A (3D-MPL) together with an aluminium salt. An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO 96/33739. A particularly potent adjuvant formulation to be used with bleb adjuvant involves QS21 3D-MPL and tocopherol in an oil in water emulsion (described in WO 95/17210) and is a preferred formulation.

The adjuvant may additionally comprise a saponin, more preferably QS21. It may also additionally comprise an oil in water emulsion and tocopherol. Unmethylated CpG containing oligo nucleotides (WO 96/02555) are also preferential inducers of a TH1 response and are suitable for use with bleb adjuvant in the present invention.

The vaccine preparations (bleb adjuvant mixed with antigen) of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Thus one aspect of the present invention is a method of immunizing a human host against a disease caused by infection of a gram-negative bacteria, which method comprises administering to the host an immunoprotective dose of a protective antigen derived from said bacterium mixed with the bleb adjuvant of the present invention. The vaccine compositions of the present invention are particularly suitable for intranasal use. Further adjuvants such as Laureth-9 may also be included.

The amount of antigen in each vaccine dose is selected as an amount which induces an immunoprotective response without significant, adverse side effects in typical vaccinees (as defined above).

Ghost or Killed Whole Cell Adjuvants

The inventors envisage that the above improvements to bleb adjuvants and resulting vaccine compositions can be easily extended to ghost or killed whole cell adjuvants preparations and vaccines (with identical advantages). The modified Gram-negative strains of the invention from which the bleb preparations are made can also be used to made ghost and killed whole cell adjuvant preparations. Methods of making ghost preparations (empty cells with intact envelopes) from Gram-negative strains are well known in the art (see for example WO 92/01791). Methods of killing whole cells to make inactivated cell preparations for use in vaccines are also well known. The terms ‘bleb adjuvant preparations’ and ‘vaccines comprising bleb adjuvant’ as well as the processes described throughout this document are therefore applicable to the terms ‘ghost adjuvant preparation’ and ‘vaccines comprising ghost adjuvant’, and ‘killed whole cell adjuvant preparation’ and ‘vaccine comprising killed whole cell adjuvant’, respectively, for the purposes of this invention.

Combinations of Methods a)-i)

It may be appreciated that one or more of the above processes may be used to produce a modified strain from which to make improved bleb adjuvant preparations of the invention. Preferably one such process is used, more preferably two or more (2, 3, 4, 5, 6, 7, 8 or 9) of the processes are used in order to manufacture the bleb adjuvant. As each additional method is used in the manufacture of the adjuvant (particularly from processes d), e) and h)), each improvement works in conjunction with the other methods used in order to make an optimised engineered bleb adjuvant preparation.

A preferred meningococcal (particularly N. meningitidis B) bleb adjuvant preparation comprises the use of processes d) and h) and/or e). Such bleb preparations are safe (no structures similar to host structures), and non-toxic, but are still potent adjuvants. All the above elements work together in order to provide an optimised bleb adjuvant.

Similarly for M. catarrhalis and non-typeable H. influenzae, preferred bleb preparations comprise the use of processes d) and/or h) and/or e).

A further aspect of the invention is thus an safe and non-toxic Gram-negative bleb, ghost, or killed whole cell adjuvant suitable for paediatric use.

By paediatric use it is meant use in infants less than 4 years old.

By non-toxic it is meant that there is a significant (24 fold, preferably 10 fold) decrease of endotoxin activity as measured by the well-known LAL and pyrogenicity assays.

Nucleotide Sequences of the Invention

A further aspect of the invention relates to the provision of new nucleotide sequences which may be used in the processes of the invention. Specific upstream regions from various genes from various strains are provided which can be used in, for instance, processes a), b), d) and h). In addition, coding regions are provided for performing process d).

General Method for the Analysis of the Non-Coding Flanking Region of a Bacterial Gene, and its Exploitation for Modulated Expression of the Gene in Blebs

The non-coding flanking regions of a specific gene contain regulatory elements important in the expression of the gene. This regulation takes place both at the transcriptional and translational level. The sequence of these regions, either upstream or downstream of the open reading frame of the gene, can be obtained by DNA sequencing. This sequence information allows the determination of potential regulatory motifs such as the different promoter elements, terminator sequences, inducible sequence elements, repressors, elements responsible for phase variation, the Shine-Dalgarno sequence, regions with potential secondary structure involved in regulation, as well as other types of regulatory motifs or sequences.

This sequence information allows the modulation of the natural expression of the gene in question. The upregulation of the gene expression may be accomplished by altering the promoter, the Shine-Dalgarno sequence, potential repressor or operator elements, or any other elements involved. Likewise, downregulation of expression can be achieved by similar types of modifications. Alternatively, by changing phase variation sequences, the expression of the gene can be put under phase variation control, or may be uncoupled from this regulation. In another approach, the expression of the gene can be put under the control of one or more inducible elements allowing regulated expression. Examples of such regulation includes, but is not limited to, induction by temperature shift, addition of inductor substrates like selected carbohydrates or their derivatives, trace elements, vitamins, co-factors, metal ions, etc.

Such modifications as described above can be introduced by several different means. The modification of sequences involved in gene expression can be done in vivo by random mutagenesis followed by selection for the desired phenotype. Another approach consists in isolating the region of interest and modifying it by random mutagenesis, or site-directed replacement, insertion or deletion mutagenesis. The modified region can then be reintroduced into the bacterial genome by homologous recombination, and the effect on gene expression can be assessed. In another approach, the sequence knowledge of the region of interest can be used to replace or delete all or part of the natural regulatory sequences. In this case, the regulatory region targeted is isolated and modified so as to contain the regulatory elements from another gene, a combination of regulatory elements from different genes, a synthetic regulatory region, or any other regulatory region, or to delete selected parts of the wild-type regulatory sequences. These modified sequences can then be reintroduced into the bacterium via homologous recombination into the genome.

In process b), for example, the expression of a gene can be modulated by exchanging its promoter with a stronger promoter (through isolating the upstream sequence of the gene, in vitro modification of this sequence, and reintroduction into the genome by homologous recombination). Upregulated expression can be obtained in both the bacterium as well as in the outer membrane vesicles shed (or made) from the bacterium.

In other preferred examples, the described approaches can be used to generate recombinant bacterial strains with improved characteristics for vaccine applications, as described above. These can be, but are not limited to, attenuated strains, strains with increased expression of selected antigens, strains with knock-outs (or decreased expression) of genes interfering with the immune response, and strains with modulated expression of immunodominant proteins.

SEQ ID NO:2-23, 25, 27-38 are all Neisserial upstream sequences (upstream of the initiation codon of various preferred genes) comprising approximately 1000 bp each. SEQ ID NO: 39-62 are all M. catarrhalis upstream sequences (upstream of the initiation codon of various preferred genes) comprising approximately 1000 bp each. SEQ ID NO: 63-75 are all H. influenzae upstream sequences (upstream of the initiation codon of various preferred genes) comprising approximately 1000 bp each. All of these can be used in genetic methods (particularly homologous recombination) for up-regulating, or down-regulating the open reading frames to which they are associated (as described before). SEQ ID NO: 76-81 are the coding regions for the HtrB and MsbB genes from Neisseria, M. catarrhalis, and Haemophilus influenzae. These can be used in genetic methods (particularly homologous recombination) for down-regulating (in particular deleting) part (preferably all) of these genes [process d)], or decreasing the activity of the gene product produced.

Another aspect of the invention is thus an isolated polynucleotide sequence which hybridises under highly stringent conditions to at least a 30 nucleotide portion of the nucleotides in SEQ ID NO: 2-23, 25, 27-81 or a complementary strand thereof. Preferably the isolated sequence should be long enough to perform homologous recombination with the chromosomal sequence if it is part of a suitable vector—namely at least 30 nucleotides (preferably at least 40, 50, 60, 70, 80, 90, 100, 200, 300, 400, or 500 nucleotides). More preferably the isolated polynucleotide should comprise at least 30 nucleotides (preferably at least 40, 50, 60, 70, 80, 90, 100, 200, 300, 400, or 500 nucleotides) of SEQ ID NO: 2-23, 25, 27-81 or a complementary strand thereof.

As used herein, highly stringent hybridization conditions include, for example, 6×SSC, 5× Denhardt, 0.5% SDS, and 100 μg/mL fragmented and denatured salmon sperm DNA hybridized overnight at 65° C. and washed in 2×SSC, 0.1% SDS one time at room temperature for about 10 minutes followed by one time at 65° C. for about 15 minutes followed by at least one wash in 0.2×SCC, 0.1% SDS at room temperature for at least 3-5 minutes.

A further aspect is the use of the isolated polynucleotide sequences of the invention in performing a genetic engineering event (such as transposon insertion, or site specific mutation or deletion, but preferably a homologous recombination event) within 1000 bp upstream of a Gram-negative bacterial chromosomal gene in order to either increase or decrease expression of the gene. Preferably the strain in which the recombination event is to take place is the same as the strain from which the upstream sequences of the invention were obtained. However, the meningococcus A, B, C, Y and W and gonococcus genomes are sufficiently similar that upstream sequence from any of these strains may be suitable for designing vectors for performing such events in the other strains. This is may also be the case for Haemophilus influenzae and non-typeable Haemophilus influenzae.

### EXAMPLES

The examples below are carried out using standard techniques, which are well known and routine to those of skill in the art, except where otherwise described in detail. The examples are illustrative, but do not limit the invention. All references are incorporated by reference herein.

### Example 1

### Construction of a Neisseiria meningitidis Serogroup B Strain Lacking Capsular Polysaccharides

The plasmid pMF121 (Frosch et al., 1990) has been used to construct a Neisseria meningitidis B strain lacking the capsular polysaccharide. This plasmid contains the flanking regions of the gene locus coding for the biosynthesis pathway of the group B polysaccharide (B PS), and the erythromycin resistance gene. Deletion of the B PS resulted in loss of expression of the group B capsular polysaccharide as well as a deletion in the active copy of gale leading to the synthesis of galactose deficient LPS.

Strain Transformation:

Neisseria meningitidis B H44/76 strain (B:15:P1.7, 16;Los 3,7,9) was selected for transformation. After an overnight CO₂ incubation on MH plate (without erythromycin), cells were collected in liquid MH containing 10 mM MgCl₂ (2 ml were used per MH plate) and diluted up to an OD of 0.1 (550 nm). To this 2 ml solution, 4 μl of the plasmid pMF121 stock solution (0.5 μg/ml) were added for a 6 hours incubation period at 37° C. (with shaking). A control group was done with the same amount of Neisseria meningitidis B bacteria, but without addition of plasmid. After the incubation period, 100 μl of culture, as such, at 1/10, 1/100 and 1/1000 dilutions, were put in MH plates containing 5, 10, 20, 40 or 80 μg erythromycin/ml before incubation for 48 hours at 37° C.

Colony Blotting:

After plate incubation, 20 colonies were grown and selected from the 10 and 20 μg erythromycin/ml MH plates, while there was no colony growth in the control group without plasmid transformation. The H44/76 wild type strain was unable to grow in the selected erythromycin plates (10 to 80 μg erythromycin/ml). The day after, all the visible colonies were placed on new MH plates without erythromycin in order to let them grow. Afterwards, they were transferred onto nitrocellulose sheets (colony blotting) for presence of B polysaccharide. Briefly, colonies were blotted onto a nitrocellulose sheet and rinsed directly in PBS-0.05% Tween 20 before cell inactivation for 1 hour at 56° C in PBS-0.05% Tween 20 (diluant buffer). Afterwards, the membrane was overlaid for one hour in the diluant buffer at room temperature (RT). Then, sheets were washed again for three times 5 minutes in the diluant buffer before incubation with the anti-B PS 735 Mab (Boerhinger) diluted at 1/3000 in the diluant buffer for 2 hours at RT. After a new washing step (3 times 5 minutes), the monoclonal antibody was detected with a biotinylated anti-mouse Ig from Amersham (RPN 1001) diluted 500 times in the diluant buffer (one hour at RT) before the next washing step (as described above). Afterwards, sheets were incubated for one hour at RT with a solution of streptavidin-peroxidase complex diluted 1/1000 in the diluant buffer. After the last three washing steps using the same method, nitrocellulose sheets were incubated for 15 min in the dark using the revelation solution (30 mg of 4-chloro-1-naphtol solution in 10 ml methanol plus 40 ml PBS and 30 mcl of H₂O₂ 37% from Merck). The reaction was stopped with a distillated water-washing step.

Whole Cell Elisas:

Whole cell Elisas were also done using the two transformed colonies (“D” and “R”) and the wild type strain (H44/76) as coated bacteria (20 μg protein/ml), and a set of different monoclonal antibodies used to characterize Neisseria meningitidis strains. The following Mabs were tested: anti-B PS (735 from Dr Frosch), and the other Mabs from NIBSC: anti-B PS (Ref 95/750) anti-P1.7 (A-PorA, Ref 4025), anti-P1.16 (A-PorA, Ref 95/720), anti-Los 3,7,9 (A-LPS, Ref 4047), anti-Los 8 (A-LPS, Ref 4048), and anti-P1.2 (A-PorA Ref 95/696).

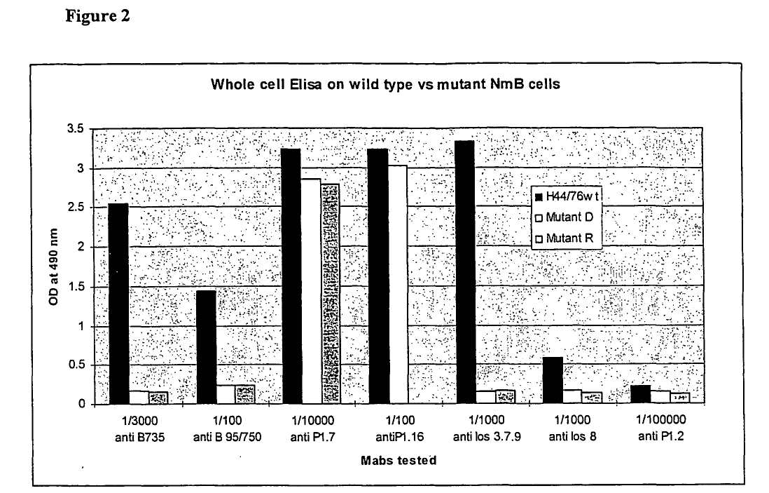

Microtiter plates (Maxisorp, Nunc) were coated with 100 μl of the recombinant meningococcal B cells solution overnight (ON) at 37° C. at around 20 μg/ml in PBS. Afterwards, plates are washed three times with 300 μl of 150 mM NaCl-0.05% Tween 20, and were overlaid with 100 μl of PBS-0.3% Casein and incubated for 30 min at room temperature with shaking. Plates were washed again using the same procedure before incubation with antibodies. Monoclonal antibodies (100 μl) were used at different dilutions (as shown in FIG. 2) in PBS-0.3% Casein 0.05% Tween 20 and put onto the microplates before incubation at room temperature for 30 min with shaking, before the next identical washing step. 100 μl of the anti-mouse Ig (from rabbit, Dakopatts E0413) conjugated to biotin and diluted at 1/2000 in PBS-0.3% Casein-0.05% Tween 20 were added to the wells to detect bound monoclonal antibodies. After the washing step (as before), plates were incubated with a streptavidin-peroxidase complex solution (100 μl of the Amersham RPN 1051) diluted at 1/4000 in the same working solution for 30 min at room temperature under shaking conditions. After this incubation and the last washing step, plates are incubated with 100 μl of the chromogen solution (4 mg orthophenylenediamine (OPD) in 10 ml 0.1 M citrate buffer pH4.5 with 5 μl H₂O₂ for 15 min in the dark. Plates are then read at 490/620 nm using a spectrophotometer.

Results:

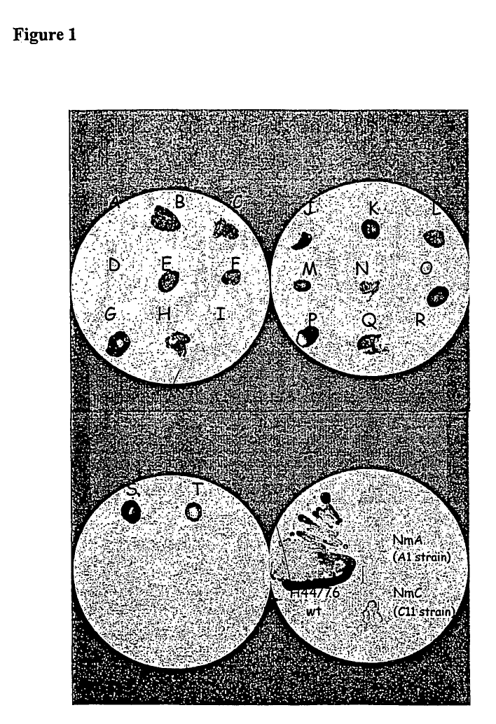

FIG. 1 shows that from the 20 isolated colonies, which were able to growth on the selected medium with erythromycin, only two (the “D” and the “R”) colonies were shown negative for presence of B polysaccharide. Among the others, 16 were clearly positive for B PS and still resistant to erythromycin. This indicated that they integrated the plasmid into their genome, but in the wrong orientation, and keeping intact the B PS and LPS gene (no double crossing-over). Positive and negative controls were also tested on the plates, and showed that the H44/76 wild type NmB strain was clearly positive for the B polysaccharide, while meningococcus A (A1) and meningococcus C (C11) strains were clearly negative with this anti-B PS 735 Mab. These results indicate that around 10% of the selected colonies correctly integrated the plasmid in their genome by making a double crossing-over, while the other strains/colonies were obtained after a simple crossing-over, keeping the B PS and LPS genes intact and expressed.

Using whole cell Elisa, results (FIG. 2 and the Table below) clearly indicate that the two “D” and “R” transformants (derived from D and R colonies) can not be recognized anymore by the anti-B PS Mabs (735 and 95/750), nor by the anti-Los 3,7,9 and anti-Los 8 Mabs. However, when using specific anti-PorA Mabs, there is a clear reaction with the anti-P1.7 and anti-P 1.16 Mabs on the cells, as also observed in the wild-type strain. No reaction was observed with a non-specific anti-PorA Mab (anti-P1.2 mab). These results confirm that the PorA protein, and specifically P1.7 and P1.16 epitopes are still present after transformation, while B polysaccharide and Los 3.7,9 and Los 8 epitopes (LPS) were not.

TABLE
Specificities of the monoclonal antibodies tested

| Mabs | Directed | |
|---|---|---|
| Tested | against | Result |
| Anti-B PS | B polysaccharide | ++ on the wild type strain |
| 735 | | (−) on the “D” and “R” mutants |
| Anti-B PS | B PS | ++ on the wild type strain |
| 95/750 from | | (−) on the “D” and “R” mutants |
| NIBSC | | |
| Anti-P1.7 | Loop 1 of | ++ on all wild type and |
| (NIBSC) | Porin A | mutants strains |
| Anti-P1.16 | Loop 4 of | ++ on all wild type and |
| (NIBSC) | Porin A | mutants strains |
| Anti-Los 3, 7, 9 | LPS | ++ on the wild type strain |
| | | (−) on the “D” and “R” mutants |
| Anti-Los 8 | LPS | +/− on the wild type strain |
| (NIBSC) | | (−) on the “D” and “R” mutants |
| Anti-P1.2 (NIBSC) | Anti-Porin A | (−) on all wild type and |
| | Sero-subtype 1.2 | mutants strains |

### Example 2

### Construction of Versatile Gene Delivery Vectors (the pCMK Series) Targeting Integration in the porA Locus of Neisseiria meningitidis

A plasmid allowing homologous recombination and stable integration of foreign DNA in the porA locus of Neisseiria meningitidis was constructed. This delivery vector (genes, operons and/or expression cassettes) is useful for constructing Neisseiria meningitidis strains producing recombinant, improved blebs. Typically, such a vector contains at least: (1) a plasmid backbone replicative in E. coli but not in Neisseria meningitidis (a suicide plasmid), (2) at least one, but preferably two regions of homology for targeting the integration in a chromosomal locus such as porA, (3) Efficient transcriptional (promoter, regulatory region and terminator) and translational (optimised ribosome binding site and initiation codon) signals functional in Neisseria meningitidis, (4) a multiple cloning site and (5) selectable gene(s) allowing the maintenance of the plasmid in E. coli and the selection of integrants in Neisseria meningitidis. Additional elements include, for example, uptake sequences to facilitate the entry of foreign DNA in Neisseiria meningitidis, and counter selectable markers such as sacB, rpsL, gltS to enhance the frequency of double cross-over events.

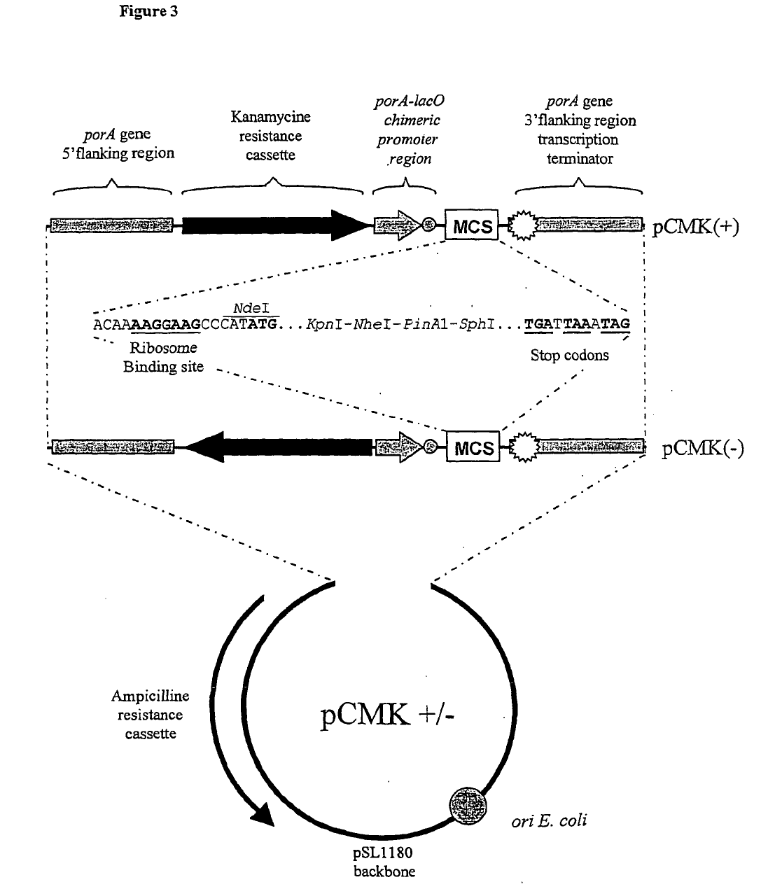

A schematic drawing of the vector constructed in this example and designated pCMK is represented in FIG. 3. Its corresponding complete nucleotide sequence is shown in SEQ. ID NO:1. pCMK derives from a pSL1180 backbone (PharmaciaBiotech, Sweeden), a high copy-number plasmid replicative in E. coli, harbouring the bla gene (and thereby conferring resistance to ampicillin). In addition to this, pCMK functionally contains two porA flanking regions (porA5′ and porA3′ containing a transcription terminator) necessary for homologous recombination, a selectable marker conferring resistance to kanamycin, two uptake sequences, a porA/lacO chimeric promoter repressed in the E. coli host expressing lacIq but transcriptionally active in Neisseria meningitidis, and a multiple cloning site (5 sites present: NdeI , KpnI, NheI, PinA1 and SphI) necessary for the insertion of foreign DNA in pCMK.

pCMK was constructed as follows. The porA5′ and porA3′ recombinogenic regions, the porA/lacO promoter were PCR amplified using the oligonucleotides listed in the table below, cloned in pTOPO and sequenced. These DNA fragments were successively excised from pTOPO and recloned in pSL1180. The kanamycin resistance cassette was excised from pUC4K (PharmaciaBiotech, Sweeden) and was introduced between the porA5′ flanking region and the porA/lacO promoter region.

TABLE
Oligonucleotides used in this work

| | | |
|---|---|---|
| Oligonucleotides | Sequence | Remark(s) |

| | | |
|---|---|---|
| PorA5′ Fwd | 5′-CCC AAG CTT GCC GTC TGA ATA CAT CCC | HindIII cloning site |
| | GTC ATT CCT CA-3′ | Uptake sequence (_) |
| PorA5′ Rev | 5′-CGA TGC TCG CGA CTC CAG AGA CCT CGT | Nru I cloning site |
| | GCG GGC C-3′ | |
| PorA3′ Fwd | 5′-GGA AGA TCT GAT TAA ATA GGC GAA AAT | Bgl II cloning site |
| | ACC AGC TAG GA-3′ | Stop codons (_) |
| PorA3′ Rev | 5′-GCC GAA TTC TTC AGA CGG C GC AGC AGG | EcoRI cloning site |
| | AAT TTA TCG G-3′ | Uptake sequence (_) |
| PoLa Rev1 | 5′-GAA TTG TTA TCC GCT CAC AAT TCC GGG | |
| | CAA ACA CCC GAT AC-3′ | |
| PoLa Rev2 | 5′-GAA TTC CAT ATG ATC GGC TTC CTT TTG | NdeI cloning site |
| | TAA ATT TGA TAA AAA CCT AAA AAC ATC GAA | |
| | TTG TTA TCG GCT C-3′ | |
| PorAlacO Fwd | 5′-AAG CTC TGC AGG AGG TCT GCG CTT GAA | PstI cloning site |
| | TTG-3′ | |
| PorAlacO Rev | 5′-CTT AAG GCA TAT GGG CTT CCT TTT GTA A- | NdeI cloning site |
| 3′ | | |
| PPA1 | 5′-GCG GCC GTT GCC GAT GTC AGC C-3′ | |
| PPA2 | 5′-GGC ATA GCT GAT GCG TGG AAC TGC-3′ | |
| N-fuIl-01: | 5′-GGG AAT TCC ATA TGA AAA AAG GAG TTG | NdeI cloning site |
| | CCA CAC-3′ | |
| Nde-NspA-3: | 5′-GGA ATT CCA TAT GTC AGA ATT TGA CGC | NdeI cloning site |
| | GCA C-3′ | |
| PNS1 | 5′-CCG CGA ATT CGG AAC CGA ACA CGG CGT | EcoRI cloning site |
| | TCG-3′ | |
| PNS1 | 5′-CGT CTA GAC GTA GCG GTA TCC GGC TGC -3′ | XbaI cloning site |
| PromD15-51X | 5′-GGG CGA ATT CGC GGC CGC CGT CAA CGG | EcoRI and NoTi cloning sites |
| | CAC ACC CGT TG-3′ | |
| PromD15-52 | 5′-GCT CTA GAG CGG AAT GCG GTT TCA GAC G- | XbaI cloning site |
| | 3′ | |
| PNS4 | 5′-AGC TTT ATT TAA ATC CTT AAT TAA CGC | SwaI and PaCI cloning sites |
| | GTC CGG AAA ATA TGC TTA TC_34 | |
| PNS5 | 5′-AGC TTT TTT TAA ACC CTG TTC CGC TGC | PmeI cloning site |
| | TTC GGC-3′ | |
| D15-S4 | 5′-GTC CGC ATT TAA ATC CTT AAT TAA GCA | SwaI and PacI cloning sites |
| | GCC GGA CAG GGC GTG G-3′ | |
| D15-S5 | 5′-AGC TTT GTT TAA AGG ATC AGG GTG TGG | PmeI cloning site |
| | TCG GGC-3′ | |

### Example 3

### Construction of a Neisseiria meningitidis Serogroup B Strain Lacking Both Capsular Polysaccharides and the Major Immunodominant Antigen PorA

Modulating the antigenic content of outer membrane blebs may be advantageous in improving their safety and efficacy in their use in vaccines, or diagnostic or therapeutic uses. Components such as the Neisseiria meningitidis serogroup B capsular polysaccharides should be removed to exclude the risk of inducing autoimmunity. (see example 1). Similarly, it is beneficial to suppress the immunodominance of major outer-membrane antigens such as PorA, which induce strain-specific bactericidal antibodies but fail to confer cross-protection. To illustrate such an approach, we used the pCMK(+) vector to construct a Neisseiria meningitidis serogroup B strain lacking both capsular polysaccharides and the immunodominant PorA outer membrane protein antigen. For this purpose, a deletion of the porA gene was introduced in the H44/76 cps− strain, described in example 1 by means of homologous recombination.

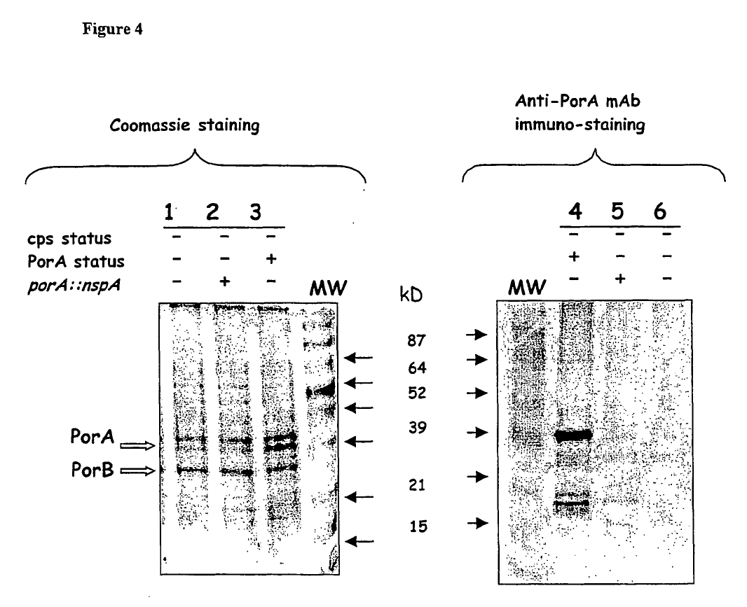

The H44/76 cps− strain was prepared competent and transformed with two 2 μg of supercoiled pCMK(+) plasmid DNA as described previously. Aliquot fractions of the transformation mixture (100 μl) were plated on Mueller-Hinton plates supplemented with Kanamycin (200 μg/ml) and incubated at 37° C. for 24 to 48 hours. Kanamycin-resistant colonies were selected, restreaked on MH-Kn and grown for an additional 24 hours at 37° C. At that stage half of the bacterial culture was used to prepare glycerol stocks (15% vol./vol.) and was kept frozen at −70° C. Another fraction (estimated to be 10⁸ bacteria) was resuspended in 15 μl of distilled water, boiled ten minutes and used as a template for PCR screening. Two porA internal primers named, PPA1 and PPA2, were synthesized and used to perform PCR amplification on boiled bacterial lysates in the conditions described by the supplier (HiFi DNA polymerase, Boehringer Mannheim, GmbH). The thermal cycling used was the following: 25 times (94° C. 1 min., 52° C. 1 min., 72° C. 3 min.) and 1 time (72° C. 10 min., 4° C. up to recovery). Since a double crossing-over between pCMK DNA and the chromosomal porA locus deletes the region required for #1 and #2 annealing, clones lacking a 1170 bp PCR amplification fragment were selected as porA deletion mutants. These PCR results were further confirmed by analyzing in parallel, the presence of PorA in the corresponding bacterial protein extracts. For that purpose, another aliquot of bacteria (estimated to be 5.108 bacteria) was re-suspended in 50 μl of PAGE-SDS buffer (SDS 5%, Glycerol 30%, Beta-mercaptoethanol 15%, Bromophenol blue 0.3mg/ml, Tris-HCl 250 mM pH6.8), boiled (100° C.)frozen(−20° C.)/boiled (100° C.) three times and was separated by PAGE-SDS electrophoresis on a 12.5% gel. Gels were then stained by Coomassie Brilliant blue R250 or transferred to a nitrocellulose membrane and probed with an anti-PorA monoclonal antibody as described in Maniatis et al. As represented in FIG. 4, both Coomassie and immunoblot staining confirmed that porA PCR negative clones do not produce detectable levels of PorA. This result confirm that the pCMK vector is functional and can be used successfully to target DNA insertion in the porA gene, abolishing concomitantly the production of the PorA outer membrane protein antigen.

### Example 4

### Up-Regulation of the NspA Outer Membrane Protein Production in Blebs Derived from a Recombinant Neisseiria meningitidis Serogroup B Strain Lacking Functional porA and cps Genes

Enriching bleb vesicles with protective antigens is advantageous for improving the efficiency and the coverage of outer membrane protein-based vaccines. In that context, recombinant Neisseria meningitidis strains lacking functional cps and porA genes were engineered so that the expressions level of the outer-membrane protein NspA was up-regulated. For that purpose, the gene coding for NspA was PCR amplified using the N01-full-NdeI and NdeI-3′oligonucleotide primers (see table in example 2). The conditions used for PCR amplification were those described by the supplier (HiFi DNA polymerase, Boehringer Mannheim, GmbH). Thermal cycling done was the following: 25 times (94° C. 1 min., 52° C. 1 min., 72° C. 3 min.) and 1 time (72° C. 10 min., 4° C. up to recovery). The corresponding amplicon was digested with NdeI and inserted in the NdeI restriction site of the pCMK(+) delivery vector. Insert orientation was checked and recombinant plasmids, designed pCMK(+)-NspA, were purified at a large scale using the QIAGEN maxiprep kit and 2 μg of this material was used to transform a Neisseiria meningitidis serogroup B strain lacking functional cps genes (strain described in example 1). Integration resulting from a double crossing-over between the pCMK(+)-NspA vector and the chromosomal porA locus were selected using a combination of PCR and Western blot screening procedures presented in example 3.

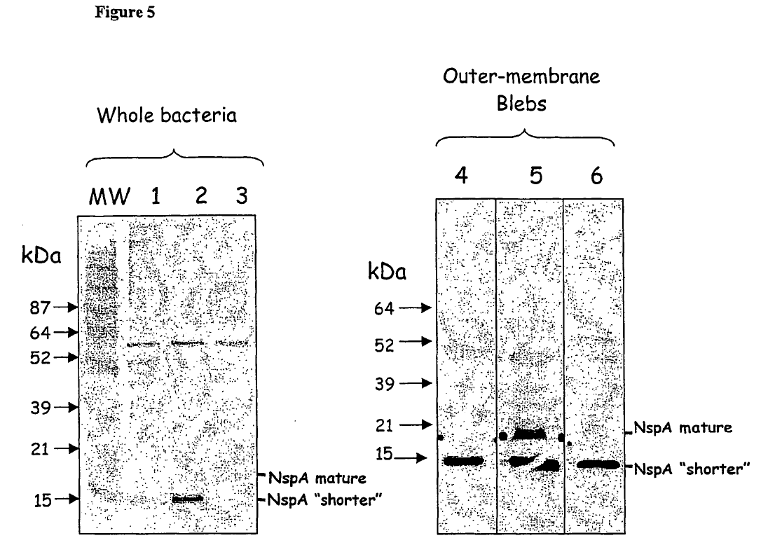

Bacteria (corresponding to about 5.10⁸ bacteria) were re-suspended in 50 μl of PAGE-SDS buffer, frozen(−20° C.)/boiled (100° C.) three times and then were separated by PAGE-SDS electrophoresis on a 12.5% gel. Gels were then stained by Coomassie Brilliant blue R250 or transferred to a nitrocellulose membrane and probed with an anti-NspA polyclonal serum. Both Coomassie (data not shown) and immunoblot staining (see FIG. 4) confirmed that porA PCR negative clones do not produce detectable levels of PorA. The expression of NspA was examined in Whole-cell bacterial lysates (WCBL) or outer-membrane bleb preparations derived from NmB [cps−, porA−] or NmB [cps−, porA−, Nspa+]. Although no difference was observable by Coomassie staining, immunoblotting with the anti-NspA polyclonal serum detected a 3-5 fold increased in the expression of NspA (with respect to the endogenous NspA level), both in WCBL and outer-membrane bleb preparations (see FIG. 5). This result confirm that the pCMK(+)-NspA vector is functional and can be used successfully to up-regulate the expression of outer membrane proteins such as NspA, abolishing concomitantly the production of the PorA outer membrane protein antigen.

### Example 5

### Up-Regulation of the D15/Omp85 Outer Membrane Protein Antigen in Blebs Derived from a Recombinant Neisseiria meningitidis Serogroup B Strain Lacking Functional cps Genes but Expressing PorA

Certain geographically isolated human populations (such as Cuba) are infected by a limited number of Neisseiria meningitidis isolates belonging largely to one or few outer membrane protein serotypes. Since PorA is a major outer-membrane protein antigen inducing protective and strain-specific bactericidal antibodies, it is then possible to confer vaccine protection using a limited number of porA serotypes in a vaccine. In such a context, the presence of PorA in outer membrane vesicles may be advantageous, strengthening the vaccine efficacy of such recombinant improved blebs. Such PorA containing vaccines, however, can be improved still further by increasing the level of other cross-reactive OMPs such as omp85/D 15.

In the following example, the pCMK(+) vector was used to up-regulate the expression of the Omp85/D15 outer membrane protein antigen in a strain lacking functional cps genes but expressing porA. For that purpose, the gene coding for Omp85/D15 was PCR amplified using the D15-NdeI and D15-NotI oligonucleotide primers. The conditions used for PCR amplification were those described by the supplier (HiFi DNA polymerase, Boehringer Mannheim, GmbH). Thermal cycling done was the following: 25 times (94° C. 1 min., 52° C. 1 min., 72° C. 3 min.) and 1 time (72° C. 10 min., 4° C. up to recovery). The corresponding amplicon was inserted in the pTOPO cloning vector according to the manufacturer's specifications and confirmatory sequencing was performed. This Omp85/D15 DNA fragment was excised from pTOPO by restriction hydrolysis using NdeI/NsiI and subsequently cloned in the corresponding restriction sites of the pCMK(+) delivery vector. Recombinant plasmids, designed pCMK(+)-D15 were purified on a large scale using the QIAGEN maxiprep kit and 2 μg of this material was used to transform a Neisseiria meningitidis serogroup B strain lacking functional cps genes (strain described in example 1). In order to preserve the expression of porA, integration resulting from a single crossing-over (either in Omp85/D15 or in porA) were selected by a combination of PCR and Western blot screening procedures. Kanamycin resistant clones testing positive by porA-specific PCR and western blot were stored at −70° C. as glycerol stocks and used for further studies.

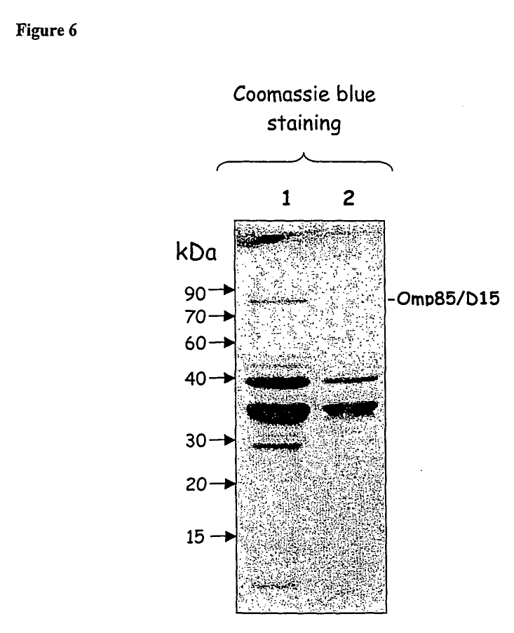

Bacteria (corresponding to about 5.10⁸ bacteria) were re-suspended in 50 μl of PAGE-SDS buffer, frozen(−20° C)/boiled (100° C.) three times and then were separated by PAGE-SDS electrophoresis on a 12.5% gel. Gels were then stained by Coomassie Brilliant blue R250 or transferred to a nitrocellulose membrane and probed with an anti-porA monoclonal antibody. As represented in FIG. 6, both Coomassie and immunoblot staining confirmed that porA PCR positive clones produce PorA.

The expression of D15 was examined using outer-membrane bleb preparations derived from NmB [cps−, porA−] or NmB [cps−, porA+, D15+]. Coomassie detected a significant increase in the expression of D15 (with respect to the endogenous D15 level), preparations (see FIG. 6). This result confirmed that the pCMK(+)-D15 vector is functional and can be used successfully to up-regulate the expression of outer membrane proteins such as D15, without abolishing the production of the major PorA outer membrane protein antigen.

### Example 6

### Construction of Versatile Promoter Delivery Vectors

Rational: The rational of this approach is represented in FIG. 7 and can be summarized in 7 essential steps. Some of these steps are illustrated below with the construction of Vector for up-regulating the expression of NspA and D I5/Omp85.

Vector for Up-Regulating the Expression of the NspA Gene.

Step 1. A DNA region (997 bp) located upstream from the, NspA coding gene was discovered (SEQ. ID NO:2) in the private Incyte PathoSeq data base containing unfinished genomic DNA sequences of the Neisseria meningitidis strain ATCC 13090. Two oligonucleotide primers referred to as PNS1 and PNS2 (see table in example 2) were designed using this sequence and synthesized. These primers were used for PCR amplification using genomic DNA extracted from the H44/76 strain. Step 2. The corresponding amplicons were cleaned-up using the Wizard PCR kit (Promega, USA) and submitted to digestion with the EcoRI/XbaI restriction enzymes for 24 hours using the conditions described by the supplier (Boehringer Mannheim, Germany). The corresponding DNA fragments were gel purified and inserted in the corresponding sites of the pUC18 cloning vector. Step 3. Recombinant plasmids were prepared on a large scale and an aliquot fraction was used as a template for inverse PCR amplification. Inverse PCR was performed using the PNS4 and PNS5 oligonucleotides using the following thermal cycling conditions: 25 times (94° C. 1 min., 50° C. 1 min., 72° C. 3 min.) and 1 time (72° C. 10 min., 4° C. up to recovery). Linearized pUC 18 vectors harbouring a deletion in the NspA upstream region insert were obtained.

Vector for Up-Regulating the Expression of the D15/omp85 Gene.

Step 1. A DNA region (1000 bp) located upstream from the D15/omp85 coding gene was discovered (SEQ. ID NO:3) in the private Incyte PathoSeq database containing unfinished genomic DNA sequences of the Neisseria meningitidis strain ATCC 13090. Two oligonucleotide primers refererred to as PromD15-51X and PromD15-S2 (see table in example 2) were designed using this sequence and synthesized. These primers were used for PCR amplification using genomic DNA extracted from the H44/6 strain. Step 2. The corresponding amplicons were cleaned-up using the Wizard PCR kit (Promega, USA) and submitted to digestion with the EcoRI/XbaI restriction enzymes for 24 hours in the conditions described by the supplier (Boehringer Mannheim, Germany). The corresponding DNA fragments were gel purified and inserted in the corresponding sites of the pUC18 cloning vector. Step 3. Recombinant plasmids were prepared on a large scale and an aliquot fraction was used as a template for inverse PCR amplification. Inverse PCR was performed using the D15-S4 and D15-S5 oligonucleotides using the following thermal cycling conditions: 25 times (94° C. 1 min., 50° C. 1 min., 72° C. 3 min.) and 1 time (72° C. 10 min., 4° C. up to recovery). Linearized pUC 18 vectors harbouring a deletion in the D15/omp85 upstream region insert were obtained.

### Example 7

### Fermentation Processes for Producing Recombinant Blebs

The examples listed below describe methods for producing recombinant blebs lacking either capsular polysaccharides or capsular polysaccharides and PorA. Such a procedure may be used for a wide range of Neisseiria meningitidis recombinant strains and may be adapted over an extended scale range.

Culture media: Neisseiria meningitidis serogroup B strains were propagated in solid (FNE 004 AA, FNE 010 AA) or liquid (FNE 008 AA) culture media. These new media for growing meningococcus are advantageiously free of animal products, and are considered a further aspect of the invention.

| | Components | | | | | |
|---|---|---|---|---|---|---|
| | FNE 004 AA | | FNE 008 AA | | FNE 010 AA | |
| Agar | 18 | g/L | — | | 18 | g/L |
| NaCl | 6 | g/L | 6 | g/L | 6 | g/L |
| Na-Glutamate | — | | 1.52 | g/L | — | |
| NaH₂PO₄.2H₂O | 2.2 | g/L | 2.2 | g/L | 2.2 | g/L |
| KCl | 0.09 | g/L | 0.09 | g/L | 0.09 | g/L |
| NH₄Cl | 1.25 | g/L | 1.25 | g/L | 1.25 | g/L |
| Glucose | 5 | g/L | 20 | g/L | 5 | g/L |
| Yeast Extract UF | — | | 2.5 | g/L | — | |
| Soy Pepton | 5 | g/L | 30 | g/L | 5 | g/L |
| CaCl₂.2H₂O | 0.015 | g/L | — | | 0.015 | g/L |
| MgSO₄.7H₂O | 0.6 | g/L | 0.6 | g/L | 0.6 | g/L |

| | | | | |
|---|---|---|---|---|
| Erythromycine: | 0.015 | g/L | — | — |

| | | | | |
|---|---|---|---|---|
| Kanamycine | — | — | 0.2 | g/L |

Flask cultivation of Neisseiria meningitidis serogroup B cps− recombinant blebs: This was performed in two steps comprising preculture on solid medium followed by liquid cultivation. Solid pre-culture A vial of seed was removed from freezer (−80° C.), thawed to room temperature and 0.1 mL was streaked into a Petri dish containing 15 mL of FNE004AA (see above).The Petri dish was incubated at 37° C. for 18±2 hours. The surface growth was resuspended in 8 mL of FNE008AA (see above) supplemented with 15 mg/L of erythromycin. Flask culture. 2 mL of resuspended solid pre-culture were added to a 2 litre flask containing 400 mL of FNE008AA supplemented with 15 mg/L of erythromycin. The flask was placed on a shaking table (200 rpm) and incubated at 37° C. for 16±2 hours. The cells were separated from the culture broth by centrifugation at 5000 g at 4° C. for 15 minutes.

Batch mode cultivation of Neisseiria meningitidis serogroup B cps− recombinant blebs: This was performed in three steps comprising preculture on solid medium, liquid cultivation and batch mode cultivation. Solid pre-culture._A vial of seed was removed from freezer (−80° C.), thawed to room temperature and 0.1 mL was streaked into a Petri dish containing 15 mL of FNE004AA (see above). The Petri dish was incubated at 37° C. for 18±2 hours. The surface growth was resuspended in 8 mL of FNE008AA (see above) supplemented with 15 mg/L of erythromycin. Liquid pre-culture._{—}2 mL of resuspended solid pre-culture were added to one 2 liters flask containing 400 mL of FNE008AA supplemented with 15 mg/L of erythromycin. The flask was placed on a shaking table (200 rpm) and incubated at 37° C. for 16±2 hours. The content of the flask was used to inoculate the 20 liters fermenter. Batch mode culture in fermenter. The inoculum (400 mL) was added to a pre-sterilized 20 liters (total volume) fermenter containing 10 L of FNE008AA supplemented with 15 mg/L of erythromycin. The pH was adjusted to and maintained at 7.0 by the automated addition of NaOH (25% w/v) and H₃PO₄ (25% v/v). The temperature was regulated at 37° C. The aeration rate was maintained at 20 L of air/min and the dissolved oxygen concentration was maintained at 20% of saturation by the agitation speed control. The overpressure in the fermenter was maintained at 300 g/cm². After 9±1 hours, the culture was in stationary phase. The cells were separated from the culture broth by centrifugation at 5000 g at 4° C. for 15 minutes.

Flask cultivation of Neisseiria meningitidis serogroup B cps−, PorA− recombinant blebs: This was performed in two steps comprising preculture on solid medium followed by liquid cultivation._Solid pre-culture. A vial of seed was removed from freezer (−80° C.), thawed to room temperature and 0.1 mL was streaked into a Petri dish containing 15 mL of FNE010AA (see above). The Petri dish was incubated at 37° C. for 18±2 hours. The surface growth was resuspended in 8 mL of FNE008AA (see above) supplemented with 200 mg/L of kanamycin. Flask culture. 2 mL of resuspended solid pre-culture were added to a 2 litre flask containing 400 mL of FNE008AA supplemented with 200 mg/L of kanamycin. The flask was placed on a shaking table (200 rpm) and incubated at 37° C. for 16±2 hours. The cells were separated from the culture broth by centrifugation at 5000 g at 4° C. for 15 minutes.

### Example 8

### Isolation and Purification of Blebs from Meningococci Devoid of Capsular Polysaccharide

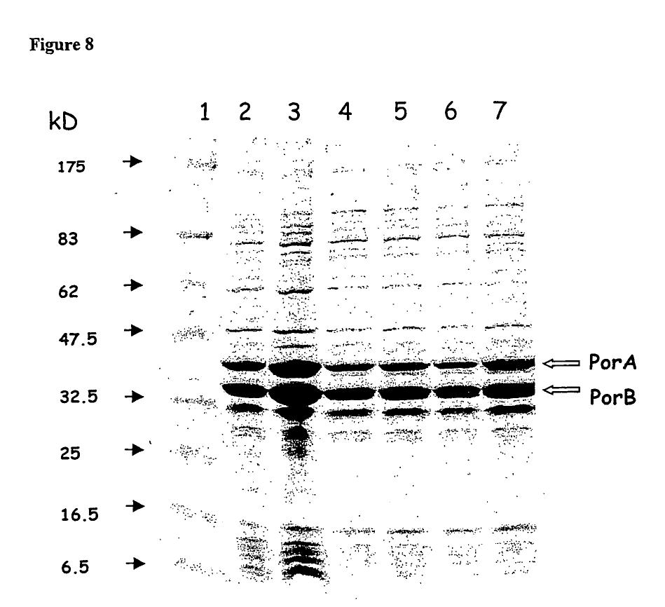

Recombinant blebs were purified as described below. The cell paste (42 gr) was suspended in 211 ml of 0.1M Tris-Cl buffer pH 8.6 containing 10 mM EDTA and 0.5% Sodium Deoxycholate (DOC). The ratio of buffer to biomass was 5/1 (V/W). The biomass was extracted by magnetic stirring for 30 minutes at room temperature. Total extract was then centrifuged at 20,000 g for 30 minutes at 4° C. (13,000 rpm in a JA-20 rotor, Beckman J2-HS centrifuge). The pellet was discarded. The supernatant was ultracentrifuged at 125,000g for 2 hours at 4° C. (40,000 rpm in a 50.2Ti rotor, Beckman L8-70M ultracentrifuge) in order to concentrate vesicles. The supernatant was discarded. The pellet was gently suspended in 25 ml of 50 mM Tris-Cl buffer pH 8.6 containing 2 mM EDTA, 1.2% DOC and 20% sucrose. After a second ultracentrifugation step at 125,000 g for 2 hours at 4° C., vesicles were gently suspended in 44 ml of 3% sucrose and stored at 4° C. All solutions used for bleb extraction and purification contained 0.01% thiomersalate. As illustrated in FIG. 8, this procedure yields protein preparations highly enriched in outer-membrane proteins such as PorA and PorB.

### Example 9

### Identification of Bacterial Promoters Suitable for Up-Regulation Antigens-Coding Genes

The use of strong bacterial promoter elements is essential to obtain up-regulation of genes coding for outer membrane proteins. In that context, we have shown previously that up-regulating the Neisseria meningitidis nspA, hsf, and omp85 genes using the porA promoter has allowed us to isolate recombinant blebs enriched in the corresponding NspA, Hsf and Omp85 proteins. Alternatives to the porA promoter may be useful to obtain different levels of up-regulation, to overcome potential porA phase variation and/or to achieve conditional gene expression (iron-regulated promoters). Here we describe a method allowing the identification of a precise transcriptional start site of strong promoter elements likely to confer high level of expression in bacteria. Since promoter regulatory elements are classically encompassed within 200 bp upstream and 50 bp dowtream from the +1 site (Collado-Vides J, Magasanik B, Gralla J D, 1991, Microbiol Rev 55(3):371-94), the result of such an experiment allows us to identify DNA fragments of about 250 bp carrying strong promoter activities. Major outer membrane proteins such as Neisseria meningitidis PorA, PorB & Rmp, Haemophilus influenzae P1, P2, P5 & P6, Moraxella catarrhalis OmpCD, OmpE, as well as some cyoplasmic and/or iron regulated proteins of these bacteria possess strong promoter elements. As a validation of this general methodology, we mapped the transcriptional start site of the strong Neisseria meningitidis porA and porB promoters using rapid amplification of cDNA elements (5′ RACE).

The principles of 5′ RACE are the following: 1) Total RNA extraction using QIAGEN “RNeasy” Kit. Genomic DNA removing by DNase treatment followed by QIAGEN purification; 2) mRNA reverse transcription with a porA specific 3′ end primer (named porA3). Expected cDNA size: 307 nt. RNA removing by alkaline hydrolysis; 3) Ligation of a single-stranded DNA oligo anchor (named DT88) to the 3′ end of the cDNA using T4 RNA ligase. Expected product size: 335 nt. Amplification of the anchor-ligated cDNA using a combination of hemi-nested PCR; 4) PCR amplification of the anchor-ligated cDNA using a complementary-sequence anchor primer as the 5′ end primer (named DT89) and a 3′end primer (named p1-2) which is internal to the 3′end RT primer porA3. Expected product size: 292 bp; 5) PCR amplification of previous PCR products using DT89 as 5′end primer and p1-1 as 3′end primer (internal to p1-2). Expected product size: 211 bp; and 6) Sequencing with p1-1 primer (expected products size can be calculated because porA transcription start site is known: 59 nt before the “ATG” translation start site).

Experimental Procedure

Total RNA was extracted from approximately 10⁹ cells of Neisseria meningitidis serogroup B cps− pora+ strain. Extraction of 1 ml of a liquid culture at appropriate optical density (OD₆₀₀=1) was performed by the QIAGEN “RNAeasy” kit according to the manufacturer's instructions. Chromosomal DNA was removed by addition of 10 U of RNase-free DNase (Roche Diagnostics, Mannheim, Germany) to the 30 μl of eluted RNA and was incubated at 37° C. for 15 min. The DNA-free RNA was purified with the same QIAGEN kit according to instructions.

Reverse transcription reactions were performed using primer porA3 and 200 U of SUPERSCRIPT II reverse transcriptase (Life Technologies). The RT reactions were performed in a 50 μl volume containing: 5 μl of 2 mM dNTP, 20 pmol of porA3 pimer, 5 μl of 10× SUPERSCRIPT II buffer, 9 μl of 25 mM MgCl2, 4 μl of 0.1M DTT, 40 U of recombinant ribonuclease inhibitor and 1 μg of total RNA. The porA3 primer was annealed stepwise (70° C. for 2 min, 65° C. for 1 min, 60° C. for 1 min, 55° C. for 1 min, 50° C. for 1 min, and 45° C. for 1 min) before the SUPERSCRIPT II was added. The RT reaction was performed at 42° C. for 30 min, followed by 5 cycles (50° C. for 1 min, 53° C. for 1 min and 56° C. for 1 min) to destabilize RNA secondary structure. Two parallel reactions were performed with the reverse transcriptase omitted from one reaction as negative control.

The RNA was removed by alkaline hydrolysis cleavage with the addition of 1 μl of 0.5M EDTA followed by 12.5 μl of 0.2 M NaOH before incubation at 68° C. for 5 min. The reactions were neutralized by adding 12.5 μl of 1 M Tris-HCl (pH7.4) and precipitated by the addition of 20 μg of glycogen (Roche Molecular Biochemicals, Mannheim, Germany), 5 μl of 3 M sodium acetate and 60 μl of isopropanol. Both samples were resuspended in 20 μl of 10:1 TE (10 mM Tris-HCl, pH 7.4; 1 mM EDTA, pH8).

T4 RNA ligase was used to anchor a 5′-phosphorylated, 3′end ddCTP-blocked anchor oligonucleotide DT88 (see table below). Two parallel ligations were performed overnight at room temperature with each containing: 1.3 μl of 10× RNA ligase buffer (Roche Molecular Biochemicals), 0.4 μM DT88, 10 μl of either cDNA or RT control sample and 3 U of T4 RNA ligase. As negative controls, a second set of ligations reactions was performed, omitting the T4 RNA ligase. The resulting ligation-reaction mixtures were used directly without purification in the subsequent PCR.

The anchor-ligated cDNA was amplified using a combination of hemi-nested and hot-started PCR approaches to increase specificity and product yield. Four separate first-round PCR were performed on the RT/ligase reaction and controls in a 30 μl volume, each containing: 3 μl of 10× Taq Platinium buffer, 3 μl of 25 mM MgCl₂, 1 μl of 10 mM dNTP, 10 pmol of each primers and 1 μl of corresponding RNA ligation reaction. The PCR were hot started by the use of Taq Platinium (Life Technologies) DNA polymerase (2 U added). The first ligation-anchored PCR (LA-PCR) was performed using 10 pmol of both the anchor-specific primer DT89 and the transcript-specific primer p1-2 (see table below) which is internal to the 3′ end RT primer porA3. The PCR was performed using an initial 95° C. for a 5 min step (for DNA polymerase activation) followed by 10 cycles at 95° C. for 10 s and 70° C. for 1 min (reducing one degree per cycle), 15 cycles at 95° C. for 10 s and 60° C. for 1 min. The second hemi-nested LA-PCR was performed under the same conditions using primer DT89 and the p1-2 internal primer, together with 10 pmol of p1-1 (see table below) and 1 μl of first-round PCR. Amplification products were purified using the QIAGEN “QIAquick PCR purification” kit according to manufacturer instructions before submitted to sequencing.

The CEQ™ Dye Terminator Cycle Sequencing kit (Beckman, France) was used to sequence the RACE PCR products using 10 pmol of primer p1-1. Sequencing reactions were performed according to the provided instructions and sequencing products were analyzed by the Ceq2000 DNA Analysis System (Beckman-Coulter).

| | |
|---|---|
| DT88 | 5′ GAAGAGAAGGTGGAAATGGCGTTTTGGC 3′ |
| DT89 | 5′ CCAAAACGCCATTTCCACCTTCTCTTC 3′ |
| porA3 | 5′ CCAAATCCTCGCTCCCCTTAAAGCC 3′ |
| p1-2 | 5′ CGCTGATTTTCGTCCTGATGCGGC 3′ |
| p1-1 | 5′ GGTCAATTGCGCCTGGATGTTCCTG 3′ |

Results for the Neisseria meningitidis porA Promoter

The start of transcription for Neisseria meningitidis serogroup B (strain H44/76) porA-mRNA was mapped 59 bp upstream of the ATG start codon using the described 5′-RACE procedure. This result confirms the mapping preformed by primer extension and published by van der Ende et al (1995). This result supports that a DNA fragment containing nucleotides −9 to −259 with regard to the porA ATG is suitable for driving strong gene expression in Neisseria meningitidis and possibly in other bacterial species such as Haemophilus, Moraxella, Pseudomonas.

Results for the Neisseria meningitidis porB Promoter

The same experimental strategy has been applied for Neisseria meningitidis serogroup B (strain H44/76) porB transcription start site mapping. Primers listed in the table below correspond to 3′ end RT primer (porB3), transcript-specific primer that is internal to the porB3 (porB2) and internal to the porB2 (porB1). porB3, porB2 and porB1 are respectively located 265 bp, 195 bp and 150 bp downstream the ATG start codon.

| | |
|---|---|
| porB1 | 5′ GGTAGCGGTTGTAACTTCAGTAACTT 3′ |
| porB2 | 5′ GTCTTCTTGGCCTTTGAAGCCGATT 3′ |
| porB3 | 5′ GGAGTCAGTACCGGCGATAGATGCT 3′ |

Using porB1 and DT89 primers a ˜200 bp PCR amplicon was obtained by performing 5′-RACE mapping. Since porB1 is located 150 bp from the porB ATG start codon, this result supports that the porB transcriptional start site is located about 50 bp (±30 bp) upstream of the porB ATG.

The exact nucleotide corresponding to transcription initiation is presently being determined by DNA sequencing. The above PCR result supports that a DNA fragment containing nucleotides −1 to −250 with regard to the porB ATG start codon is suitable for driving strong gene expression in Neisseria meningitidis and possibly in other bacterial species such as Haemophilus, Moraxella, Pseudomonas.

### Example 10

### Up-Regulation of the N. meningitidis Serogroup B Omp85 Gene by Promoter Replacement

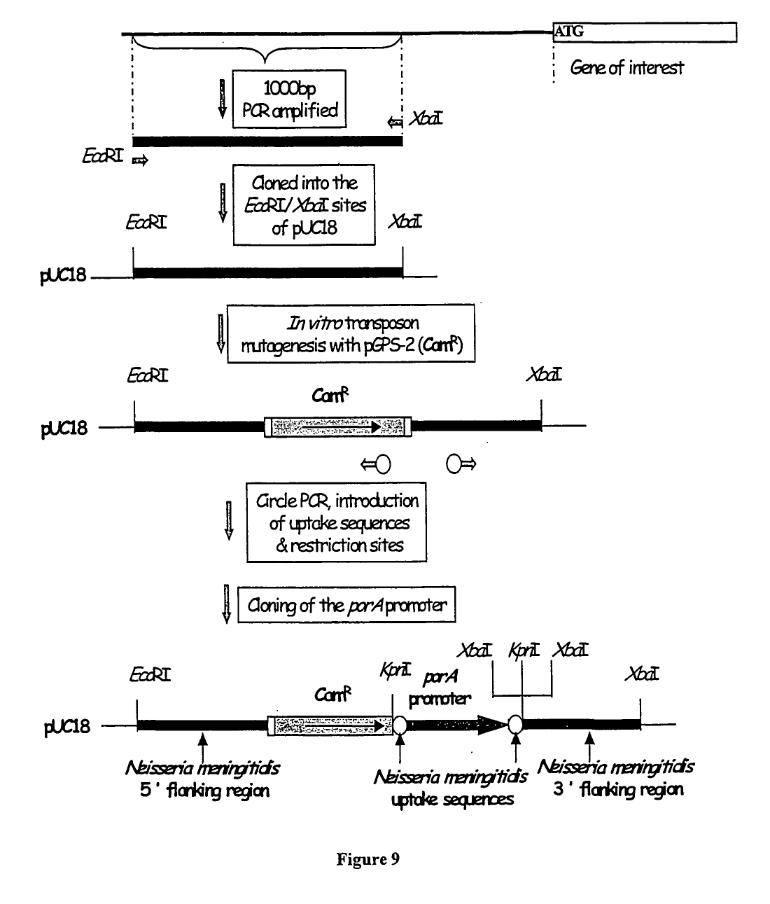

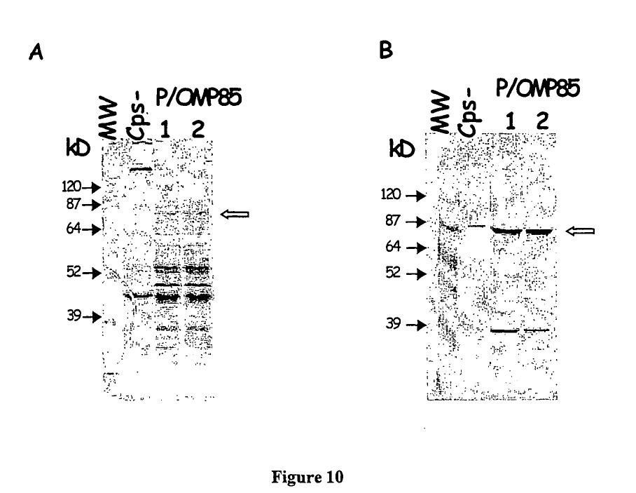

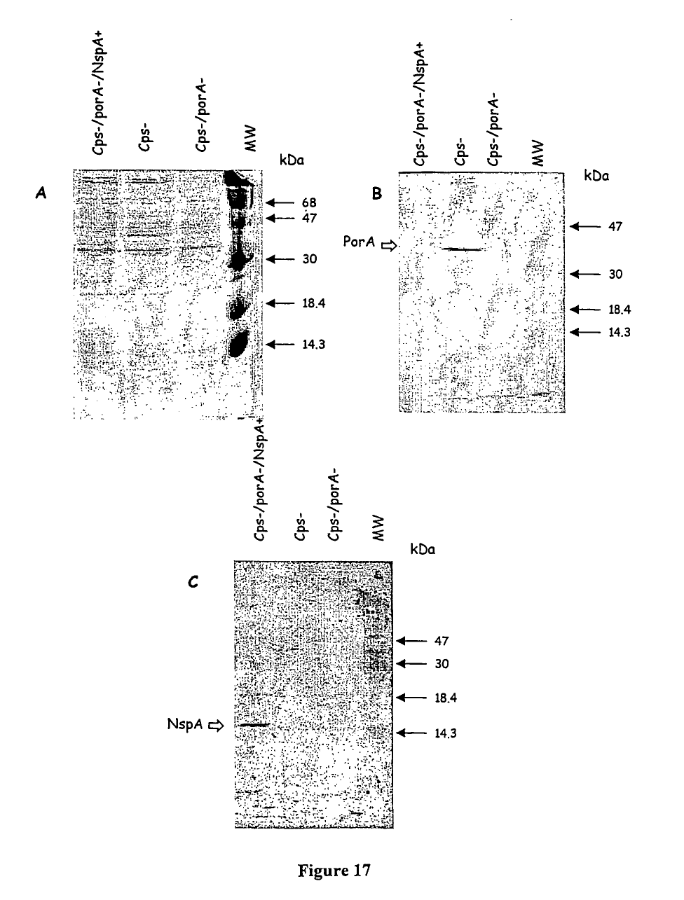

The aim of the experiment was to replace the endogenous promoter region of the D15/Omp85 gene by the strong porA promoter in order to up-regulate the production of the D15/Omp85 antigen. For that purpose, a promoter replacement plasmid was constructed using E. coli cloning methodologies. A DNA region (1000 bp) located upstream from the D15/omp85 coding gene was discovered (SEQ ID NO:3) in the private Incyte PathoSeq data base containing unfinished genomic DNA sequences of the Neisseria meningitidis strain ATCC 13090. The main steps of this procedure are represented in FIG. 9. Briefly, a DNA fragment (1000 bp) covering nucleotides −48 to −983 with respect to the D15/Omp85 gene start codon (ATG) was PCR amplified using oligonucleotides ProD15-51X (5′-GGG CGA ATT CGC GGC CGC CGT CAA CGG CAC ACC GTT G-3′) and ProD15-52 (5′-GCT CTA GAG CGG AAT GCG GTT TCA GAC G-3′) containing EcoRI and XbaI restriction sites (underlined) respectively. This fragment was submitted to restriction and inserted in pUC18 plasmid restricted with the same enzymes. The construct obtained was submitted to in vitro mutagenesis using the Genome Priming system (using the pGPS2 donor plasmid) commercialized by New England Biolabs (MA, USA). Clones having inserted a mini-transposon (derived from Tn7 and harboring a chloramphenicol resistance gene) were selected. One clone containing a mini-transposon insertion located in the D15/Omp85 5′ flanking region, 401 bp downstream from the EcoRI site was isolated and used for further studies. This plasmid was submitted to circle PCR mutagenesis (Jones & Winistofer (1992), Biotechniques12: 528-534) in order to (i) delete a repeated DNA sequence (Tn7R) generated by the transposition process, (ii) insert meningococcal uptake sequences required for transformation, and (iii) insert suitable restriction sites allowing cloning of foreign DNA material such as promoters. The circle PCR was performed using the TnRD15-KpnI/XbaI+US (5′-CGC CGG TAC CTC TAG AGC CGT CTG AAC CAC TCG TGG ACA ACC C-3′) & TnR03Cam(KpnI) (5′-CGC CGG TAC CGC CGC TAA CTA TAA CGG TC-3′) oligonucleotides containing uptake sequences and suitable restriction sites (KpnI and XbaI) underlined. The resulting PCR fragment was gel-purified, digested with Asp718 (isoschizomer of KpnI) and ligated to a 184 bp DNA fragment containing the porA promoter and generated by PCR using the PorA-01 (5′-CGC CGG TAC CGA GGT CTG CGC TTG AAT TGT G-3′) and PorA02 (5′-CGC CGG TAC CTC TAG ACA TCG GGC AAA CAC CCG-3′) oligonucleotides containing KpnI restriction sites. Recombinant clones carrying a porA promoter inserted in the correct orientation (transcription proceeding in the EcoRI to XbaI direction) were selected and used to transform a strain of Neisseria meningitidis serogroup B lacking capsular polysaccharide (cps−) and one of the major outer membrane proteins—PorA (porA−). Recombinant Neisseria meningitidis clones resulting from a double crossing over event (PCR screening using oligonucleotides Cam-05 (5′-GTA CTG CGA TGA GTG GCA GG-3′) & proD15-52) were selected on GC medium containing 51 μg/ml chloramphenicol and analyzed for D15/Omp85 expression. As represented in FIG. 10, the production of D15/Omp85 was significantly increased in the total protein extracts of Nm strains resulting from promoter replacement, when compared to parental strain (cps−). This result was also observed when analyzing outer-membrane blebs prepared from the same strains (see FIG. 17). These results are attributable to the replacement of the endogenous D15 promoter by the strong porA promoter. In addition, it was surprisingly found that expression, where the porA promoter was introduced approximately 400 bp upstream of the initiator codon, was approximately 50 times greater than when the promoter was placed approximately 100 bp upstream. Altogether, these experiments support that the promoter replacement strategy works and allows the up-regulation of the synthesis of integral outer-membrane proteins in outer-membrane blebs.

Certain geographically isolated human populations (such as Cuba) are infected by a limited number of Neisseiria meningitidis isolates belonging largely to one or few outer membrane protein serotypes. Since PorA is a major outer-membrane protein antigen which can induce protective and strain-specific bactericidal antibodies, it may be possible to confer vaccine protection in such a population using a limited number of porA serotypes. Moreover, PorA may interact with or stabilize some other outer membrane proteins. In this context, the presence of PorA in outer membrane vesicles may be advantageous, strengthening the vaccine efficacy of such recombinant improved blebs.

For such a reason, it may be desirable to up-regulate the expression of D15/Omp85 outer membrane protein in a Neisseria meningitidis serogroup B strain lacking functional cps genes but expressing PorA. Genomic DNA was extracted from the recombinant Neisseria meningitidis serogroup B cps−, porA−, D15/Omp85+ strain using the QIAGEN Genomic Tips 100-G kit. 10 μgr of this material was linearized and used to transform Neisseria meningitidis serogroup B cps− following a classical transformation protocol. Recombinant Neisseria were obtained on GC agar plates containing 5 μgr/ml chloramphenicol.

Integrations resulting from a double crossing-over upstream of the D15 gene were screened by PCR as described previously. As homologous recombinations can occur everywhere in the chromosome, a second PCR screening was performed to control the integrity of the porA locus in the recombinant strain. For this purpose, internal porA primers PPA1 (5-GCG GCC GTT GCC GAT GTC AGC C-3′) and PpA2 (5-GGC ATA GCT GAT GCG TGG AAC TGC-3′) were used in a PCR screening experiment. The amplification of an 1170 bp fragment confirms the presence of the porA gene in the recombinant bacteria.

Recombinant bacteria (corresponding to about 5.10⁸ bacteria) can be re-suspended in 50 μl of PAGE-SDS buffer, frozen(−20° C.)/boiled (100° C.) three times and then separated by PAGE-SDS electrophoresis on a 12.5% gel. Gels can then be stained by Coomassie Brilliant blue R250 or transferred to a nitrocellulose membrane and probed either with an anti-porA monoclonal antibody or with an anti-D15/Omp85 rabbit polyclonal antibody. Analysis of outer-membrane blebs prepared from the same strains can also be performed.

### Example 11

### Up-Regulation of the Hsf Protein Antigen in a Recombinant Neisseiria meningitidis Serogroup B Strain Lacking Functional cps Genes but Expressing PorA

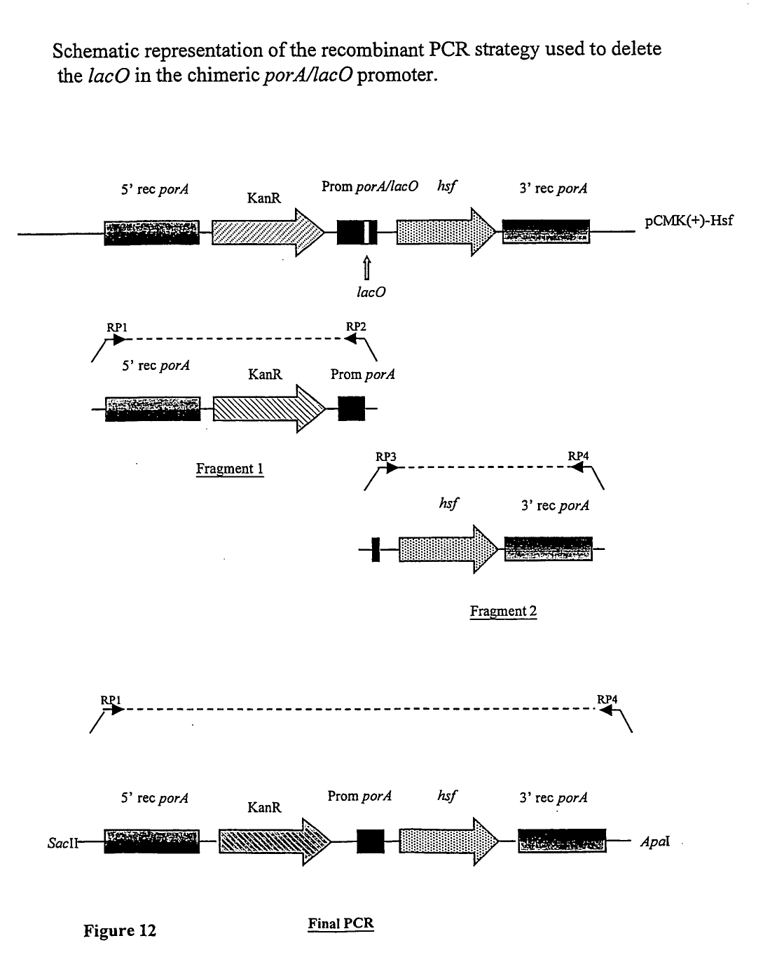

As described above, in certain countries, the presence of PorA in outer membrane vesicles may be advantageous, and can strengthen the vaccine efficacy of recombinant improved blebs. In the following example, we have used a modified pCMK(+) vector to up-regulate the expression of the Hsf protein antigen in a strain lacking functional cps genes but expressing PorA The original pCMK(+) vector contains a chimeric porA/lacO promoter repressed in E. coli host expressing lacIq but transcriptionally active in Neisseria meningitidis. In the modified pCMK(+), the native porA promoter was used to drive the transcription of the hsf gene. The gene coding for Hsf was PCR amplified using the HSF 01-NdeI and HSF 02-NheI oligonucleotide primers, presented in the table below. Because of the sequence of the HSF 01-NdeI primer the Hsf protein expressed will contain two methionine residues at the 5′ end. The conditions used for PCR amplification were those described by the supplier (HiFi DNA polymerase, Boehringer Mannheim, GmbH). Thermal cycling was the following: 25 times (94° C. 1 min., 48° C. 1 min., 72° C. 3 min.) and 1 time (72° C. 10 min., 4° C. up to recovery). The corresponding amplicon was subsequently cloned in the corresponding restriction sites of pCMK(+) delivery vector. In this recombinant plasmid, designed pCMK(+)-Hsf, we deleted the lacO present in the chimeric porA/lacO promoter by a recombinant PCR strategy (See FIG. 12). The pCMK(+)-Hsf plasmid was used as a template to PCR amplify 2 separate DNA fragments:

fragment 1 contains the porA 5′ recombinogenic region, the Kanamycin resistance gene and the porA promoter. Oligonucleotide primers used, RP1(SacII) and RP2, are presented in the table below. RP1 primer is homologous to the sequence just upstream of the lac operator.

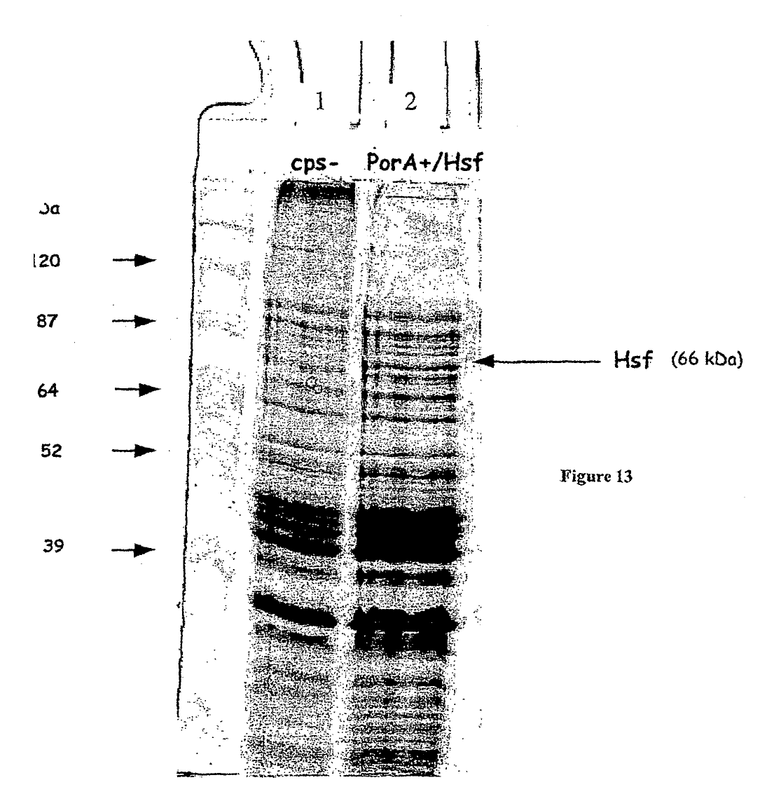

fragment 2 contains the Shine-Dalgarno sequence from the porA gene, the hsf gene and the porA 3′ recombinogenic region. Oligonucleotide primers used, RP3 and RP4(ApaI), are presented in the table below. RP3 primer is homologous to the sequence just downstream of the lac operator. The 3′ end of fragment 1 and the 5′end of fragment 2 have 48 bases overlapping. 500 ng of each PCR (1 and 2) were used for a final PCR reaction using primers RP1 and RP4. The final amplicon obtained was subcloned in pSL1180 vector restricted with SacII and ApaI. The modified plasmid pCMK(+)-Hsf was purified at a large scale using the QIAGEN maxiprep kit and 2 μg of this material was used to transform a Neisseiria meningitidis serogroup B strain lacking functional cps genes (the strain described in example 1). In order to preserve the expression of porA, integration resulting from a single crossing-over was selected by a combination of PCR and Western blot screening procedures. Kanamycin resistant clones testing positive by porA-specific PCR and western blot were stored at −70° C. as glycerol stocks and used for further studies. Bacteria (corresponding to about 5.10⁸ bacteria) were re-suspended in 50 μl of PAGE-SDS buffer, frozen (−20° C.)/boiled (100° C.) three times and then were separated by PAGE-SDS electrophoresis on a 12.5% gel. The expression of Hsf was examined in Whole-cell bacterial lysates (WCBL) derived from NmB [Cps−, PorA+] or NmB [Cps−, PorA+, Hsf+]. Coomassie staining detected a significant increase in the expression of Hsf (with respect to the endogenous Hsf level) (See in FIG. 13). This result confirms that the modified pCMK(+)-Hsf vector is functional and can be used successfully to up-regulate the expression of outer membrane proteins, without abolishing the production of the major PorA outer membrane protein antigen.

Oligonucleotides Used in This Work

Oligonucleotides used in this work

| | | |
|---|---|---|
| Oligonucleotides | Sequence | Remark(s) |

| | | |
|---|---|---|
| Hsf 01-Nde | 5′-GGA ATT CCA TAT GAT GAA CAA | NdeI cloning site |
| | AAT ATA CCG C-3′ | |
| Hsf 02-Nhe | 5′-GTA GCT AGC TAG CTT ACC ACT | Nhe I cloning site |
| | GAT AAC CGA C-3′ | |
| GFP-mut-Asn | 5′-AAC TGC AGA ATT AAT ATG AAA | AsnI cloning site |
| | GGA GAA GAA CTT TTC-3′ | Compatible with NdeI |
| GFP-Spe | 5′-GAC ATA CTA GTT TAT TTG TAG | SpeI cloning site |
| | AGC TCA TCC ATG-3′ | Compatible with NHeI |
| RP1 (SacII) | 5′-TCC CCG CGG GCC GTC TGA ATA | SacII cloning site |
| | CAT CCC GTC-3′ | |
| RP2 | 5′-CAT ATG GGC TTC CTT TTG TAA | |
| | ATT TGA GGG CAA ACA CCC GAT ACG | |
| | TCT TCA-3′ | |
| RP3 | 5′-AGA CGT ATC GGG TGT TTG CCC | |
| | TCA AAT TTA CAA AAG GAA GCC CAT | |
| | ATG-3′ | |
| RP4(ApaI) | 5′-GGG TAT TCC GGG CCC TTC AGA | ApaI cloning site |
| | CGG CGC AGC AGG-3′ | |

### Example 12

### Expression of the Green Fluorescent Protein in a Recombinant Neisseria meningitidis Serogroup B Strain Lacking Functional cps Genes but Expressing PorA

In the following example, the pCMK vector was used to test the expression of a cytoplasmic heterologous protein in Neisseria meningitidis. The Green Fluorescent Protein was amplified from the pKen-Gfpmut2 plasmid with the primers GFP-Asn-mut2 and GFP-Spe (see table in Example 11). AsnI gives cohesive ends compatible with NdeI, SpeI gives cohesive ends compatible with NheI. The conditions used for PCR amplification were those described by the supplier (HiFi DNA polymerase, Boehringer Mannheim, GmbH). Thermal cycling was the following: 25 times (94° C. 1 min., 48° C. 1 min., 72° C. 3 min.) and 1 time (72° C. 10 min., 4° C. up to recovery). The corresponding amplicon was subsequently cloned in the pCMK(+) delivery vector digested with NdeI and NheI restriction enzymes. In this recombinant plasmid, designed pCMK(+)-GFP, we deleted the lacO present in the chimeric porA/lacO promoter by a recombinant PCR strategy. The pCMK(+)-GFP plasmid was used as template to PCR amplify 2 separate DNA fragments:

fragment 1 contained the porA 5′ recombinogenic region, the Kanamycin resistance gene and the porA promoter. Oligonucleotide primers used, RP 1 (SacII) and RP2 (see table in example 11). RP1 primer is homologous to the sequence just upstream of the lac operator.

fragment 2 contains the PorA Shine-Dalgamo sequence, the gfp gene and the porA 3′ recombinogenic region. Oligonucleotide primers used, RP3 and RP4(ApaI), are presented in the table in example 11. RP3 primer is homologous to the sequence just downstream of the lac operator.

The 3′end of fragment 1 and the 5′end of fragment 2 have 48 bases overlapping. 500 ng of each PCR (1 and 2) were used for a final PCR reaction using primers RP1 and RP4. Twenty μg of this PCR fragment were used to transform a Neisseiria meningitidis serogroup B strain lacking functional cps genes.

Transformation with linear DNA is less efficient than with circular plasmid DNA but all the recombinants obtained performed a double crossing-over (confirmed by a combination of PCR and Western blot screening procedures). Kanamycin resistant clones were stored at −70° C. as glycerol stocks and used for further studies. Bacteria (corresponding to about 5.10⁸ bacteria) were re-suspended in 50 μl of PAGE-SDS buffer, frozen (−20° C.)/boiled (100° C.) three times and then were separated by PAGE-SDS electrophoresis on a 12.5% gel.

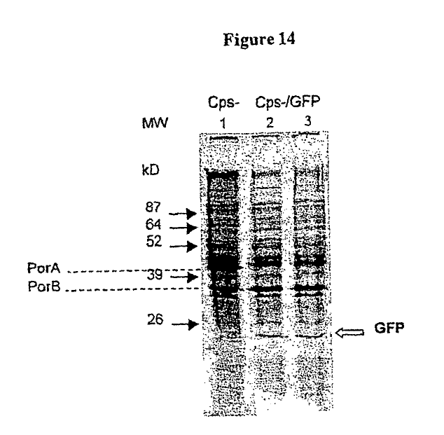

The expression of GFP was examined in Whole-cell bacterial lysates (WCBL) derived from NmB [Cps−, PorA+] or NmB [Cps−, PorA−, GFP+]. Coomassie staining detected an expression of GFP absent in the recipient Neisseria meningitidis strain (see FIG. 14).

### Example 13

### Up-Regulation of the N. meningitidis Serogroup B NspA Gene by Promoter Replacement

The aim of the experiment was to replace the endogenous promoter region of the NspA gene by the strong porA promoter, in order to up-regulate the production of the NspA antigen. For that purpose, a promoter replacement plasmid was constructed using E. coli cloning methodologies. A DNA region (924 bp) located upstream from the NspA coding gene was discovered (SEQ ID NO: 7) in the private Incyte PathoSeq data base containing unfinished genomic DNA sequences of the Neisseria meningitidis strain ATCC 13090. A DNA fragment (675 bp) covering nucleotides −115 to −790 with respect to the NspA gene start codon (ATG) was PCR amplified using oligonucleotides PNS1′ (5′-CCG CGA ATT CGA CGA AGC CGC CCT CGA C-3′) and PNS2 (5′-CGT CTA GAC GTA GCG GTA TCC GGC TGC-3′) containing EcoRI and XbaI restriction sites (underlined) respectively. The PCR fragment was submitted to restriction with EcoRI and XbaI and inserted in pUC18. This plasmid was submitted to circle PCR mutagenesis (Jones & Winistofer (1992), Biotechniques12: 528-534) in order to insert meningococcal uptake sequences required for transformation, and suitable restriction sites allowing cloning of a CmR/PorA promoter cassette. The circle PCR was performed using the BAD01-2 (5′-GGC GCC CGG GCT CGA GCT TAT CGA TGG AAA ACG CAG C-3′) & BAD02-2 (5′-GGC GCC CGG GCT CGA GTT CAG ACG GCG CGC TTA TAT AGT GGA TTA AC-3′) oligonucleotides containing uptake sequences and suitable restriction sites (XmaI and XhoI) underlined. The resulting PCR fragment was gel-purified and digested with XhoI. The CmR/PorA promoter cassette was amplified from the pUC D15/Omp85 plasmid previously described, using primers BAD 15-2 (5′-GGC GCC CGG GCT CGA GTC TAG ACA TCG GGC AAA CAC CCG-3′) & BAD 03-2 (5′-GGC GCC CGG GCT CGA GCA CTA GTA TTA CCC TGT TAT CCC-3′) oligonucleotides containing suitable restriction sites (XmaI, XbaI, SpeI and XhoI) underlined. The PCR fragment obtained was submitted to digestion and inserted in the circle PCR plasmid restricted with the corresponding enzymes. 10 μg of the recombinant plasmid were linearized and used to transform a strain of Neisseria meningitidis serogroup B lacking capsular polysaccharide (cps−) and one of the major outer membrane proteins—PorA (porA−). Recombinant Neisseria meningitidis clones resulting from a double crossing over event [PCR screening using oligonucleotides BAD 25 (5′-GAG CGA AGC CGT CGA ACG C-3′) & BAD08 (5′-CTT AAG CGT CGG ACA TTT CC-3′)] were selected on GC agar plates containing 5 μg/ml chloramphenicol and analyzed for NspA expression. Recombinant bacteria (corresponding to about 5.10⁸ bacteria) were re-suspended in 50 μl of PAGE-SDS buffer, frozen (−20° C.)/boiled (100° C.) three times and then were separated by PAGE-SDS electrophoresis on a 12.5% gel. Gels were then stained by Coomassie Brilliant blue R250 or transferred to a nitrocellulose membrane and probed either with an anti-PorA monoclonal antibody or with anti-NspA polyclonal antibody (FIG. 17). As for Omp85, there is a surprising indication that insertion of the promoter approximately 400 bp upstream of the NspA initiation codon expresses more protein than if placed approximately 100 bp upstream.

The same recombinant pUC plasmid can be used to up-regulate the expression of NspA in a Neisseria meningitidis serogroup B strain lacking functional cps gene but still expressing PorA.

### Example 14

### Up-Regulation of the N. meningitidis Serogroup B pldA (omplA) Gene by Promoter Replacement

The aim of the experiment was to replace the endogenous promoter region of the pldA (omplA) gene by the strong porA promoter in order to up-regulate the production of the PldA (OmplA1) antigen. For that purpose, a promoter replacement plasmid was constructed using E. coli cloning methodologies. A DNA region (373 bp) located upstream from the pldA coding sequence was discovered (SEQ ID NO: 18) in the private Incyte PathoSeq data base of the Neisseria meningitidis strain ATCC 13090. This DNA contains the sequence coding for a putative rpsT gene. The stop codon of rpsT is located 169 bp upstream the pldA ATG. To avoid the disruption of this potentially important gene, we decided to insert the CmR/PorA promoter cassette just upstream of the ATG of pldA. For that purpose, a DNA fragment of 992 bp corresponding to the the rpsT gene, the 169 bp intergenic sequence and the 499 first nucleotides of pldA gene was PCR amplified from Neisseria meningitidis serogroup B genomic DNA using oligonucleotides PLA1 Amo5 (5′-GCC GTC TGA ATT TAA AAT TGC GCG TTT ACA G-3′) and PLA1 Amo3 (5′-GTA GTC TAG ATT CAG ACG GCG CAA TTT GGT TTC CGC AC-3′) containing uptake sequences (underlined). PLA1 Amo3 contains also a XbaI restriction site. This PCR fragment was cleaned with a High Pure Kit (Roche, Mannheim, Germany) and directly cloned in a pGemT vector (Promega, USA). This plasmid was submitted to circle PCR mutagenesis (Jones & Winistofer (1992)) in order to insert suitable restriction sites allowing cloning of a CmR/PorA promoter cassette. The circle PCR was performed using the CIRC1-Bgl (5′CCT AGA TCT CTC CGC CCC CCA TTG TCG-3′) & either CIRC1-XH-RBS/2 (5′-CCG CTC GAG TAC AAA AGG AAG CCG ATA TGA ATA TAC GGA ATA TGC G-3′) or CIRC2-XHO/2 (5′-CCG CTC GAG ATG AAT ATA CGG AAT-3′) oligonucleotides containing suitable restriction sites (BglII and XhoI) underlined. The CmR/PorA promoter cassette was amplified from the pUC D15/Omp85 plasmid previously described, using primers BAD20 (5′-TCC CCC GGG AGA TCT CAC TAG TAT TAC CCT GTT ATC CC-3′) and CM-PORA-3 (5′-CCG CTC GAG ATA AAA ACC TAA AAA CAT CGG GC-3′) containing suitable restriction sites (BglII and XhoI) underlined. This PCR fragment was cloned in the circle PCR plasmid obtained with primers CIRC1-Bgl and CIRC1-XH—RBS/2. This plasmid can be used to transform Neisseria meningitidis serogroup B [cps−] and [cps− porA−] strains. Integration by double crossing-over in the upstream region of pldA will direct the insertion of the porA promoter directly upstream of the pldA ATG. Another cassette was amplified from the genomic DNA of the recombinant Neisseria meningitidis serogroup B [cps−, porA−, D15/Omp85+] over-expressing D15/Omp85 by promoter replacement. This cassette contains the cmR gene, the porA promoter and 400 bp corresponding to the 5′ flanking sequence of the D15/Omp85 gene. This sequence has been proven to be efficacious for up-regulation of the expression of D15/Omp85 in Neisseria and will be tested for the up-regulation of the expression of other Neisseria antigens. Primers used for the amplification were BAD 20 and CM-PORA-D15/3 (5′-CGG CTC GAG TGT CAG TTC CTT GTG GTG C-3′) containing XhoI restriction sites (underlined). This PCR fragment was cloned in the circle PCR plasmid obtained with primers CIRC1-Bgl and CIRC2-XHO/2. This plasmid will be used to transform Neisseria meningitidis serogroup B [cps−] and [cps−, porA−] strains. Integration by double crossing-over in the upstream region of pldA will direct the insertion of the porA promoter 400 bp upstream the pldA ATG.

### Example 15

### Up-Regulation of the N. meningitidis Serogroup B tbpA Gene by Promoter Replacement

The aim of the experiment was to replace the endogenous promoter region of the tbpA gene by the strong porA promoter, in order to up-regulate the production of the TbpA antigen. For that purpose, a promoter replacement plasmid was constructed using E. coli cloning methodologies. A DNA region (731 bp) located upstream from the tbpA coding sequence was discovered (SEQ ID NO: 17) in the private Incyte PathoSeq data base of the Neisseria meningitidis strain ATCC 13090. This DNA contains the sequence coding for TbpB antigen. The genes are organized in an operon. The tbpB gene will be deleted and replaced by the CmR/porA promoter cassette. For that purpose, a DNA fragment of 3218 bp corresponding to the 509 bp 5′ flanking region of tbpB gene, the 2139 bp tbpB coding sequence, the 87 bp intergenic sequence and the 483 first nucleotides of tbpA coding sequence was PCR amplified from Neisseria meningitidis serogroup B genomnic DNA using oligonucleotides BAD16 (5′-GGC CTA GCT AGC CGT CTG AAG CGA TTA GAG TTT CAA AAT TTA TTC-3′) and BAD17 (5′-GGC CAA GCT TCA GAC GGC GTT CGA CCG AGT TTG AGC CTT TGC-3′) containing uptake sequences and NheI and HindIII restriction sites (underlined). This PCR fragment was cleaned with a High Pure Kit ( Boerhinger Mannheim, Germany) and directly cloned in a pGemT vector (Promega, USA). This plasmid was submitted to circle PCR mutagenesis (Jones & Winistofer (1992)) in order to (i) insert suitable restriction sites allowing cloning of a CmR/PorA promoter cassette and (ii) to delete 209 bp of the 5′ flanking sequence of tbpB and the tbpB coding sequence. The circle PCR was performed using the BAD 18 (5′-TCC CCC GGG AAG ATC TGG ACG AAA AAT CTC AAG AAA CCG-3′) & the BAD 19 (5′-GGA AGA TCT CCG CTC GAG CAA ATT TAC AAA AGG AAG CCG ATA TGC AAC AGC AAC ATT TGT TCC G-3′) oligonucleotides containing suitable restriction sites XmaI, BglII and XhoI (underlined). The CmR/PorA promoter cassette was amplified from the pUC D15/Omp85 plasmid previously described, using primers BAD21 (5′-GGA AGA TCT CCG CTC GAG ACA TCG GGC AAA CAC CCG-3′) & BAD20 (5′-TCC CCC GGG AGA TCT CAC TAG TAT TAC CCT GTT ATC CC-3′) containing suitable restriction sites XmaI, SpeI, BglII and XhoI (underlined). This PCR fragment was cloned in the circle PCR plasmid. This plasmid will be used to transform Neisseria meningitidis serogroup B [cps−] and [cps− porA−] strains. Integration by double crossing-over in the upstream region of tbpA will direct the insertion of the porA promoter directly upstream of the tbpA ATG.

### Example 16

### Up-Regulation of the N. meningitidis Serogroup B pilQ Gene by Promoter Replacement

The aim of the experiment was to replace the endogenous promoter region of the pilQ gene by the strong porA promoter, in order to up-regulate the production of the PilQ antigen. For that purpose, a promoter replacement plasmid was constructed using E. coli cloning methodologies. A DNA region (772 bp) located upstream from the pilQ coding gene was discovered (SEQ ID NO: 12) in the private Incyte PathoSeq data base of the Neisseria meningitidis strain ATCC 13090. This DNA contains the sequence coding for PilP antigen. The pilQ gene is part of an operon we do not want to disturb, pilins being essential elements of the bacteria The CmR/porA promoter cassette was introduced upstream the pilQ gene following the same strategy described for the up-regulation of the expression of the pldA gene. For that purpose, a DNA fragment of 866 bp corresponding to the 3′ part of the pilP coding sequence, the 18 bp intergenic sequence and the 392 first nucleotides of pilQ gene was PCR amplified from Neisseria serogroup B genomic DNA using PQ-rec5-Nhe (5′-CTA GCT AGC GCC GTC TGA ACG ACG CGA AGC CAA AGC-3′) and PQ-rec3-Hin (GCC AAG CTT TTC AGA CGG CAC GGT ATC GTC CGA TTC G-3′) oligonucleotides containing uptake sequences and NheI and HindIII restriction sites (underlined). This PCR fragment was directly cloned in a pGemT vector (Promega, USA). This plasmid was submitted to circle PCR mutagenesis (Jones & Winistofer (1992)) in order to insert suitable restriction sites allowing cloning of a CmR/PorA promoter cassette. The circle PCR was performed using the CIRC1-PQ-Bgl (5′-GGA AGA TCT AAT GGA GTA ATC CTC TTC TTA-3′) & either CIRC1-PQ-XHO (5′-CCG CTC GAG TAC AAA AGG AAG CCG ATA TGA TTA CCA AAC TGA CAA AAA TC-3′) or CIRC2-PQ-X (5′-CCG CTC GAG ATG AAT ACC AAA CTG ACA AAA ATC-3′) oligonucleotides containing suitable restriction sites BglII and XhoI (underlined). The CmR/PorA promoter cassette was amplified from the pUC D15/Omp85 plasmid previously described, using primers BAD20 (5′-TCC CCC GGG AGA TCT CAC TAG TAT TAC CCT GTT ATC CC-3′) and CM-PORA-3 (5′-CCG CTC GAG ATA AAA ACC TAA AAA CAT CGG GCA AAC ACC C-3′) containing suitable restriction sites BglII and XhoI (underlined). This PCR fragment was cloned in the circle PCR plasmid obtained with primers CIRC1-PQ-Bgl and CIRC1-PQ-XHO. This plasmid can be used to transform Neisseria meningitidis serogroup B [cps−] and [cps−, porA−] strains. Integration by double crossing-over in the upstream region of pilQ will direct the insertion of the porA promoter directly upstream of the pilQ ATG.

Another cassette was amplified from the genomic DNA of the recombinant Neisseria meningitidis serogroup B [cps−, porA−, D15/Omp85+] over-expressing D15/Omp85 by promoter replacement. This cassette contains the cmR gene, the porA promoter and 400 bp corresponding to the 5′ flanking sequence of the D15/Omp85 gene. This sequence has been proven to be efficacious for up-regulation of the expression of D15/Omp85 in Neisseria meningitidis and will be tested for the up-regulation of the expression of other Neisseria antigens. Primers used for the amplification were BAD 20 and CM-PORA-D153 (5′-GGG CTC GAG TGT CAG TTC CTT GTG GTG C-3′) containing XhoI restriction sites (underlined). This PCR fragment was cloned in the circle PCR plasmid obtained with primers CIRC1-PQ-Bgl and CIRC2-PQ-X. This plasmid can be used to transform Neisseria meningitidis serogroup B [cps−] and [cps−, porA−] strains. Integration by double crossing-over in the upstream region of pilQ will direct the insertion of the porA promoter 400 bp upstream the pilQ ATG.

### Example 17

### Construction of a kanR/sacB Cassette for Introducing “Clean”, Unmarked Mutations in the N. meningitidis Chromosome

The aim of the experiment is to construct a versatile DNA cassette containing a selectable marker for the positive screening of recombination in the chromosome of Neisseria meningitidis (ie: kanR gene), and a counter selectable marker to delete the cassette from the chromosome after recombination (ie: sacB gene). By this method, any heterologous DNA introduced during homologous recombination will be removed from the Neisseria chromosome.

A DNA fragment containing the neoR gene and the sacB gene expressed under the control of its own promoter was obtained by restriction of the pIB 279 plasmid (Blomfield I C, Vaughn V, Rest R F, Eisenstein B I (1991), Mol Microbiol 5:1447-57) with BamHI restriction enzyme. The recipient vector was derived from plasmid pCMK, previously described. The kanR gene of the pCMK was deleted by restriction with enzymes NruI and EcoRV. This plasmid was named pCMKs. The neoR/sacB cassette was inserted in the pCMKs at a Bg/II restriction site compatible with BamHI ends.

E. coli harboring the plasmid is unable to grow in the presence of 2% sucrose in the culture medium, confirming the functionality of the sacB promoter. This plasmid contains recombinogenic sequences allowing the insertion of the cassette at the porA locus in the chromosome of Neisseria meningitidis serogroup B. Recombinant Neisseria were obtained on GC agar plates containing 200 μg/ml of kanamycin. Unfortunately, the sacB promoter was not functional in Neisseria meningitidis: no growth difference was observed on GC agar plates containing 2% sucrose.

A new cassette was constructed containing the sacB gene under the control of the kanR promoter. A circle PCR was performed using the plasmid pUC4K ((Amersham Pharmacia Biotech, USA)) as a template with CIRC-Kan-Nco (5′-CAT GCC ATG GTT AGA AAA ACT CAT CGA GCA TC-3′) & CIRC-Kan-Xba (5′-CTA GTC TAG ATC AGA ATT GGT TAA TTG GTT G-3′) oligonucleotides containing NcoI and XbaI restriction sites (underlined). The resulting PCR fragment was gel-purified, digested with NcoI and ligated to the sacB gene generated by PCR from the pIB279 plasmid with SAC/NCO/NEW5 (5′-CAT GCC ATG GGA GGA TGA ACG ATG AAC ATC AAA AAG TTT GCA A-3′) oligonucleotide containing a NcoI restriction site (underlined) and a RBS (bold) & SAC/NCO/NEW3 (5′-GAT CCC ATG GTT ATT TGT TAA CTG TTA ATT GTC-3′) oligonucleotide containing a NcoI restriction site (underlined). The recombinant E. coli clones can be tested for their sensitivity on agar plates containing 2% sucrose. The new kanR/sacB cassette can be subcloned in the pCMKs and used to transform a Neisseria meningitidis serogroup B cps− strain. The acquired sucrose sensitivity will be confirmed in Neisseria. The pCMKs plasmid will be used to transform the recombinant kanR/SacB Neisseria to delete the entire cassette inserted in the chromosome at the porA locus. Clean recombinant Neisseria will be obtained on GC agar plates containing 2% sucrose.

### Example 18

### Use of Small Recombinogenic Sequences (43 bp) to Allow Homologous Recombination in the Chromosome of Neisseria meningitidis

The aim of the experiment is to use small recombinogenic sequences (43 bp) to drive insertions, modifications or deletions in the chromosome of Neisseria. The achievement of this experiment will greatly facilitate future work, in terms of avoiding subcloning steps of homologous sequences in E. coli (recombinogenic sequences of 43 bp can easily be added in the PCR amplification primer). The kanR gene was PCR amplified from plasmid pUC4K with oligonucleotides Kan-PorA-5 (5′-GCC GTC TGA ACC CGT CAT TCC CGC GCA GGC GGG AAT CCA GTC CGT TCA GTT TCG GGA AAG CCA CGT TGT GTC-3′) containing 43 bp homologous to the 5′ flanking sequence of NmB porA gene (bold) and an uptake sequence (underlined) & Kan-PorA-3 (5′-TTC AGA CGG CGC AGC AGG AAT TTA TCG GAA ATA ACT GAA ACC GAA CAG ACT AGG CTG AGG TCT GCC TCG-3′) containing 43 bp homologous to the 3′ flanking sequence of NmB porA gene (bold) and an uptake sequence (underlined). The 1300 bp DNA fragment obtained was cloned in pGemT vector (Promega, USA). This plasmid can be used to transform a Neisseria meningitidis serogroupB cps− strain. Recombinant Neisseria will be obtained on GC plates containing 200 μg/ml kanamycin. Integrations resulting from a double crossing-over at the porA locus will be screened by PCR with primers PPA1 & PPA2 as described previously.

### Example 19

### Active Protection of Mice Immunized with WT and Recombinant Neisseria meningitidis Blebs

Animals were immunised three times (IP route) with 5 μg of the different OMVs adsorbed on Al(OH)3 on days 0, 14 and 28. Bleedings were done on days 28 (day 14 Post II) and 35 (day 7 post III), and they were challenged on day 35 (IP route). The challenge dose was 20× LD50 (˜10⁷ CFU/mouse). Mortality rate was monitored for 7 days after challenge.

OMVs injected were:

Group1: Cps−, PorA+ blebs

Group2: Cps−, PorA− blebs

Group3: Cps−, PorA−, NspA+ blebs

Group4: Cps−, PorA−, Omp85+ blebs

Group5: Cps−, PorA−, Hsf+ blebs

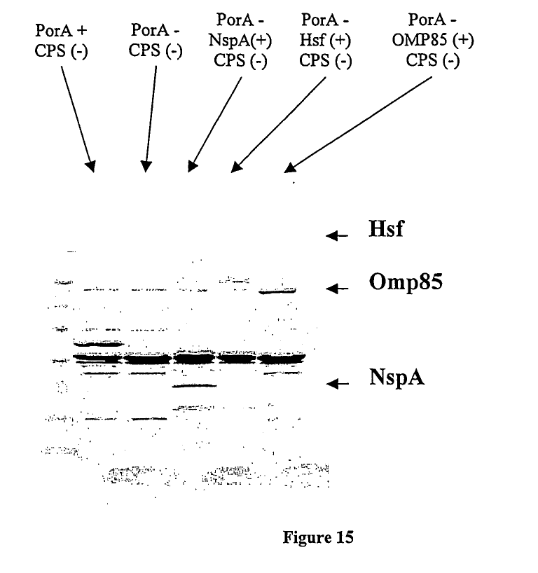

FIG. 15 illustrates the pattern of these OMVs by analyzed SDS Page (Coomassie staining).

24 hours after the challenge, there was 100% mortality (8/8) in the negative control group (immunised with Al(OH)₃ alone) while mice immunised with the 5 different OMVs preparations were still alive (7 to 8/8 mice survived). Sickness was also monitored during the 7 days and the mice immunised with the NSPA over-expressed blebs appeared to be less sick than the other groups. PorA present in PorA+ blebs is likely to confer extensive protection against infection by the homologous strain. However, protection induced by PorA− up-regulated blebs is likely to be due at least to some extent, to the presence of increased amount of NspA, Omp85 or Hsf.

### Example 20

### Immunogenicity of Recombinant Blebs Measured by Whole Cell & Specific ELISA Methods

To measure the ability of the antibodies to recognize the antigens present on the MenB cell surface, the pooled mice sera (from Example 19) were tested by whole cell ELISA (using tetracyclin inactivated cells), and titers were expressed as mid-point titers. All types of bleb antibodies induce a high whole cell Ab titer while the negative control group was clearly negative.

WCE(H44/76)
mid-point titer

| Blebs | 14P2 | 14P3 |
|---|---|---|
| CPS(−) | 23849 | 65539 |
| PorA(+) | | |
| CPS(−) | 20130 | 40150 |
| PorA(−) | | |
| CPS(−) | 8435 | 23846 |
| PorA(−) | | |
| NSPA(+) | | |
| CPS(−) | 4747 | 16116 |
| PorA(−) | | |
| OMP85(+) | | |
| CPS(−) | 6964 | 22504 |
| PorA(−) | | |
| HSF(+) | | |
| (−) | 51 | 82 |

The specific Ab response to available recombinant HSF protein was carried out. Microplates were coated with 1 μg/ml full length HSF molecule.

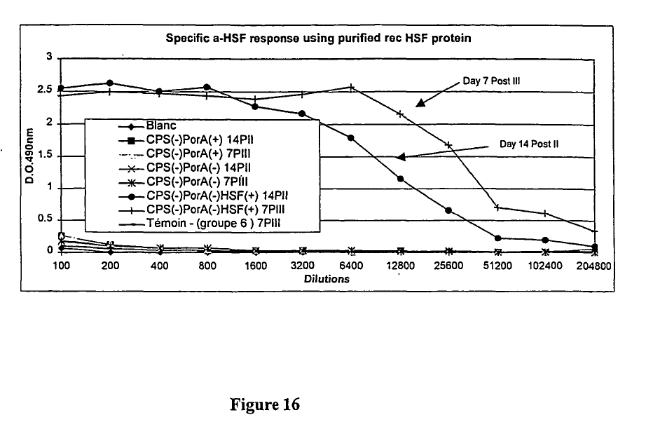

The results illustrated in FIG. 16 show that there was a good specific HSF response when HSF over-expressed OMVs were used to immunize mice (using purified recombinant HSF on the plates). The HSF over-expressed blebs induce a good level of specific antibodies.

### Example 21

### The Immunostimulant Effect of Moraxella catarrhalis Outer Membrane Vesicles (OMV or Blebs) Evaluated on Haemophilus influenzae Protein D (PD), Alone or Conjugated to Streptococcus pneumoniae Polysaccharides (Spn 11V-PD)

The immunostimulant effect of Moraxella catarrhalis outer membrane vesicles (OMV or Blebs) was evaluated on Haemophilus influenzae protein D (PD), alone or conjugated to Streptococcus pneumoniae polysaccharides (Spn 11V-PD).

Experimental Procedure

Groups of 18 mice were subcutaneously immunized on day 0 and 14. Protein D (10 μg) and the Spn 11V-PD conjugate (1 human dose) were injected either alone or adjuvanted with Moraxella blebs (10 μg). On day 20, 27 or 35, mice were bled and anti-protein D titres were measured in an ELISA using purified recombinant protein D. The titres are defined as mid-point titres calculated by 4-parameter logistic model using the XL Fit software.

Results

Serum antibody titers against PD

| Antigens | Geometric mean titre (CI 95%) |
|---|---|
| PD^{a} | 228 (138-376) |
| PD + M. catarrhalis Blebs^{a} | 2871 (1476-5586) |
| M. catarrhalis Blebs^{a} | 52 (19-139) |
| Spn 11V-PD^{a} | 2161 (989-4719) |
| Spn 11V-PD + M. catarrhalis Blebs^{a} | 11518 (6960-19060) |
| M. catarrhalis Blebs^{a} | 71 (22-230) |
| Spn 11V-PD^{b} | 39498 (28534-54676) |
| Spn 11V-PD + M. catarrhalis Blebs^{b} | 55110 (45188-67210) |
| M. catarrhalis Blebs^{b} | 66 (53-81) |
| Spn 11V-PD^{c} | 94570 (65387-136778) |
| Spn 11V-PD + M. catarrhalis Blebs^{c} | 63310 (48597-82478) |
| M. catarrhalis Blebs^{c} | 58 (42-80) |

^{a}animals were bled on day 21
^{b}animals were bled on day 27
^{c}animals were bled on day 35

It can be observed that when antigens are formulated with a bleb adjuvant in a vaccine, this vaccine can induce a faster immune response against the antigen (as well as a larger response). The adjuvant is therefore particularly suitable for vaccines for the elderly (over 55 years of age). The PD immunogenicity (and protective capacity against Haemophilus influenzae) may be significantly enhanced by the presence of blebs as an adjuvant.

| SEQ. ID NO:1 | |
|---|---|
| Nucleotide sequence of the pCMK(+) vector | |
| TCTTCCGCTTCCTCGCTCACTGACTCGCTGCGCTCGGTCGTTCGGCTGCGGCGAGCGGTATCAGCTCACTCAAAGGCGGT | |
| AATACGGTTATCCACAGAATCAGGGGATAACGCAGGAAAGAACATGTGAGCAAAAGGCCAGCAAAAGGCCAGGAACCGTA | |
| AAAAGGCCGCGTTGCTGGCGTTTTTCCATAGGCTCCGCCCCCCTGACGAGCATCACAAAAATCGACGCTCAAGTCAGAGG | |
| TGGCGAAACCCGACAGGACTATAAAGATACCAGGCGTTTCCCCCTGGAAGCTCCCTCGTGCGCTCTCCTGTTCCGACCCT | |
| GCCGCTTACCGGATACCTGTCCGCCTTTCTCCCTTCGGGAAGCGTGGCGCTTTCTCATAGCTCACGCTGTAGGTATCTCA | |
| GTTCGGTGTAGGTCGTTCGCTCCAAGCTGGGCTGTGTGCACGAACCCCCCGTTCAGCCCGACCGCTGCGCCTTATCCGGT | |
| AACTATCGTCTTGAGTCCAACCCGGTAAGACACGACTTATCGCCACTGGCAGCAGCCACTGGTAACAGGATTAGCAGAGC | |
| GAGGTATGTAGGCGGTGCTACAGAGTTCTTGAAGTGGTGGCCTAACTACGGCTACACTAGAAGAACAGTATTTGGTATCT | |
| GCGCTCTGCTGAAGCCAGTTACCTTCGGAAAAAGAGTTGGTAGCTCTTGATCCGGCAAACAAACCACCGCTGGTAGCGGT | |
| GGTTTTTTTGTTTGCAAGCAGCAGATTACGCGCAGAAAAAAAGGATCTCAAGAAGATCCTTTGATCTTTTCTACGGGGTC | |
| TGACGCTCAGTGGAACGAAAACTCACGTTAAGGGATTTTGGTCATGAGATTATCAAAAAGGATCTTCACCTAGATCCTTT | |
| TAAATTAAAAATGAAGTTTTAAATCAATCTAAAGTATATATGAGTAAACTTGGTCTGACAGTTACCAATGCTTAATCAGT | |
| GAGGCACCTATCTCAGCGATCTGTCTATTTCGTTCATCCATAGTTGCCTGACTCCCCGTCGTGTAGATAACTACGATACG | |
| GGAGGGCTTACCATCTGGCCCCAGTGCTGCAATGATACCGCGAGACCCACGCTCACCGGCTCCAGATTTATCAGCAATAA | |
| ACCAGCCAGCCGGAAGGGCCGAGCGCAGAAGTGGTCCTGCAACTTTATCCGCCTCCATCCAGTCTATTAATTGTTGCCGG | |
| GAAGCTAGAGTAAGTAGTTCGCCAGTTAATAGTTTGCGCAACGTTGTTGCCATTGCTACAGGCATCGTGGTGTCACGCTC | |
| GTCGTTTGGTATGGCTTCATTCAGCTCCGGTTCCCAACGATCAAGGCGAGTTACATGATCCCCCATGTTGTGCAAAAAAG | |
| CGGTTAGCTCCTTCGGTCCTCCGATCGTTGTCAGAAGTAAGTTGGCCGCAGTGTTATCACTCATGGTTATGGCAGCACTG | |
| CATAATTCTCTTACTGTCATGCCATCCGTAAGATGCTTTTCTGTGACTGGTGAGTACTCAACCAAGTCATTCTGAGAATA | |
| GTGTATGCGGCGACCGAGTTGCTCTTGCCCGGCGTCAATACGGGATAATACCGCGCCACATAGCAGAACTTTAAAAGTGC | |
| TCATCATTGGAAAACGTTCTTCGGGGCGAAAACTCTCAAGGATCTTACCGCTGTTGAGATCCAGTTCGATGTAACCCACT | |
| CGTGCACCCAACTGATCTTCAGCATCTTTTACTTTCACCAGCGTTTCTGGGTGAGCAAAAACAGGAAGGCAAAATGCCGC | |
| AAAAAAGGGAATAAGGGCGACACGGAAATGTTGAATACTCATACTCTTCCTTTTTCAATATTATTGAAGCATTTATCAGG | |
| GTTATTGTCTCATGAGCGGATACATATTTGAATGTATTTAGAAAAATAAACAAATAGGGGTTCCGCGCACATTTCCCCGA | |
| AAAGTGCCACCTGACGTCTAAGAAACCATTATTATCATGACATTAACCTATAAAAATAGGCGTATCACGAGGCCCTTTCG | |
| TCTCGCGCGTTTCGGTGATGACGGTGAAAACCTCTGACACATGCAGCTCCCGGAGACGGTCACAGCTTGTCTGTAAGCGG | |
| ATGCCGGGAGCAGACAAGCCCGTCAGGGCGCGTCAGCGGGTGTTGGCGGGTGTCGGGGCTGGCTTAACTATGCGGCATCA | |
| GAGCAGATTGTACTGAGAGTGCACCATAAAATTGTAAACGTTAATATTTTGTTAAAATTCGCGTTAAATTTTTGTTAAAT | |
| CAGCTCATTTTTTAACCAATAGGCCGAAATCGGCAAAATCCCTTATAAATCAAAAGAATAGCCCGAGATAGGGTTGAGTG | |
| TTGTTCCAGTTTGGAACAAGAGTCCACTATTAAAGAACGTGGACTCCAACGTCAAAGGGCGAAAAACCGTCTATCAGGGC | |
| GATGGCCCACTACGTGAACCATCACCCAAATCAAGTTTTTTGGGGTCGAGGTGCCGTAAAGCACTAAATCGGAACCCTAA | |
| AGGGAGCCCCCGATTTAGAGCTTGACGGGGAAAGCCGGCGAACGTGGCGAGAAAGGAAGGGAAGAAAGCGAAAGGAGCGG | |
| GCGCTAGGGCGCTGGCAAGTGTAGCGGTCACGCTGCGCGTAACCACCACACCCGCCGCGCTTAATGCGCCGCTACAGGGC | |
| GCGTACTATGGTTGCTTTGACGTATGCGGTGTGAAATACCGCACAGATGCGTAAGGAGAAAATACCGCATCAGGCGCCAT | |
| TCGCCATTCAGGCTGCGCAACTGTTGGGAAGGGCGATCGGTGCGGGCCTCTTCGCTATTACGCCAGCTGGCGAAAGGGGG | |
| ATGTGCTGCAAGGCGATTAAGTTGGGTAACGCCAGGGTTTTCCCAGTCACGACGTTGTAAAACGACGGCCAGTGCCAAGC | |
| TTGCCGTCTGAATACATCCCGTCATTCCTCAAAAACAGAAAACCAAAATCAGAAACCTAAAATCCCGTCATTCCCGCGCA | |
| GGCGGGAATCCAGTCCGTTCAGTTTCGGTCATTTCCGATAAATTCCTGCTGCTTTTCATTTCTAGATTCCCACTTTCGTG | |
| GGAATGACGGCGGAAGGGTTTTGGTTTTTTCCGATAAATTCTTGAGGCATTGAAATTCTAGATTCCCGCCTGCGCGGGAA | |
| TGACGGCTGTAGATGCCCGATGGTCTTTATAGCGGATTAACAAAAATCAGGACAAGGCGACGAAGCCGCAGACAGTACAG | |
| ATAGTACGGAACCGATTCACTTGGTGCTTCAGCACCTTAGAGAATCGTTCTCTTTGAGCTAAGGCGAGGCAACGCCGTAC | |
| TTGTTTTTGTTAATCCACTATAAAGTGCCGCGTGTGTTTTTTTATGGCGTTTTAAAAAGCCGAGACTGCATCCGGGCAGC | |
| AGCGCATCGGCCCGCACGAGGTCTCTGGAGTCGCGAGCATCAAGGGCGAATTCTGCAGGGGGGGGGGGGAAAGCCACGTT | |
| GTGTCTCAAAATCTCTGATGTTACATTGCACAAGATAAAAATATATCATCATGAACAATAAAACTGTCTGCTTACATAAA | |
| CAGTAATACAAGGGGTGTTATGAGCCATATTCAACGGGAAACGTCTTGCTCGAGGCCGCGATTAAATTCCAACATGGATG | |
| CTGATTTATATGGGTATAAATGGGCTCGCGATAATGTCGGGCAATCAGGTGCGACAATCTATCGATTGTATGGGAAGCCC | |
| GATGCGCCAGAGTTGTTTCTGAAACATGGCAAAGGTAGCGTTGCCAATGATGTTACAGATGAGATGGTCAGACTAAACTG | |
| GCTGACGGAATTTATGCCTCTTCCGACCATCAAGCATTTTATCCGTACTCCTGATGATGCATGGTTACTCACCACTGCGA | |
| TCCCCGGGAAAACAGCATTCCAGGTATTAGAAGAATATCCTGATTCAGGTGAAAATATTGTTGATGCGCTGGCAGTGTTC | |
| CTGCGCCGGTTGCATTCGATTCCTGTTTGTAATTGTCCTTTTAACAGCGATCGCGTATTTCGTCTCGCTCAGGCGCAATC | |
| ACGAATGAATAACGGTTTGGTTGATGCGAGTGATTTTGATGACGAGCGTAATGGCTGGCCTGTTGAACAAGTCTGGAAAG | |
| AAATGCATAAGCTTTTGCCATTCTCACCGGATTCAGTCGTCACTCATGGTGATTTCTCACTTGATAACCTTATTTTTGAC | |
| GAGGGGAAATTAATAGGTTGTATTGATGTTGGACGAGTCGGAATCGCAGACCGATACCAGGATCTTGCCATCCTATGGAA | |
| CTGCCTCGGTGAGTTTTCTCCTTCATTACAGAAACGGCTTTTTCAAAAATATGGTATTGATAATCCTGATATGAATAAAT | |
| TGCAGTTTCATTTGATGCTCGATGAGTTTTTCTAATCAGAATTGGTTAATTGGTTGTAACACTGGCAGAGCATTACGCTG | |
| ACTTGACGGGACGGCGGCTTTGTTGAATAAATCGAACTTTTGCTGAGTTGAAGGATCAGATCACGCATCTTCCCGACAAC | |
| GCAGACCGTTCCGTGGCAAAGCAAAAGTTCAAAATCACCAACTGGTCCACCTACAACAAAGCTCTCATCAACCGTGGCTC | |
| CCTCACTTTCTGGCTGGATGATGGGGCGATTCAGGCCTGGTATGAGTCAGCAACACCTTCTTCACGAGGCAGACCTCAGC | |
| GCCCCCCCCCCCCTGCAGGAGGTCTGCGCTTGAATTGTGTTGTAGAAACACAACGTTTTTGAAAAAATAAGCTATTGTTT | |
| TATATCAAAATATAATCATTTTTAAAATAAAGGTTGCGGCATTTATCAGATATTTGTTCTGAAAAATGGTTTTTTGCGGG | |
| GGGGGGGGTATAATTGAAGACGTATCGGGTGTTTGCCCGGAATTGTGAGCGGATAACAATTCGATGTTTTTAGGTTTTTA | |
| TCAAATTTACAAAAGGAAGCCCATATGCATCCTAGGCCTATTAATATTCCGGAGTATACGTAGCCGGCTAACGTTAACAA | |
| CCGGTACCTCTAGAACTATAGCTAGCATGCGCAAATTTAAAGCGCTGATATCGATCGCGCGCAGATCTGATTAAATAGGC | |
| GAAAATACCAGCTACGATCAAATCATCGCCGGCGTTGATTATGATTTTTCCAAACGCACTTCCGCCATCGTGTCTGGCGC | |
| TTGGCTGAAACGCAATACCGGCATCGGCAACTACACTCAAATTAATGCCGCCTCCGTCGGTTTGCGCCACAAATTCTAAA | |
| TATCGGGGCGGTGAAGCGGATAGCTTTGTTTTTGACGGCTTCGCCTTCATTCTTTGATTGCAATCTGACTGCCAATCTGC | |
| TTCAGCCCCAAACAAAAACCCGGATACGGAAGAAAAACGGCAATAAAGACAGCAAATACCGTCTGAAAGATTTTCAGACG | |
| GTATTTCGCATTTTTGGCTTGGTTTGCACATATAGTGAGACCTTGGCAAAAATAGTCTGTTAACGAAATTTGACGCATAA | |
| AAATGCGCCAAAAAATTTTCAATTGCCTAAAACCTTCCTAATATTGAGCAAAAAGTAGGAAAAATCAGAAAAGTTTTGCA | |
| TTTTGAAAATGAGATTGAGCATAAAATTTTAGTAACCTATGTTATTGCAAAGGTCTCGAATTGTCATTCCCACGCAGGCG | |
| GGAATCTAGTCTGTTCGGTTTCAGTTATTTCCGATAAATTCCTGCTGCGCCGTCTGAAGAATTCGTAATCATGGTCATAG | |
| CTGTTTCCTGTGTGAAATTGTTATCCGCTCACAATTCCACACAACATACGAGCCGGAAGCATAAAGTGTAAAGCCTGGGG | |
| TGCCTAATGAGTGAGCTAACTCACATTAATTGCGTTGCGCTCACTGCCCGCTTTCCAGTCGGGAAACCTGTCGTGCCAGC | |
| TGCATTAATGAATCGGCCAACGCGCGGGGAGAGGCGGTTTGCGTATTGGGCGC | |
| SEQ. ID NO:2 | |
| Nucleotide sequence of DNA region (997 bp) up-stream from the NspA gene in | |
| the Neisseria meningitidis serogroup A strain Z2491. | |
| GGAACCGAACACGCCGTTCGGTCATACGCCGCCGAAAGGTTTGCCGCAAGACGAAGCCGCCCTCGACATCGAAGACGCGG | |
| TACACGGCGCGCTGGAAAGCGCGGGTTTTGTCCACTACGAAACATCGGCTTTTGCGAAACCAGCCATGCAGTGCCGCCAC | |
| AATTTGAACTACTGGCAGTTCGGCGATTATTTAGGCATAGGCGCGGGCGCGCACGGCAAAATTTCCTATCCCGACCGCAT | |
| CGAGCGCACCGTCCGCCGCCGCCACCCCAACGACTACCTCGCCTTAATGCAAAACCGACCGAGCGAAGCCGTCGAACGCA | |
| AAACCGTCGCCGCCGAAGATTTGCCGTTCGAATTCATGATGAACGCCCTGCGCCTGACCGACGGCGTACCCACCGCGATG | |
| TTGCAGGAGCGCACGGGCGTACCGAGTGCCAAAATCATGGCGCAAATCGAAACGGCAAGGCAAAAAGGCCTGCTGGAAAC | |
| CGACCCCGCCGTATTCCGCCCGACCGAAAAAGGACGCTTGTTTTTAAACGATTTGCTGCAGTGTTTTTTATAGTGGATTA | |
| ACAAAAACCAGTACGGCGTTGCCTCGCCTTAGCTCAAAGAGAACGATTCTCTAAGGTGCTGAAGCACCAAGTGAATCGGT | |
| TCCGTACTATCTGTACTGTCTGCGGCTTCGTCGCCTTGTCCTGATTTTTGTTAATCCACTATATAAGCGCAAACAAATCG | |
| GCGGCCGCCCGGGAAAACCCCCCCGAACGCGTCCGGAAAATATGCTTATCGATGGAAAACGCAGCCGCATCCCCCGCCGG | |
| GCGTTTCAGACGGCACAGCCGCCGCCGGAAATGTCCGACGCTTAAGGCACAGACGCACACAAAAAACCGTATGCCTGCAC | |
| CTGCAACAATCCGACAGATACCGCTGTTTTTTCCAAACCGTTTGCAAGTTTCACCCATCCGCCGCGTGATGCCGCCACCA | |
| CCATTTAAAGGCAACGCGCGGGTTAACGGCTTTGCCG | |
| SEQ. ID NO:3 | |
| Nucleotide sequence of DNA region (1000 bp) up-stream from the D15/Omp85 | |
| gene in the Neisseria meningitidis serogroup B strain ATCC13090. | |
| ACCATTGCCGCCCGCGCCGGCTTCCAAAGCGGCGACAAAATACAATCCGTCAACGGCACACCCGTTGCAGATTGGGGAG | |
| CGCGCAAACCGAAATCGTCCTCAACCTCGAAGCCGGCAAAGTCGCCGTCGGGTTCAGACGGCATCAGGCGCGCAAACGT | |
| CCGCACCATCGATGCCGCAGGCACGCCGGAAGCCGGTAAAATCGCAAAAAACCAAGGCTACATCGGACTGATGCCCTTTA | |
| AAATCACAACCGTTGCCGGTGCCGTGGAAAAAGGCAGCCCCGCCGAAAAAGCAGGCCTGAAACCGGGCGACAGGCTGACT | |
| GCCGCCGACGGCAAACCCATTACCTCATGGCAAGAATGGGCAAACCTGACCCGCCAAAGCCCCGGCAAAAAAATCACCCT | |
| GAACTACGAACGCGCCGGACAAACCCATACCGCCGACATCCGCCCCGATACTGTCGAACAGCCCGACCACACCCTGATCG | |
| GGCGCGTCGGCCTCCGTCCGCAGCCGGACAGGGCGTGGGACGCGCAAATCCGCCGCAGCTACCGTCCGTCTGTTATCCGC | |
| GCATTCGGCATGGGCTGGGAAAAAACCGTTTCCCACTCGTGGACAACCCTCAAATTTTTCGGCAAACTAATCAGCGGCAA | |
| CGCCTCCGTCAGCCATATTTCCGGGCCGCTGACCATTGCCGACATTGCCGGACAGTCCGCCGAACTCGGCTTGCAAAGTT | |
| ATTTGGAATTTTTGGCACTGGTCAGCATCAGCCTCGGCGTGCTGAACCTGCTGCCCGTCCCCGTTTTGGACGGCGGCCAC | |
| CTCGTGTTTTATACTGCCGAATGGATACGCGGCAAACCTTTGGGCGAACGCGTCCAAAACATCGGTTTGCGCTTCGGGCT | |
| TGCCCTCATGATGCTGATGATGGCGGTCGCCTTCTTCAACGACGTTACCCGGCTGCTCGGTTAGATTTTACGTTTCGGAA | |
| TGCCGTCTGAAACCGCATTCCGCACCACAAGGAACTGACA | |
| SEQ. ID NO:4 | |
| Nucleotide sequence of DNA region (1000 bp) up-stream from the Hsf-like gene | |
| from Neisseria meningitidis | |
| ATTCCCGCGCAGGCGGGAATCCAGAAACGCAACGCAACAGGAATTTATCGGAAAAAACAGAAACCTCACCGCCGTCATTC | |
| CCGCAAAAGCGGGAATCTAGAAACACAACGCGGCAGGACTTTATCAGAAAAAACAGAAACCCCACCGCCGTCATTCCCGC | |
| AAAAGCGGGAATCCAGACCCGTCGGCACGGAAACTTACCGGATAAAACAGTTTCCTTAGATTCCACGTCCTAGATTCCCG | |
| CTTTCGCGGGAATGACGAGATTTTAGATTATGGGAATTTATCAGGAATGATTGAATCCATAGAAAAACCACAGGAATCTA | |
| TCAGAAAAAACAGAAACCCCCACCGCGTCATTCCCGCGCAGGCGGGAATCCAGAAACACAACGCGGCAGGACTTTATCGG | |
| AAAAAACCGAAACCCCACCGACCGTCATTCCCGCAAAAGTTGGAATCCAAAAACGCAACGCAACAGGAATTTATCGGAAA | |
| AAACAGAAACCCCCACCGCGTCATTCCCGCGCAGGCGGGAATCCAGAAACACAACGCAACAGGAATTTATCGGAAAAAAC | |
| AGAAACCCCACCGACCGTCATTCCCGCAAAAGCGGGAATCCAGCAACCGAAAAACCACAGGAATCTATCAGCAAAAACAG | |
| AAACCCCCACCGACCGTCATTCCCGCGCAGGCGGGAATCCAGAAACACAACGCGGCAGGACTTTATCGGAAAAAACAGAA | |
| ACCCCACCGACCGTCATTCCCGCAAAAGCTGGAATCCAAAAACGCAACGCAACAGGAATTTATCGGAAAAAACAGAAACC | |
| CCACCGCCGTCATTCCCGCAAAAGCGGGAATCCAGACCCGTCGGCACGGAAACTTACCGGATAAAACAGTTTCCTTAGAT | |
| TCCACGTCCCAGATTCCCGCCTTCGCGGGAATGACGAGATTTTAAGTTGGGGGAATTTATCAGAAAACCCCCAACCCCCA | |
| AAAACCGGGCGGATGCCGCACCATCCGCCCCCAAACCCCGATTTAACCATTCAAACAAACCAAAAGAAAAAACAAA | |
| SEQ. ID NO:5 | |
| Nucleotide sequence of DNA region (772 bp) up-stream from the PilQ gene from | |
| Neisseria meningitidis | |
| GCGATGTCGGGAAGCCTTCTCCCGAATCATTACCCCTTGAGTCGCTGAAAATCGCCCAATCTCCGGAAAACGGCGGCAAT | |
| CATGACGGCAAGAGCAGCATCCTGAACCTCAGTGCCATTGCCACCACCTACCAAGCAAAATCCGTAGAAGAGCTTGCCGC | |
| AGAAGCGGCACAAAATGCCGAGCAAAAATAACTTACGTTAGGGAAACCATGAAACACTATGCCTTACTCATCAGCTTTCT | |
| GGCTCTCTCCGCGTGTTCCCAAGGTTCTGAGGACCTAAACGAATGGATGGCACAAACGCGACGCGAAGCCAAAGCAGAAA | |
| TCATACCTTTCCAAGCACCTACCCTGCCGGTTGCGCCGGTATACAGCCCGCCGCAGCTTACAGGGCCGAACGCATTCGAC | |
| TTCCGCCGCATGGAAACCGACAAAAAAGGGGAAAATGCCCCCGACACCAAGCGTATTAAAGAAACGCTGGAAAAATTCAG | |
| TTTGGAAAATATGCGTTATGTCGGCATTTTGAAGTCTGGACAGAAAGTCTCCGGCTTCATCGAGGCTGAAGGTTATGTCT | |
| ACACTGTCGGTGTCGGCAACTATTTGGGACAAAACTACGGTAGAATCGAAAGCATTACCGACGACAGCATCGTCCTGAAC | |
| GAGCTGATAGAAGACAGCACGGGCAACTGGGTTTCCCGTAAAGCAGAACTGCTGTTGAATTCTTCCGACAAAAACACCGA | |
| ACAAGCGGCAGCACCTGCCGCAGAACAAAATTAAGAAGAGGATTACTCCATT | |
| SEQ. ID NO:6 | |
| Nucleotide sequence of DNA region (1000 bp) up-stream from the Hap gene | |
| from Neisseria meningitidis | |
| GTGCGGCAAAAAACAGCAAAAGCCCGCTGTCGATTGCCTGACCGTCCGCGTCCGTAAAATCAGCATAGGTTGCCACGCGC | |
| GGCTTGGGCGTTTTCCCACACAAAGCCTCTGCCATCGGCAGCAGGTTTTTCCCCGATATGCGTATCACGCCCACGCCGCC | |
| GCGCCCGGGTGCGGTAGCGACTGCCGCAATCGTTGGAACGTTATCCGACATAAAACCCCCGAAAATTCAAAACAGCCGCG | |
| ATTATAGCAAATGCCGTCTGAAGTCCGACGGTTTGGCTTTCAGACGGCATAAAACCGCAAAAATGCTTGATAAATCCGTC | |
| CGCCTGACCTAATATAACCATATGGAAAAACGAAACACATACGCCTTCCTGCTCGGTATAGGCTCGCTGCTGGGTCTGTT | |
| CCATCCCGCAAAAACCGCCATCCGCCCCAATCCCGCCGACGATCTCAAAAACATCGGCGGCGATTTTCAACGCGCCATAG | |
| AGAAAGCGCGAAAATGACCGAAAACGCACAGGACAAGGCGCGGCAGGCTGTCGAAACCGTCGTCAAATCCCCGGAGCTTG | |
| TCGAGCAAATCCTGTCCGACGAGTACGTGCAAATAATGATAGCCCGGCGTTTCCATTCGGGATCGTTGCCGCCGCCGTCC | |
| GACTTGGCGCAATACAACGACATTATCAGCAACGGGGCAGACCGCATTATGGCAATGGCGGAAAAAGAACAAGCCGTCCG | |
| GCACGAAACCATACGGCAAGACCAAACCTTCAACAGGCGCGGGCAACTGTACGGCTTCATCAGCGTCATCCTGATACTGC | |
| TTTTTGCCGTCTTCCTCGTATGGAGCGGCTACCCCGCAACCGCCGCCTCCCTTGCCGGCGGCACAGTGGTTGCCTTGGCG | |
| GGTGCTTTCGTGATTGGAAGAAGCCGAGACCAAGGCAAAAATTAATTGCAAATCCTAGGGCGTGCTTCATATCCGCCCGA | |
| ACGCCGAACCGCACATATAGGCACATCCCGCGCGCCGCCGGAAGCGGAAGCCGCGCCCTCCCAAACAAACCCGAATCCCG | |
| TCAGATAAGGAAAAATA | |
| SEQ. ID NO:7 | |
| Nucleotide sequence of DNA region (924 bp) up-stream from the NspA gene from | |
| Neisseria meningitidis (serogroup B) (ATCC13090) | |
| GGAACCGAACACGCCGTTCGGTCATACGCCGCCGAAAGGTTTGCCGCAAGACGAAGCCGCCCTCGACATCGAAGACGCGG | |
| TACACGGCGCGCTGGAAGGCGCGGGTTTTGTCCACTACGAAACATCGGCTTTTGCGAAACCAGCCATGCAGTGCCGCCAC | |
| AATTTGAACTACTGGCAGTTCGGCGATTATTTAGGCATAGGCGCGGGCGCTCACGGCAAAATTTCCTATCCCGACCGCAT | |
| CGAGCGCACCGTCCGCCGCCGCCACCCCAACGACTACCTCGCCTTAATGCAAAGCCAACCGAGTGAAGCCGTCGAACGCA | |
| AAACCGTTGCCGCCGAAGATTTGCCGTTTGAGTTCATGATGAACGCCCTGCGCCTGACCGACGCGTACCCGCCGCGATGT | |
| TGCAGGAGCGCACGGGCGTACCGAGTGCCAAAATCATGGCGCAAATCGAAACGGCAAGGCAAAAAGGCCTGCTGGAAACC | |
| GACCCCGCCGTATTCCGCCCGACCGAAAAAGGACGCTTGTTTTTAAACGATTTGCTGCAGTGTTTTTTATAGTGGATTAA | |
| CAAAAACCAGTACGGCGTTGCCTCGCCTTAGCTCAAAGAGAACGATTCTCTAAGGTGCTGAAGCACCAAGTGAATCGGTT | |
| CCGTACTATTTGTACTGTCTGCGGCTTCGTCGCCTTGTCCTGATTTTTGTTAATCCACTATATAAGCGCAAACAAATCGG | |
| CGGCCGCCCGGGAAAACCCGCCCCGAACGCGTCCGGAAAATATGCTTATCGATGGAAAACGCAGCCGCATCCCCCGCCGG | |
| GCGTTTCAGACGGCACAGCCGCCGCCGGAAATGTCCGACGCTTAAGGCACAGACGCACACAAAACCGTATGCCTGCACCT | |
| GCAACAATCCGACAGATACCGCTGTTTTTTCCAAACCGTTTGCA | |
| SEQ. ID NO:8 | |
| Nucleotide sequence of DNA region (1000 bp) up-stream from the FrpB gene | |
| from Neisseria meningitidis (serogroup B) | |
| AAGTGGGAATCTAAAATGAAAAGCAACAGGAATTTATCGGAAATGACCGAAACTGAACGGACTGGATTCCCGCTTTCGC | |
| GGGAATGACGGCGACAGGGTTGCTGTTATAGTGGATGAACAAAAACCAGTACGTCGTTGCCTCGCCTTAGCTCAAGAGA | |
| ACGATTCTCTAAGGTGCTGAAGCACCAAGTGAATCGGTTCCGTCCTATTTGTACTGTCTGCGGCTTCGTCGCCTTGTCCT | |
| GATTTCTGTTCGTTTTCGGTTATTCCCGATAAATTACCGCCGTTTCTCGTCATTTCTTTAACCCTTCGTCATTCCCGCGC | |
| AGGCGGGAATCTAGTTTTTTTGAGTTCCAGTTGTTTCTGATAAATTCTTGCAGCTTTGAGTTCCTAGATTCCCACTTTCG | |
| TGGGAATGACGGTGGAAAAGTTGCCGTGATTTCGGATAAATTTTCGTAACGCATAATTTCCGTTTTACCCGATAAATGCC | |
| CGCAATCTCAAATCCCGTCATTCCCCAAAAACAAAAAATCAAAAACAGAAATATCGTCATTCCCGCGCAGGCGGGAATCT | |
| AGACCTTAGAACAACAGCAATATTCAAAGATTATCTGAAAGTCCGAGATTCTAGATTCCCACTTTCGTGGGAATGACGAA | |
| TTTTAGGTTTCTGTTTTTGGTTTTCTGTCCTTGCGGGAATGATGAAATTTTAAGTTTTAGGAATTTATCGGAAAAAACAG | |
| AAACCGCTCCGCCGTCATTCCCGCACAGGCTTCGTCATTCCCGCGCAGGCTTCGTCATTCCCGCATTTGTTAATCCACTA | |
| TATTCCCGCCGTTTTTTACATTTCCGACAAAACCTGTCAACAAAAAACAACACTTCGCAAATAAAAACGATAATCAGCTT | |
| TGCAAAAATCCCCCCCCCCTGTTAATATAAATAAAAATAATTAATTAATTATTTTTCCTATCCTGCCAAATCTTAACGGT | |
| TTGGATTTACTTCCCTTCATACACTCAAGAGGACGATTGA | |
| SEQ. ID NO:9 | |
| Nucleotide sequence of DNA region (1000 bp) up-stream from the FrpA gene | |
| from Neisseria meningitidis (serogroup B) | |
| CTATAAAGATGTAAATAAAAATCTCGGTAACGGTAACACTTTGGCTCAGCAAGGCAGCTACACCAAAACAGACGGTACAA | |
| CCGCAAAAATGGGGGATTTACTTTTAGCAGCCGACAATCTGCACAGCCGCTTCACGAACAAAATGCTATCCATTAGCCAT | |
| GTTTCGGGAAAACACGATTTCCCCGTTTGTTTTAGGCTGTCTAAACAATAACCATAAATGTATATCATTATTTAAAATAA | |
| ATAAAAGTATTTAACTATTATTGACGAAATTTTAGAGAAAGAGTAGACTGTCGATTAAATGACAAACAATAGTGAGAAAG | |
| GAAATATTTACTATCCGAGCACAGAGCATATTTTAGGTAGCCTGTAACTGTTCCTGCTGGCGGAAGAGGATGAAGGTGGA | |
| CTTACCCGAGAATAAATGTCCTGTTGTGTGATATGGATGCCATGCCGCGAAGCAATTGATGCAATCACGGCAGTCCTACT | |
| TGAATGAAACCTGTCGTTGCAGAATTTGAAAACGCTATTTTTAAGAAAGGATAAAGGGAGAAAGAATTTTTGGTTTTTAA | |
| GCTGCATGAAACCGTGTTGGAATAAATGCACACCTACGATAATTAATAATTTTCGTTTTTTATTCTACAAGCTATTTATA | |
| TATGATTGCTAAAAGTTTATTTTTTAGATGCCAAAAAATATATTTTATATACTTCATATTGTTTATATGTCTTTATTTGA | |
| ATATATCTTACGATGGGGAAATATTTATATATTTTATAATAAATTTTACTCATTTGCTAATATGTCATGGAATATTACTT | |
| GTATTTTGTAGAATTTTTCCATATGAAAATATTCCATTTACTATTTTTCTGAACTTTATTAGTTTATTTTTAATATTTTT | |
| ACCTCTTATATTTACCATAAGAGAGCTAATTGATTCATATTATATTGAGTCGATAATTAATTTATTCTTAATTTTAATTC | |
| CTCACGTTATTTTTTTAATTTACTTGAAAGGAAAGCAGAT | |
| SEQ. ID NO:10 | |
| Nucleotide sequence of DNA region (1000 bp) up-stream from the FrpC gene | |
| from Neisseria meningitidis (serogroup B) | |
| GGAAACAGAGAAAAAAGTTTCTCTTCTATCTTGGATAAATATATTTACCCTCAGTTTAGTTAAGTATTGGAATTTATACC | |
| TAAGTAGTAAAAGTTAGTAAATTATTTTTAACTAAAGAGTTAGTATCTACCATAATATATTCTTTAACTAATTTCTAGGC | |
| TTGAAATTATGAGACCATATGCTACTACCATTTATCAACTTTTTATTTTGTTTATTGGGAGTGTTTTTACTATGACCTCA | |
| TGTGAACCTGTGAATGAAAAGACAGATCAAAAAGCAGTAAGTGCGCAACAGGCTAAAGAACAAACCAGTTTCAACAATCC | |
| CGAGCCAATGACAGGATTTGAACATACGGTTACATTTGATTTTCAGGGCACCAAAATGGTTATCCCCTATGGCTATCTTG | |
| CACGGTATACGCAAGACAATGCCACAAAATGGCTTTCCGACACGCCCGGGCAGGATGCTTACTCCATTAATTTGATAGAG | |
| ATTAGCGTCTATTACAAAAAAACCGACCAAGGCTGGGTTCTTGAGCCATACAACCAGCAAAACAAAGCACACTTTATCCA | |
| ATTTCTACGCGACGGTTTGGATAGCGTGGACGATATTGTTATCCGAAAAGATGCGTGTAGTTTAAGTACGACTATGGGAG | |
| AAAGATTGCTTACTTACGGGGTTAAAAAAATGCCATCTGCCTATCCTGAATACGAGGCTTATGAAGATAAAAGACATATT | |
| CCTGAAAATCCATATTTTCATGAATTTTACTATATTAAAAAAGGAGAAAATCCGGCGATTATTACTCATCGGAATAATCG | |
| AATAAACCAAACTGAAGAAGATAGTTATAGCACTAGCGTAGGTTCCTGTATTAACGGTTTCACGGTACAGTATTACCCGT | |
| TTATTCGGGAAAAGCAGCAGCTCACACAGCAGGAGTTGGTAGGTTATCACCAACAAGTAGAGCAATTGGTACAGAGTTTT | |
| GTAAACAATTCAAATAAAAAATAATTTAAAGGATCTTATT | |
| SEQ. ID NO:11 | |
| Nucleotide sequence of DNA region (1000 bp) up-stream from the omp85 gene | |
| from Neisseria meningitidis (serogroup B) | |
| ACGTCCGAACCGTGATTCCGCAACGCCGCGCCCAAAACCAAAGCCCAAGCCAAAATGCCGATATAGTTGGCATTGGCAAT | |
| CGCGTTAATCGGGTTGGCGACCAGGTTCATCAGCAGCGATTTCAACACTTCCACAATGCCGGAAGGCGGCGCGGCGGACA | |
| CATCGCCCGCGCCCGCCAAAACAATGTGCGTCGGGAAAACCATACCGGCGATGACGGCGGTCAGGGCTGCGGAAAACGTA | |
| CCAATGAGGTAAAGGATGATAATCGGCCTGATATGCGCCTTGTTGCCTTTTTGGTGCTGCGCGATTGTGGCCGCCACCAA | |
| AATAAATACCAAAACCGGCGCGACCGCTTTGAGCGCGCCGACAAACAGGCTGCCGAACAAGCCTGCCGCCAAGCCCAGTT | |
| GCGGGGAAACCGAACCGATTACGATGCCCAACGCCAAACCGGCGGCAATCTGCCTGACCAGGCTGACGCGGCCGATCGCA | |
| TGAAATAAGGATTTGCCGAACGCCATAATTCTTCCTTATGTTGTGATATGTTAAAAAATGTTGTATTTTAAAAGAAAACT | |
| CATTCTCTGTGTTTTTTTTATTTTTCGGCTGTGTTTTAAGGTTGCGTTGATTTGCCCTATGCAGTGCCGGACAGGCTTTG | |
| CTTTATCATTCGGCGCAACGGTTTAATTTATTGAACCAAAATAAATTTATTTAATCCTGCCTATTTTCCGGCACTATTCC | |
| GAAACGCAGCCTGTTTTCCATATGCGGATTGGAAACAAAATACCTTAAAACAAGCAGATACATTTCCGGCGGGCCGCAAC | |
| CTCCGAAATACCGGCGGCAGTATGCCGTCTGAAGTGTCCCGCCCCGTCCGAACAACACAAAAACAGCCGTTCGAAACCCT | |
| GTCCGAACAGTGTTAGAATCGAAATCTGCCACACCGATGCACGACACCCGTACCATGATGATCAAACCGACCGCCCTGCT | |
| CCTGCCGGCTTTATTTTTCTTTCCGCACGCATACGCGCCT | |
| SEQ. ID NO:12 | |
| Nucleotide sequence of DNA region (772 bp) up-stream from the PilQ gene from | |
| Neisseria meningitidis (serogroup B) (ATCC13090) | |
| GCGATGTCGGGAAGCCTTCTCCCGAATCATTACCCCTTGAGTCGCTGAAAATCGCCCAATCTCCGGAAAACGGCGGCAAT | |
| CATGACGGCAAGAGCAGCATCCTGAACCTCAGTGCCATTGCCACCACCTACCAAGCAAAATCCGTAGAAGAGCTTGCCGC | |
| AGAAGCGGCACAAAATGCCGAGCAAAAATAACTTACGTTAGGGAAACCATGAAACACTATGCCTTACTCATCAGCTTTCT | |
| GGCTCTCTCCGCGTGTTCCCAAGGTTCTGAGGACCTAAACGAATGGATGGCACAAACGCGACGCGAAGCCAAAGCAGAAA | |
| TCATACCTTTCCAAGCACCTACCCTGCCGGTTGCGCCGGTATACAGCCCGCCGCAGCTTACAGGGCCGAACGCATTCGAC | |
| TTCCGCCGCATGGAAACCGACAAAAAAGGGGAAAATGCCCCCGACACCAAGCGTATTAAAGAAACGCTGGAAAAATTCAG | |
| TTTGGAAAATATGCGTTATGTCGGCATTTTGAAGTCTGGACAGAAAGTCTCCGGCTTCATCGAGGCTGAAGGTTATGTCT | |
| ACACTGTCGGTGTCGGCAACTATTTGGGACAAAACTACGGTAGAATCGAAAGCATTACCGACGACAGCATCGTCCTGAAC | |
| GAGCTGATAGAAGACAGCACGGGCAACTGGGTTTCCCGTAAAGCAGAACTGCTGTTGAATTCTTCCCACAAAAACACCGA | |
| ACAAGCGGCAGCACCTGCCGCAGAACAAAATTAAGAAGAGGATTACTCCATT | |
| SEQ. ID NO:13 | |
| Nucleotide sequence of DNA region (1000 bp) up-stream from the Hsf-like gene | |
| from Neisseria meningitidis (serogroup B) | |
| TTTGTTTTTTCTTTTGGTTTGTTTGAATGGTTAAATCGGGGTTTGGGGGCGGATGGTGCGGCATCCGCCCGGTTTTTGGG | |
| GGTTGGGGGTTTTCTGATAAATTCCCCCAACTTAAAATCTCGTCATTCCCGCGAAGGCGGGAATCTGGGACGTGGAATCT | |
| AAGGAAACTGTTTTATCCGGTAAGTTTCCGTGCCGACGGGTCTGGATTCCCGCTTTTGCGGGAATGACGGCGGTGGGGTT | |
| TCTGTTTTTTCCGATAAATTCCTGTTGCGTTGCGTTTTTGGATTCCAGCTTTTGCGGGAATGACGGTCGGTGGGGTTTCT | |
| GTTTTTTCCGATAAAGTCCTGCCGCGTTGTGTTTCTGGATTCCCGCCTGCGCGGGAATGACGGTCGGTGGGGGTTTCTGT | |
| TTTTGCTGATAGATTCCTGTGGTTTTTCGGTTGCTGGATTCCCGCTTTTGCGGGAATGACGGTCGGTGGGGTTTCTGTTT | |
| TTTCCGATAAATTCCTGTTGCCTTGTGTTTCTGGATTCCCGCCTGCGCGGGAATGACGCGGTGGGGGTTTCTGTTTTTTC | |
| CGATAAATTCCTGTTGCGTTGCGTTTTTGGATTCCAACTTTTGCGGGAATGACGGTCGGTGGGGTTTCGGTTTTTTCCGA | |
| TAAAGTCCTGCCGCGTTGTGTTTCTGGATTCCCGCCTGCGCGGGAATGACGCGGTGGGGGTTTCTGTTTTTTCTGATAGA | |
| TTCCTGTGGTTTTTCTATGGATTCAATCATTCCTGATAAATTCCCATAATCTAAAATCTCGTCATTCCCGCGAAAGCGGG | |
| AATCTAGGACGTGGAATCTAAGGAAACTGTTTTATCCGGTAAGTTTCCGTGCCGACGGGTCTGGATTCCCGCTTTTGCGG | |
| GAATGACGGCGGTGGGGTTTCTGTTTTTTCTGATAAAGTCCTGCCGCGTTGTGTTTCTAGATTCCCGCTTTTGCGGGAAT | |
| GACGGCGGTGAGGTTTCTGTTTTTTCCGATAAATTCCTGT | |
| SEQ. ID NO:14 | |
| Nucleotide sequence of DNA region (1000 bp) up-stream from the Hap gene | |
| from Neisseria meningitidis (serogroup B) | |
| AATCAGCATAGGTTGCCACGCGCGGCTTGGGCGTTTTCCCACACAAAGCCTCTGCCATCGGCAGCAGGTTTTTCCCCGAT | |
| ATGCGTATCACGCCCACGCCGCCGCGCCCGGGTGCGGTAGCGACTGCCGCAATCGTTGGAACGTTATCCGACATAAAACC | |
| CCCGAAAATTCAAAACAGCCGCGATTATAGCAAATGCCGTCTGAAGTCCGACGGTTTGGCTTTCAGACGGCATAAAACCG | |
| CAAAAATGCTTGATAAATCCGTCCGCCTGACCTAATATAACCATATGGAAAAACGAAACACATACGCCTTCCTGCTCGGT | |
| ATAGGCTCGCTGCTGGGTCTGTTCCATCCCGCAAAAACCGCCATCCGCCCCAATCCCGCCGACGATCTCAAAAACATCGG | |
| CGGCGATTTTCAACGCGCCATAGAGAAAGCGCGAAAATGACCGAAAACGCACAGGACAAGGCGCGGCAGGCTGTCGAAAC | |
| CGTCGTCAAATCCCCGGAGCTTGTCGAGCAAATCCTGTCCGACGAGTACGTGCAAATAATGATAGCCCGGCGTTTCCATT | |
| CGGGATCGTTGCCGCCGCCGTCCGACTTGGCGCAATACAACGACATTATCAGCAACGGGGCAGACCGCATTATGGCAATG | |
| GCGGAAAAAGAACAAGCCGTCCGGCACGAAACCATACGGCAAGACCAAACCTTCAACAGGCGCGGGCAACTGTACGGCTT | |
| CATCAGCGTCATCCTGATACTGCTTTTTGCCGTCTTCCTCGTATGGAGCGGCTACCCCGCAACCGCCGCCTCCCTTGCCG | |
| GCGGCACAGTGGTTGCCTTGGCGGGTGCTTTCGTGATTGGAAGAAGCCGAGACCAAGGCAAAAATTAATTGCAAATCCTA | |
| GCGCGTGCTTCATATCCGCCCGAACGCCGAACCGCACATATAGGCACATCCCGCGCGCCGCCGGAAGCGGAAGCCGCCCC | |
| CTCCCAAACAAACCCGAATCCCGTCAGATAAGGAAAAATA | |
| SEQ. ID NO:15 | |
| Nucleotide sequence of DNA region (1000 bp) up-stream from the LbpA gene | |
| from Neisseria meningitidis (serogroup B) | |
| GATTTTGGTCATCCCGACAAGCTTCTTGTCGAAGGGCGTGAAATTCCTTTGGTTAGCCAAGAGAAAACCATCAAGCTTGC | |
| CGATGGCAGGGAAATGACCGTCCGTGCTTGTTGCGACTTTTTGACCTATGTGAAACTCGGACGGATAAAAACCGAACGCC | |
| CGGCAAGTAAACCAAAGGCGGAAGATAAAAGGGAGGATGAAGAGAGTGCAGGCGTTGGTAACGTCGAAGAAGGCGAAGGC | |
| GAAGTTTCCGAAGATGAAGGCGAAGAAGCCGAAGAAATCGTCGAAGAAGAACCCGAAGAAGAAGCTGAAGAGGAAGAAGC | |
| TGAACCCAAAGAAGTTGAAGAAACCGAAGAAAAATCGCCGACAGAAGAAAGCGGCAGCGGTTCAAACGCCATCCTGCCTG | |
| CCTCGGAAGCCTCTAAAGGCAGGGACATCGACCTTTTCCTGAAAGGTATCCGCACGGCGGAAGCCGACATTCCAAGAACC | |
| GGAAAAGCACACTATACCGGCACTTGGGAAGCGCGTATCGGCACACCCATTCAATGGGACAATCAGGCGGATAAAGAAGC | |
| GGCAAAAGCAGAATTTACCGTTAATTTCGGCGAGAAATCGATTTCCGGAACGCTGACGGAGAAAAACGGTGTACAACCTG | |
| CTTTCTATATTGAAAACGGCAAGATTGAGGGCAACGGTTTCCACGCAACAGCACGCACTCGTGAGAACGGCATCAATCTT | |
| TCGGGAAATGGTTCGACCAACCCCAGAACCTTCCAAGCTAGTGATCTTCGTGTAGAAGGAGGATTTTACGGCCCGCAGCG | |
| GAGGAATTGGGCGGTATTATTTTCAATAAGGATGGGAAATCTCTTGGTATAACTGAAGGTACTGAAAATAAAGTTGAAGT | |
| TGAAGCTGAAGTTGAAGTTGAAGCTGAAACTGGTGTTGTCGAACAGTTAGAACCTGATGAAGTTAAACCCCAATTCGGCG | |
| TGGTATTCGGTGCGAAGAAAGATAATAAAGAGGTGGAAAA | |
| SEQ. ID NO:16 | |
| Nucleotide sequence of DNA region (1000 bp) up-stream from the LbpB gene | |
| from Neisseria meningitidis (serogroup A) | |
| CGGCGTTAGAGTTTAGGGCAGTAAGGGCGCGTCCGCCCTTAGATCTGTAAGTTACGATTCCGTTAAATAACTTTTACTGA | |
| CTTTGAGTTTTTTGACCTAAGGGTGAAAGCACCCTTACTGCTTAAAGTCCAACGACAAAAACCAAAAGACAAAAACACTT | |
| TTATTACCCTAAAATCGAACACCCATAAATGACCTTTTTTGTCTTTGGCGAGGCGGCAGTAAGGGCGCGTCCGCCCTTAG | |
| ATCTGTAAGTTATGATTCCGTTAAATAGCCTTTACTGACTTTGAGTTTTTTGACCTAAGGGCGGACGCGCCCTTACTGCT | |
| TCACCTTCAATGGGCTTTGAATTTTGTTCGCTTTGGCTTGCTTGACCTAAGGGTGAAAGCACCCTTACTGCCGCCTCGCC | |
| AAAGACGAAAAGGGTTATTTACGGGGGTTGGATTTTAGGCAGTAAGGGCGCGTCCGCCCTTAGATCTGTAAGTTATGATT | |
| CCGTTAAATAGCCTTTACTGACTTTGAGTTTTTTGACCTAAGGGTGAAAGCACCCTTACTGCTTCACCTTCAATGGGCTT | |
| TGAATTTTGTTCGCTTTGGCTTGCTTGATCTAAGGGTGAAAGCACCCTTACTGCCGTCTCGCCGAAGACAACGAGGGCTA | |
| TTTACGGCGTTAGAGTTTAGGGCAGTAAGGGCGCGTCCGCCCTTAGATCCAGACAGTCACGCCTTTGAATAGTCCATTTT | |
| GCCAAAGAACTCTAAAACGCAGGACCTAAGGGTGAAAGCACCCTTACTGCCTTACATCCAAGCACCCTTACTGCACCACG | |
| TCCACGCACCCTTACTGCCCTACGTCCACGCACCCTTACTGCCCTACATCCAAGCACCCTTACTGCCTTACATAGACATG | |
| ACAGACGCCGAGCAGCGGAACAGGACTAAAAACAATTAAGTGATATTTTTGCCCAACTATAATAGACATGTATAATTATA | |
| TTACTATTAATAATAATTAGTTTATCCTCCTTTTCATCCC | |
| SEQ. ID NO:17 | |
| Nucleotide sequence of DNA region (731 bp) up-stream from the TbpA gene | |
| from Neisseria meningitidis (serogroup B) (ATCC13090) | |
| TATGAAGTCGAAGTCTGCTGTTCCACCTTCAATTATCTGAATTACGGAATGTTGACGCGC | |
| AAAAACAGCAAGTCCGCGATGCAGGCAGGAGAAAGCAGTAGTCAAGCTGATGCTAAAACG | |
| GAACAAGTTGGACAAAGTATGTTCCTCCAAGGCGAGCGCACCGATGAAAAAGAGATTCCA | |
| AACGACCAAAACGTCGTTTATCGGGGGTCTTGGTACGGGCATATTGCCAACGGCACAAGC | |
| TGGAGCGGCAATGCTTCCGATAAAGAGGGCGGCAACAGGGCGGACTTTACTGTGAATTTC | |
| GGTACGAAAAAAATTAACGGCACGTTAACCGCTGACAACAGGCAGGCGGCAACCTTTACC | |
| ATTGTGGGCGATATTGAGGGCAACGGTTTTTCCGGTACGGCGAAAACTGCTGACTCAGGT | |
| TTTGATCTCGATCAAAGCAATAACACCCGCACGCCTAAGGCATATATCACAAACGCCAAG | |
| GTGCAGGGCGGTTTTTACGGGCCCAAAGCCGAAGAGTTGGGCGGATGGTTTGCCTATTCG | |
| GACGATAAACAAACGAAAAATGCAACAGATGCATCCGGCAATGGAAATTCAGCAAGCAGT | |
| GCAACTGTCGTATTCGGTGCGAAACGCCAAAAGCCTGTGCAATAAGCACGGTTGCCGAAC | |
| AATCAAGAATAAGGCCTCAGACGGCACCGCTCCTTCCGATACCGTCTGAAAGCGAAGAGT | |
| AGGGAAACACT | |
| SEQ. ID NO:18 | |
| Nucleotide sequence of DNA region (373 bp) up-stream from the OmplA gene | |
| from Neisseria meningitidis (serogroup B) (ATCC13090) | |
| CGTACCGCATTCCGCACTGCAGTGAAAAAAGTATTGAAAGCAGTCGAAGCAGGCGATAAAGCTGCCGCACAAGCGGTTTA | |
| CCAAGAGTCCGTCAAAGTCATCGACCGCATCGCCGACAAGGGCGTGTTCCATAAAAACAAAGCGGCTCGCCACAAAACCC | |
| GTTTGTCTCAAAAAGTAAAACCTTGGCTTGATTTTTGCAAAACCTGCAATCCGGTTTTCATCGTCGATTCCGAAAACCCC | |
| TGAAGCCCGACGGTTTCGGGGTTTTCTGTATTGCGGGGACAAAATCCCGAAATGGCGGAAAGGGTGCGGTTTTTTATCCG | |
| AATCCGCTATAAAATGCCGTCTGAAAACCAATATGCCGACAATGGGGGTGGAG | |
| SEQ. ID NO:19 | |
| Nucleotide sequence of DNA region (1000 bp) up-stream from the Pla1 gene | |
| from Neisseria meningitidis (serogroup B) | |
| TTTTGGCTTCCAGCGTTTCATTGTTTTCGTACAAGTCGTAAGTCAGCTTCAGATTGTTGG | |
| CTTTTTTAAAGTCTTCGACCGTACTCTCATCAACATAGTTCGACCAGTTGTAGATGTTCA | |
| GAGTATCGGTGGCAGCGGCTTCGGCATTGGCAGCAGACGCAGCGTCTGCTTGAGGTTGCA | |
| CGGCGTTTTTTTCGCTGCCGCCGCAGGCTGCCAGAGACAGCGCGGCCAAAACGGCTAATA | |
| CGGATTTTTTCATACGGGCAGATTCCTGATGAAAGAGGTTGGAAAAAAAGAAATCCCCGC | |
| GCCCCATCGTTACCCCGGCGCAAGGTTTGGGCATTGTAAAGTAAATTTGTGCAAACTCAA | |
| AGCGATATTGGACTGATTTTCCTAAAAAATTATCCTGTTTCCAAAAGGGGAGAAAAACGT | |
| CCGCCCGATTTTGCCGTTTTTTTGCGCTGTCAGGGTGTCCGACGGGCGGATACAGAGAAA | |
| AGGCTTGCATATAATGTAAACCCCCTTTAAAATTGCGCGTTTACAGAATTTATTTTTCTT | |
| CCAGGAGATTCCAATATGGCAAACAGCGCACAAGCACGCAAACGTGCCCGCCAGTCCGTC | |
| AAACAACGCGCCCACAATGCTAGCCTGCGTACCGCATTCCGCACCGCAGTGAAAAAAGTA | |
| TTGAAAGCAGTCGAAGCACGCGATAAAGCTGCCGCACAAGCGGTTTACCAAGAGTCCGTC | |
| AAAGTCATCGACCGCATCGCCGACAAGGGCGTGTTCPACAAAAACAAAGCGGCACGCCAC | |
| AAAAGCCGTCTGTCTGCAAAAGTAAAAGCCTTGGCTTGATTTTTGCAAAACCGCCAAGGC | |
| GGTTGATACGCGATAAGCGGAAAACCCTGAAGCCCGACGGTTTCGGGGTTTTCTGTATTG | |
| CGGGGGCAAAATCCCGAAATGGCGGAAAGGGTGCGATTTTTTATCCGAATCCGCTATAAA | |
| ATGCCGTTTGAAAACCAATATGCCGACAATGGGGGCGGAG | |
| SEQ. ID NO:20 | |
| Nucleotide sequence of DNA region (1000 bp) up-stream from the FhaB gene | |
| from Neisseria meningitidis (serogroup B) | |
| TACGGAAACTGCAAGCGGATCCAGAAGTTACAGCGTGCATTATTCGGTGCCCGTAAAAAAATGGCTGTTTTCTTTTAATC | |
| ACAATGGACATCGTTACCACGAAGCAACCGAAGGCTATTCCGTCAATTACGATTACAACGGCAAACAATATCAGAGCAGC | |
| CTGGCCGCCGAGCGCATGCTTTGGCGTAACAGACTTCATAAAACTTCAGTCGGAATGAAATTATGGACACGCCAAACCTA | |
| TAAATACATCGACGATGCCGAAATCGAAGTGCAACGCCGCCGCTCTGCAGGCTGGGAAGCCGAATTGCGCCACCGTGCTT | |
| ACCTCAACCGTTGGCAGCTTGACGGCAAGTTGTCTTACAAACGCGGGACCGGCATGCGCCAAAGTATGCCTGCACCGGAA | |
| GAAAACGGCGGCGATATTCTTCCAGGTACATCTCGTATGAAAATCATTACTGCCGGTTTGGACGCAGCCGCCCCATTTAT | |
| TTTAGGCAAACAGCAGTTTTTCTACGCAACCGCCATTCAAGCTCAATGGAACAAAACGCCGTTGGTTGCCCAAGATAAAT | |
| TGTCAATCGGCAGCCGCTACACCGTTCGCGGATTTGATGGGGAGCAGAGTCTTTTCGGAGAGCGAGGTTTCTACTGGCAG | |
| AATACTTTAACTTGGTATTTTCATCCGAACCATCAGTTCTATCTCGGTGCGGACTATGGCCGCGTATTTGGCGAAAGTGC | |
| ACAATATGTATCGGGCAAGCAGCTGATGGGTGCAGTGGTCGGCTTCAGAGGAGGGCATAAAGTAGGCGGTATGTTTGCTT | |
| ATGATCTGTTTCCCGGCAAGCCGCTTCATAAACCCAAAGGCTTTCAGACGACCAACACCGTTTACGGCTTCAACTTGAAT | |
| TACAGTTTCTAACCTCTGAATTTTTTACTGATATTTAGACGGTCTTTCCTTATCCTCAGACCGTCAAACTTTACCTACGT | |
| ACTTGGCGCGCAGTACGTTCATCTTCAAAATGGAATAGAC | |
| SEQ. ID NO:21 | |
| Nucleotide sequence of DNA region (1000 bp) up-stream from the Lipo02 gene | |
| from Neisseria meningitidis (serogroup B) | |
| TTATCTTGGTGCAAAACTTTGTCGGGGTCGGACTGGCTACGGCTTTGGGTTTGGACCCGCTCATCGGTCTGATTACCGGT | |
| TCGGTGTCGCTGACGGGCGGACACGGTACGTCAGGTGCGTGGGGACCTAATTTTGAAACGCAATACGGCTTGGTCGGCGC | |
| AACCGGTTTGGGTATTGCATCGGCTACTTTCGGGCTGGTGTTCGGCGGCCTGATCGGCGGGCCGGTTGCGCGCCGCCTGA | |
| TCAACAAAATGGGCCGCAAACCGGTTGAAAACAAAAAACAGGATCAGGACGACAACGCGGACGACGTGTTCGAGCAGGCA | |
| AAACGCACCCGCCTGATTACGGCGGAATCTGCCGTTGAAACGCTTGCCATGTTTGCCCCGTGTTTGGCGTTTGCCGAGAT | |
| TATGGACGGCTTCGACAAAGAATATCTGTTCGACCTGCCCAAATTCGTGTGGTGTCTGTTTGGCGGCGTGGTCATCCGCA | |
| ACATCCTCACTGCCGCATTCAAGGTCAATATGTTCGACCGCGCCATCGATGTGTTCGGCAATGCTTCGCTTTCGCTTTTC | |
| TTGGCAATGGCGTTGCTGAATTTGAAACTGTGGGAGCTGACCGGTTTGGCGGGGCCTGTAACCGTGATTCTTGCCGTACA | |
| AACCGTGGTGATGGTTTTGTACGCGACTTTTGTTACCTATGTCTTTATGGGGCGCGACTATGATGCGGCAGTATTGGCTG | |
| CCGGCCATTGCGGTTTCGGCTTGGGTGCAACGCCGACGGCGGTGGCAAATATGCAGTCCGTCACGCATACTTTCGGCGCG | |
| TCGCATAAGGCGTTTTTGATTGTGCCTATGGTCGGCGCGTTCTTCGTCGATTTGATTAATGCCGCGATTCTCACCGGTTT | |
| TGTGAATTTCTTTAAAGGCTGATTTTCCGCCTTTCCGACAAAGCACCTGCAAGGTTTACCGCCTGCAGGTGCTTTTGCTA | |
| TGATAGCCGCTATCGGTCTGCACCGTTTGGAAGGAACATC | |
| SEQ. ID NO:22 | |
| Nucleotide sequence of DNA region (1000 bp) up-stream from the Tbp2 gene | |
| from Neisseria meningitidis (serogroup B) | |
| CCTACTCCACCGATTCCAATATGCTCGGCGCGACCCACGAAGCCAAAGACTTGGAATTTTTGAACTCGGGCATCAAAATC | |
| GTCAAACCCATTATGGGCGTTGCCTTTTGGGACGAAAACGTTGAAGTCAGCCCCGAAGAAGTCAGCGTGCGCTTTGAAGA | |
| AGGCGTGCCGGTTGCACTGAACGGCAAAGAATACGCCGACCCCGTCGAACTCTTCCTCGAAGCCAACCGCATCGGCGGCC | |
| GCCACGGCTTGGGTATGAGCGACCAAATCGAAAACCGCATCATCGAAGCCAAATCGCGCGGCATCTACGAAGCCCCGGGT | |
| ATGGCGTTGTTCCACATCGCCTACGAACGCTTGGTGACCGGCATCCACAACGAAGACACCATCGAACAATACCGCATCAA | |
| CGGCCTGCGCCTCGGCCGTTTGCTCTACCAAGGCCGCTGGTTCGACAGCCAAGCCTTGATGTTGCGCGAAACCGCCCAAC | |
| GCTGGGTCGCCAAAGCCGTTACCGGCGAAGTTACCCTCGAACTGCGGCGCGGCAACGACTACTCGATTCTGAACACCGAA | |
| TCGCCCAACCTGACCTACCAACCCGAACGCCTGAGTATGGAAAAAGTCGAAGGTGCGGCGTTTACCCCGCTCCACCGCAT | |
| CGGACAGCTCACGATGCGCAACCTCGACATCACCGACACCCGCGCCAAACTGGGCATCTACTCGCAAAGCGGTTTGCTGT | |
| CGCTGGGCGAAGGCTCGGTATTACCGCAGTTGGGCAATAAGAAATAAGGTTTGCTGTTTTGCATCATTAGCAACTTAAGG | |
| GGTCGTCTGAAAAGATGATCCCTTATGTTAAAAGGAATCCTATGAAAGAATACAAAGTCGTCATTTATCAGGAAAGCCAG | |
| TTGTCCAGCCTGTTTTTCGGCGCGGCAAAGGTCAACCCCGTCAATTTCAGCGCGTTCCTCAACAAACAAACCCCCCGAAG | |
| GCTGGCGGGTCGAGACCTTTGCAATAACATAGGTTACTAA | |
| SEQ. ID NO:23 | |
| Nucleotide sequence of DNA region (1000 bp) up-stream from the PorA gene | |
| from Neisseria meningitidis (serogroup B) | |
| GAATGACAATTCATAAGTTTCCCGAAATTCCAACATAACCGAAACCTGACAATAACCGTAGCAACTGAACCGTCATTCCC | |
| GCAAAAGCGGGAATCCAGTCCGTTCAGTTTCGGTCATTTCCGATAAATGCCTGTTGCTTTTCATTTCTAGATTCCCACTT | |
| TCGTGGGAATGACGGCGGAAGGGTTTTGGTTTTTTCCGATAAATTCTTGAGGCATTGAAATTCCAAATTCCCGCCTGCGC | |
| GGGAATGACGGCTGCAGATGCCCGACGGTCTTTATAGTGGATTAACAAAAATCAGGACAAGGCGACGAGCTGCAGACAGT | |
| ACAGATAGTACGGAACCGATTCACTTAGTGCTTCAGTATCTTAGAGAATCGTTCTCTTTGAGCTAAGGCGAGGCAACGTC | |
| GTACTGGTTTTTGTTCATCCACTATATATGACACGGAAAACGCCGCCGTCCAAACCATGCCGTCTGAAGAAAACTACACA | |
| GATACCGCCGCTTATATTACAATCGCCGCCCCGTCGTTCGAAAACCTCCCACACTAAAAAACTAAGGAAACCCTATGTCC | |
| CGCAACAACGAAGAGCTGCAAGGTATCTCGCTTTTGGGTAATCAAAAAACCCAATATCCGGCCGAATACGCGCCCGAAAT | |
| TTTGGAAGCGTTCGACAACAAACATCCCGACAACGACTATTTCGTCAAATTCGTCTGCCCAGAGTTCACCAGCCTCTGCC | |
| CCATGACCGGGCAGCCCGACTTCGCCACCATCGTCATCCGCTACATTCCGCACATCAAAATGGTGGAAAGCAAATCCCTG | |
| AAACTCTACCTCTTCAGCTTCCGCAACCACGGCGATTTTCATGAAGACTGCGTCAACATCATCATGAAAGACCTCATTGC | |
| CCTGATGGATCCGAAATACATCCAAGTATTCGGCGAGTTCACACCGCGCGGCGGCATCGCCATTCATCCTTTCGCCAATT | |
| ACGGCAAAGCAGGCACCGAGTTTGAAGCATTGGCGCGTAA | |
| SEQ. ID NO:24 | |
| Neisseria meningitidis (serogroup B) PorA Promoter Region | |
| GATATCGAGGTCTGCGCTTGAATTGTGTTGTAGAAACACAACGTTTTTGAAAAAATAAGCTATTGTTTTATATCAAAATA | |
| TAATCATTTTTAAAATAAAGGTTGCGGCATTTATCAGATATTTGTTCTGAAAAATGGTTTTTTGCGGGGGGGGGGGTATA | |
| ATTGAAGACGTATCGGGTGTTTGCCCGATGTTTTTAGGTTTTTATCAAATTTACAAAAGGAAGCCCAT | |
| SEQ. ID NO:25 | |
| Nucleotide sequence of DNA region (1000 bp) up-stream from the PorB gene | |
| from Neisseria meningitidis (serogroup A) | |
| gttttctgtttttgagggaatgacgggatgtaggttcgtaagaatgacgggatataggtttccgtgcggatggattcgtc | |
| attcccgcgcaggcgggaatctagaacgtggaatctaagaaaccgttttatccgataagtttccgtgcggacaagtttgg | |
| attcccgcctgcgcgggaatgacgggattttaggtttctaattttggttttctgtttttgagggaatgacgggatgtagg | |
| ttcgtaggaatgacgggatataggtttccgtgcggatggattcgtcattcccgcgcaggcgggaatctagaccttagaac | |
| aacagcaatattcaaagattatctgaaagtccgagattctagattcccgcctgagcgggaatgacgaaaagtggcgggaa | |
| tgacggttagcgttgcctcgccttagctcaaagagaacgattctctaaggtgctgaagcaccaagtgaatcggttccgta | |
| ctatttgtactgtctgcggcttcgtcgccttgtcctgatttttgttaatccactatctcctgccgcaggggcgggttttg | |
| catccgcccgttccgaaagaaaccgcgtgtgcgttttttgccgtctttataacccccggtttgcaatgccctccaatacc | |
| ctcccgagtaagtgttgtaaaaatgcaaatcttaaaaaatttaaataaccatatgttataaaacaaaaaatacccataat | |
| atctctatccgtccttcaaaatgcacatcgaattccacacaaaaacaggcagaagtttgttttttcagacaggaacatct | |
| atagtttcagacatgtaatcgccgagcccctcggcggtaaatgcaaagctaagcggcttggaaagcccggcctgcttaaa | |
| tttcttaaccaaaaaaggaatacagcaatgaaaaaatccctgattgccctgactttggcagcccttcctgttgcagcaat | |
| ggctgacgttaccctgtacggcaccatcaaaaccggcgta | |
| SEQ. ID NO:26 | |
| Neisseria meningitidis (serogroup B) PorB Promoter Region | |
| GTTTTCTGTTTTTGAGGGAATGACGGGATGTAGGTTCGTAAGAATGACGGGATATAGGTTTCCGTGCGGATGGATTCGTC | |
| ATTCCCGCGCAGGCGGGAATCTAGAACGTGGAATCTAAGAAACCGTTTTATCCGATAAGTTTTCCGTGCGGACAAGTTTG | |
| GATTCCCGCCTGCGCGGGAATGACGGGATTTTAGGTTTCTAATTTTGGTTTTCTGTTTTTGAGGGAATGACGGGATGTAG | |
| GTTCGTAGGAATGACGGGATATAGGTTTCCGTGCGGATGGATTCGTCATTCCCGCGCAGGCGGGAATCCAGACCTTAGAA | |
| CAACAGCAATATTCAAAGATTATCTGAAAGTCCGAGATTCTAGATTCCCGCCTGAGCGGGAATGACGAAAAGTGGCGGGA | |
| ATGACGGTTAGCCTTGCCTCGCCTTAGCTCAAAGAGAACGATTCTCTAAGGTGCTGAAGCACTAAGTGAATCGGTTCCGT | |
| ACTATTTGTACTGTCTGCGGCTTCGTCGCCTTGTCCTGATTTTTGTTAATCCACTAT | |
| SEQ. ID NO:27 | |
| Nucleotide sequence of DNA region (1000 bp) up-stream from the siaABC gene | |
| from Neisseria meningitidis (serogroup B) | |
| ATACGGCCAATGGCTTCAGAAAGCGATAAGCCTCTGGCTGAAAAACCGATTTCTTGTGTTCTCCCCACCGCACCCATAGA | |
| CGTAAAGGTATAGGGATTGGTAATCATGGTAACCACATCACCGCGACGCAGCAAAATATTTTGTCGCGGATTTGCAACTA | |
| AATCTTCCAAGGCAACAGTTCGTACTACATTGCCACGTGTCAGCTGCACATTCGTATCCTGCACATTTGCCGTTGAACCA | |
| CCTACCGCACCCACCGCATCCAACACACGCTCACCGGCTGCCGTCAGCGGCATACGCACACTATTCCCAGCACGAATCAC | |
| CGACACATTCGCCGCATTATTCTGCACCAAACGCACCATCACTTGTGGCTGATTGGCCATTTTTTTCAGGCGGCCTTTAA | |
| TAATTTCCTGAACCTGACCAGGCGTTTTACCGACCACCGAAATATCGCCAACAAACGGCACAGAAACCGTACCACGTGCC | |
| GTGACCAACTGCTCTGGCAACTTAGTTTGATGCGCACTACCCGAGCCCATCGAAGAAAGGCCACCACCAAACAATACTGC | |
| CGGCGGCGCTTCCCAAATCATAATATCCAATACATCACCAATATTTAGCGTACCAGCCGAAGCATAACCATCGCCAAACT | |
| GAGTCAATGACTGATTTATCTGAGCCTTATATAATAACTGAGCAACCGTATGATTCACATCAATCAGCTCCACTTCAGGA | |
| ATTTGAACTTCAGATTGTTGCCCTAAAGAGACAATTTTTTTTGCGCTGGGGCCTGATGAAGGAATCGCAGAGCATCCTAC | |
| AATTAAACTTCCACACAATAATAATACTGCGTGACGAATATAAAATTTCACTTTAAACACAAGCCAAATCCTAATATAAT | |
| TATAAATGGCCTAATTATAGCACTTAATCGAAATAAATTTATGAGTACGTAGAGTATAATTAGTATTCTTCTTTCCAACT | |
| TCCTTATACTTATATATATATACTTATAGATTCTAAAATC | |
| SEQ. ID NO:28 | |
| Nucleotide sequence of DNA region (1000 bp) up-stream from the lgt gene from | |
| Neisseria meningitidis (serogroup B) | |
| GCCAAAGCATTGGGCGCGGATGCCGCCGCTGCCGAACGCGCCGCGCGTCTTGCCAAAGCCGACTTGGTAACCGAAATGGT | |
| CGGCGAGTTCCCCGAACTGCAAGGCACGATGGGCAAATACTATGCCTGTTTGGACGGCGAAACCGAAGAAATTGCCGAAG | |
| CCGTCGAGCAGCACTATCAGCCGCGTTTTGCCGGCGACAAGCTGCCCGAAAGCAAAATTGCCGCCGCCGTGGCACTGGCC | |
| GACAAACTAGAAACCTTGGTCGGCATTTGGGGCATCGGTCTGATTCCGACCGGCGACAAAGACCCCTACGCCCTGCGCCG | |
| CGCTGCCTTGGGTATTTTGCGTATGCTGATGCAGTATGGTTTGGACGTGAACGAACTGATTCAGACGGCATTCGACAGCT | |
| TCCCCAAAGGTTTGCTCAACGAAAAAACGCCGTCTGAAACCGCCGACTTTATGCAGGCGCGCCTTGCCGTGTTGCTGCAA | |
| AACGATTATCCGCAAGACATCGTTGCCGCCGTACTCGCCAAACAGCCGCGCCGTTTGGACGATTTGACCGCCAAACTGCA | |
| GGCCGTTGCCGCGTTCAAACAACTGCCCGAAGCCGCCGCGCTCGCCGCCGCCAACAAACGCGTGCAAAACCTGCTGAAAA | |
| AAGCCGATGCCGAGTTGGGCGCGGTTAACGAAAGCCTGTTGCAACAGGACGAAGAAAAAGCCCTCTTTGCCGCCGCGCAA | |
| GGCTTGCAGCCGAAAATCGCCGCCGCCGTCGCCGAAGGCAATTTCCAAACCGCCTTGTCCGAACTGGCTTCCGTCAAACC | |
| GCAAGTCGATGCATTCTTTGACGGCGTGATGGTAATGGCGGAAGATGCCGCCGTAAAACAAAACCGCCTGAACCTGCTGA | |
| ACCGCTTGGCAGAGCAAATGAACGCGGTAGCCGACATCGCGCTTTTGGGCGAGTAACCGTTGTACAGTCCAAATGCCGTC | |
| TGAAGCCTTCAGACGGCATCGTGCCTATCGGGAGAATAAA | |
| SEQ. ID NO:29 | |
| Nucleotide sequence of DNA region (1000 bp) up-stream from the TbpB gene | |
| from Neisseria meningitidis (strain MC58) | |
| GAACGAACCGGATTCCCACTTTCGTGGGAATGACGAATTTCAGGTTACTGTTTTTGGTTTTCTGTTTTTGTGAAAATAAT | |
| GGGATTTCAGCTTGTGGGTATTTACCGGAAAAAACAGAAACCGCTCCGCCGTCATTCCCGCGCAGGCGGGAATCTAGGTC | |
| TGTCGGTGCGGAAACTTATCGGATAAAACGGTTTCTTGAGATTTTTCGTCCTGGATTCCCACTTTCGTGGGAATGACGCG | |
| AACAGAAACCGCTCCGCCGTCATTCCCGCGCAGGCGGGAATCTAGACATTCAATGCTAAGGCAATTTATCGGGAATGACT | |
| GAAACTCAAAAAACTGGATTCCCACTTTCGTGGGAATGACGTGGTGCAGGTTTCCGTATGGATGGATTCGTCATTCCCGC | |
| GCAGGCGGGAATCTAGACCTTCAATACTAAGGCAATTTATCGGAAATGACTGAAACTCGAAAAACTGGATTCCCACTTTT | |
| GTGGGAATGACGCGATTAGAGTTTCAAAATTTATTCTAAATAGCTGAAACTCAACACACTGGATTCCCGCCTGCGCGGGA | |
| ATGACGAAGTGGAAGTTACCCGAAACTTAAAACAAGCGAAACCGAACGAACTGGATTCCCACTTTCGTGGGAATCACGGA | |
| ATGTAGGTTCGTGGGAATGACGGCGGAGCGGTTTCTGCTTTTTCCAATAAATGACCCCAACTTAAAATCCCGTCATTCCC | |
| GCGCAGGCGGGAATCTAGGTCTGTCGGTGCGGAAACTTATCGGGTAAAACGGTTTCTTGAGATTTTGCGTCCTGGATTCC | |
| CACTTTCGTGGGAATGACGGAATGTAGGTTCGTGGGAATGACGGGATATAGCTTTCCGTGCGGACGCGTTCGGATTCATG | |
| ACTGCGCGGGAATGACGGGATTTTGGTGTATTCCCTAAAAAAATAAAAAAGTATTTGCAAATTTGTTAAAAATAAATAAA | |
| ATAATAATCCTTATCATTCTTTAATTGAATTGGATTTATT | |
| SEQ. ID NO:30 | |
| Nucleotide sequence of DNA region (1000 bp) up-stream from the opc gene from | |
| Neisseria meningitidis (serogroup A) | |
| CAAAGGCTACGACAGTGCGGAAAACCGGCAACATCTGGAAGAACATCAGTTGTTGGACGGCATTATGCGCAAAGCCTGCC | |
| GCAACCGTCCGCTGTCGGAAACGCAAACCAAACGCAACCGGTATTTGTCGAAGACCCGTTATAGTGCATTAAATTTAAAT | |
| CAGGACAAGGCGACGAAGCCGCAGACAGTACAAATAGTACGGCAAGGCGAGGCAACGCCGTACTGGTTTAAATTTAATCC | |
| ACTATATGTGGTCGAACAGAGCTTCGGTACGCTGCACCGTAAATTCCGCTATGCGCGGGCAGCCTATTTCGGACTGATTA | |
| AAGTGAGTGCGCAAAGCCATCTGAAGGCGATGTGTTTGAACCTGTTGAAAGCCGCCAACAAGCTAAGTGCGCCCGCTGCC | |
| GCCTAAAAGGAGACCGGATGCCTGATTATCGGGTATCCGGGGAGGGTTAAGGGGGTATTTGGGTAAAATTAGGAGGTATT | |
| TGGGGCGAAAATAGACGAAAACCTGTGTTTGGGTTTCGGCTGTCGGGAGGGAAAGGAATTTTGCAAAGATCTCATCCTGT | |
| TATTTTCACAAAAACAGAAAACCAAAAACAGCAACCTGAAATTCGTCATTCCCGCGCAGGCGGGAATCCAGACCCCCAAC | |
| GCGGCAGGAATCTATCGGAAATAACCGAAACCGGACGAACCTAGATTCCCGCTTTCGCGGGAATGACGGCAGAGTGGTTT | |
| CAGTTGCTCCCGATAAATGCCGCCATCTCAAGTCTCGTCATTCCCTTAAAACAGAAAACCGAAATCAGAAACCTAAAATT | |
| TCGTCATTCCCATAAAAAACAGAAAACCAAGTGAGAATAACAATTCGTTGTAAACAAATAACTATTTGTTAATTTTTATT | |
| AATATATGTAAAATCCCCCCCCCCCCCCCCCGAAAGCTTAAGAATATAATTGTAAGCGTAACGATTATTTACGTTATGTT | |
| ACCATATCCGACTACAATCCAAATTTTGGAGATTTTAACT | |
| SEQ. ID NO:31 | |
| Nucleotide sequence of DNA region (1000 bp) up-stream from the siaD gene | |
| from Neisseria meningitidis (serogroup B) | |
| ATAATGCAGGCGCTGAAGTTGTTAACATCAAACACACATCGTTGAAGACGAAATGTCTGATGAGGCCAAACAAGTCATT | |
| CCAGGCAATGCAGATGTCTCTATTTATGAAATTATGGAACGTTGCGCCCTGAATGAAGAAGATGAGATTAATTAAAAGA | |
| ATACGTAGAGAGTAAGGGTATGATTTTTATCAGTACTCCTTTCTCTCGTGCAGCTGCTTTACGATTACAACGTATGGATA | |
| TTCCAGCATATAAAATCGGCTCTGGCGAATGTAATAACTACCCATTAATTAAACTGGTGGCCTCTTTTGGTAAGCCTATT | |
| ATTCTCTCTACCGGCATGAATTCTATTGAAAGCATCAAAAAGTCGGTAGAAATTATTCGAGAAGCAGGGGTACCTTATGC | |
| TTTGCTTCACTGTACCAACATCTACCCAACCCCTTACGAAGATGTTCGATTGGGTGGTATGAACGATTTATCTGAAGCCT | |
| TTCCAGACGCAATCATTGGCCTGTCTGACCATACCTTAGATAACTATGCTTGCTTAGGAGCAGTAGCTTTAGGCGGTTCG | |
| ATTTTAGAGCGTCACTTTACTGACCGCATGGATCGCCCAGGTCCGGATATTGTATGCTCTATGAATCCGGATACTTTTAA | |
| AGAGCTCAAGCAAGGCGCTCATGCTTTAAAATTGGCACGCGGCGGCAAAAAAGACACGATTATCGCGGGAGAAAAGCCAA | |
| CTAAAGATTTCGCCTTTGCATCTGTCGTAGCAGATAAAGACATTAAAAAAGGAGAACTGTTGTCCGGAGATAACCTATGG | |
| GTTAAACGCCCAGGCAATGGAGACTTCAGCGTCAACGAATATGAAACATTATTTGGTAAGGTCGCTGCTTGCAATATTCG | |
| CAAAGGTGCTCAAATCAAAAAAACTGATATTGAATAATGCTTATTAACTTAGTTACTTTATTAACAGAGGATTGGCTATT | |
| ACATATAGCTAATTCTCATTAATTTTTAAGAGATACAATA | |
| SEQ. ID NO:32 | |
| Nucleotide sequence of DNA region (1000 bp ) up-stream from the ctrA gene | |
| from Neisseria meningitidis (serogroup B) | |
| ATACCTGCACTTGAGTTGCCGACCATAAATTTAGCATGTTTCAATAAGACTAAAAAATATTCAAATCGAATGGAAGGAAA | |
| TGCAATAAATTTATCAGATTGATATTTTAATAATTCTTGCAGAATACTTTCAGTGCCAGTGTCATTATTAGGGTAGATGC | |
| TAATGATATTTTGGCCACTTAATTCTAATCCTTTGAAATATTGGGCCGCATATTGTGGCATTAAATGTGCTTCTGTAGTC | |
| ACGGGGTGAAACATAGAAATACCATAATTTTCGTATGGTAAACCGTAATATTCTTTGACTTCTTCTAAGGATGGGAGGGT | |
| GGAAGAGGCCATAACATCTAAATCGGGGGAGCCGATGATGTGAATATGCTTTCTTTTTTCTCCCATTTGCACTAGGCGAG | |
| TGACAGCTTGTTCATTTGCTACCAAGTGGATATGAGAAAGTTTACTAATAGAATGACGAATGGAGTCATCTACTGTACCA | |
| GATAGTTCACCACCTTCGATATGGCAAACTAAACGGCTGCTTAATGCACCTACAGCTGCGCCTGCTAGTGCTTCTAAACG | |
| GTCGCCGTGAATCATGACCATATCAGGTTCAATTTCATCAGATAGACGAGAGATAAACGTAATGGTATTGCCTAAAACGG | |
| CACCCATTGGTTCACCTTGGATTTGATTTGAAAACAGATATGTATGTTGATAGTTTTCTCGAGTTACTTCCTTGTAGGTT | |
| CTGCCATATGTTTTCATCATATGCATACCAGTTACAATCAAATGCAATTCAAGGTCTGGGTGATTTTCAATATAGGCTAA | |
| TAAAGGTTTTAGCTTGCCGAAGTCGGCTCTGGTACCTGTAATGCAAAGAATTCTTTTCATGATTTTAGAATCTATAAGTA | |
| TATATATATAAGTATAAGGAAGTTGGAAAGAAGAATACTAATTATACTCTACGTACTCATAAATTTATTTCGATTAAGTG | |
| CTATAATTAGGCCATTTATAATTATATTAGGATTTGGCTT | |
| SEQ. ID NO:33 | |
| Nucleotide sequence of DNA region (1000 bp) up-stream from the lgtF gene | |
| from Neisseria meningitidis (serogroup A) | |
| TCTTTTTCGGACTGAAAGGACGCATCATCCCGACATCGAGCGCGTGTTCGTCCGGCAGCCAAGGCATAGGTTATGCCTAC | |
| GAAGCCATCAAATACGGTCTGACCGATATGATGCTGGCGGGCGGAGGCGAAGAATTTTTCCCGTCCGAAGTGTATGTTTT | |
| CGACTCGCTTTATGCCGCCAGCCGCCGCAACGGCGAACCGGAAAAAACCCCGCGCCCATACGACGCGAACCGCGACGGGC | |
| TGGTCATCGGCGAAGGCGCGGGGATTTTCGTGCTGGAAGAATTGGAACACGCCAAACGGCGCGGTGCGATAATTTACGCC | |
| GAACTCGTCGGCTACGGAGCCAACAGCGATGCCTACCATATTTCCACGCCCCGCCCCGACGCGCAAGGCGCAATCCTTGC | |
| CTTTCAGACGGCATTGCAACACGCAGACCTTGCGCCCGAAGACATCGGCTGGATTAATCTGCACGGCACCGGGACGCACC | |
| ACAACGACAGTATGGAAAGCCGCGCCGTTGCAGCGGTTTTCGGCAACAATACGCCCTGCACGTCCACCAAGCCGCAAACC | |
| GGACACACGCTGGGCGCGGCGGGCGCAATCGAAGCCGCGTTCGCGTGGGGCATTGCTGACCGGAAAAGCAATCCCGAAGG | |
| GAAACTTCCGCCCCAGCTTTGGGACGGGCAGAACGATCCCGACCTTCCCGCCATCAACCTGACCGGCAGCGGCAGCCGCT | |
| GGGAAACCGAAAAACGCATTGCCGCCAGCTCGTCGTTTGCCTTCGGAGGAAGCAACTGCGTTTTACTCATCGGATGAAAT | |
| AAGTTTGTCAATCCCACCGCTATGCTATACAATACGCGCCTACTCTTGATGGGTCTGTAGCTCAGGGGTTAGAGCAGGGG | |
| ACTCATAATCCCTTGGTCGTGGGTTCGAGCCCCACCGGACCCACCAATTCCCAAGCCCGGACGTATGTTTGGGCTTTTTT | |
| GCCGCCCTGTGAAACCAAAATGCTTTGAGAAACCTTGATA | |
| SEQ. ID NO:34 | |
| Nucleotide sequence of DNA region (1000 bp) up-stream from the lgtB gene | |
| from Neisseria meningitidis (serogroup B) | |
| TAGAAAAATATTTCGCCCAATCATTAGCCGCCGTCGTGAATCAGACTTGGCGCAACTTGGAGATTTTGATTGTCGATGAC | |
| GGCTCGACAGACGGTACGCTTGCCATTGCCAAGGATTTTCAAAAGCGGGACAGCCGTATCAAAATCCTTGCACAAGCTCA | |
| AAATTCCGGCCTGATTCCCTCTTTAAACATCGGGCTGGACGAATTGGCAAAGTCAGGAATGGGGGAATATATTGCACGCA | |
| CCGATGCCGACGATATTGCCGCCCCCGACTGGATTGAGAAAATCGTGGGCGAGATGGAAAAAGACCGCAGCATCATCGCG | |
| ATGGGCGCGTGGCTGGAAGTTTTGTCGGAAGAAAAGGACGGCAACCGGCTGGCGCGGCATCACAGGCACGGCAAAATTTG | |
| GAAAAAGCCGACCCGGCACGAAGATATTGCCGACTTTTTCCCTTTCGGCAACCCCATACACAACAACACGATGATTATGA | |
| GGCGCAGCGTCATTGACGGCGGTTTGCCTTACAACACCGAGCGGGATTGGGCGGAAGATTACCAATTTTGGTACGATGTC | |
| AGCAAATTGGGCAGGCTGGCTTATTATCCCGAAGCCTTGGTCAAATACCGCCTTCACGCCAATCAGGTTTCATCCAAATA | |
| CAGCATCCGCCAACACGAAATCGCGCAAGGCATCCAAAAAACCGCCAGAAACGATTTTTTGCAGTCTATGGGTTTTAAAA | |
| CCCGGTTCGACAGCCTTGAATACCGCCAAATAAAAGCAGTAGCGTATGAATTGCTGGAGAAACATTTGCCGGAAGAAGAT | |
| TTTGAACGCGCCCGCCGGTTTTTGTACCAATGCTTCAAACGGACGGACACGCTGCCCGCCGGCGCGTGGCTGGATTTTGC | |
| GGCAGACGGCAGGATGCGGCGGCTGTTTACCTTGAGGCAATACTTCGGCATTTTGCACCGATTGCTGAAAAACCGTTGAA | |
| AAACGCCGCTTTATCCAACAGACAAAAAACAGGATAAATT | |
| SEQ. ID NO:35 | |
| Nucleotide sequence of DNA region (1000 bp) up-stream from the 1st gene from | |
| Neisseria meningitidis (serogroup B) | |
| GCGCACGGCTTTTTCTTCATCGGTTTGAGGGTCGGCAGGATAATCGGGGACGGCAAAGCCTTTAGACTGCAATTCTTTAA | |
| TCGCGGCGGTCAGTTGAGGTACGGATGCGCTGATGTTCGGCAGTTTGATTACGTTTGCATCGGGCTGTTTCACCAGTTCG | |
| CCCAATTCGGCAAGCGCGTCGGGTACGCGCTGCGCTTCGGTCAGATATTCGGGGAATGCCGCCAAAATACGGCCGGACAG | |
| GGAAATGTCGGCAGTTTTGACATCAATATCGGCGTGGCGGGCAAACGCCTGCACAATCGGCAGCAGCGATTGGGTCGCCA | |
| GCGCGGGGGCTTCGTCGGTATGGGTATAAACAATGGTGGATTTTTGAGTCATAGGATTATTCTCTTGTAGGTTGGTTTTT | |
| TCTTTTGGAACACATTGCGCGGGGAATGTGCGCGGCTATTATGGCATATTTTGGCGGCTTTGTTCGCGCTTTGTTCGATC | |
| TTGGCGTGTTTGAACGCGGCAGCGTGAAAGGAAGGGGGAAATGCTTTTCCCGCGTTTGGCGGCGGTGTCGGAGGTGCTGT | |
| GCCTGATGTGCGGCGGCATATTTTCGGTGAAATTGATTTTATAGTGGTTTAAATTTAAACCAGTACAGCGTTGCCTCGCC | |
| TTGTCGTACTATCTGTACTGTCTGCGGCTTCGTTGCCTTGTCCTGATTTAAATTTAAACCACTATAATATTCGCTAACTG | |
| TCGGAATATCTGCTAAAATTCCGCATTTTTCCGCCTCGGGACACTCGGGGCGTATGTTTAATTTGTCGGAATGGAGTTTT | |
| AGGGAT | |
| SEQ. ID NO:36 | |
| Nucleotide sequence of DNA region (1000 bp) up-stream from the msbB gene | |
| from Neisseria meningitidis (serogroup B) | |
| GCCCGACGGCGAACAGACACGTCGTGAAATCAACCGCTTGGACAGTACGGCGGCGCAATACGACATGCTTGCAGGTTATC | |
| TTGAAAGACTTGCCGGAAAAACCGACCGTTGGGCGTGCGCCTACCGCCAAAATGCCGTCTGAACACCCGATTATCCTTTT | |
| GAAAGCGCGATTATGCCCCATACCCTTCCCGATATTTCCCAATGTATCAGACAAAATTTGGAACAATATTTCAAAGACCT | |
| GAACGGTACCGAACCTTGCGGCGTGTACGATATGGTCTTGCATCAGGTGGAAAAACCGCTGCTGGTGTGCGTGATGGAAC | |
| AATGCGGCGGCAACCAGTCCAAAGCCTCCGTCATGTTGGGACTGAACCGCAATACTTTGCGTAAAAAACTGATTCAACAC | |
| GGTTTGCTGTGAATATGTCGGCAACCGTCCGTATCTTGGGTATTGACCCGGGCAGTCGCGTAACGGGTTTCGGTGTCATC | |
| GATGTCAGGGGGCGCGATCATTTTTACGTCGCCTCCGGCTGCATCAAAACGCCTGCCGATGCGCCTCTGGCAGACAGGAT | |
| TGCCGTGATTGTGCGGCATATCGGCGAAGTCGTTACCGTTTACAAGCCTCAACAGGCGGCAGTGGAACAGGTGTTCGTCA | |
| ACGTCAATCCGGCATCGACGCTGATGCTCGGTCAGGCTAGGGGCGCGGCATTGGCGGCATTGGTCAGCCATAAGCTGCCC | |
| GTTTCGGAATACACGGCCTTGCAGGTCAAACAGGCGGTAGTCGGCAAGGGCAAGGCGGCAAAAGAACAGGTGCAGCATAT | |
| GGTGGTGCAGATGCTGGGGCTTTCGGGAACGCCGCAGGANTGGCGGCGGACGGTCTTGCCGTCGCGCTGACCCACGCCTT | |
| ACGCAACCACGGGCTTGCCGCCAAACTCAATCCTTCGGGGATGCAGGTCAAGCGCGGCAGGTTTCAATAGTTTCAGACGG | |
| CATTTGTATTTTGCCGTCTGAAAAGAAAATGTGTATCGAG | |
| SEQ. ID NO:37 | |
| Nucleotide sequence of DNA region (1000 bp) up-stream from the htrB gene | |
| from Neisseria meningitidis (serogroup B) | |
| CCGCCAAGCGTTTCCCCCTTTGTCGGGCTTAACATTTGCTTTGTACGGCAGACTTTTTCCCTTCATAACGCCGCCTTTCC | |
| GAAAAGACGATGGTAGGCGCGACGTAATTCTCAACCCTTAAGGTACGGTTGGACGAAAAGTTTTCCTTTTCATTCCACCT | |
| GCCAACTTTTCGGCTACACCGAGTGGTCTCGTTAGGTTTGGGCGAACTACGCCCTTAAAAAAACGGACATTCTTTGCATG | |
| CCCGTCTCTAAGGTTTCACGGTAAGTTTACCCTTATAAAGAGTTGACTTACCATACTTATCCCTTTAAAACGATATAAAG | |
| GGCGACAGCTGTAATACAAGTATGTTGTACGGCAGACTTCTTCTACCAAACAAAAAGTTCCTTTTAGAGTTACTCGCTTA | |
| TAGACAAATGAAGGCTTAGCCATAGGCTTCCGGTAGGCCTATTTCAACGGCTGGTTCACAGGCTACGCTAAAACCTACGG | |
| TAGAACCGCGTTCTGGGGTTTCGCGCACAGCGGCGTCTTTGGAACCAGTTGTGTCCGAACACGCATAACCGCCCGCTTTA | |
| ATGGTGGTGGCGGGTTCACCTGATGTAGTTTCAGCGTGCGCTTTGGTAGTTTGCGTAGCCGATGTTGAGGAGGCTCGACC | |
| CGAAACTACGGTTGCCGACGCGCCAGCCGCACATGATGCTGGTCGTTAGAGGCCTGTAGCGGGTTCCGCACTTGCTTCCG | |
| CTTCCGTAACTGAACTTGGTTCCGCGACCGCTGGTTCCAAACTACAAGCCGATACGGACGCTGCTTTGGGGCTGGCACTA | |
| CGGCAAACGGTAGATAATGTCGGTGGCGGACTACGTCGCAGTTTCGCTTAATGCGTTTCTGCCGGAGGACGGAACCGACG | |
| CAGGGCTGCGTTTTCGGGTTGACTGGCACCAAATGCTATCGCTTAGGCCGTTTCATTTTGCGTAACTATGGCAGCAGGAG | |
| AGATACGTTGTGCTGGGCCTTTAGCCAATACTTCTCAACT | |
| SEQ. ID NO:38 | |
| Nucleotide sequence of DNA region (1000 bp) up-stream from the MltA gene | |
| from Neisseria meningitidis (serogroup B) | |
| CACAAAAACCAAGTTATGACGGGAATAAGGTACAGCAGCCAAACCAAGGCCTCGCCCTGC | |
| GTCGGATGGTCGGTATAGCCGAAAAATCCGCCGAGCAGCACGCCCAACGGGCTGTCTTCG | |
| TGCAAATATTTTGATGAGTCGAACACAATGTCCTGAAGCGCGTTCCAAATGCCTGCTTCG | |
| TGCAGCGCACGCAGCGAACCGGCAAGCAGACCAGCGGCAACGATAATCAGAAACGCCCCT | |
| GTCCAACGGAAAAACTTCGCCAGATTCAGGCGCATCCCACCCTGATAAATCAACGCGCCA | |
| ATCACGGCGGCAGCCAAAACCCCCGCTACCGCACCGGCCGGCATCTGCCACGTCGGGCTC | |
| TGTTTGAATACGGCAAGCAGGAAAAAAACGCTCTCCAAACCTTCGCGCGCCACGGCAAGA | |
| AACGCCATACCGACCAAGGCCCATCCTTGACCGCTGCCACGGTTCAAAGCCGCCTGCACA | |
| GAATCCTGAAGCTGCCGCTTCATCGAACGGGCGGCTTTTTTCATCCATAAAATCATATAA | |
| GTCAGCATCGCGACAGCAACCAAACCGATAATGCCGACGACGAACTCCTGCTGCTTCTGG | |
| GGAATCTCGCCCGTTGCCGAATGGATTCCGTACCCCAGCCCCAAACACATCAAAGAAGCA | |
| AGAACAACCCCGAACCAGACCTTAGGCATCAGTTTGGAATGTCCGGACTGTTTCAGAAAA | |
| CCGGCAACGATGCCGACGATGAGCGCGGCTTCGATACCCTCGCGCAACATAATTAAAAAA | |
| GCGACCAGCATAAACGCGAACGAACAAGGATGATGAATAATATATTATCGGAATATTTTC | |
| ATTGCTTGTAAATACAAATGCAAGTTATTTTTATCTGCAGTACCGCGCGGCGGAAAGTTC | |
| CGCAGCTGCAGCTGCGCCCTGTGTTAAAATCCCCTCTCCACGGCTGCCGCAACGCCGCCC | |
| GAAACCATCTTTCTTATTACTGCCGGCAACATTGTCCATT | |
| SEQ. ID NO:39 | |
| Nucleotide sequence of DNA region (1000 bp) up-stream from the ompCD gene | |
| from Moraxella catarrhalis | |
| GCTGATTTGTGAGCAAGCGGGCGCATCAGGGATTACCTTGCATTTGCGAGAAGATCGTCG | |
| ACATATTCAAGATGAAGATGTTTATGAATTGATTGGGCAATTGACAACACGCATGAATCT | |
| TGAGATGGCAGTCACTGATGAGATGCTAAATATTGCCCTAAAGGTACGACCAGCATGGGT | |
| GTGTTTAGTACCAGAAAAACGCCAAGAGCTGACTACAGAAGGTGGGCTTGATATCGCCAA | |
| TTTATCAAATATTCAAGCATTTATACACAGTCTTCAGCAGGCGGATATTAAGGTTTCTTT | |
| ATTCATCGATCCAGATCCGCATCAAATTGATGCTGCAATTGCTTTGGGTGCTGATGCGAT | |
| TGAGCTGCATACGGGAGCTTATGCTCAAGCGACTTTACAAAATAATCAAAAGCTTGTTGA | |
| TAAAGAGCTTGACCGTATTCAAAAAGCCGTTGCAATGGCACAAAAAAAATCATCATTATT | |
| GATTAATGCAGGTCATGGTTTGACGCGTGATAATGTTGCAGCGATTGCCCAAATTGATGG | |
| TATTCATGAGCTGAATATCCGGCATGCATTGATTTCAGATGCGATATTTATGGGGCTTGA | |
| TAATGCAGTCAAGGCAATGAAAATGGCTTTTATTCAAGATAAAACGACCAATCATTGATG | |
| CGTTAGAAAGAAAATCGTAAATAATGATGACTATTGTGTAATATTATGTATTTTTGTTCA | |
| AAAAAAGGTTGTAAAAAAATTCATTTACCATTAAGCTAAGCCCACAAGCCACAATGAATA | |
| CCTATTGGTTTGACTCATTAGTCACTAAGAATCTGCAAAATTTTGTAACAGATTATTGGC | |
| AGGTCTTGGATCGCTATGCTAAAATAGGTGCGGTAATCTTGAAAAACCAACCATTCCTTG | |
| GAGGAATTTATGAAAAAGGGATATAAACGCTCTTGCGGTCATCGCAGCCGTTGCAGCTCC | |
| AGTTGCAGCTCCAGTTGCTGCTCAAGCTGGTGTGACAGTC | |
| SEQ. ID NO:40 | |
| Nucleotide sequence of DNA region (1000 bp) up-stream from the copB gene | |
| from Moraxella catarrhalis | |
| GATGCTGTTAAAGTGGGTATTGGTCCTGGTTCTATTTGTACAACCCGTATTGTTGCAGGC | |
| ATTGGCGTCCCGCAGATAAGTGCCATTGATAGTGTGGCAAGTGCGTTAAAAGATCGCATT | |
| CCTTTGATTGCCGATGGCGGTATTCGTTTTTCGGGTGATATCGCCAAAGCCATCGCAGCA | |
| GGCGCTTCATGTATTATGGTGGGTAGCTTGTTGGCAGGTACCGAAGAAGCACCTGGTGAG | |
| GTGGAATTATTCCAAGGTCGTTATTATAAGGCTTATCGTGGTATGGGCAGCTTGGGGGCA | |
| ATGTCTGGTCAAAATGGCTCATCGGATCGTTATTTTCAAGATGCCAAAGATGGTGTTGAA | |
| AAACTGGTTCCAGAGGGTATCGAAGGCCGTGTTCCTTATAAAGGCCCTGTGGCAGGCATC | |
| ATCGGTCAATTGGCAGGTGGTCTAAGATCATCCATGGGTTATACAGGTTGCCAGACCATC | |
| GAACAGATGCGTAAGAATACCAGCTTTGTCAAAGTGACTTCCGCAGGCATGAAGGAATCG | |
| CATGTACACGATGTACAGATTACCAAAGAAGCACCCAATTATCGCCAAAATTAACTCTAT | |
| TAATAGCAAATACAAGCACTCATTAGATAGGGTGGGTGCTTTTTAGAGCATAAAAAATAA | |
| ACTGACACATGACTTATTGTCATATTTTTAAAATGCTTTTAATTTAGATTTTTAATTTAG | |
| ATAATGGCTAAAAATAACAGAATATTAATTTAAAGTTTTCAAAATCAAGCGATTAGATGA | |
| AATTATGAAAATAAATAACAATAATTCTGATTTATTTTAACCAATAATATCAATTATCAT | |
| TTACAAGAAAAATTTTTTTTGATAAAATTCTTACTTGTACCTTGCTATTTTTTCTTATTT | |
| ATCATTTTTGGCGGTATTTTCGTTGATTTTAGTAAGTAGATGAGCAAGGGATAATTTGAC | |
| AAAAACAAATTTGATTTCAAGCCTCATAATCGGAGTTATT | |
| SEQ. ID NO:41 | |
| Nucleotide sequence of DNA region (1000 bp) up-stream from the D15 gene | |
| from Moraxella catarrhalis | |
| AAAACTGGTGATGTCTTCACTGCTATTCATGGTGAACCAATCAATGATTGGCTAAGTGCC | |
| ACCAAGATTATTCAGGCAAATCCAGAAACCATGCTTGATGTGACAGTCATGCGTCAAGGT | |
| AAGCAGGTTGATTTAAAATTAATGCCCCGTGGTGTAAAGACACAAAACGGCGTAGTCGGT | |
| CAACTGGGTATTCGCCCCCAGATTGATATCGATACGCTCATTCCTGATGAATATCGTATG | |
| ACGATTCAATATGATGTCGGTGAGGCATTTACTCAAGCCATCCGACGAACTTATGATTTA | |
| TCAATAATGACCTTAGATGCGATGGGTAAGATGATTACAGGATTGATTGGCATTGAAAAT | |
| CTATCAGGTCCCATTGCCATTGCCGATGTTTCTAAGACCAGTTTTGAGTTGGGATTTCAA | |
| GAAGTGTTATCGACAGCCGCAATCATCAGTTTAAGCTTGGCAGTACTGAATCTTTTACCC | |
| ATTCCAGTGTTAGATGGCGGGCATTTGGTATTTTATACTTATGAATGGATTATGGGCAAA | |
| TCTATGAATGAAGCGGTGCAGATGGCAGCATTTAAAGCGGGTGCGTTATTGCTTTTTTGT | |
| TTCATGTTACTTGCAATCAGTAACGATATCATGCGATTTTTTGGCTAAGTTCTGATTTAT | |
| CGTACCATTAACAAAATTTTTGGCTTTTTTAAGCTGAAATACTTGCCAAATTTAACTTTT | |
| TGGCTTACCTTTACACAATATAAATTTGGGTGTAGAAAATTTTGGATACATTTTTATACC | |
| TTATTTTTAGAAATTTTAAAAATTAAGTTTGGATAGACTTATGCGTAATTCATATTTTAA | |
| AGGTTTTCAGGTCAGTGCAATGACAATGGCTGTCATGATGGTAATGTCAACTCATGCACA | |
| AGCGGCGGATTTTATGGCAAATGACATTGCCATCACAGGACTACAGCGAGTGACCATTGA | |
| AAGCTTACAAAGCGTGCTGCCGTTTCGCTTGGGTCAAGTG | |
| SEQ. ID NO:42 | |
| Nucleotide sequence of DNA region (1000 bp) up-stream from the omplA gene | |
| from Moraxella catarrhalis | |
| ACTTGGCGAAAATACCATTTATATCGATTGTGATGTTATACAGGCAGATGGCGGTACACG | |
| CACAGCCAGTATCAGTGGTGCTGCGGTGGCACTTATTGATGCTTTAGAACACTTGCAGCG | |
| TCGTAAAAAGCTTACCCAAGATCCGCTTTTGGGCTTGGTGGCAGCGGTTTCTGTGGGTGT | |
| TAATCAAGGCCGTGTATTGCTTGATTTGGATTATGCTGAAGATTCAACTTGTGATACCGA | |
| TTTAAATGTGGTCATGACGCAGGCAGGTGGGTTTATTGAGATTCAAGGCACAGCAGAAGA | |
| AAAGCCATTTACTCGTGCTGAAGCTAATGCGATGCTTGATTTGGCAGAGCTGGGAATTGG | |
| GCAGATTATCGAAGCCCAAAAGCAAGTATTAGGCTGGTGATATGCTAATCGTTGAAGATA | |
| ATGGCGTGATCATCACATTAAATGGACAAGTAAAAGACCCATTATTTTGGTGGTCGATGA | |
| TATTGCTGCTGCTGGGTGTCTTGGTGGCAATCATTTGTTTGATTGCACCCGTTTTTTATG | |
| CAATCGGTGCGTTGGCTTTATTTGCAGTTGTGGTATTTGTGTTTAATATTCAAAGGCAAA | |
| AAGCCAAAACTTGTCATATGTTTTCACAAGGTCGCTTGAAGATTACGTCCAAACGCTTTG | |
| AGATTCATAACAAATCACTAACCTTATCAGCATCGGCAACAATATCTGCTAAAGATAACA | |
| AAATGACAATTGTTGATCGGGGCATTGAATATCATTTTACAGGTTTTGCTGATGACCGTG | |
| AAATTAATATAGCCAAACAGGTACTTTTGGGAAAGTCAATCAAAACCAATGCGGTGGCGG | |
| TAACATTGGCTAAGTAGTTGTTGTGATACAGACAGGTTGGATGGTCTTTAACTCCACCCA | |
| CCTAACTTTTTCTTTGTTTGGATTTAAGAGTATGTTATGATGGGCAGGATTTTATTTTAA | |
| GTCATCATTTAATGCAATCAGTTGTCCAGAGTAGCCGTTC | |
| SEQ. ID NO:43 | |
| Nucleotide sequence of DNA region (1000 bp) up-stream from the hly3 gene | |
| from Moraxella catarrhails | |
| GTGATCGGCAACACCCCACCATTCAGGAGCAACCAAAATTGCCCGTGCCTTGCCTGTCTT | |
| GGTGGTATCATTTGGCAGGGCAATGTGGCTAAGTAGTGGTGTGCCATCAGGTGCGGTGGT | |
| GGTGAGTGTACGATTCGTTATTGTCATAAAATTATCCTTTTGGGTTGGATGATATCAATG | |
| AAATACCCTACGGTTGTATGGAATTTTATCCATTGTACCACGGTATTGGTCTTTTTAAAT | |
| TAACAAGCAGCTTCTAGCAAGTCAAAGTTTTTATGCCTATTTTTTCAGATTTTAAGGTAC | |
| AATAAAGCCAATTGTTAATAATATGGTATTGTCATGATTTATGATGAATTGCGACCAAAA | |
| TTTTGGGAAAATTATCCCTTAGATGCGTTAACAGATGCTGAATGGGAAGCATTATGTGAC | |
| GGATGTGGCGCGTGTTGTTTGGTGAAATTTCTTGATGATGACAATGTTAAATTGACCGAA | |
| TATACCGATGTTGCCTGCCAGCTATTGGATTGCTCAACiGGATTTTGCCAAAACTATGCC | |
| AAGCGTCAAACGATTGTGCCAGATTGTATTCGCTTAACACCTGATATGCTGCCTGATATG | |
| CTGTGGTTGCCACGCCATTGTGCTTATAAGCGGTTGTATCTTGGGCAAAATCTGCCAGCA | |
| TGGCACAGGCTCATTAAACATAGCCAAAACCATGGTGCAGGATTTGCGAAAGTTTCAACT | |
| GCTGGGCGATGTGTGAGTGAGCTTGGTATGAGTGATGAAGACATAGAAAGGCGAGTGGTG | |
| AAATGGGTTAAACCTTGACATGATTGTTGACATGATTGACAGACAATAAAAATTGGCAAA | |
| TTTGATAAAATTGGTGTATGTGTGTGATTTTATCAAAAGCACTTGAATAAAACCGAGTGA | |
| TACGCTAAATTGTAGCAAACCAATCAATTCATCATAATTTTAATGAACACGACGTTAAAT | |
| TATACTGTCTATGTCTGATGACAATTCAAGCACTTGGTCG | |
| SEQ. ID NO:44 | |
| Nucleotide sequence of DNA region (1000 bp) up-stream from the lbpA gene | |
| from Moraxella catarrhalis | |
| TAACAAAGGCAACCCAACACGCAGTTATTTTGTGCAAGGCGGTCAAGCGGATGTCAGTAC | |
| TCAGCTGCCCAGTGCAGGTAAATTCACCTATAATGGTCTTTGGGCAGGCTACCTGACCCA | |
| GAAAAAAGACAAAGGTTATAGCAAAGATGAGGATACCATCAAGCAAAAAGGTCTTAAAGA | |
| TTATATATTGACCAAAGACTTTATCCCACAAGATGACGATGACGATGACGATGACGATAG | |
| TTTGACCGCATCTGATGATTCACAAGATGATAATACACATGGCGATGATGATTTGATTGC | |
| ATCTGATGATTCACAAGATGATGACGCAGATGGCGATGACGATTCAGATGATTTGGGTGA | |
| TGGTGCAGATGATGACGCCGCAGGCAAAGTGTATCATGCAGGTAATATTCGCCCTGAATT | |
| TGAAAACAAATACTTGCCCATTAATGAGCCTACTCATGAAAAAACCTTTGCCCTAGATGG | |
| TAAAAATAAGGCTAAGTTTGATGTAAACTTTGACACCAACAGCCTAACTGGTAAATTAAA | |
| CGATGAGAGAGGTGATATCGTCTTTGATATCAAAAATGGCAAAATTGATCGCACAGGATT | |
| TACCGCCAAAGCCGATGTGCCAAACTATCGTGAAGAAGTGGGTAACAACCAAGGTGGCGG | |
| TTTCTTATACAACATCAAAGATATTGATGTTAAGGGGCAATTTTTTGGCACAAATGGCGA | |
| AGAGTTGGCAGGACGGTTACATCATGACAAAGGCGATGGCATCACTGACACCGCCGAAAA | |
| AGCAGGGGCTGTCTTTGGGGCTGTTAAAGATAAATAAAGCCCCCCTCATCATCGTTTAGT | |
| CGCTTGACCGACAGTTGATGACGCCCTTGGCAATGTCTTAAAACAGCACTTTGAAACAGT | |
| GCCTTGGGCGAATTCTTGGATAAATGCACCAGATTTGCCTCGGGCTAATATCTTGATAAA | |
| ACATCGCCATAAAATAGAAAATAAAGTTTAGGATTTTTTT | |
| SEQ. ID NO:45 | |
| Nucleotide sequence of DNA region (1000 bp) up-stream from the lbpB gene | |
| from Moraxella catarrhalis | |
| CAGCTTGTACCATTTGGTGAATATATACCATTTGGTGGTTTGTTG~ATATTTTACCAGGG | |
| CTTGAGGGTGTCGCTAGCCTAAGCCGTGGCGATGATAAGCAACCACCGCTCAAATTGGGC | |
| GGCGGCGTGGGCGATACGATTGGTGCGGCAATTTGTTATGAGGTGGCATATCCTGAGACG | |
| ACGCGTAAAAATGCACTTGGCAGTAATTTTTTATTAACCGTCTCAAACGATGCTTGGTTT | |
| GGTACAACAGCAGGTCCTTTGCAGCATTTACAAATGGTGCAAATGCGAAGCTTGGAGACG | |
| GGGCGATGGTTTGTGCGTGCAACAAACAACGGAGTGACTGCATTAATTGACCATCAAGGA | |
| CGGATTATCAAGCAGATACCGCAGTTTCAGCGAGATATTTTGCGAGGTGATGTACCCAGT | |
| TATGTTGGACACACGCCTTATATGGTTTGGGGGCATTATCCCATGTTGGGGTTTTCTTTG | |
| GTGCTGATTTTTCTTAGTATCATGGCAAAGAAAATGAAAAATACCACCGCCAAACGAGAA | |
| AAATTTTATACCGCTGATGGTGTGGTAGACCGCTGAATTGTGCCACTTTGGGCGTTAGAG | |
| CATGAGCAAGATTAGGCGTTGGGTGAGCTTTGGTTGTATTACTCATCAGCCTACCCGAAA | |
| CCTGCCAAACATCACCGCCCAAAACCTAAACATACAATGGCTAAAAATATCAGAAAATAA | |
| CTTGCTGTATTGTAAATTCTTATGTTATCATGTGATAATAATTATCATTAGTACCAAGAT | |
| ATCCATTACTAAACTTCATCCCCCATCTTAACAGTTACCAAGCGGTGAGCGGATTATCCG | |
| ATTGACAGCAAGCTTAGCATGATGGCATCGGCTGATTGTCTTTTTGCCTTGTTGTGTGTT | |
| TGTGGGAGTTGATTGTACTTACCTTAGTGGTGGATGCTTGGGCTGATTTAATTAAATTTG | |
| ATCAAAGCGGTCTTCACAACACACCAAACGAGATATCACC | |
| SEQ. ID NO:46 | |
| ucleotide sequence of DNA region (1000 bp) up-stream from the tbpB gene | |
| from Moraxella catarrhalis | |
| AGTTTGCCCTGATTTTGAGAGCCACTGCCATCATGAATTTGTTGGCGTAAACACCACTCG | |
| TATTCTTCTTCGGTTTCCCCTTTCCATGCAAACACAGGGATACCAGCGGCCGCCATGGCA | |
| GCGGCGGCGTGGTCTTGGGTGCTAAAAATATTGCATGATGTCCAGCGAACTTCTGCACCC | |
| AAGCCAACCAAAGTCTCAATCAGCACCGCTCTTTGAATGGTCATGTGGATACAGCCTAGG | |
| ATTTTAGCACCCTTAAGTGGTTGCTGGTCTTGATAGCGTTTTCTTAACCCCATCAGGGCT | |
| GGCATCTCAGCTTCTGCCAAGGCAATCTCACGGCGACCATAATCGGCTAAACGGATATCA | |
| GCGACTTTATAATCGGTGAAGTTTTGGGTGGTACTTGGATTGATTGAGGTAGGCATATCT | |
| TTATTCCTAAGCTATTTTAAAGTATTTTTAACAATAATTTTGATGAATTTGAGATAATTG | |
| ATGCTAAAAGGTTGAATGACCAAACCATCGCTAACAATCAAGAAAAGACATTTTAAGCAT | |
| AAAAAGCAAATGTGTCTTGATGGCTTATTATAACAGTTATTATGATAAATTTGGGTAGAA | |
| AGTTAAATGGATCGTTGGGTAAGTTTGTTGGCTATCCTTAATTAATTATAATTTTTTAAT | |
| AATGCTTTTACTTTATTTTAAAAATAGAGTAAAAAATGGTTGGCTTTGGGTTTTTATCTC | |
| ACTATGGTAGATAAAATTGATACAAAATGGTTTGTATTATCACTTGTATTTGTATTATAA | |
| TTTTACTTATTTTTACAAACTATACACTAAAATCAAAAATTAATCACTTTGGTTGGGTGG | |
| TTTTAGCAAGCAAATGGTTATTTTGGTAAACAATTAAGTTCTTAAAAACGATACACGCTC | |
| ATAAACAGATGGTTTTTGGCATCTGCAATTTGATGCCTGCCTTGTGATTGGTTGGGGTGT | |
| ATCGGTGTATCAAAGTGCAAAAGCCAACAGGTGGTCATTG | |
| SEQ. ID NO:47 | |
| Nucleotide sequence of DNA region (1000 bp) up-stream from the tbpA gene | |
| from Moraxella catarrhalis | |
| TTGGGGGCGGATAAAAAGTGGTCTTTGCCCAAAGGGGCATATGTGGGAGCGAACACCCAA | |
| ATCTATGGCAAACATCATCAAAATCACAAAAAATACAACGACCATTGGGGCAGACTGGGG | |
| GCAAATTTGGGCTTTGCTGATGCCAAAAAAGACCTTAGCATTGAGACCTATGGTGAAAAA | |
| AGATTTTATGGGCATGAGCGTTATACCGACACCATCGGCATACGCATGTCGGTTGATTAT | |
| AGAATCAACCCAAAATTTCAAAGCCTAAACGCCATAGACATATCACGCCTAACCAACCAT | |
| CGGACGCCCAGGGCTGACAGTAATAACACTTTATACAGCACATCATTGATTTATTACCCA | |
| AATGCCACACGCTATTATCTTTTGGGGGCAGACTTTTATGATGAAAAAGTGCCACAAGAC | |
| CCATCTGACAGCTATGAGCGTCGTGGCATACGCACAGCGTGGGGGCAAGAATGGGCGGGT | |
| GGTCTTTCAAGCCGTGCCCAAATCAGCATCAACAAACGCCATTACCAAGGGGCAAACCTA | |
| ACCAGTGGCGGACAAATTCGCCATGATAAACAGATGCAAGCGTCTTTATCGCTTTGGCAC | |
| AGAGACATTCACAAATGGGGCATCACGCCACGGCTGACCATCAGTACAAACATCAATAAA | |
| AGCAATGACATCAAGGCAAATTATCACAAAAATCAAATGTTTGTTGAGTTTAGTCGCATT | |
| TTTTGATGGGATAAGCACGCCCTACTTTTGTTTTTGTAAAAAAATGTGCCATCATAGACA | |
| ATATCAAGAAAAAATCAAGAAAAAAAGATTACAAATTTAATGATAATTGTTATTGTTTAT | |
| GTTATTATTTATCAATGTAAATTTGCCGTATTTTGTCCATCACAAACGCATTTATCATCA | |
| ATGCCCAGACAAATACGCCAAATGCACATTGTCAACATGCCAAAATAGGCATTAACAGAC | |
| TTTTTTAGATAATACCATCAACCCATCAGAGGATTATTTT | |
| SEQ. ID NO:48 | |
| Nucleotide sequence of DNA region (1000 bp) up-stream from the ompE gene | |
| from Moraxella catarrhalis | |
| AAAGACATTACACATCATCATTCAAACGCCCAACCATGTACCTCTGCCCCGTGGTCGCAC | |
| GCCAACGCTTTTTGATGCGGTGCGTTGGGTTCAGATGGCTTGTCAATCATTTGGTTTTAT | |
| TAAAATTCATACCTTTGGTAGTTTGGCTTTACCTGATATGTCATTTGATTATCGAAACAA | |
| TACGCAGTTGACCAAACATCAATTTTTAGCCATTTGCCAAGCACTCAATATTACCGCTCA | |
| TACGACCATGCTTGGTATTAAATCATCACATAAAGATACTTTACATCCATTTGAATTGAC | |
| ATTACCCAAATACGGCCATGCCTCAAATTATGATGATGAATTGGTGCAAAACAATCCATT | |
| GGCTTATTTTCATCAACTGTCTGCCGTCTGCCGATATTTTTATACCCAAACGGTTTGTAT | |
| TGTTGGCGGTGAAAGCTCAGGGAAAACTACCTTGGTGCAAAAACTTGCCAATTATTATGG | |
| TGCCAGCATCGCACCTGAAATGGGTCGATTATACACACACTCCCATCTCGGCGGTAGCGA | |
| ACTTGCCCTTCAATACAGCGACTACGCATCCATTGCCATCAATCACGCCAACGCTATCGA | |
| AACCGCTCGTACCACTGCCAGCTCTGCTGTTACACTGATTGATACTGATTTTGCGACAAC | |
| GCAAGCATTTTGTCAAATTTATGAAGGGCGAACGCATCCGCTTGTCGCAGAATTTGCTAA | |
| ACAAATGCGATTGGATTTTACGATTTATTTAGATAATAATGTTGCTTGGGTCGCTGATGG | |
| CATGCGTAGGCTTGGTGATGATCATCAACGCAGTTTGTTCGCCAATAAATTGCTTGAGAT | |
| TTTGGCACGATATGATATTAGTTATCATATCATTAATGACACCGACTACCACAAACGCTA | |
| TCTACAAGCATTAAGCTTGATAGACAATCATATTTTTAATCATTTTACAAAAATTCATGA | |
| CAATTAATTAGGGAAAATCTGATGAAAATTGATATTTTAG | |
| SEQ. ID NO:49 | |
| Nucleotide sequence of DNA region (1000 bp) up-stream from the uspa1 gene | |
| from Moraxella catarrhalis | |
| GGATGTGGCATATCTGCCCATCGACCCAATACACATCGCTCGAGGCTATCAAGATGTGGT | |
| ACGAATTAATAGCCAGTCAGGTAAGGGCGGTGCTGCGTATATCTTGCAGCGGCATTTTGG | |
| TTTTAATTTACCACGCTGGACACAGATTGATTTTGCTCGTGTGGTACAGGCTTATGCAGA | |
| AAGTATGGCGCGTGAACTAAAAACTGATGAGCTGCTTGAAATTTTTACCCAAGCGTATCT | |
| TAAGCAAGATAAATTCCGCCTAAGTCACTATACCATCAGCAATAAAGGCGATGCTGTCAG | |
| CTTCCAAGGCCAAGTAGCGACACCCAAAGCGGTGTTTGAGGTGATTGGTCAAGGCAATGG | |
| TGCGTTATCTGCGTTCATTGATGGCTTGGTGAAATCCACAGGCAGACAGATTCATGTCAC | |
| CAATTACGCCGAACACGCCATCGATAACAAAACCCATCAAAAAACCGATACGGATAACCA | |
| AACCGATGCCGCCGTGCCGCTTATATCCAGCTGTCGGTAGAGGGGCAGATTTATTCAGGC | |
| ATCGCCACTTGCCATAGCACCGTATCCGCCATGCTAAAAGGTGCATTATCCGCTTTGGCA | |
| CAGGCGTGGTAATCTGACCCAATCAAAATCCTGCATGATGGCAGGATTTTATTATTTAGT | |
| GGGCTGCCCAACAATGATGATCATCAGCATGTGAGCAAATGACTGGCGTAAATGACTGAT | |
| GAGTGTCTATTTAATGAAAGATATCAATATATAAAAGTTGACTATAGCGATGCAATACAG | |
| TAAAATTTGTTACGGCTAAACATAACGACGGTCCAAGATGGCGGATATCGCCATTTACCA | |
| ACCTGATAATCAGTTTGATAGCCATTAGCGATGGCATCAAGTTGTGTTGTTGTATTGTCA | |
| TATAAACGGTAAATTTGGTTTGGTGGATGCCCCATCTGATTTACCGTCCCCCTAATAAGT | |
| GAGGGGGGGGGAGACCCCAGTCATTTATTAGGAGACTAAG | |
| SEQ. ID NO:50 | |
| Nucleotide sequence of DNA region (1000 bp) up-stream from the uspa2 gene | |
| from Moraxella catarrhalis | |
| CCCCAAGCTTTCCGTTTGTGTGCCTGCTGGTGTCGGGCGGTCATACCATGCTGGTGCGTG | |
| CCGATGGTGTGGGCGTGTATCAGATATTGGGCGAGTCTATCGATGATGCGGTGGGTGAAT | |
| GCTTTGATAAAACGGCAAAAATGCTCAAACTGCCCTATCCTGGTGGCCCAAATATCGAAA | |
| AATTAGCCAAAAACGGCAACCCACACGCCTATGAGCTGCCAAGACCCATGCAGCATAAAG | |
| GGCTGGATTTTTCGTTCAGTGGCATGAAAACCGCCATTCATAATCTCATCAAAGACACAC | |
| CAAACGCCCAAAGCGACCCCGCCACACGAGCAGACATCGCCGCAAGCTTTGAGTATGCGG | |
| TGGTGGATACTTTGGTCAAAAAATGCACCAAAGCACTACAGATGACAGGCATTCGCCAGC | |
| TGGTGGTCGCAGGGGGCGTCTCTGCCAATCAGATGCTACGCCGCACCCTGACCGAGACGC | |
| TCCGCCAAATCGATGCGTCGGTGTACTATGCCCCGACCGAGCTATGCACGGATAATGGTG | |
| CGATGATCGCCTATGCTGGCTTTTGTCGGCTCAGCTGTGGACAGTCGGATGACTTGGCGG | |
| TTCGCTGTATTCCCCGATGGGATATGACGACGCTTGGCGTATCGGCTCATAGATAGCCAC | |
| ATCAATCATACCAACCAAATCGTACAAACGGTTGATACATGCCAAAAATACCATATTGAA | |
| AGTAGGGTTTGGGTATTATTTATGTAACTTATATCTAATTTGGTGTTGATACTTTGATAA | |
| AGCCTTGCTATACTGTAACCTAAATGGATATGATAGAGATTTTTCCATTTATGCCAGCAA | |
| AAGAGATAGATAGATAGATAGATAGATAGAACTCTGTCTTTTATCTGTCCGCTGATGCTT | |
| TCTGCCTGCCACCGATGATATCATTTATCTGCTTTTTAGGCATCAGTTATTTCACCGTGA | |
| TGACTGATGTGATGACTTAACCACCAAAAGAGAGTGCTAA | |
| SEQ. ID NO:51 | |
| Nucleotide sequence of DNA region (1000 bp) up-stream from the omp21 gene | |
| from Moraxella catarrhalis | |
| GAGTGAACTTTATTGTAAAATATGATTCATTAAAGTATCAAAATCATCAAACGCAGCATC | |
| AGGGTTTGCTAAATCAATTTTTTCACCATAATTATAGCCATAACGCACAGCAAGCGTAGT | |
| TATGCCAGCGGCTTGCCCTGATAAAATATCATTTTTGGAATCACCAACCATAATGGCATC | |
| AGTCGGTGCGATGCCCAGTGATTGACACAGGTATAATAAAGGCGTTGGGTCGGGCTTTTT | |
| GACGCTGAGCGTATCACCGCCAATCACTTGGTCAAACAGTGTCAGCCATCCAAAATGTGA | |
| TAAAATTTTAGGCAAATAACGCTCAGGCTTATTGGTACAAATTGCCAAATAAAACCCCGC | |
| TGCTTTTAATCGTTCAAGCCCTTGTATAACCCCTGCATAGCTTTGCGTATTTTCAATTGT | |
| TTTATGGGCATATTCTGCCAAAAATAACTCATGGGCATGGTGAATCATAGTCGTATCATA | |
| GATATGATGTGCTTGCATTGCTCGCTCAACCAATTTTAGCGAACCATTGCCCACCCAGCT | |
| TTTGATGATATCAATTGGCATAGGCGGTAAGTTAAGCTTGGCATACATGCCATTGACCGC | |
| CGCCGCCAAATCAGGGGCACTATCGATAAGCGTACCATCCAAATCAAATATAATCAGTTT | |
| TTTGCCAGTCATTGACAGTGTTTGCATGCTTTTTCCTTATTCTTAAAATTGGCGGCTGTT | |
| TGGTATTTTTTAAATCAGTCAATTTTTACCATTTGTCATATAATGACAAAGTACAAATTT | |
| AGCAATATTTTAGTGCATTTTTTGGCGAAGTTTTATGAAAACTGGTCATTGGTTGCAAAA | |
| CTTTACACAGTACCTATAAAACTTGCACAGTTAATAAGAAATATTTTGTTACTATAGGGG | |
| CGTCATTTGGAACAAGACAGTTATTTGTAAATAGTTATTTGCAAAAGACGGCTAAAAGAC | |
| AGAACAGCGTTTGTTTCAGTGATTAACTAGGAGAAAAACA | |
| SEQ. ID NO:52 | |
| Nucleotide sequence of DNA region (1000 bp) up-stream from the omp106 gene | |
| from Moraxella catarrhalis | |
| TTGATCGGTTTTGCCCCACTGTTTCATGATTTACTCAAAACAGGCGGCTTGATCGTGCTG | |
| GCAGGTCTGACCCAAAACCAAACCCAAGCGGTCATCGATGCCTACTCGCCTTATGTTACG | |
| CTTGATACGCCATTTTGTTATGCAGATGCCCAAGACTGCCATTGGCAACGCCTAAGCGGC | |
| ATCAAACCTACCAACCCATAAGCGATATGCCATGAGCCACAAACCTAAGCCAACACCGCT | |
| ATATCAACAAGTTGAGCAGACCGCCAAGCGTTATTTTGAGACATTGGGCGATGCTCATAC | |
| TCATGATGTCTATGCCACTTTTTTGGCCGAATTTGAAAAACCGCTGCTCATCGCCGCACT | |
| CAATCACACGCACGGCAATCAGTCAAAAACCGCCCAAATCCTTGGTATCAATCGTGGCAC | |
| ATTACGCACCAAAATGAAAACCCATCACTTACTTTAGACCGCCAGTTATCGCCATGGATA | |
| TGGGCAGGTGTGCTCGCCTGCCGTATGATGGCGATGACACCCCATTTGCCCCATATCTGC | |
| ACGATTTGACATGATTTAACATGTGATATGATTTAACATGTGACATGATTTAACATTGTT | |
| TAATACTGTTGCCATCATTACCATAATTTAGTAACGCATTTGTAAAAATCATTGCCCCCT | |
| TTTTTTATGTGTATCATATGAATAGAATATTATGATTGTATCTGATTATTGTATCAGAAT | |
| GGTGATGCCTACGAGTTGATTTGGGTTAATCACTCTATTATTTGATATGTTTTGAAACTA | |
| ATCTATTGACTTAAATCACCATATGGTTATAATTTAGCATAATGGTAGGCTTTTTGTAAA | |
| AATCACATCGCAATATTGTTCTACTGTTACCACCATGCTTGAATGACGATCCAAATCACC | |
| AGATTCATTCAAGTGATGTGTTTGTATACGCACCATTTACCCTAATTATTTCAATCAAAT | |
| GCCTATGTCAGCATGTATCATTTTTTTAAGGTAAACCACC | |
| SEQ. ID NO:53 | |
| Nucleotide sequence of DNA region (1000 bp) up-stream from the HtrB gene | |
| from Moraxella catarrhalis | |
| ACTATTCTGCTTTTTGTTTTTCACGAATGCCAATGCCCAACTCACGCAACTGGCGATTAT | |
| CAACTTCAGCAGGTGCTTCGGTCAATGGGCAATCTGCCGTCTTGGTTTTTGGGAAGGCGA | |
| TCACATCACGGATTGAGCTGGCACCAACCATCAGCATAATCAGGCGATCTAGACCAAATG | |
| CCAAACCACCGTGCGGCGGTGCACCAAAACGCAATGCATCCATCAAAAACTTAAACTTAA | |
| GCTCTGCTTCTTCTTTAGAAATACCCAAGGCATCAAATACCGCCTCTTGCATGTCAACCG | |
| TATTAATACGCAGCGAACCGCCACCAATTTCTGTGCCATTTAGTACCATGTCATAGGCAA | |
| TGGATAGGGCGGTTTCGGGACTTTGTTTGAGTTCCTCAACCGAGCCTTTTGGGCGTGTAA | |
| AAGGATGATGAACTGATGTCCACTTACCATCATCAGTTTCCTCAAACATTGGAAAATCAA | |
| CGACCCAAAGCGGTGCCCATTCACAGGTAAATAAATTTAAATCAGTACCGATTTTAACAC | |
| GCAATGCACCCATAGCATCATTGACGATTTTGGCTTTATCGGCACCAAAGAAAATGATAT | |
| CGCCAGTTTGGGCATCGGTACGCTCAATCAGCTCAATCAAAACCTCATCGGTCATATTTT | |
| TAATGATGGGTGATTGTAATCCTGATTCTTTTTCAACGCCATTATTGATATTGCTTGCGT | |
| CATTGACCTTAATATATGCCAATCCACGAGCGCCATAAATACCAACAAATTTGGTGTACT | |
| CATCAATCTGCTTGCGACTCATGTTACCGCCATTTGGAATGCGTAAGGCAACAACACGGC | |
| CTTTAGGATCTTGGGCGGGCCCTGAAAATACTTTAAATTCAACATGTTGCATGATGTCAG | |
| CAACATCAATAAGTTTTAAGGGAATGCGTAAATCAGGCTTATCTGAGGCATAATCACGCA | |
| TGGCATCTGCGTAAGTCATGCGGGGGAAGGTATCAAACTCA | |
| SEQ. ID NO:54 | |
| Nucleotide sequence of DNA region (1000 bp) up-stream from the MsbB gene | |
| from Moraxella catarrhalis | |
| TGGATCATATTCTTTATTAATGGTACTGTTTAAACCTGTATTTTAAAGTTTATTGGGTCA | |
| TATTTTCAAGCTCATCCCATCGCTCAAGCTTCATCATCAAAAGCTCATCAATCTCTACCA | |
| ATCGCTCACCAGCCTTCGTTGCTGCCGCCAAATCGGTATTAAACCATGAACCATCTTCAA | |
| TCTTTTTGGCAAGCTGTGCCTGCTCTTGTTCAAGTGCAGCAATTTCATTAGGCAAATCTT | |
| CAAGTTCACGCTGCTCTTTATAGCTGAGTTTGCGTTTTTGGGCAACGCCTGATTGAGGTG | |
| GTTTGATTTGGATGGGTTCAGCGGGTTTTGTCGCCTTAGGTTTATTGTCTGTGGCGTGAT | |
| GAGCAAGCCATCTTTCATGCTGTTGTACATAGTCTTCATAACCGCCAACATATTCCAAAA | |
| CGATACCGTCGCCGTACTTATCAGTATCAAATACCCAAGTTTGGGTAACAACATTATCCA | |
| TAAAAGCACGGTCATGGCTGATGAGTAATACCGTGCCTTTAAAATTGACCACAAAATCTT | |
| CTAAAAGCTCAAGTGTTGCCATATCCAAATCATTGGTAGGCTCATCAAGCACCAAAACAT | |
| TGGCAGGTTTTAGCAATAATTTGGCCAATAAAACGCGTGCTTTTTCACCGCCTGATAGTG | |
| CTTTAACAGGTGTGCGAGCACGATTTGGCGTGAATAAAAAATCTTGCAAATAGCTTAAAA | |
| TGTGCGTAGTTTTTCCACCAACATCGACATGGTCAGAGCCTTCTGAAACATTATCTGCGA | |
| TAGATTTTTCAGGGTCTAGGTCGTCTTTGAGTTGGTCAAAAAAAGCAATATTTAGATTGG | |
| TGCCAAGCTTAACTGAACCTGACTGAATCGCTGAATCATCCAAACCCAAAATGCTTTTAA | |
| TTAAGGTTGTTTTACCAACGCCATTTTTGCCAATGATACCAACTTTATCACCACGAACAA | |
| GCAGCGTTGAAAAATCCTTAACTAAGGTTTTATTGTCGTAT | |
| SEQ. ID NO:55 | |
| Nucleotide sequence of DNA region (1000 bp) up-stream from the PilQ gene | |
| from Moraxella catarrhalis | |
| CAACTTGAAAATCAGCTCAATGCTCTGCCACGCACAGCACCGATGAGCGAGATTATCGGA | |
| ATGATAAATACCAAAGCACAAGCGGTTAATGTGCAGGTGGTGAGTGCATCAGTTCAAGCA | |
| GGTCGTGAACAGGATTATTATACCGAACGCCCTATCGCAGTGAGTGCGACAGGGGATTAT | |
| CATGCTTTGGGTCGATGGTTACTTGAGTTGTCAGAGGCTAACCATTTGCTGACAGTGCAT | |
| GATTTTGATCTGAAGGCTGGTTTGAACCATCAGCTGATGATGATTGTTCAGATCAAAACT | |
| TATCAAGCGAACAAACGCCCAAAACCAGTTGCTCAGCAGGTGCCTGATGTTCAATGAATA | |
| TTATCGGTGGGGCATTTTGGGTGCTTGGATTTGGGTTGGGATTGGATGTGCTGATAGCAC | |
| CAGTCAAGTTGTTGATGATAAGCTTGCACATATTACCCATGAAGAGCGTATGGCGATCAG | |
| TGAGCCTGTGCCGATACCCTTATCTGTGCCGATGATATATCACCAAGGCAAAGATCCTTT | |
| TATCAATCCTTATAGAAATGTTGAGGTTCTTGATACCAATCATGCCGCTGATCAGCAAGA | |
| TGAGCCAAAAACCGAATCTACCAAAGCTTGGCCTATGGCAGACACTATGCCATCTCAGCC | |
| ATCTGATACTCATCAGTCTGCCAAGGCTCAGGCACAAGTCTTCAAAGGCGATCCGATAGT | |
| CATTGATACCAACCGTGTTCGAGAGCCTTTAGAAAGCTATGAGTTATCAAGCCTACGCTA | |
| TCATGGTCGTATTTTTGATGATGTTAGACTTGTGGCACTCATTATGAGTCCTGATGGCAT | |
| CGTTCATCGTGTGAGTACTGGACAATATCTTGGTAAAAATCACGGAAAAATTACCCATAT | |
| TGACAGTCGTACGATACATCTGATTGAAGCGGTCGCTGATACACAAGGTGGCTATTATCG | |
| CCGTGATGTAAACATTCATTTTATTCATAAGCAATGACAC | |
| SEQ. ID NO:56 | |
| Nucleotide sequence of DNA region (1000 bp) up-stream from the lipo18 gene | |
| from Moraxella catarrhails | |
| TTCATGCAACAAGCGACCATCTTGGCCGATGATACCATCCTGCTCACCTAAGAAAATCAG | |
| TTTATCAGCTTGCAGGGCAATGGCTGTGGTCAGTGCTACATCTTCTGCCAATAGATTAAA | |
| AATTTCGCCCGTAACCGAAAAACCTGTCGGTCCTAGTAGGACAATATGGTCATTATCCAA | |
| ATTATGGCGAATGGCATCGACATCAATTGAGCGTACCTCACCTGTCATCTGATAATCCAT | |
| ACCATCTCTGATGCCGTAAGGGCGAGCGGTGACAAAATTACCCGAAATGGCATCAATACG | |
| AGATCCGTACATTGGGGAGTTAGCAAGCCCCATCGACAGCCGAGCTTCGATTTGTAGACG | |
| AATTGAGCCGACTGCCTCCAAGATGGCAGGCATAGATTCATACGGTGTTACACGCACATT | |
| CTCATGTAGGTTTGATATCAGCTTGCGATTTTGTAAATTTTTTTCCACTTGTGGGCGTAC | |
| ACCATGCACAAGCACCAATTTGATGCCCAAGCTGTGTAGCAGTGCAAAATCATGAATCAG | |
| CGTACTAAAATTGTCACGAGCGACCGCCTCATCACCAAACATAACCACAAAGGTTTTGCC | |
| ACGATGGGTGTTAATGTACGGGGCAGAATTACGAAACCAATGCACAGGTGTGAGTGCAGG | |
| AGTGTTCTGATAGGTGCTGACAGAATTCATGAATGCTCCAAAGAGTCAATGGCTGGTAAA | |
| ATAAGAATGGCGAACAATATATGGCGAGAGCGTCTGATGTTGGTCAAATGTCCCATTAAT | |
| AACTATCAAGATACCATCATACCATAGCAAAGTTTTGGGCAGATGCCAAGCGAATTTATC | |
| AGCTTGATAAGGTTGGCATATGATAAAATCTACCATCATCGTCGCCAGTTTTGAGCATGT | |
| GTAAGTAGTTACCATAATTAAACAGTCAAGAAATTCACACCGTCAATCAGCTGTGCTATG | |
| CTTATGGGCACATAAAACTTGACCAACACAGGATAAATTTA | |
| SEQ. ID NO:57 | |
| Nucleotide sequence of DNA region (1000 bp) up-stream from the lipo11 gene | |
| from Moraxella catarrhalis | |
| GGCATACTTTTGCCATGCTTTATTTTGGCATAACTGCTATAAGCCCATTGCTACTTTTTA | |
| TCATTTATCCATATGTCCAATAATGTGCTTTATGTAATTTAGGCACACTATTAACTCGTG | |
| CCACTGTTAACATTCAGCATAAAAATCTTAACAATGAATCAAAGCATCGTATTGGCTGTT | |
| AAATGATAAGCTTATATTTATTTAAATTCAGACTAAATGATTGTAATATGGACATATCAA | |
| GGTTGAAATCAAAAATTTTGGAGAGTTATGTACGATAATGATAAAAAATTGACCACCATC | |
| GTAGGGGTGTTGTATACGGTGTCTTATATTGCCATATGGTTGGTCAGTGGCTATATTTTA | |
| TGGGGCTGGATTGGTGTGACAGGATTTACTCGTGCGATACTTTGGCTGATCGCTTGGATG | |
| ATTGTGGGTACGATTGCTGATAGAATTCTGATACCGATTATTTTGACCGTCGTGGTTGGG | |
| TTATTTTCTATCTTTTTTGAAAAAAGGCGATAATTTGGTTATTTTTTCACAAAAAATCAT | |
| GATTTTTTTTGTAAACTATCTAAAATATCAATTATGTTATATTATGTGATAAAAGATGGG | |
| CATGCTTAAGTTTTGGATTGCAAAAATCCTAATATCATCACTGACCAAAGCTGTGATGAT | |
| ATCAAAACTTTATCAAAGTTCTTAGGGTATTATCAAGATATCATACCAAATGAATACTTA | |
| CCCAACTTACTATAAAAATCAAATGATATGACTGTGATTTTATTATCATAGATACAAAAA | |
| TCAAAACGCATGAGCCAAAGGTATGATGAATGAATACAAAATTTCGCACACATTATGACA | |
| ATCTAAATGTCGCCAGAAACGCTGACATTGCGGTGATTTGGTGGGATAGGGGTCAAGCCA | |
| GTGCGATTAAGCTAAATTTTTATGTGGGCAATCCCTGACTTTATTTTATTTGTGCCAGTT | |
| GGAACAATTCGTGGTCTAATGTATTTATTTTAAGGAGATAA | |
| SEQ. ID NO:58 | |
| Nucleotide sequence of DNA region (1000 bp) up-stream from the lipo10 gene | |
| from Moraxella catarrhalis | |
| TCTGGTCTACATCCCAAACTATTTACACAAGAAACACTAAAGACAGTGGAGCAGATGACG | |
| CTCAAAAAGGCATCTTATAGTAATTTGACAGTTAATTTTCGTCAAGTGCTTGTACAAAAA | |
| TACACCATCGTGCAAGAAGTTTGTACCAATTTAAGCACAATCATTTTGGCACACACTGTC | |
| AAGCAATGCTTCAGGCAAATTAGCTGCTGGTAAAGATACTTGGGTCATCATGCAATCGCA | |
| TCAACCCTTCTTGCTGCGTTGAAGCGATAAGTTTGCCATCTTGCCAAAATTGACCATGGT | |
| TTAGACCCTTGGCGTGGCTTGTGGTATCGCTCCACATGTCGTAGAGTAGATATTCGGTCA | |
| TATCAAAAGGGCGATGGAAATGTATGGAATGGTCAATACTAGCCATTTGTAGACCTTGTG | |
| TCATCAGGCTTAGCCCATGACTCATTAAACCTGTGCTGACCAAATAATAATCAGACACAA | |
| ACGCAAGTAGTGCTTGATGAATGGCAACTGGCTGCTCCCCAATATCAGCGATACGCACCC | |
| AATTGGCTTGGCGTGGACGCTCAGGCTTGGGTGTCACAGGGTCTCGTGGTGTGACGGGGC | |
| GGATTTCGACATGACGCTGACGCATAAATCTTGCTTTGAGTGGTTCGGGAATTTTATGTA | |
| AATAATCCGCTTTGAGTTCTTGCTCGGTTTTTAGGCTTTCAGGGGGTGGATAATCAGGCA | |
| TGGTTTCTTGGTAATCAAGCCCGCCTTCCATGGGTGAAAATGAGGCAATCATCGAAAAAA | |
| TGACCTGTTCATTGGTCGTATGATTACCGTTTTTGTCGGTGGTTGGCACATATTGCACCG | |
| CAATGACTTCTCGAGCTGATAAACTGCGTCCATCACGTAAGCGGCGTACTTGATAGATGA | |
| CTGGTAGACGAATATCGCCACCTCGTAAAAAATAACCATGTAGGCTATGACAAGGTTTAT | |
| CAATCGTTAATGTGTTAGCACCAGCAAGCAGCGCTTGGGCA | |
| SEQ. ID NO:59 | |
| Nucleotide sequence of DNA region (1000 bp) up-stream from the lipo2 gene | |
| from Moraxella catarrhails | |
| TAAAATGACCTTACAAAATAAAATTATATGTTCAAAAATCGCTTAAGTATTGAAAAAAGC | |
| TATAAAAACTTATCTATTAAAGCATAAAAGATATTAAAGCATAAAAGACGAGAAAAGAGC | |
| AAGCGTCAATGATGATATTTCATATAAAAACTTATGAAATTTTTCAATTTTTTATCGATT | |
| GATTCAGCTTGGCTATCGGTGGTCAACTTTGGCTGCCAAGACATCGCCGGCTTTTTGAAA | |
| AATCATCACAATGGCAACAATGATGATGGTTGAAATCCACTTGACATATACCATGTTGCG | |
| ATGCTCACCATAGTTAATCGCAAGGCTTCCCAAGCCACCACCGCCAACCACACCTGCCAT | |
| TGCAGAATAACCAATCAAAGACACCAAGGTCAATGTGACCGCATTAATCAAAATGGGCAG | |
| GCTTTCAGCAAAATAGTATTTGCTGACAACCTGCCAATGCGTTGCACCCATAGATTTGGC | |
| AGCTTCGGTCAGTCCTGTGGGTACTTCTAATAAAGCATTGGCACTCAAGCGTGCAAAAAA | |
| TGGAATTGCTGCCACACTCAAAGGGACGATGGCGGCTGTTGTGCCAAGGGTTGTTCCCAC | |
| CAAAAATCGTGTGACTGGCATGAGAATAATGAGCAAAATAATAAAAGGAACGGAGCGACC | |
| AATATTAATAATAACATCCAAAATTACAAATACACTGCGATTTTCAAGGATACGCCCTTT | |
| ATCGGTTAAAAATGCCAAAAACCCTATCGGTAGCCCAACCAAAACAGCGATGGCAGTGGC | |
| AGCAAGCCCCATATAGATGGTTTCCCAAGTGGATTGGGCAACCATCTCCCACATTCTTGG | |
| GTGCATTTCACTGACAAATTTTGTGACGATTTCATTCCACATAGCCGATAATCTCAATAT | |
| TGACCCCATGGGTGGTTAAAAATTCTATTGCTTGCATGACCGAGGTGCCTTCACCGATAA | |
| GCTCAGCAATGGTAAAGCCAAATTTTATATCACCTGCATAA | |
| SEQ. ID NO:60 | |
| Nucleotide sequence of DNA region (1000 bp) up-stream from the lipo7 gene | |
| from Moraxella catarrhalis | |
| AGTAAACAATGGTAACAAATACAGCAGTGTCGCACAGTCCTCAGTACGATGATTCTGAAT | |
| TTGAATATGCAGGATTTTGGATACGATTTGTGGCATGTCTTGTCGATAATTTAATTGTTA | |
| TGATTATAATTGCACCGTATTGGTTTTATAATTATCAGCAAATGATGGCCATGCCTGCTG | |
| ACCAAATACCGTTTTATAGTGTTGGGGATGCCATCCTTTATAGTGCTGGGGATGCTATCC | |
| TAAACTTAGTGATGGCGGCGGCGGTTGTTTGGTTTTGGGTAAAAAAAGGTGCAACACCAG | |
| GTAAAATGCTCTTTGGGCTGCAAGTCCGTGATGCCAAAACAGGGCAATTTATCACTGTGC | |
| CAAGGGCATTATTGCGATATTTTAGTTATCTGATTTCATCCGTGATTCTTTGTTTGGGAC | |
| TTATTTGGGTTGGTTTTGATAAGAAAAAACAAGGCTGGCATGATAAAATTGCCAAAACTG | |
| TTGTGGTAAAACGCATTCGCTGATGGGTCGCCAGTTAAACAATAAAACCATCAAACGCAA | |
| GCAGGGCGATGTGTTTGAGCACTTGGCGGTAGATAAGCTAAAACAAGCAGGCTATGAAAT | |
| TATTTTAACCAACTTTACCACCCCATTTGTTGGTGAGATTGATATTATCGCCAGACAGCC | |
| TTTGGAGCAATCGCACCGTTTGGTGCAGCCAAGATTTTGTACGGTATTTGTTGAAGTGCG | |
| TAGCCGAACAAGTTCTGTGTATGGTACAGCGCTTGAGAGTGTTACCTCAAAAAAGCAGGC | |
| AAAAATCTACCGAACAGCAGAACGATTTTTAATCAATTATCCCAAATATATTGATGATGC | |
| ATACCGTTTTGATGTCATGGTTTTTGATTTGGTTGATGGATTGATTGAACATGAATGGAT | |
| AAAAAATGCGTTTTGATTGGCTCAATGGTCGTGAATTAAAATCAATCAAGCAATCCGTAG | |
| CTTTACTATAAGATATATCCCAGTAATATGGAAACATAGCA | |
| SEQ. ID NO:61 | |
| Nucleotide sequence of DNA region (1000 bp) up-stream from the lipo6 gene | |
| from Moraxella catarrhalis | |
| CGTTTAGCTTCATACGCAGACCTTGTGCACCTTCGGGCAACCGAAGCATCACGCCAGCAT | |
| CACGCATCCGCACAAAACCCATCATGCCATCAATTTCGCTGCTGATATGATATACCCCCA | |
| CCAAAGTAAACCGCTTAAATCGTGGAATAACGCCTGCTGCTGAGGGTGAGGCTTCAGGCA | |
| AAACCAAGGTAACCTTATCCCCCAACTTAAGTCCCATGTCAGAGACAATGGACTCACCTA | |
| ATATAATACCAAACTCGCCGATATGTAAATCATCCAAATTGCCTGCGGTCATATGCTCAT | |
| CAATGATAGAAACTTGCTTTTCGTAATCAGGCTCAATGCCAGAAACCACGATTCCAGTCA | |
| CCTGACCTTCAGCGGTTAACATACCTTGTAGTTGAATATAAGGCGCAACTGCTTGCACTT | |
| CTGGATTTTGCATTTTGATTTTTTCGGCAAGTTCTTGCCAATTTGTCAAAATTTCTGTTG | |
| AGGTAACTGAAGCTTGAGGCACCATGCCAAGAATGCGTGATTTAATTTCACGGTCAAAGC | |
| CATTCATGACCGACAAAACCGTGATAAGCACTGCAACCCCAAGCGTAAGCCCAATGGTTG | |
| AGATAAAAGAAATAAAGGAAATAAAGCCATTTTTACGCTTAGCTTTGGTATATCTAAGCC | |
| CAATAAATAACGCCAAGGGACGAAACATAAGCTGTGTTCCAAACGACCCAACCGTGCTAG | |
| TTTAGCACTTTTTTGGACAAATACCAAACATCACATAACAAATGAATCATCAGGTTGCTT | |
| TTGTTGCGCTTGTGTATCTGTATGATAAGTTTCTTGCTAAAACAGCTTTTTTATGTCAGA | |
| ATACAGAAAAGGTATATACTTATATTTTTAACTTTAAATAGATCTGCTTTTTTATACCGA | |
| TGATTTGGCATGAAGTTTATCGGTCTGATATGCTGGATATAAGTTTATCGGCTTGATATA | |
| AATTTTAATTAATCATCAAATTTTTAAGGAATTTATCATTA | |
| SEQ. ID NO:62 | |
| Nucleotide sequence of DNA region (1000 bp) up-stream from the P6 gene from | |
| Moraxella catarrhalis | |
| TAAGGATACCAGATTTTGGCTTGTCAATCGTTGTGTTAATCATTGTAACGGTTTATAGTG | |
| ATTGTCAATTAATAAGGGTAAAAAAGTATTTATCAAGTAATAATCTTTCTTATATGTGAA | |
| TATAATGACAAATTTATCACATTTTTACAAGGATTTTTTATCAAGATTAGGATATGTTCC | |
| AGCTTAATTATTAGTGATGAGCGTGTGATTATTTGGCATCGTTAAATTTATGAGTGCTAA | |
| AATTGCCAAATGATTAAAATTTTGCTAACATGATAGCCCCTTTGGTAGGCTTTATTTGGT | |
| ATTGATGAGCAATAATAATATACCGAGTTAAATGGATTAACTTAACATACGCCAAAAACT | |
| TAACAACGAAAAGTAGATGATTATGACAGATACAGTACAAAAAGATACAGCACAGTCCCC | |
| CAAAAAAGTTTATCTAAAAGACTACACGCCGCCAGTATATGCAGTTAATAAAGTGGATTT | |
| GGATATCCGCTTGTTTGATGATCATGCTGTCGTTGGTGCCAAACTTAAAATGACACGAGC | |
| ACACGCAGGCGAGCTTCGGCTTCTTGGGCGAGATTTAAAGCTTAAAAGCATTCACCTAAA | |
| TGGTCAGGAATTAGAGTCGCAGGCGTATCATCTTGATAAGGAAGGCTTAACAATTTTAGA | |
| TGCACCAGATGTCGCAGTGATTGAGACATTGGTTGAGATTTCACCACAAACCAACACAAC | |
| ACTTGAAGGGCTATATCAAGCAGGAACAGGTGATGATAAGATGTTTGTGACACAATGCGA | |
| ACCTGAGGGTTTTCGCAAAATCACCTTTTTCCCTGACCGCCCTGATGTTTTGACAGAATA | |
| CACCACACGCCTAGAAGCACCAAAGCATTTTAAAACCTTGCTTGCCAATGGTAATTTGGT | |
| TGAGTCAGGAGATGTGGATGAAAATCGCCATTATACCATTTGGCATGATCCTACCAAAAA | |
| ACCCAGCTATCTATTCGCCGCTGTCATTGCCAATCTAGAAG | |
| SEQ. ID NO:63 | |
| Nucleotide sequence of DNA region (1000 bp) up-stream from the MsbB gene | |
| from Haemophilus influenzae (HiRd) | |
| AAATCAAGCGCCTGTGCCTGCTGGTGATGGTTGTGGAGACGAATTATATTCTTGGTTTGA | |
| ACCGCCAAAACCAGGCACTTCAGTGAGCAAACCTAAAGTTACACCGCCTGAGCCGTTTTT | |
| GTGCCAACAGATTTTGAACTCACCGAATCGGAGAGAATGGTTAGAATAGCATTGAGGTAA | |
| ATCAATATGGATATCGGCATTGATCTTTTAGCAATATTGTTTTGTGTTGGTTTTGTCGCA | |
| TCATTTATCGATGCAATTGCTGGCGGTGGTGGATTAATCACCATTCCAGCGTTACTCATG | |
| ACAGGTATGCCACCAGCAATGGCGTTAGGCACCAACAAATTGCAAGCTATGGGCGGTGCA | |
| TTATCCGCAAGCCTTTATTTCTTGCGAAAAAGAGCGGTCAATTTACGCGATATTTGGTTT | |
| ATTTTGATTTGGGTTTTCTTAGGTTCTGCCCTAGGTACATTATTAATTCAATCAATTGAC | |
| GTGGCGATTTTCAAAAAAATGCTTCCTTTTTTGATTTTAGCCATTGGTCTATATTTTTTA | |
| TTTACTCCTAAATTAGGTGATGAAGATCGAAAACAACGATTAAGTTATCTGTTATTTGGT | |
| CTTTTAGTTAGCCCATTTTTAGGTTTTTATGATGGCTTCTTTGGGCCAGGGACTGGCTCA | |
| ATCATGAGTTTAGCCTGTGTTACTTTGCTAGGATTTAATCTCCCGAAAGCGGCAGCACAT | |
| GCAAAAGTGATGAACTTCACTTCGAACCTTGCTTCTTTTGCACTTTTCTTATTGGGCGGA | |
| CAAATTCTTTGGAAAGTGGGTTTCGTGATGATGGCTGGGAGCATTTTAGGTGCAAATTTA | |
| GGTGCCAAAATGGTGATGACGAAAGGTAAAACCTTGATTCGACCGATGGTTGTTATCATG | |
| TCTTTTATGATGACGGCTAAAATGGTTTACGATCAGGGTTGGTTTCATTTTTAATTCGGA | |
| AAGCGCGCAAAAGTGCGGTTAAAATTAATTACATTTTATTA | |
| SEQ. ID NO:64 | |
| Nucleotide sequence of DNA region (1000 bp) up-stream from the HtrB gene | |
| from Haemophilus influenzae (HiRd) | |
| TTGAAGTCCCCAATTTACCCACCACAATTCCTGCGGCAACATTGGCTAGGTAACAAGATT | |
| CTTCGAAAGAACGTCCATCTGCTAATGTGGTTGCTAATACACTAATGACAGTGTCACCGG | |
| CTCCCGTCACATCAAACACTTCTTTTGCAACGGTTGGCAAATGATAAGGCTCTTGATTTG | |
| GGCGTAATAATGTCATGCCTTTTTCAGAACGCGTCACCAAAAGTGCGGTTAATTCAATAT | |
| CAGAAATTAATTTTAAACCTTTCTTAATAATCTCTTCTTCTGTATTACATTTACCTACAA | |
| CGGCTTCAAATTCAGACATATTGGGTGTCAATAATGTAGCCCCACGATAACGTTCAAAAT | |
| CAGTTCCCTTTGGATCGATCAACACAGGCACATTCGCTTTGCGTGCAATTTGAATCATTT | |
| TCTGAACATCTTTAAGCGTGCCTTTGCCGTAATCAGAAAGAATCAAAGCACCGTAATTTT | |
| TCACCGCACTTTCTAACTTCGCTAATAAATCCTTGCAATCTACATTATTGAAATCTTCTT | |
| CAAAATCAAGGCGGAGCAGCTGTTGATGACGAGATAAAATACGTAATTTAGTAATGGTTG | |
| GATGGGTTTCTAATGCAACAAAATTACAATCAATCTTTTGTTTTTCTAATAAGTGGGAAA | |
| GTGCAGAACCTGTCTCATCTTGTCCAATCAATCCCATTAACTGAACGGGTACATTGAGTG | |
| AAGCAATATTCATCGCCACATTTGCAGCACCGCCCGCGCGTTCTTCATTTTCTTGTACGC | |
| GAACTACTGGCACTGGTGCTTCTGGTGAAATACGGTTGGTTGCACCGAACCAATAACGAT | |
| CAAGCATCACATCGCCTAATACAAGTACTTTTGCTTGCTTAAATTCTGCTGAATATTGAG | |
| CCATTTTAAAATCTCTCTATTTGAATAACCAAAATTGTGGCGATTTTACCACAACTCAAA | |
| TTTACGATAAACTACGCCCCTAACTTACGTGGAAAGAACAA | |
| SEQ. ID NO:65 | |
| Nucleotide sequence of DNA region (1000 bp) up-stream from the protein D | |
| gene from Haemophilus influenzae (HiRd) | |
| AGCAATAATTATAGCTGGAATATTCTTTAAAGATGAAAGAGATCGTATAAGACAAAAAGA | |
| ATTTTATATTGGAGAATTATTAGCAATTATTGGTTCGCTAATATTCGTAATAAATAGTTC | |
| AAATAATGATGGAAATACAGACTTTTTTCTTGGGGCAATATTTCTTTTTACAGCTATTTT | |
| TATTCAATCTGTACAGAATTTAATTGTAAAAAAAGTAGCCAAAAAGATAAATGCTGTTGT | |
| AATAAGTGCATCGACAGCAACAATTTCAGGAGTATTATTTTTATGTTTAGCTTTTAATAC | |
| TAAACAAATATATTTATTACAAGATGTTGGCATTGGAATGTTGATAGGTTTAGTTTGCGC | |
| TGGCTTTTATGGGATGCTAACAGGGATGTTGATGGCTTTTTATATTGTTCAAAAACAGGG | |
| AATCACTGTTTTTAACATTTTGCAATTATTAATTCCTCTTTCAACTGCGATAATAGGTTA | |
| CTTAACATTAGATGAAAGAATAAATATCTATCAGGGAATTAGCGGTATTATTGTAATTAT | |
| TGGTTGTGTATTGGCATTAAAAAGAAAAAACAAGGAGTGTTGATATATAAAGTAGATGAT | |
| GTTGGTGGAATAGGTATAGTTAAATATCTGGTTCAATTGGTTTTATTAAGGGCGTTAGCA | |
| ATTCTCCATTTAAGTTTATGTTTGAATTAGATATTTTGGGAAAAGATGGAAGAATAAAGC | |
| TGTTAAATAATGCTGAAACATATGAACTATACCAATACTCAAATAAAAATAATTCTGCTG | |
| GAAATGATTATAAATCTCTAATTCTAACTTGTAGAGAGGATAATGACTATCAATCAGAAA | |
| GAATGATTAAAGCCATTAAAAATATTATTCATTGTATGACTAATAATCATCAACCTATTT | |
| CAAGTGCTGAAACATCTTTAGAAACTATTAAAATTATTCACGGAATAATTAATTC2GTTA | |
| AAATAGGTAATGATCCTAACAATATATAAGGAGAATAAGT | |
| SEQ. ID NO:66 | |
| Nucleotide sequence of DNA region (1000 bp) up-stream from the Hin47 gene | |
| from Haemophilus influenzae (HiRd) | |
| TAAATACTCCAAAATAAATTTCAGATAACGTGGTCTGTAAGACAAAAAAATAAAAAAAAT | |
| GTTCAATAAGAGGAGAGCAAATTATCTTGTTTAAAAGGAAATCGGAGCAGTACAAAAACG | |
| GTCTTACAAGTAGCAAATTCTATAAATTTATGTTCTAATACGCGCAATTTTCTAGTCAAT | |
| AAAAAGGTCAAAAAATGAGCTGGATTAACCGAATTTTTAGTAAAAGTCCTTCTTCTTCCA | |
| CTCGAAAAGCCAATGTGCCAGAAGGCGTATGGACAAAATGTACTGCTTGTGAACAAGTAC | |
| TTTATAGTGAAGAACTCAAACGTAATCTGTATGTTTGCCCGAAATGTGGTCATCATATGC | |
| GTATTGATGCTCGTGAGCGTTTATTAAATTTATTGGACGAAGATTCAAGCCAAGAAATTG | |
| CGGCAGATTTAGAACCAAAAGATATTTTAAAATTCAAAGATTTAAAGAAATATAAAGATC | |
| GTATCAATGCGGCGCAAAAAGAAACGGGCGAGAAAGATGCGCTAATTACTATGACAGGTA | |
| CACTTTATAATATGCCAATCGTTGTGGCTGCATCGAACTTTGCTTTTATGGGCGGTTCAA | |
| TGGGTTCTGTAGTTGGTGCAAAATTTGTTAAAGCGGCTGAAAAAGCGATGGAAATGAATT | |
| GTCCATTTGTGTGTTTCTCTGCGAGTGGTGGTGCTCGTATGCAGGAAGCATTATTCTCTT | |
| TAATGCAAATGGCAAAAACTAGTGCCGTACTTGCTCAAATGCGTGAAAAGGGTGTGCCAT | |
| TTATTTCAGTATTAACGGATCCGACTTTAGGCGGCGTATCAGCCAGTTTTGCGATGTTAG | |
| GGGATTTAAATATTGCCGAGCCAAAAGCCTTAATTGGTTTTGCAGGGCCACGCGTTATTG | |
| AACAAACTGTGCGTGAAAAATTGCCAGAAGGTTTCCAACGTAGTGAGTTTCTACTTGAGA | |
| AAGGGGCAATTGATATGATCGTGAAACGTTCAGAAATGCGT | |
| SEQ. ID NO:67 | |
| Nucleotide sequence of DNA region (1000 bp) up-stream from the P5 gene from | |
| Haemophilus influenzae (HiRd) | |
| TCACTTAATTCAAGCGCATCAATGTTTTCTAAAACATCAACAGAATTGACCGCACTTGTA | |
| TCTAAAATTTCGCCATTTATTAAGACTGCGCGTAATGCCAAAACATGATTAGAGGTTTTA | |
| CCATATTGCAATGAGCCTTGCCCAGAGGCATCGGTGTTAATCATTCCACCTAAAGTCGCT | |
| CGATTGCTGGTGGACAGTTCTGGGGCAAAGAACAAACCATGTGGTTTTAAAAATTGATTA | |
| AGTTGATCTTTTACTACGCCTGCTTGTACTCGAACCCAACGTTCTTTTACATTGAGTTCT | |
| AAGATGGCTGTCATATGACGAGAAAGATCCACTATTATATTGTTATTGATGGATTGCCCA | |
| TTTGTGCCAGTGCCTCCACCGCGAGGCGTAAAGCTGATTGATTGATATTCAGGTAAATTT | |
| GCCAATTTTGTTATCCGCACTATATCAGCAACCGTTTTCGGAAAAAGAATTGCTTGTGGA | |
| AGTTGTTGGTAAACGCTGTTATCCGTAGCCAGACTTAATCTATCTGCATAGTTTGTCGCA | |
| ATATCCCCCTCAAAATGTTGGCATTGAAGATCATCAAGATAATCAAGTACATATTGTTCA | |
| ACTTGAGGAATGCGATTTAGATTTGGCAACATAGTATTTGACCCATTTAAACATATCAGA | |
| TGGAGGCTTTGATAATATCCTAAGGCTAGAATAATGTCGATTAGGAAAGAGAGAGGAGAA | |
| AGTAAAAAGTCTGTTTAAGAAAGTGTTATTTTGGATAAAAACTAAACAAAAAATTCAAAA | |
| GAATTTGATCTTTTCAATTTTTATAGGATAATAAGCGCACTTTTGAACGTTCCTTTGGGG | |
| TAAACATAAGCAAAGGAATTGAATTTGTCAAAAGGTAATAAAGTAGGGCAAATTCAAAAC | |
| CCTAGTTAAGTGACTGTTTATAATGTAGCTTTAATTAAAAGTTCAGTATAAACAAGGACA | |
| CTTTTTATTACTATTCGATCACTAAATAGAGGACATCAAAA | |
| SEQ. ID NO:68 | |
| Nucleotide sequence of DNA region (1000 bp) up-stream from the D15 gene | |
| from Haemophilus influenzae (HiRd) | |
| TCGATTGTATCCTATATAAATTATAGACGTAAAAAATCATTAAATAATGCAAACACCGTT | |
| AAGCTTAATAACAGTGCTGCGCCAATTCGATAACAGATCCTTTGCACCCGCTCAGAAACA | |
| GGTTTTCCTTTAACAGCTTCCATTGTTAAAAAAACTAAATGACCGCCATCTAATACTGGT | |
| AATGGAAATAAATTCATAATCCCTAAATTTACACTAATCAATGCCATAAAACTTAAAAAA | |
| TACACCAATCCAATATTTGCTGATGCGCCAGCACCTTTTGCAATAGAAATTGGCCCACTT | |
| AAATTATTTAATGACAAATCGCCAGTAAGTAATTTCCCTAATATTTTCAAGGTTAAAAGG | |
| GAAAGCTGTCCTGTTTTTTCAATGCCTTTTTGTAAAGATTCAAGAATACCATATTTTAAT | |
| TCAGTACGGTATTCATCCGCTAATTTTGTTAAGGCTGGGCTAACCCCAACAAACCATTTG | |
| CCATTTTGATTACGCACTGGAGTTAGGACTTTGTCAAATGTTTCTCCATTACGTTCAACT | |
| TTAATAGAAAAAGATTCGCCTTGTTCGACCTGTTTTATAAAATCTTGCCAAGGAAGTGCG | |
| GTTAAATTTTCTTTTAAAATTTTATCACCGATTTGTAAACCAGCTTTCTCAGCGGGAGAA | |
| TTTTGAACAACTTTAGAAAGCACCATTTCAATTTTAGGACGCATAGGCATAATCCCTAAT | |
| GCCTCAAAAGCACTTTCTTTTTCAGGATCGAATGTCCAATTTGTAAGATTTAAAGTCCGT | |
| TGTTGTTCAATATTAGAATTGAAAGGAGAAAGGCTAATCTCAACATTAGGCTCCCCCATT | |
| TTTGTGGCAAGTAGCATATTGATGGTTTCCCAATCTTGAGTTTCTTCGCCATCAATTGTA | |
| AGAATTTGCGTATTGGGTTCAATGTGGGCTTGTGCTGCGATTGAGTTTGGTGTTATTGAT | |
| TCAATCACTGGTTTAACCGTTGGCATTCCATAAAGGTAAAT | |
| SEQ. ID NO:69 | |
| Nucleotide sequence of DNA region (1000 bp) up-stream from the Omp26 gene | |
| from Haemophilus influenzae (HiRd) | |
| TTTGATAAATATCCTTAATTAAATGATGGGTTTAATATTTTCTCTGCCCAATTAAATTAG | |
| GCAGAGAACGTTGTTTTTGAGTTCTGATGAAGAAAAAAGTTCAATTTATTAGAAAGAACC | |
| TCCAATACTAAATTGGAACTGTTCGACATCATCATTTTCATATTTTTTAATTGGTTTGGC | |
| ATAAGACAATACCAATGGCCCAATAGGAGATTGCCATTGGAATCCGACACCTGTAGAGGC | |
| GCGAATACGGCTTGATTTGCCATAATCGGGTAAGCTTTTTAATACATTGTTATCTAACCC | |
| ACTCTTATCCGATTTCCACTTAGTATTCCAAACACTTGCCGCATCAACAAATAGGGAGGT | |
| TCGGACTGTATTTTGGCTTTTATCACTCACAAACGGTGTTGGTACAATAAGTTCTGCACT | |
| CGCAGTTGTGATTGCATTACCACCAATCACATCAGAACTTATCTTCTTAAAAGTACCATT | |
| ACCATTACCATGTTCTGCATAAATTGCGTTAGGTCCAATACTACCATAAGCAAAACCACG | |
| TAATGAACCGATGCCACCCGCTGTATAAGTTTGATAGAACGGTAAACGCTTGTTTCCAAA | |
| ACCATTTGCATATCCTGCAGATGCTTTTGCAGATACAACCCAGAGGTGATCTCTGTCTAA | |
| TGGGTAGAAACCCTGTACGTCTGCACTTAGTTTGTAGTATTTGTTATCAGAACCTGGAAT | |
| AGTAACTCGTCCACCAAGACTTGCTTTAACCCCTTTAGTTGGGAAATAGCCTCTATTAAG | |
| GCTGTTATAGTTCCAACCAAAAGAAAAATCAAAGTCATTTGTTTTAATGCCATTACCTTT | |
| AAATTTCATTGATTGAATATATAAATTACGGTTATATTCTAGAGCAAAGTTACTAATTTT | |
| ATTATAGGTATGGCCTAATCCTACATAATAGGAGTTATTTTCATTTACAGGGAAACCTAA | |
| AGTAACATTACTTCCATAAGTCGTACGCTTATAGTTAGAGG | |
| SEQ. ID NO:70 | |
| Nucleotide sequence of DNA region (1000 bp) up-stream from the P6 gene from | |
| Haemophilus influenzae (HiRd) | |
| TTAGATTTCTCCTAAATGAGTTTTTTATTTAGTTAAGTATGGAGACCAAGCTGGAAATTT | |
| AACTTGACCATCACTTCCTGGAACGCTCGCCTTAAAGCGACCATCTGCGGAAACCAATTG | |
| TAGCACCTTTCCTAAGCCCTGTGTAGAACTATAAATAATCATAATTCCATTTGGAGAGAG | |
| GCTTGGGCTTTCGCCTAGAAAAGATGTACTAAGTACCTCTGAAACGCCCGTTGTGAGATC | |
| TTGTTTAACTACATTATTGTTACCATTAATCATCACAAGTGTTTTTCCATCTGCACTAAT | |
| TTGTGCGCTACCGCGACCACCCACTGCTGTTGCACTACCACCGCTTGCATCCATTCGATA | |
| AACTTGTGGCGAACCACTTCTATCGGATGTAAATAAAATTGAATTTCCGTCTGGCGACCA | |
| CGCTGGTTCAGTATTATTACCCGCACCACTCGTCAATTGAGTAGGTGTACCGCCATTTGC | |
| TCCCATAACGTAAATATTCAGAACACCATCACGAGAAGAAGCAAAAGCTAAACGAGAACC | |
| ATCTGGCGAAAAGGCTGGTGCGCCATTATGCCCTTGAAAAGATGCCACTACTTTACCTGC | |
| GCCAGAATTTAAATCCTGTACAACAAGTTGTGATTTTTTATTTTCAAACGATACATAAGC | |
| CAAACGCTGGCCGTCTGGAGACCAAGCTGGAGACATAATTGGTTGGGCACTACGATTGAC | |
| GATAAATTGATTATAGCCATCATAATCTGCTACACGAACTTCATAAGGTTGCGAACCGCC | |
| ATTTTTTTGCACAACATAAGCGATACGAGTTCTAAAGGCACCACGGATCGCAGTTAATTT | |
| TTCAAAAACTTCATCGCTCACAGTATGCGCGCCATAGCGTAACCATTTATTTGTTACTGT | |
| ATAGCTATTTTCCATTAATACAGTCCCTGGCGTACCTGATGCACCAACCGTATCAATTAA | |
| TTGATAAGTAATACTATAACCATTACCCGATGGAACCACTT | |
| SEQ. ID NO:71 | |
| Nucleotide sequence of DNA region (1000 bp) up-stream from the TbpA gene | |
| from Haemophilus influenzae (non-typeable) | |
| GGCGATAACCGAGTTTTTGGGGTATTTAGTGCCAAAGAAGACCCACAAAACCCAAAATTA | |
| TCCAGAGAAACCTTAATTGATGGCAAGCTAACTACTTTTAAAAGAACTGATGCAAAAACC | |
| AATACAACAGCCGATACAACAACCAATAAAACAACCAATGCAATAACCGATGAAAAAAAC | |
| TTTAAGACGGAAGATATACTAAGTTTTGGTGAAGCTGATTATCTTTTAATTGACAATCAG | |
| CCTGTTCCGCTTTTACCTGAAAAAAATACTGATGATTTCATAAGTAGTAGGCATCATACT | |
| GTAGGAAATAAACGCTATAAAGTGGAAGCATGTTGCAAGAATCTAAGCTATGTAAAATTT | |
| GGTATGTATTATGAAGACCCACTTAAAGAAGAAGAAAAAGAAAAAGAAAAAGAAAAAGAC | |
| CAAGAAAAAAAAGAAAAAGAAAAACAAACGACGACAACATCTATCGAGACTTATTATCAA | |
| TTCTTATTAGGTCACCGTACTGCCAAGGCCGACATACCTGCAACGGGAAACGTGAAATAT | |
| CGCGGTAATTGGTTTGGTTATATTGGTGATGACACGACATCTTACTCCACTACTGGAGAT | |
| AAAAATGCTCTCGCCGAGTTTGATGTAAATTTTGCCGATAAAAAGCTAACAGGCGAATTA | |
| AAACGACACGATAATGGAAATACCGTATTTAAAATTACTGCAGACCTTCAAAGTGGTAAG | |
| AATGACTTCACTGGTACAGCAACCGCAACAAATTTTGTAATAGATGGTAACAATAGTCAA | |
| ACTGGAAATACCCAAATTAATATTAAAACTGAAGTAAATGGGGCATTTTATGGACCTAAG | |
| GCTACAGAATTAGGCGGTTATTTCACCTATAACGGAAATTCTACAGCTAAAAATTCCTCA | |
| ACCGTACCTTCACCACCCAATTCACCAAATGCAAGAGCTGCAGTTGTGTTTGGACCTAAA | |
| AAACAACAAGTAGAAACAACCAAGTAATGGAATACTAAAAA | |
| SEQ. ID NO:72 | |
| Nucleotide sequence of DNA region (1000 bp) up-stream from the TbpB gene | |
| from Haemophilus influenzae (HiRd) | |
| TAGAATTATATTCTTATACAAAATTGATAATTGTTCGCATTATCATTTTTTTTTTGTAAT | |
| AATGTCAACTTATAATTTTTTAAGTTCATGGATAAAATATGAAAAATGGCGTAAAACAAC | |
| TTTTTCTCTTATCATTAATAGGCTTATCATTAACGAATGTAGCTTGGGCAGAAGTTGCAC | |
| GTCCTAAAAATGATACATTGACAAATACGATTCAAAGTGCGGAATTAAAAACCTCCTCTT | |
| TTTCCTCTATGCCTAAGAAAGAAATACCAAATAGGCATATTATTTCTCTTTCCAAAAGCC | |
| AATTAGCGCACCATCCAAGGCTTGTTTTGCGTGGGTTAATTCCTGCTTTATATCAAAATA | |
| ACACTCAGGCAGTTCAACTGTTATTACCACTATATAAACAATTTCCTCAACAAGATAATT | |
| TCTTACTAACTTGGGCAAAGGCTATTGAAGCTCGTGAACAAGGTGATTTAACTCAATCTA | |
| TTGCTTATTATCGTGAATTATTCGCTCGAGACGCATCTTTACTACCTTTACGTTATTAAT | |
| TAGCTCAAGCTCTATTTTTTAACTATGAAAATGAAGCTGCCAAAATTCAATTTGAAAAAT | |
| TACGTACAGAGGTAGATGATGAAAAATTTTTAGGTGTTATTGATCAGTATCTTTTAACAC | |
| TAAATCAGCGGAATCAATGGATATGGCAAGTAGGATTAAATTTTTTAAATGATGATAATT | |
| TGAATAACGCTCCAAAAAGTGGCACAAAAATTGGTAGTTGGACCGCTTGGGAAAAAGAAA | |
| GTGGGCAGGGGGTAGGGTATTCTTTATCAGTAGAAAAAAAATGGCCATGGGCAGATCATT | |
| TTTTTAGTAAAACTATGTTTAATGGGAATGGAAAATATTATTGGGATAATAAAAAATACA | |
| ATGAGGCTACTGTGCCTATAGGTGGTGGTTTAGGCTATCAAACTGCCTCAGTTGAAGTCT | |
| CGTTGTTTCCTTTTCAAGAAAAACGCTGGTATGCAGGCGGT | |
| SEQ. ID NO:73 | |
| Nucleotide sequence of DNA region (1000 bp) up-stream from the HifA (pilin) | |
| gene from Haemophilus influenzae (LKP serotype 1 genome) | |
| TAATAAATTGCTCCATAAAGAGGTTTGTGCCTTATAAATAAGGCAATAAAGATTAATATA | |
| AACCGTTTATTAAAATGCCAAAGGCTTAATAAACAGCAAACTTTGTTTTCCCAAAAAAAC | |
| TAAAAAACTCTTCCATTATATATATATATATATATAATTAAAGCCCTTTTTGAAAAATTT | |
| CATATTTTTTTGAATTAATTCGCTGTAGGTTGGGTTTTTGCCCACATGGAGACATATAAA | |
| AAAGATTTGTAGGGTGGGCGTAAGCCCACGCGGAACATCATCAAACAACTGTAATGTTGT | |
| ATTAGGCACGGTGGGCTTATGCCTCGCCTACGGGGAAATGAATAAGGATAAATATGGGCT | |
| TAGCCCAGTTTATGGATTTAATTATGTTGAAATGGGGAAAACAATGTTTAAAAAAACACT | |
| TTTATTTTTTACCCCACTATTTTTTGCCGCACTTTGTGCATTTTCAGCCAATGCAGATGT | |
| GATTATCACTGGCACCAGAGTGATTTATCCCGCTGGGCAAAAAAATGTTATCGTGAAGTT | |
| AGAAAACAATGATGATTCGGCAGCATTGGTGCAAGCCTGGATTGATAATGGCAATCCAAA | |
| TGCCGATCCAAAATACACCAAAACCCCTTTTGTGATTACCCCGCCTGTTGCTCGAGTGGA | |
| AGCGAAATCAGGGCAAAGTTTGCGGATTACGTTCACAGGCAGCGAGCCTTTACCTGATGA | |
| TCGCGAAAGCCTCTTTTATTTTAATTTGTTAGATATTCCGCCGAAACCTGATGCGGCATT | |
| TCTGGCAAAACACGGCAGCTTTATGCAAATTGCCATTCGCTCACGTTTGAAGTTGTTTTA | |
| TCGCCCTCCGAAACTCTCGATGGATTCTCGTGATGCAATGAAAAAAGTAGTGTTTAAAGC | |
| CACACCTGAAGGGGTGTTGGTGGATAATCAAACCCCTTATTATATGAACTACATTGGTTT | |
| GTTACATCAAAATAAACCTGCGAAAAATGTCAAAATGGTTG | |
| SEQ. ID NO:73 | |
| Nucleotide sequence of DNA region (1000 bp) up-stream from the HifE (tip | |
| pilin) gene from Haemophilus influenzae (LKP serotype 1 genome) | |
| TAGTAGATTTCCGCACGGGCAAAAATACAATGGTGTTATTTAACCTCACTTTGCCAAATG | |
| GCGAGCCAGTGCCAATGGCATCCACCGCACAAGATAGCGAAGGGGCATTTGTGGGCGATG | |
| TGGTGCAAGGTGGTGTGCTTTTCGCTAATAAACTTACCCAGCCAAAAGGCGAGTTAATCG | |
| TCAAATGGGGTGAGCGAGAAAGCGAACAATGCCGTTTCCAATATCAAGTTGATTTGGATA | |
| ACGCACAAATACAAAGTCACGATATTCAATGCAAAACCGCAAAATAAATAATTGAAGAGG | |
| ATTTATGCAAAAAACACCCAAAAAATTAACCGCGCTTTTCCATCAAAAATCCACTGCTAC | |
| TTGTAGTGGAGCAAATTATAGTGGAGCAAATTATAGTGGCTCAAAATGCTTTAGGTTTCA | |
| TCGTCTGGCTCTGCTTGCTTGCGTGGCTCTGCTTGATTGCATTGTGGCACTGCCTGCTTA | |
| TGCTTACGATGGCAGACTGACCTTTCAAGGGGAGATTTTAAGTGATGGCACTTGTAAAAT | |
| TGAAACAGACAGCCAAAATCGCACGGTTACCCTGCCAACAGTGGGAAAAGCTAATTTAAG | |
| CCACGCAGGGCAAACCGCCGCCCCTCTGCCTTTTTCCATCACGTTAAAAGAATGCAATGC | |
| AGATGATGCTATGAAAGCTAATCTGCTATTTAAAGGGGGAGACAACACAACAGGGCAATC | |
| TTATCTTTCCAATAAGGCAGGCAACGGCAAAGCCACCAACGTGGGCATTCAAATTGTCAA | |
| AGCCGATGGCATAGGCACGCCTATCAAGGTGGACGGCACCGAAGCCAACAGCGAAAAAGC | |
| CCCCGACACAGGTAAAGCGCAAAACGGCACAGTTATTCAACCCCGTTTTGGCTACTTTGG | |
| CTCGTTATTACGCCACAGGTGAAGCCACCGCAGGCGACGTTGAAGCCACTGCAACTTTTG | |
| AAGTGCAGTATAACTAAAATATTTATTATCCAGTGAAAAAA | |
| SEQ. ID NO:75 | |
| Nucleotide sequence of DNA region (1000 bp) up-stream from the P2 gene from | |
| Haemophilus influenzae (HiRd) | |
| 1 | TTATCCGCTA ACATTTCATC AGTAATTCCA TGAACTTTAA TCGCATCAGG |
| 51 | ATCANCGGGG CGATCTGGCT TAATATAAAT ATGAYAATTA TTACCTGTGT |
| 101 | AACGACGATT TATTAATTCA ACTGCACCAA TTTCAATAAT GCAGTGTCCT |
| 151 | TCATAATGCG CGCCAAGCTG ATTCATACCT GTAGTTTCAG TATCTAATAC |
| 201 | AATTTGGCGA TTGGGATTAA TCATTTGTTC AACCTATCTC TTTCCATTAA |
| 251 | AATACTTGCC ATTCTACACA ACAACCTTTT TGTTATGCCK AAACAGATTG |
| 301 | AAATTTTTAC TGATGGATCT TGCTTAGGTA ATCCAGGGGC GGGCGGAATT |
| 351 | GGTGCCGTAT TGCGTTATAA ACAACATGAA AAAACACTCT CCAAAGGCTA |
| 401 | TTTCCAAACC ACCAATAATC GAATGGAATT ACGCGCTGTC ATTGAAGCAT |
| 451 | TAAATACATT AAAAGAACCT TGCTTGATCA CGCTTTATAG TGATAGCCAA |
| 501 | TATATGAAAA ATGGCATAAC CAAATGGATC TTTAACTGGA AAAAAAATAA |
| 551 | TTGGAAAGCA AGTTCTGGAA AGCCTGTAAA AAACCAAGAT TTATGGATAG |
| 601 | CCTTAGATGA ATCCATCCAA CGTCATAAAA TTAATTGGCA ATGGGTAAAA |
| 651 | GGCCATGCTG GACACAGAGA AAATGAAATT TGCGATGAAT TAGCAAAAAA |
| 701 | AGGGGCAGAA AATCCGACAT TGGAAGATAT GGGGTACATA GAAGAATAAT |
| 751 | ACAACTGATA TAACGTCATA TTTTTCGATA CCTAAAAATA TTTAATACTT |
| 801 | AAACCTAAAA CAGAATAAAA AATAATCAAA TTCATTTAAA AAATGTGATC |
| 851 | TCGATCAGAT TTCAAGAAAA TTAAAATTTT GGAGTATTGA CATCAAAAAT |
| 901 | TTTTTTTGTA AAGATGCAGC TCGTCCGTTT TGGCGATTGG ACAATTCTAT |
| 951 | TGGAGAAAAG TTCAATCATA GATAGTAAAC AACCATAAGG AATACAAATT |
| 1001 | A |
| SEQ. ID NO:76 | |
| Nucleotide sequence of DNA coding region (partial) of the Moraxella Catarrhalis | |
| HtrB gene | |
| 1 | TCAGTGCTTG GTTTTTTAAG ATATGTACCG CTGTCAGTCC TGCATGGATT |
| 51 | GGCGGCGTGT GCGTCTTATA TTTCCTATCA TTGCAGGCTT AGTATTTATC |
| 101 | GCAGCATCCA AGCCAATTTA ATCTTGGTTC ACCCCAAGAT GCCAGACGCA |
| 151 | CAGCGGCAAA AACTCGCCAA ACAAATCCTA AAAAATCAGC TCATCAGTGC |
| 201 | AGTCGACAGT CTTAAAACTT GGGCAATGCC ACCAAAATGG TCTATCGCAC |
| 251 | AAATTAAAAC GGTTCATCAT GAAGATATCC TAATCAAAGC ACTTGCCAAT |
| 301 | CCAAGTGGTA TGCTTGCCAT TGTGCCTCAT ATCGGCACTT GGGAGATGAT |
| 351 | GAATGCTTGG CTCAATACCT TTGGCTCCCC TACTATCATG TATAAGCCCA |
| 401 | TCAAAAATGC GGCGGTAGAT CGCTTTGTTT TACAGGGGCG TGAAAGACTA |
| 451 | AATGCCAGCC TTGTACCCAC AGATGCTAGT GGTGTTAAGG CAATTTTTAA |
| 501 | AACACTCAAA GCAGGTGGAT TTAGTATCAT ACTGCCCGAC CATGTACCTG |
| 551 | ATCCATCAGG TGGTGAGATT GCTCCTTTTT TTGGTATTAA AACCCTAACC |
| 601 | AGTACGCTGG CGTCAAAGCT TGCTGCAAAA ACTGGTTGTG CTCTTGTTGG |
| 651 | CTTAAGCTGT ATTCGGCGTG AAGATGGCGA TGGTTTTGAA ATTTTTTGTT |
| 701 | ATGAATTAAA TGATGAACAA CTTTATTCAA AAAATACCAA AATTGCAACC |
| 751 | ACTGCTTTAA ATGGTGCGAT GGAACAAATG ATTTATCCAC ATTTTTTGCA |
| 801 | TTATATGTGG AGCTATCGTC GGTTCAAGCA TACACCACTA TTAAATAATC |
| 851 | CTTATTTACT TAATGAAAAT GAGCTAAAAA AAATAGCCAT AAAGCTTCAA |
| 901 | GCCATGTCAA AGGATAGTTA TGAG |
| Protein Seq: 25% identity and 35% similarity with HtrB from E. coli | |
| +TA,1 | SVLGFLRYVP LSVLHGLAAC ASYISYHCRL SIYRSIQANL ILVHPKMPDA |
| 51 | QRQKLAKQIL KNQLISAVDS LKTWAMPPKW SIAQIKTVHH EDILIKALAN |
| 101 | PSGMLAIVPH IGTWEMMNAW LNTFGSPTIM YKPIKNAAVD RFVLQGRERL |
| 151 | NASLVPTDAS GVKAIFKTLK AGGFSIILPD HVPDPSGGEI APFFGIKTLT |
| 201 | STLASKLAAK TGCALVGLSC IRREDGDGFE IFCYELNDEQ LYSKNTKIAT |
| 251 | TALNGAMEQM IYPHFLHYMW SYRRFKHTPL LNNPYLLNEN ELKKIAIKLQ |
| 301 | AMSKDSYE |
| SEQ. ID NO:77 | |
| Nucleotide sequence of DNA coding region of the Neisseria (meningococcus B) | |
| HtrB gene | |
| +TA,1 | ATGTTTCGTT TACAATTCGG GCTGTTTCCC CCTTTGCGAA CCGCCATGCA |
| 51 | CATCCTGTTG ACCGCCCTGC TCAAATGCCT CTCCCTGCTG CCACTTTCCT |
| 101 | GTCTGCACAC GCTGGGAAAC CGGCTCGGAC ATCTGGCGTT TTACCTTTTA |
| 151 | AAGGAAGACC GCGCGCGCAT CGTCGCCAAT ATGCGTCAGG CAGGCATGAA |
| 201 | TCCCGACCCC AAAACAGTCA AAGCCGTTTT TGCGGAAACG GCAAAAGGCG |
| 251 | GTTTGGAACT TGCCCCCGCG TTTTTCAGAA AACCGGAAGA CATAGAAACA |
| 301 | ATGTTCAAAG CGGTACACGG CTGGGAACAT GTGCAGCAGG CTTTGGACAA |
| 351 | ACACGAAGGG CTGCTATTCA TCACGCCGCA CATCGGCAGC TACGATTTGG |
| 401 | GCGGACGCTA CATCAGCCAG CAGCTTCCGT TCCCGCTGAC CGCCATGTAC |
| 451 | AAACCGCCGA AAATCAAAGC GATAGACAAA ATCATGCAGG CGGGCAGGGT |
| 501 | TCGCGGCAAA GGAAAAACCG CGCCTACCAG CATACAAGGG GTCAAACAAA |
| 551 | TCATCAAAGC CCTGCGTTCG GGCGAAGCAA CCATCGTCCT GCCCGACCAC |
| 601 | GTCCCCTCCC CTCAAGAAGG CGGGGAAGGC GTATGGGTGG ATTTCTTCGG |
| 651 | CAAACCTGCC TATACCATGA CGCTGGCGGC AAAATTGGCA CACGTCAAAG |
| 701 | GCGTGAAAAC CCTGTTTTTC TGCTGCGAAC GCCTGCCTGG CGGACAAGGT |
| 751 | TTCGATTTGC ACATCCGCCC CGTCCAAGGG GAATTGAACG GCGACAAAGC |
| 801 | CCATGATGCC GCCGTGTTCA ACCGCAATGC CGAATATTGG ATACGCCGTT |
| 851 | TTCCGACGCA GTATCTGTTT ATGTACAACC GCTACAAAAT GCCG |

Protein Sequence - 30% identity and 38% similarity with Htrb E. coli

| 1 | MFRLQFGLFP PLRTAMHILL TALLKCLSLL PLSCLHTLGN RLGMLAFYLL | |
|---|---|---|
| 51 | KEDRARIVAN MRQAGMNPDP KTVKAVFAET AKGGLELAPA FFRKPEDIET | |
| 101 | MFKAVHGWEH VQQALDKHEG LLFITPHIGS YDLGGRYISQ QLPFPLTAMY | |
| 151 | KPPKIKAIDK IMQAGRVRGK GKTAPTSIQG VKQIIKALRS GEATIVLPDH | |
| 201 | VPSPQEGGEG VWVDFFGKPA YTMTLAAKLA HVKGVKTLFF CCERLPGGQG | |
| 251 | FDLHIRPVQG ELNGDKAHDA AVFNRNAEYW IRRFPTQYLF MYNRYKMP | |
| SEQ. ID NO:78 | | |
| Nucleotide sequence of DNA coding region of the Haemophilus influenzae | | |
| (non-typeable) HtrB gene | | |
| 1 | ATGAAAAACG AAAAACTCCC TCAATTTCAA CCGCACTTTT TAGCCCCAAA | |
| 51 | ATACTGGCTT TTTTGGCTAG GCGTGGCAAT TTGGCGAAGT ATTTTATGTC | |
| 101 | TTCCCTATCC TATTTTGCGC CATATTGGTC ATGGTTTCGG TTGGCTGTTT | |
| 151 | TCACATTTAA AAGTGGGTAA ACGTCGAGCT GCCATTGCAC GCCGTAATCT | |
| 201 | TGAACTTTGT TTCCCTGATA TGCCTGAAAA CGAACGTGAG ACGATTTTGC | |
| 251 | AAGAAAATCT TCGTTCAGTA GGCATGGCAA TTATCGAAAC TGGCATGGCT | |
| 301 | TGGTTTTGGT CGGATTCACG TATCAAAAAA TGGTCGAAAG TTGAAGGCTT | |
| 351 | ACATTATCTA AAAGAAAATC AAAAAGATGG AATTGTTCTC GTCGGTGTTC | |
| 401 | ATTTCTTAAC GCTAGAACTT | GGCGCACGCA TCATTGGTTT ACATCATCCT |
| 451 | GGCATTGGTG TTTATCGTCC AAATGATAAT CCTTTGCTTG ATTGGCTACA | |
| 501 | AACACAAGGC CGTTTACGCT CCAATAAAGA TATGCTTGAT CGTAAAGATT | |
| 551 | TACGCGGAAT GATCAAAGCT TTACGCCACG AAGAAACCAT TTGGTATGCG | |
| 601 | CCTGATCACG ATTACGGCAG AAAAAATGCC GTTTTTGTTC CTTTTTTTGC | |
| 651 | AGTACCTGAC ACTTGCACTA CTACTGGTAG TTATTATTTA TTGAAATCCT | |
| 701 | CGCAAAACAG CAAAGTGATT CCATTTGCGC CATTACGCAA TAAAGATGGT | |
| 751 | TCAGGCTATA CCGTGAGTAT TTCAGCGCCT GTTGATTTTA CGGATTTACA | |
| 801 | AGATGAAACG GCGATTGCTG CGCGAATGAA TCAAATCGTA GAAAAGGAAA | |
| 851 | TCATGAAGGG CATATCACAA TATATGTGGC TACATCGCCG TTTTAAAACA | |
| 901 | CGTCCAGATG AAAATACGCC TAGTTTATAC GATTAA | |
| Protein Sequence - 57% identity and 66% similarity with HtrB E. coli | | |
| 1 | MKNEKLPQFQ PHFLAPKYWL FWLGVAIWRS ILCLPYPILR HIGHGFGWLF | |
| 51 | SHLKVGKRRA AIARRNLELC FPDMPENERE TILQENLRSV GMAIIETGMA | |
| 101 | WFWSDSRIKK WSKVEGLHYL KENQKDGIVL VGVHFLTLEL GARIIGLHHP | |
| 151 | GIGVYRPNDN PLLDWLQTQG RLRSNKDMLD RKDLRGMIKA LRHEETIWYA | |
| 201 | PDHDYGRKNA VFVPFFAVPD TCTTTGSYYL LKSSQNSKVI PFAPLRNKDG | |
| 251 | SGYTVSISAP VDFTDLQDET AIAARMNQIV EKEIMKGISQ YMWLHRRFKT | |
| 301 | RPDENTPSLY D* | |
| SEQ. ID NO:79 | | |
| Nucleotide sequence of DNA coding region of the Haemophilus influenzae | | |
| (non-typeable) MsbB gene | | |
| 1 | ATGTCGGATA ATCAACAAAA TTTACGTTTG ACGGCGAGAG TGGGCTATGA | |
| 51 | AGCGCACTTT TCATGGTCGT ATTTAAAGCC TCAATATTGG GGGATTTGGC | |
| 101 | TTGGTATTTT CTTTTTATTG TTGTTAGCAT TTGTGCCTTT TCGTCTGCGC | |
| 151 | GATAAATTGA CGGGAAAATT AGGTATTTGG ATTGGGCATA AAGCAAAGAA | |
| 201 | ACAGCGTACG CGTGCACAAA CTAACTTGCA ATATTGTTTC CCTCATTGGA | |
| 251 | CTGAACAACA ACGTGAGCAA GTGATTGATA AAATGTTTGC GGTTGTCGCT | |
| 301 | CAGGTTATGT TTGGTATTGG TGAGATTGCC ATCCGTTCAA AGAAACATTT | |
| 351 | GCAAAAACGC AGCGAATTTA TCGGTCTTGA ACATATCGAA CAGGCAAAAG | |
| 401 | CTGAAGGAAA GAATATTATT CTTATGGTGC CACATGGCTG GGCGATTGAT | |
| 451 | GCGTCTGGCA TTATTTTGCA CACTCAAGGC ATGCCAATGA CTTCTATGTA | |
| 501 | TAATCCACAC CGTAATCCAT TGGTGGATTG GCTTTGGACG ATTACACGCC | |
| 551 | AACGTTTCGG CGGAAAAATG CATGCACGCC AAAATGGTAT TAAACCTTTT | |
| 601 | TTAAGTCATG TTCGTAAAGG CGAAATGGGT TATTACTTAC CCGATGAAGA | |
| 651 | TTTTGGGGCG GAACAAAGCG TATTTGTTGA TTTCTTTGGG ACTTATAAAG | |
| 701 | CGACATTACC AGGGTTAAAT AAAATGGCAA AACTTTCTAA AGCCGTTGTT | |
| 751 | ATTCCAATGT TTCCTCGTTA TAACGCTGAA ACGGGCAAAT ATGAAATGGA | |
| 801 | AATTCATCCT GCAATGAATT TAAGTGATGA TCCTGAACAA TCAGCCCGAG | |
| 851 | CAATGAACGA AGAAATAGAA TCTTTTGTTA CGCCAGCGCC AGAGCAATAT | |
| 901 | GTTTGGATTT TGCAATTATT GCGTACAAGG AAAGATGGCG AAGATCTTTA | |
| 951 | TGATTAA | |
| Protein Sequence - 45% identity and 56% similarity with MsbB E. coli | | |
| 1 | MSDNQQNLRL TARVGYEAHF SWSYLKPQYW GIWLGIFFLL LLAFVPFRLR | |
| 51 | DKLTGKLGIW IGHKAKKQRT RAQTNLQYCF PHWTEQQREQ VIDKMFAVVA | |
| 101 | QVMFGIGEIA IRSKKHLQKR SEFIGLEHIE QAKAEGKNII LMVPHGWAID | |
| 151 | ASGIILHTQG MPMTSMYNPH RNPLVDWLWT ITRORFGGKM HARQNGIKPF | |
| 201 | LSHVRKGENG YYLPDEDFGA EQSVFVDFFG TYKATLPGLN KMAKLSKAVV | |
| 251 | IPMFPRYNAE TGKYEMEIHP AMNLSDDPEQ SARAMNEEIE SFVTPAPEQY | |
| SEQ. ID NO:80 | | |
| Nucleotide sequence of DNA coding region of the Moraxelia catarrhalis MsbB | | |
| gene | | |
| 1 | ATGAGTTGCC ATCATCAGCA TAAGCAGACA CCCAAACACG CCATATCCAT | |
| 51 | TAAGCATATG CCAAGCTTGA CAGATACTCA TAAACAAAGT AGCCAAGCTG | |
| 101 | AGCCAAAATC GTTTGAATGG GCGTTTTTAC ATCCCAAATA TTGGGGAGTT | |
| 151 | TGGCTGGCTT TTGCGTTGAT TTTACCGCTG ATTTTTCTAC CGCTGCGTTG | |
| 201 | GCAGTTTTGG ATCGGCAAGC GTCTTGGCAT TTTGGTACAT TACTTAGCTA | |
| 251 | AAAGCCGAGT TCAAGACACT CTAACCAACC TGCAGCTTAC CTTCCCAAAT | |
| 301 | CAACCAAAAT CAAAACACAA GGCCACCGCA CGGCAAGTAT TTATTAATCA | |
| 351 | AGGTATTGGT ATTTTTGAAA GTTTATGTGC ATGGTTTCGC CCTAATGTCT | |
| 401 | TTAAACGCAC TTTTAGCATT TCTGGTTTAC AGCATTTGAT TGATGCCCAA | |
| 451 | AAACAAAATA AAGCGGTGAT TTTACTTGGT GGACATCGCA CGACGCTTGA | |
| 501 | TTTGGGCGGT CGGTTATGTA CACAGTTTTT TGCGGCGGAC TGCGTGTATC | |
| 551 | GCCCACAAAA CAACCCTTTG CTTGAATGGT TTATCTATAA TGCACGCCGC | |
| 601 | TGTATCTTTG ATGAGCAAAT CTCAAATCGT GATATGAAAA AACTCATCAC | |
| 651 | TCCGCTCAAA CAAGGTCGGA TAATTTGGTA TTCACCTGAT CAAGATTTTG | |
| 701 | GTCTTGAGCA TGGCGTGATG GCGACCTTTT TTGGTGTGCC TGCAGCAACG | |
| 751 | ATTACCGCTC AGCGTCGTCT TATTAAGCTG GGTGATAAAG CCAATCCTCC | |
| 801 | TGTCATCATC ATGATGGATA TGCTCAGACA AACGCCCGAT TATATCGCAA | |
| 851 | AAGGTCACCG TCCACATTAT CACATCAGCC TAAGCGCTGT GTTAAAAAAT | |
| 901 | TATCCCAGCG ATGACGAAAC CGCCGATGCT GAACGCATCA ATCGACTGAT | |
| 951 | TGAGCAAAAT ATTCAAAAAG ATTTAACCCA GTGGATGTGG TTTCATCGCC | |
| 1001 | GCTTTAAAAC TCAAGCCGAT GACACCAATT ACTATCAACA TTAATG | |
| Protein Sequence - 28% identity and 37 similarity with MsbB of E. coli | | |
| 1 | MSCHHQHKQT PKHAISIKHM PSLTDTHRQS SQAEPKSFEW AFLHPKYWGV | |
| 51 | WLAFALILPL IFLPLRWQFW IGKRLGILVH YLAKSRVQDT LTNLQLTFPN | |
| 101 | QPKSKHKATA RQVFINQGIG IFESLCAWFR PNVFKRTFSI SGLQHLIDAQ | |
| 151 | KQNKAVILLG GHRTTLDLGG RLCTQFFAAD CVYRPQNNPL LEWFIYNARR | |
| 201 | CIFDEQISNR DMKKLITRLK QGRIIWYSPD QDFGLEHGVM ATFFGVPAAT | |
| 251 | ITAQRRLIKL GDKANPPVII MMDMLRQTPD YIAKGHRPHY HISLSAVLKN | |
| 301 | YPSDDETADA ERINRLIEQN IQKDLTQWMW FHRRFKTQAD DTNYYQH* | |
| SEQ. ID NO:81 | | |
| Nucleotide sequence of DNA coding region of the Neisseria (meningococcus B) | | |
| MsbB gene | | |
| 1 | ATGAAATTTA TATTTTTTGT ACTGTATGTT TTGCAGTTTC TGCCGTTTGC | |
| 51 | GCTGCTGCAC AAACTTGCCG ACCTGACGGG TTTGCTCGCC TACCTTTTGG | |
| 101 | TCAAACCCCG CCGCCGTATC GGCGAAATCA ATTTGGCAAA ATGCTTTCCC | |
| 151 | GAGTGGGACG GAAAAAAGCG CGAAACCGTA TTGAAGCAGC ATTTCAAACA | |
| 201 | TATGGCGAAA CTGATGCTTG AATACGGCTT ATATTGGTAC GCGCCTGCCG | |
| 251 | GGCGTTTGAA ATCGCTGGTG CGTTACCGCA ATAAGCATTA TTTGGACGAC | |
| 301 | GCGCTGGCGG CGGGGGAAAA AGTCATCATT CTGTACCCGC ACTTCACCGC | |
| 351 | GTTCGAGATG GCGGTGTACG CGCTTAATCA GGATGTACCG CTGATCAGTA | |
| 401 | TGTATTCCCA CCAAAAAAAC AAGATATTGG ACGCACAGAT TTTGAAAGGC | |
| 451 | CGCAACCGCT ACGACAATGT CTTCCTTATC GGGCGCACCG AAGGCGTGCG | |
| 501 | CGCCCTCGTC AAACAGTTCC GCAAAAGCAG CGCGCCGTTT CTGTATCTGC | |
| 551 | CCGATCAGGA TTTCGGACGC AACGATTCGG TTTTTGTGGA TTTTTTCGGT | |
| 601 | ATTCAGACGG CAACGATTAC CGGCTTGAGC CGCATTGCCG CGCTTGCAAA | |
| 651 | TGCAAAAGTG ATACCCGCCA TCCCCGTCCG CGAGGCGGAC AATACGGTTA | |
| 701 | CATTGCATTT CTACCCGGCT TGGGAATCCT TTCCGAGTGA AGATGCGCAG | |
| 751 | GCCGACGCGC AGCGCATGAA CCGTTTTATC GAGGAACCGT GCGCGAACAT | |
| 801 | CCCGAGCAGT ATTTTTGGCT GCACAAGCGT TTCAAAACCC GTCCGGAAGG | |
| 851 | CAGCCCCGAT TTTTACTGAT ACGTAA | |
| Protein Sequence - 25% identity and 36% identity with MsbB E. coli | | |
| 1 | MKFIFFVLYV LOFLPFALLH KLADLTGLLA YLLVKPRRRI GEINLAKCFP | |
| 51 | EWDGKKRETV LKQHFKHMAK LMLEYGLYWY APAGRLKSLV RYRNKHYLDD | |
| 101 | ALAAGEKVII LYPHFTAFEM AVYALNQDVP LISMYSHQKN KILDAQILKG | |
| 151 | RNRYDNVFLI GRTEGVRALV KQFRKSSAPF LYLPDQDFGR NDSVFEVDFFG | |
| 201 | IQTATITGLS RIAALANAKV IPAIPVREAD NTVTLHFYPA WESFPSEDAQ | |
| 251 | ADAQRMNRFI EEPCANIPSS IFGCTSVSKP VRKAAPIFTD T* | |

We claim:

1. An immunogenic composition comprising an antigen derived from a pathogen which is capable of protecting a host against said pathogen, mixed with an adjuvant comprising a bleb preparation derived from a Gram-negative bacterial strain, with the proviso that an immunogenic composition consisting of N. meningitidis B blebs and N. meningitidis C polysaccharide antigen is not claimed.

2. The immunogenic composition comprising an antigen comprising 1 or more conjugated meningococcal capsular polysaccharides selected from a group comprising: A, Y or W, mixed with an adjuvant comprising a bleb preparation from meningoccocus B.

3. The immunogenic composition of claim 1, wherein the antigen and the Gram-negative bacterial bleb preparation are from different pathogens.

4. The immunogenic composition of claim 3, wherein the antigen is a conjugated capsular polysaccharide from H. influenzae b, and the bleb preparation is from meningoccocus B.

5. The immunogenic composition of claim 3, wherein the antigen is one or more conjugated capsular polysaccharide(s) from Streptococcus pneumoniae selected from the group consisting of: 1, 2, 3, 4, 5, 6B, 7F, 8, 9N, 9V, 10A, 11A, 12F, 14, 15B, 17F, 18C, 19A, 19F, 20, 22F, 23F and 33F, and the bleb preparation is from meningoccocus B.

6. The immunogenic composition of claim 2, 4 or 5, wherein the bleb preparation is derived from a strain which has a detoxified lipid A portion of bacterial LPS, due to the strain having been engineered to reduce or switch off expression of one or more genes selected from the group consisting of: htrB, msbB and lpxK.

7. The immunogenic composition of claim 2, 4, 5 or 6, wherein the bleb preparation is derived from a strain which has a detoxified lipid A portion of bacterial LPS, due to the strain having been engineered to express at a higher level one or more genes selected from the group consisting of: pmrA, pmrB, pmrE and pmrF.

8. The immunogenic composition of claim 2, 4, 5, 6 or 7, wherein the bleb preparation is derived from a strain which does not produce B capsular polysaccharide, due to the strain having been engineered to reduce or switch off expression of one or more genes selected from the group consisting of: galE, siaA, siaB, siaC, siaD, ctrA, ctrB, ctrC and ctrD.

9. The immunogenic composition of claim 3, wherein the antigen is from H. influenzae, and the bleb preparation is from Moraxella catarrhalis.

10. The immunogenic composition of claim 9, wherein the antigen is a conjugated capsular polysaccharide from H. influenzae b.

11. The immunogenic composition of claim 3, wherein the antigen is from Streptococcus pneumoniae, and the bleb preparation is from Moraxella catarrhalis.

12. The immunogenic composition of claim 11, wherein the antigen is one or more conjugated capsular polysaccharide(s) from Streptococcus pneumoniae selected from the group consisting of: 1, 2, 3, 4, 5, 6B, 7F, 8, 9N, 9V, 10A, 11A, 12F, 14, 15B, 17F, 18C, 19A, 19F, 20, 22F, 23F and 33F.

13. The immunogenic composition of claim 11, wherein the antigen is one or more proteins from Streptococcus pneumoniae capable of protecting a host against pneumococcal disease.

14. The immunogenic composition of claims 9-13, wherein the bleb preparation is derived from a strain which has a detoxified lipid A portion of bacterial LPS, due to the strain having been engineered to reduce or switch off expression of one or more genes selected from the group consisting of: htrB, msbB and lpxK.

15. The immunogenic composition of claims 9-14, wherein the bleb preparation is derived from a strain which has a detoxified lipid A portion of bacterial LPS, due to the strain having been engineered to express at a higher level one or more genes selected from the group consisting of: pmrA, pmrB, pmrE and pmrF.

16. The immunogenic composition of claim 3, wherein the antigen is a conjugated capsular polysaccharide from H. influenzae b, and the bleb preparation is from non-typeable H. influenzae.

17. The immunogenic composition of claim 3, wherein the antigen is from Streptococcus pneumoniae, and the bleb preparation is from non-typeable H. influenzae.

18. The immunogenic composition of claim 17, wherein the antigen is one or more conjugated capsular polysaccharide(s) from Streptococcus pneumoniae selected from the group consisting of: 1, 2, 3, 4, 5, 6B, 7F, 8, 9N, 9V, 10A, 11A, 12F, 14, 15B, 17F, 18C, 19A, 19F, 20, 22F, 23F and 33F.

19. The immunogenic composition of claim 17, wherein the antigen is one or more proteins from Streptococcus pneumoniae capable of protecting a host against pneumococcal disease.

20. The immunogenic composition of claim 3, wherein the antigen is from Moraxella catarrhalis, and the bleb preparation is from non-typeable H. influenzae.

21. The immunogenic composition of claim 20, wherein the antigen is one or more proteins from Moraxella catarrhalis capable of protecting a host against disease caused by Moraxella catarrhalis.

22. The immunogenic composition of claims 13-21, wherein the bleb preparation is derived from a strain which has a detoxified lipid A portion of bacterial LPS, due to the strain having been engineered to reduce or switch off expression of one or more genes selected from the group consisting of: htrB, msbB and lpxK.

23. The immunogenic composition of claims 13-22, wherein the bleb preparation is derived from a strain which has a detoxified lipid A portion of bacterial LPS, due to the strain having been engineered to express at a higher level one or more genes selected from the group consisting of: pmrA, pmrB, pmrE and pmrF.

24. A vaccine comprising the immunogenic composition of claims 1-23, and a pharmaceutically acceptable excipient or carrier.

25. A method of inducing a faster protective immune response against the antigen contained in the immunogenic composition of claims 1-23, comprising the step of administering to a host an effective amount of the immunogenic composition of claims 1-23.

26. A method of inducing an enhanced immune response against the antigen contained in the immunogenic composition of claims 1-23, comprising the step of administering to a host an effective amount of the immunogenic composition of claims 1-23.

27. A method of protecting an elderly patient against a pathogen by administering to said patient an effective amount of the immunogenic composition of claims 1-23 in which the antigen is derived from said pathogen.

28. Use of the immunogenic preparation of claims 1-23 in the manufacture of a medicament for the treatment of a disease caused by the pathogen from which the antigen is derived.

29. Use of bleb derived from Moraxella catarrhalis as an adjuvant in an immunogenic composition comprising one or more pneumococcal capsular polysaccharides.

30. Use of bleb derived from Moraxella catarrhalis as an adjuvant in an immunogenic composition comprising one or more pneumococcal protein antigens.

31. Use of bleb derived from non-typeable H. influenzae as an adjuvant in an immunogenic composition comprising one or more pneumococcal capsular polysaccharides.

32. Use of bleb derived from non-typeable H. influenzae as an adjuvant in an immunogenic composition comprising one or more pneumococcal protein antigens.

33. A process for making an immunogenic composition comprising the step of mixing an antigen derived from a pathogen which is capable of protecting a host against said pathogen, with an adjuvant comprising a bleb preparation derived from a Gram-negative bacterial strain.


SEQUENCE LISTING (156 sequences)

SEQ ID NO: 1 - DNA, 5893 bp, Artificial Sequence
  pCMK(+) vector
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgccgtctgaatacatcccgtcattcctcaaaaacagaaaaccaaaatcagaaacctaaaatcccgtcattcccgcgcaggcgggaatccagtccgttcagtttcggtcatttccgataaattcctgctgcttttcatttctagattcccactttcgtgggaatgacggcggaagggttttggttttttccgataaattcttgaggcattgaaattctagattcccgcctgcgcgggaatgacggctgtagatgcccgatggtctttatagcggattaacaaaaatcaggacaaggcgacgaagccgcagacagtacagatagtacggaaccgattcacttggtgcttcagcaccttagagaatcgttctctttgagctaaggcgaggcaacgccgtacttgtttttgttaatccactataaagtgccgcgtgtgtttttttatggcgttttaaaaagccgagactgcatccgggcagcagcgcatcggcccgcacgaggtctctggagtcgcgagcatcaagggcgaattctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggaggtctgcgcttgaattgtgttgtagaaacacaacgtttttgaaaaaataagctattgttttatatcaaaatataatcatttttaaaataaaggttgcggcatttatcagatatttgttctgaaaaatggttttttgcgggggggggggtataattgaagacgtatcgggtgtttgcccggaattgtgagcggataacaattcgatgtttttaggtttttatcaaatttacaaaaggaagcccatatgcatcctaggcctattaatattccggagtatacgtagccggctaacgttaacaaccggtacctctagaactatagctagcatgcgcaaatttaaagcgctgatatcgatcgcgcgcagatctgattaaataggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattctaaatatcggggcggtgaagcggatagctttgtttttgacggcttcgccttcattctttgattgcaatctgactgccaatctgcttcagccccaaacaaaaacccggatacggaagaaaaacggcaataaagacagcaaataccgtctgaaagattttcagacggtatttcgcatttttggcttggtttgcacatatagtgagaccttggcaaaaatagtctgttaacgaaatttgacgcataaaaatgcgccaaaaaattttcaattgcctaaaaccttcctaatattgagcaaaaagtaggaaaaatcagaaaagttttgcattttgaaaatgagattgagcataaaattttagtaacctatgttattgcaaaggtctcgaattgtcattcccacgcaggcgggaatctagtctgttcggtttcagttatttccgataaattcctgctgcgccgtctgaagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 2 - DNA, 997 bp, Neisseria meningitidis
ggaaccgaacacgccgttcggtcatacgccgccgaaaggtttgccgcaagacgaagccgccctcgacatcgaagacgcggtacacggcgcgctggaaagcgcgggttttgtccactacgaaacatcggcttttgcgaaaccagccatgcagtgccgccacaatttgaactactggcagttcggcgattatttaggcataggcgcgggcgcgcacggcaaaatttcctatcccgaccgcatcgagcgcaccgtccgccgccgccaccccaacgactacctcgccttaatgcaaaaccgaccgagcgaagccgtcgaacgcaaaaccgtcgccgccgaagatttgccgttcgaattcatgatgaacgccctgcgcctgaccgacggcgtacccaccgcgatgttgcaggagcgcacgggcgtaccgagtgccaaaatcatggcgcaaatcgaaacggcaaggcaaaaaggcctgctggaaaccgaccccgccgtattccgcccgaccgaaaaaggacgcttgtttttaaacgatttgctgcagtgttttttatagtggattaacaaaaaccagtacggcgttgcctcgccttagctcaaagagaacgattctctaaggtgctgaagcaccaagtgaatcggttccgtactatctgtactgtctgcggcttcgtcgccttgtcctgatttttgttaatccactatataagcgcaaacaaatcggcggccgcccgggaaaacccccccgaacgcgtccggaaaatatgcttatcgatggaaaacgcagccgcatcccccgccgggcgtttcagacggcacagccgccgccggaaatgtccgacgcttaaggcacagacgcacacaaaaaaccgtatgcctgcacctgcaacaatccgacagataccgctgttttttccaaaccgtttgcaagtttcacccatccgccgcgtgatgccgccaccaccatttaaaggcaacgcgcgggttaacggctttgccg

SEQ ID NO: 3 - DNA, 1000 bp, Neisseria meningitidis
accattgccgcccgcgccggcttccaaagcggcgacaaaatacaatccgtcaacggcacacccgttgcagattggggcagcgcgcaaaccgaaatcgtcctcaacctcgaagccggcaaagtcgccgtcgggttcagacggcatcaggcgcgcaaaccgtccgcaccatcgatgccgcaggcacgccggaagccggtaaaatcgcaaaaaaccaaggctacatcggactgatgccctttaaaatcacaaccgttgccggtggcgtggaaaaaggcagccccgccgaaaaagcaggcctgaaaccgggcgacaggctgactgccgccgacggcaaacccattacctcatggcaagaatgggcaaacctgacccgccaaagccccggcaaaaaaatcaccctgaactacgaacgcgccggacaaacccataccgccgacatccgccccgatactgtcgaacagcccgaccacaccctgatcgggcgcgtcggcctccgtccgcagccggacagggcgtgggacgcgcaaatccgccgcagctaccgtccgtctgttatccgcgcattcggcatgggctgggaaaaaaccgtttcccactcgtggacaaccctcaaatttttcggcaaactaatcagcggcaacgcctccgtcagccatatttccgggccgctgaccattgccgacattgccggacagtccgccgaactcggcttgcaaagttatttggaatttttggcactggtcagcatcagcctcggcgtgctgaacctgctgcccgtccccgttttggacggcggccacctcgtgttttatactgccgaatggatacgcggcaaacctttgggcgaacgcgtccaaaacatcggtttgcgcttcgggcttgccctcatgatgctgatgatggcggtcgccttcttcaacgacgttacccggctgctcggttagattttacgtttcggaatgccgtctgaaaccgcattccgcaccacaaggaactgaca

SEQ ID NO: 4 - DNA, 1036 bp, Neisseria meningitidis
attcccgcgcaggcgggaatccagaaacgcaacgcaacaggaatttatcggaaaaaacagaaacctcaccgccgtcattcccgcaaaagcgggaatctagaaacacaacgcggcaggactttatcagaaaaaacagaaaccccaccgccgtcattcccgcaaaagcgggaatccagacccgtcggcacggaaacttaccggataaaacagtttccttagattccacgtcctagattcccgctttcgcgggaatgacgagattttagattatgggaatttatcaggaatgattgaatccatagaaaaaccacaggaatctatcagaaaaaacagaaacccccaccgcgtcattcccgcgcaggcgggaatccagaaacacaacgcggcaggactttatcggaaaaaaccgaaaccccaccgaccgtcattcccgcaaaagttggaatccaaaaacgcaacgcaacaggaatttatcggaaaaaacagaaacccccaccgcgtcattcccgcgcaggcgggaatccagaaacacaacgcaacaggaatttatcggaaaaaacagaaaccccaccgaccgtcattcccgcaaaagcgggaatccagcaaccgaaaaaccacaggaatctatcagcaaaaacagaaacccccaccgaccgtcattcccgcgcaggcgggaatccagaaacacaacgcggcaggactttatcggaaaaaacagaaaccccaccgaccgtcattcccgcaaaagctggaatccaaaaacgcaacgcaacaggaatttatcggaaaaaacagaaaccccaccgccgtcattcccgcaaaagcgggaatccagacccgtcggcacggaaacttaccggataaaacagtttccttagattccacgtcccagattcccgccttcgcgggaatgacgagattttaagttgggggaatttatcagaaaacccccaacccccaaaaaccgggcggatgccgcaccatccgcccccaaaccccgatttaaccattcaaacaaaccaaaagaaaaaacaaa

SEQ ID NO: 5 - DNA, 772 bp, Neisseria meningitidis
gcgatgtcgggaagccttctcccgaatcattaccccttgagtcgctgaaaatcgcccaatctccggaaaacggcggcaatcatgacggcaagagcagcatcctgaacctcagtgccattgccaccacctaccaagcaaaatccgtagaagagcttgccgcagaagcggcacaaaatgccgagcaaaaataacttacgttagggaaaccatgaaacactatgccttactcatcagctttctggctctctccgcgtgttcccaaggttctgaggacctaaacgaatggatggcacaaacgcgacgcgaagccaaagcagaaatcatacctttccaagcacctaccctgccggttgcgccggtatacagcccgccgcagcttacagggccgaacgcattcgacttccgccgcatggaaaccgacaaaaaaggggaaaatgcccccgacaccaagcgtattaaagaaacgctggaaaaattcagtttggaaaatatgcgttatgtcggcattttgaagtctggacagaaagtctccggcttcatcgaggctgaaggttatgtctacactgtcggtgtcggcaactatttgggacaaaactacggtagaatcgaaagcattaccgacgacagcatcgtcctgaacgagctgatagaagacagcacgggcaactgggtttcccgtaaagcagaactgctgttgaattcttccgacaaaaacaccgaacaagcggcagcacctgccgcagaacaaaattaagaagaggattactccatt

SEQ ID NO: 6 - DNA, 1057 bp, Neisseria meningitidis
gtgcggcaaaaaacagcaaaagcccgctgtcgattgcctgaccgtccgcgtccgtaaaatcagcataggttgccacgcgcggcttgggcgttttcccacacaaagcctctgccatcggcagcaggtttttccccgatatgcgtatcacgcccacgccgccgcgcccgggtgcggtagcgactgccgcaatcgttggaacgttatccgacataaaacccccgaaaattcaaaacagccgcgattatagcaaatgccgtctgaagtccgacggtttggctttcagacggcataaaaccgcaaaaatgcttgataaatccgtccgcctgacctaatataaccatatggaaaaacgaaacacatacgccttcctgctcggtataggctcgctgctgggtctgttccatcccgcaaaaaccgccatccgccccaatcccgccgacgatctcaaaaacatcggcggcgattttcaacgcgccatagagaaagcgcgaaaatgaccgaaaacgcacaggacaaggcgcggcaggctgtcgaaaccgtcgtcaaatccccggagcttgtcgagcaaatcctgtccgacgagtacgtgcaaataatgatagcccggcgtttccattcgggatcgttgccgccgccgtccgacttggcgcaatacaacgacattatcagcaacggggcagaccgcattatggcaatggcggaaaaagaacaagccgtccggcacgaaaccatacggcaagaccaaaccttcaacaggcgcgggcaactgtacggcttcatcagcgtcatcctgatactgctttttgccgtcttcctcgtatggagcggctaccccgcaaccgccgcctcccttgccggcggcacagtggttgccttggcgggtgctttcgtgattggaagaagccgagaccaaggcaaaaattaattgcaaatcctagggcgtgcttcatatccgcccgaacgccgaaccgcacatataggcacatcccgcgcgccgccggaagcggaagccgcgccctcccaaacaaacccgaatcccgtcagataaggaaaaata

SEQ ID NO: 7 - DNA, 924 bp, Neisseria meningitidis
ggaaccgaacacgccgttcggtcatacgccgccgaaaggtttgccgcaagacgaagccgccctcgacatcgaagacgcggtacacggcgcgctggaaggcgcgggttttgtccactacgaaacatcggcttttgcgaaaccagccatgcagtgccgccacaatttgaactactggcagttcggcgattatttaggcataggcgcgggcgctcacggcaaaatttcctatcccgaccgcatcgagcgcaccgtccgccgccgccaccccaacgactacctcgccttaatgcaaagccaaccgagtgaagccgtcgaacgcaaaaccgttgccgccgaagatttgccgtttgagttcatgatgaacgccctgcgcctgaccgacgcgtacccgccgcgatgttgcaggagcgcacgggcgtaccgagtgccaaaatcatggcgcaaatcgaaacggcaaggcaaaaaggcctgctggaaaccgaccccgccgtattccgcccgaccgaaaaaggacgcttgtttttaaacgatttgctgcagtgttttttatagtggattaacaaaaaccagtacggcgttgcctcgccttagctcaaagagaacgattctctaaggtgctgaagcaccaagtgaatcggttccgtactatttgtactgtctgcggcttcgtcgccttgtcctgatttttgttaatccactatataagcgcaaacaaatcggcggccgcccgggaaaacccgccccgaacgcgtccggaaaatatgcttatcgatggaaaacgcagccgcatcccccgccgggcgtttcagacggcacagccgccgccggaaatgtccgacgcttaaggcacagacgcacacaaaaccgtatgcctgcacctgcaacaatccgacagataccgctgttttttccaaaccgtttgca

SEQ ID NO: 8 - DNA, 1000 bp, Neisseria meningitidis
aagtgggaatctaaaaatgaaaagcaacaggaatttatcggaaatgaccgaaactgaacggactggattcccgctttcgcgggaatgacggcgacagggttgctgttatagtggatgaacaaaaaccagtacgtcgttgcctcgccttagctcaaagagaacgattctctaaggtgctgaagcaccaagtgaatcggttccgtcctatttgtactgtctgcggcttcgtcgccttgtcctgatttctgttcgttttcggttattcccgataaattaccgccgtttctcgtcatttctttaacccttcgtcattcccgcgcaggcgggaatctagtttttttgagttccagttgtttctgataaattcttgcagctttgagttcctagattcccactttcgtgggaatgacggtggaaaagttgccgtgatttcggataaattttcgtaacgcataatttccgttttacccgataaatgcccgcaatctcaaatcccgtcattccccaaaaacaaaaaatcaaaaacagaaatatcgtcattcccgcgcaggcgggaatctagaccttagaacaacagcaatattcaaagattatctgaaagtccgagattctagattcccactttcgtgggaatgacgaattttaggtttctgtttttggttttctgtccttgcgggaatgatgaaattttaagttttaggaatttatcggaaaaaacagaaaccgctccgccgtcattcccgcacaggcttcgtcattcccgcgcaggcttcgtcattcccgcatttgttaatccactatattcccgccgttttttacatttccgacaaaacctgtcaacaaaaaacaacacttcgcaaataaaaacgataatcagctttgcaaaaatcccccccccctgttaatataaataaaaataattaattaattatttttcttatcctgccaaatcttaacggtttggatttacttcccttcatacactcaagaggacgattga

SEQ ID NO: 9 - DNA, 1000 bp, Neisseria meningitidis
ctataaagatgtaaataaaaatctcggtaacggtaacactttggctcagcaaggcagctacaccaaaacagacggtacaaccgcaaaaatgggggatttacttttagcagccgacaatctgcacagccgcttcacgaacaaaatgctatccattagccatgttcgggaaaacacgatttccccgtttgttttaggctgtctaaacaaataaccataaatgtatatcattatttaaaataaataaaagtatttaactattattgacgaaattttagagaaagagtagactgtcgattaaatgacaaacaatagtgagaaaggaaatatttactatccgagcacagagcatattttaggtagcctgtaactgttcctgctggcggaagaggatgaaggtggacttacccgagaataaatgtcctgttgtgtgatatggatgccatgccgcgaagcaattgatgcaatcacggcagtcctacttgaatgaaacctgtcgttgcagaatttgaaaacgctatttttaagaaaggataaagggagaaagaatttttggtttttaagctgcatgaaaccgtgttggaataaatgcacacctacgataattaataattttcgttttttattctacaagctatttatatatgattgctaaaagtttattttttagatgccaaaaaatatattttatatacttcatattgtttatatgtctttatttgaatatatcttacgatggggaaatatttatatattttataataaattttactcatttgctaatatgtcatggaatattacttgtattttgtagaatttttccatatgaaaatattccatttactatttttctgaactttattagtttatttttaatatttttacctcttatatttaccataagagagctaattgattcatattatattgagtcgataattaatttattcttaattttaattcctcacgttatttttttaatttacttgaaaggaaagcagat

SEQ ID NO: 10 - DNA, 1000 bp, Neisseria meningitidis
ggaaacagagaaaaaagtttctcttctatcttggataaatatatttaccctcagtttagttaagtattggaatttatacctaagtagtaaaagttagtaaattatttttaactaaagagttagtatctaccataatatattctttaactaatttctaggcttgaaattatgagaccatatgctactaccatttatcaactttttattttgtttattgggagtgtttttactatgacctcatgtgaacctgtgaatgaaaagacagatcaaaaagcagtaagtgcgcaacaggctaaagaacaaaccagtttcaacaatcccgagccaatgacaggatttgaacatacggttacatttgattttcagggcaccaaaatggttatcccctatggctatcttgcacggtatacgcaagacaatgccacaaaatggctttccgacacgcccgggcaggatgcttactccattaatttgatagagattagcgtctattacaaaaaaaccgaccaaggctgggttcttgagccatacaaccagcaaaacaaagcacactttatccaatttctacgcgacggtttggatagcgtggacgatattgttatccgaaaagatgcgtgtagtttaagtacgactatgggagaaagattgcttacttacggggttaaaaaaatgccatctgcctatcctgaatacgaggcttatgaagataaaagacatattcctgaaaatccatattttcatgaattttactatattaaaaaaggagaaaatccggcgattattactcatcggaataatcgaataaaccaaactgaagaagatagttatagcactagcgtaggttcctgtattaacggtttcacggtacagtattacccgtttattcgggaaaagcagcagctcacacagcaggagttggtaggttatcaccaacaagtagagcaattggtacagagttttgtaaacaattcaaataaaaaataatttaaaggatcttatt

SEQ ID NO: 11 - DNA, 1000 bp, Neisseria meningitidis
acgtccgaaccgtgattccgcaacgccgcgcccaaaaccaaagcccaagccaaaatgccgatatagttggcattggcaatcgcgttaatcgggttggcgaccaggttcatcagcagcgatttcaacacttccacaatgccggaaggcggcgcggcggacacatcgcccgcgcccgccaaaacaatgtgcgtcgggaaaaccataccggcgatgacggcggtcagggctgcggaaaacgtaccaatgaggtaaaggatgataatcggcctgatatgcgccttgttgcctttttggtgctgcgcgattgtggccgccaccaaaataaataccaaaaccggcgcgaccgctttgagcgcgccgacaaacaggctgccgaacaagcctgccgccaagcccagttgcggggaaaccgaaccgattacgatgcccaacgccaaaccggcggcaatctgcctgaccaggctgacgcggccgatcgcatgaaataaggatttgccgaacgccataattcttccttatgttgtgatatgttaaaaaatgttgtattttaaaagaaaactcattctctgtgttttttttatttttcggctgtgttttaaggttgcgttgatttgccctatgcagtgccggacaggctttgctttatcattcggcgcaacggtttaatttattgaacgaaaataaatttatttaatcctgcctattttccggcactattccgaaacgcagcctgttttccatatgcggattggaaacaaaataccttaaaacaagcagatacatttccggcgggccgcaacctccgaaataccggcggcagtatgccgtctgaagtgtcccgccccgtccgaacaacacaaaaacagccgttcgaaaccctgtccgaacagtgttagaatcgaaatctgccacaccgatgcacgacacccgtaccatgatgatcaaaccgaccgccctgctcctgccggctttatttttctttccgcacgcatacgcgcct

SEQ ID NO: 12 - DNA, 772 bp, Neisseria meningitidis; identical to SEQ ID NO: 5

SEQ ID NO: 13 - DNA, 1000 bp, Neisseria meningitidis
tttgttttttcttttggtttgtttgaatggttaaatcggggtttgggggcggatggtgcggcatccgcccggtttttgggggttgggggttttctgataaattcccccaacttaaaatctcgtcattcccgcgaaggcgggaatctgggacgtggaatctaaggaaactgttttatccggtaagtttccgtgccgacgggtctggattcccgcttttgcgggaatgacggcggtggggtttctgttttttccgataaattcctgttgcgttgcgtttttggattccagcttttgcgggaatgacggtcggtggggtttctgttttttccgataaagtcctgccgcgttgtgtttctggattcccgcctgcgcgggaatgacggtcggtgggggtttctgtttttgctgatagattcctgtggtttttcggttgctggattcccgcttttgcgggaatgacggtcggtggggtttctgttttttccgataaattcctgttgcgttgtgtttctggattcccgcctgcgcgggaatgacgcggtgggggtttctgttttttccgataaattcctgttgcgttgcgtttttggattccaacttttgcgggaatgacggtcggtggggtttcggttttttccgataaagtcctgccgcgttgtgtttctggattcccgcctgcgcgggaatgacgcggtgggggtttctgttttttctgatagattcctgtggtttttctatggattcaatcattcctgataaattcccataatctaaaatctcgtcattcccgcgaaagcgggaatctaggacgtggaatctaaggaaactgttttatccggtaagtttccgtgccgacgggtctggattcccgcttttgcgggaatgacggcggtggggtttctgttttttctgataaagtcctgccgcgttgtgtttctagattcccgcttttgcgggaatgacggcggtgaggtttctgttttttccgataaattcctgt

SEQ ID NO: 14 - DNA, 1000 bp, Neisseria meningitidis
aatcagcataggttgccacgcgcggcttgggcgttttcccacacaaagcctctgccatcggcagcaggtttttccccgatatgcgtatcacgcccacgccgccgcgcccgggtgcggtagcgactgccgcaatcgttggaacgttatccgacataaaacccccgaaaattcaaaacagccgcgattatagcaaatgccgtctgaagtccgacggtttggctttcagacggcataaaaccgcaaaaatgcttgataaatccgtccgcctgacctaatataaccatatggaaaaacgaaacacatacgccttcctgctcggtataggctcgctgctgggtctgttccatcccgcaaaaaccgccatccgccccaatcccgccgacgatctcaaaaacatcggcggcgattttcaacgcgccatagagaaagcgcgaaaatgaccgaaaacgcacaggacaaggcgcggcaggctgtcgaaaccgtcgtcaaatccccggagcttgtcgagcaaatcctgtccgacgagtacgtgcaaataatgatagcccggcgtttccattcgggatcgttgccgccgccgtccgacttggcgcaatacaacgacattatcagcaacggggcagaccgcattatggcaatggcggaaaaagaacaagccgtccggcacgaaaccatacggcaagaccaaaccttcaacaggcgcgggcaactgtacggcttcatcagcgtcatcctgatactgctttttgccgtcttcctcgtatggagcggctaccccgcaaccgccgcctcccttgccggcggcacagtggttgccttggcgggtgctttcgtgattggaagaagccgagaccaaggcaaaaattaattgcaaatcctagggcgtgcttcatatccgcccgaacgccgaaccgcacatataggcacatcccgcgcgccgccggaagcggaagccgcgccctcccaaacaaacccgaatcccgtcagataaggaaaaata

SEQ ID NO: 15 - DNA, 1000 bp, Neisseria meningitidis
gattttggtcatcccgacaagcttcttgtcgaagggcgtgaaattcctttggttagccaagagaaaaccatcaagcttgccgatggcagggaaatgaccgtccgtgcttgttgcgactttttgacctatgtgaaactcggacggataaaaaccgaacgcccggcaagtaaaccaaaggcggaagataaaagggaggatgaagagagtgcaggcgttggtaacgtcgaagaaggcgaaggcgaagtttccgaagatgaaggcgaagaagccgaagaaatcgtcgaagaagaacccgaagaagaagctgaagaggaagaagctgaacccaaagaagttgaagaaaccgaagaaaaatcgccgacagaagaaagcggcagcggttcaaacgccatcctgcctgcctcggaagcctctaaaggcagggacatcgaccttttcctgaaaggtatccgcacggcggaagccgacattccaagaaccggaaaagcacactataccggcacttgggaagcgcgtatcggcacacccattcaatgggacaatcaggcggataaagaagcggcaaaagcagaatttaccgttaatttcggcgagaaatcgatttccggaacgctgacggagaaaaacggtgtacaacctgctttctatattgaaaacggcaagattgagggcaacggtttccacgcaacagcacgcactcgtgagaacggcatcaatctttcgggaaatggttcgaccaaccccagaaccttccaagctagtgatcttcgtgtagaaggaggattttacggcccgcagcggaggaattgggcggtattattttcaataaggatgggaaatctcttggtataactgaaggtactgaaaataaagttgaagttgaagctgaagttgaagttgaagctgaaactggtgttgtcgaacagttagaacctgatgaagttaaaccccaattcggcgtggtattcggtgcgaagaaagataataaagaggtggaaaa

SEQ ID NO: 16 - DNA, 1000 bp, Neisseria meningitidis
cggcgttagagtttagggcagtaagggcgcgtccgcccttagatctgtaagttacgattccgttaaataacttttactgactttgagttttttgacctaagggtgaaagcacccttactgcttaaagtccaacgacaaaaaccaaaagacaaaaacacttttattaccctaaaatcgaacacccataaatgaccttttttgtctttggcgaggcggcagtaagggcgcgtccgcccttagatctgtaagttatgattccgttaaatagcctttactgactttgagttttttgacctaagggcggacgcgcccttactgcttcaccttcaatgggctttgaattttgttcgctttggcttgcttgacctaagggtgaaagcacccttactgccgcctcgccaaagacgaaaagggttatttacgggggttggattttaggcagtaagggcgcgtccgcccttagatctgtaagttatgattccgttaaatagcctttactgactttgagttttttgacctaagggtgaaagcacccttactgcttcaccttcaatgggctttgaattttgttcgctttggcttgcttgatctaagggtgaaagcacccttactgccgtctcgccgaagacaacgagggctatttacggcgttagagtttagggcagtaagggcgcgtccgcccttagatccagacagtcacgcctttgaatagtccattttgccaaagaactctaaaacgcaggacctaagggtgaaagcacccttactgccttacatccaagcacccttactgcaccacgtccacgcacccttactgccctacgtccacgcacccttactgccctacatccaagcacccttactgccttacatagacatgacagacgccgagcagcggaacaggactaaaaacaattaagtgatatttttgcccaactataatagacatgtataattatattactattaataataattagtttatcctccttttcatccc

SEQ ID NO: 17 - DNA, 731 bp, Neisseria meningitidis
tatgaagtcgaagtctgctgttccaccttcaattatctgaattacggaatgttgacgcgcaaaaacagcaagtccgcgatgcaggcaggagaaagcagtagtcaagctgatgctaaaacggaacaagttggacaaagtatgttcctccaaggcgagcgcaccgatgaaaaagagattccaaacgaccaaaacgtcgtttatcgggggtcttggtacgggcatattgccaacggcacaagctggagcggcaatgcttccgataaagagggcggcaacagggcggactttactgtgaatttcggtacgaaaaaaattaacggcacgttaaccgctgacaacaggcaggcggcaacctttaccattgtgggcgatattgagggcaacggtttttccggtacggcgaaaactgctgactcaggttttgatctcgatcaaagcaataacacccgcacgcctaaggcatatatcacaaacgccaaggtgcagggcggtttttacgggcccaaagccgaagagttgggcggatggtttgcctattcggacgataaacaaacgaaaaatgcaacagatgcatccggcaatggaaattcagcaagcagtgcaactgtcgtattcggtgcgaaacgccaaaagcctgtgcaataagcacggttgccgaacaatcaagaataaggcctcagacggcaccgctccttccgataccgtctgaaagcgaagagtagggaaacact

SEQ ID NO: 18 - DNA, 373 bp, Neisseria meningitidis
cgtaccgcattccgcactgcagtgaaaaaagtattgaaagcagtcgaagcaggcgataaagctgccgcacaagcggtttaccaagagtccgtcaaagtcatcgaccgcatcgccgacaagggcgtgttccataaaaacaaagcggctcgccacaaaacccgtttgtctcaaaaagtaaaaccttggcttgatttttgcaaaacctgcaatccggttttcatcgtcgattccgaaaacccctgaagcccgacggtttcggggttttctgtattgcggggacaaaatcccgaaatggcggaaagggtgcggttttttatccgaatccgctataaaatgccgtctgaaaaccaatatgccgacaatgggggtggag

SEQ ID NO: 19 - DNA, 1000 bp, Neisseria meningitidis
ttttggcttccagcgtttcattgttttcgtacaagtcgtaagtcagcttcagattgttggcttttttaaagtcttcgaccgtactctcatcaacatagttcgaccagttgtagatgttcagagtatcggtggcagcggcttcggcattggcagcagacgcagcgtctgcttgaggttgcacggcgtttttttcgctgccgccgcaggctgccagagacagcgcggccaaaacggctaatacggattttttcatacgggcagattcctgatgaaagaggttggaaaaaaagaaatccccgcgccccatcgttaccccggcgcaaggtttgggcattgtaaagtaaatttgtgcaaactcaaagcgatattggactgattttcctaaaaaattatcctgtttccaaaaggggagaaaaacgtccgcccgattttgccgtttttttgcgctgtcagggtgtccgacgggcggatagagagaaaaggcttgcatataatgtaaaccccctttaaaattgcgcgtttacagaatttatttttcttccaggagattccaatatggcaaacagcgcacaagcacgcaaacgtgcccgccagtccgtcaaacaacgcgcccacaatgctagcctgcgtaccgcattccgcaccgcagtgaaaaaagtattgaaagcagtcgaagcaggcgataaagctgccgcacaagcggtttaccaagagtccgtcaaagtcatcgaccgcatcgccgacaagggcgtgttccacaaaaacaaagcggcacgccacaaaagccgtctgtctgcaaaagtaaaagccttggcttgatttttgcaaaaccgccaaggcggttgatacgcgataagcggaaaaccctgaagcccgacggtttcggggttttctgtattgcgggggcaaaatcccgaaatggcggaaagggtgcgattttttatccgaatccgctataaaatgccgtttgaaaaccaatatgccgacaatgggggcggag

SEQ ID NO: 20 - DNA, 1000 bp, Neisseria meningitidis
tacggaaactgcaagcggatccagaagttacagcgtgcattattcggtgcccgtaaaaaaatggctgttttcttttaatcacaatggacatcgttaccacgaagcaaccgaaggctattccgtcaattacgattacaacggcaaacaatatcagagcagcctggccgccgagcgcatgctttggcgtaacagacttcataaaacttcagtcggaatgaaattatggacacgccaaacctataaatacatcgacgatgccgaaatcgaagtgcaacgccgccgctctgcaggctgggaagccgaattgcgccaccgtgcttacctcaaccgttggcagcttgacggcaagttgtcttacaaacgcgggaccggcatgcgccaaagtatgcctgcaccggaagaaaacggcggcgatattcttccaggtacatctcgtatgaaaatcattactgccggtttggacgcagccgccccatttattttaggcaaacagcagtttttctacgcaaccgccattcaagctcaatggaacaaaacgccgttggttgcccaagataaattgtcaatcggcagccgctacaccgttcgcggatttgatggggagcagagtcttttcggagagcgaggtttctactggcagaatactttaacttggtattttcatccgaaccatcagttctatctcggtgcggactatggccgcgtatttggcgaaagtgcacaatatgtatcgggcaagcagctgatgggtgcagtggtcggcttcagaggagggcataaagtaggcggtatgtttgcttatgatctgtttgccggcaagccgcttcataaacccaaaggctttcagacgaccaacaccgtttacggcttcaacttgaattacagtttctaacctctgaattttttactgatatttagacggtctttccttatcctcagaccgtcaaactttacctacgtacttggcgcgcagtacgttcatcttcaaaatggaatagac

SEQ ID NO: 21 - DNA, 1000 bp, Neisseria meningitidis
ttatcttggtgcaaaactttgtcggggtcggactggctacggctttgggtttggacccgctcatcggtctgattaccggttcggtgtcgctgacgggcggacacggtacgtcaggtgcgtggggacctaattttgaaacgcaatacggcttggtcggcgcaaccggtttgggtattgcatcggctactttcgggctggtgttcggcggcctgatcggcgggccggttgcgcgccgcctgatcaacaaaatgggccgcaaaccggttgaaaacaaaaaacaggatcaggacgacaacgcggacgacgtgttcgagcaggcaaaacgcacccgcctgattacggcggaatctgccgttgaaacgcttgccatgtttgccgcgtgtttggcgtttgccgagattatggacggcttcgacaaagaatatctgttcgacctgcccaaattcgtgtggtgtctgtttggcggcgtggtcatccgcaacatcctcactgccgcattcaaggtcaatatgttcgaccgcgccatcgatgtgttcggcaatgcttcgctttcgcttttcttggcaatggcgttgctgaatttgaaactgtgggagctgaccggtttggcggggcctgtaaccgtgattcttgccgtacaaaccgtggtgatggttttgtacgcgacttttgttacctatgtctttatggggcgcgactatgatgcggcagtattggctgccggccattgcggtttcggcttgggtgcaacgccgacggcggtggcaaatatgcagtccgtcacgcatactttcggcgcgtcgcataaggcgtttttgattgtgcctatggtcggcgcgttcttcgtcgatttgattaatgccgcgattctcaccggttttgtgaatttctttaaaggctgattttccgcctttccgacaaagcacctgcaaggtttaccgcctgcaggtgcttttgctatgatagccgctatcggtctgcaccgtttggaaggaacatc

SEQ ID NO: 22 - DNA, 1000 bp, Neisseria meningitidis
cctactccaccgattccaatatgctcggcgcgacccacgaagccaaagacttggaatttttgaactcgggcatcaaaatcgtcaaacccattatgggcgttgccttttgggacgaaaacgttgaagtcagccccgaagaagtcagcgtgcgctttgaagaaggcgtgccggttgcactgaacggcaaagaatacgccgaccccgtcgaactcttcctcgaagccaaccgcatcggcggccgccacggcttgggtatgagcgaccaaatcgaaaaccgcatcatcgaagccaaatcgcgcggcatctacgaagccccgggtatggcgttgttccacatcgcctacgaacgcttggtgaccggcatccacaacgaagacaccatcgaacaataccgcatcaacggcctgcgcctcggccgtttgctctaccaaggccgctggttcgacagccaagccttgatgttgcgcgaaaccgcccaacgctgggtcgccaaagccgttaccggcgaagttaccctcgaactgcggcgcggcaacgactactcgattctgaacaccgaatcgcccaacctgacctaccaacccgaacgcctgagtatggaaaaagtcgaaggtgcggcgtttaccccgctcgaccgcatcggacagctcacgatgcgcaacctcgacatcaccgacacccgcgccaaactgggcatctactcgcaaagcggtttgctgtcgctgggcgaaggctcggtattaccgcagttgggcaataagaaataaggtttgctgttttgcatcattagcaacttaaggggtcgtctgaaaagatgatcccttatgttaaaaggaatcctatgaaagaatacaaagtcgtcatttatcaggaaagccagttgtccagcctgtttttcggcgcggcaaaggtcaaccccgtcaatttcagcgcgttcctcaacaaacaaaccccccgaaggctggcgggtcgagacctttgcaataacataggttactaa

SEQ ID NO: 23 - DNA, 1000 bp, Neisseria meningitidis
gaatgacaattcataagtttcccgaaattccaacataaccgaaacctgacaataaccgtagcaactgaaccgtcattcccgcaaaagcgggaatccagtccgttcagtttcggtcatttccgataaatgcctgttgcttttcatttctagattcccactttcgtgggaatgacggcggaagggttttggttttttccgataaattcttgaggcattgaaattccaaattcccgcctgcgcgggaatgacggctgcagatgcccgacggtctttatagtggattaacaaaaatcaggacaaggcgacgagctgcagacagtacagatagtacggaaccgattcacttagtgcttcagtatcttagagaatcgttctctttgagctaaggcgaggcaacgtcgtactggtttttgttcatccactatatatgacacggaaaacgccgccgtccaaaccatgccgtctgaagaaaactacacagataccgccgcttatattacaatcgccgccccgtggttcgaaaacctcccacactaaaaaactaaggaaaccctatgtcccgcaacaacgaagagctgcaaggtatctcgcttttgggtaatcaaaaaacccaatatccggccgaatacgcgcccgaaattttggaagcgttcgacaacaaacatcccgacaacgactatttcgtcaaattcgtctgcccagagttcaccagcctctgccccatgaccgggcagcccgacttcgccaccatcgtcatccgctacattccgcacatcaaaatggtggaaagcaaatccctgaaactctacctcttcagcttccgcaaccacggcgattttcatgaagactgcgtcaacatcatcatgaaagacctcattgccctgatggatccgaaatacatcgaagtattcggcgagttcacaccgcgcggcggcatcgccattcatcctttcgccaattacggcaaagcaggcaccgagtttgaagcattggcgcgtaa

SEQ ID NO: 24 - DNA, 228 bp, Neisseria meningitidis
gatatcgaggtctgcgcttgaattgtgttgtagaaacacaacgtttttgaaaaaataagctattgttttatatcaaaatataatcatttttaaaataaaggttgcggcatttatcagatatttgttctgaaaaatggttttttgcgggggggggggtataattgaagacgtatcgggtgtttgcccgatgtttttaggtttttatcaaatttacaaaaggaagcccat

SEQ ID NO: 25 - DNA, 1000 bp, Neisseria meningitidis
gttttctgtttttgagggaatgacgggatgtaggttcgtaagaatgacgggatataggtttccgtgcggatggattcgtcattcccgcgcaggcgggaatctagaacgtggaatctaagaaaccgttttatccgataagtttccgtgcggacaagtttggattcccgcctgcgcgggaatgacgggattttaggtttctaattttggttttctgtttttgagggaatgacgggatgtaggttcgtaggaatgacgggatataggtttccgtgcggatggattcgtcattcccgcgcaggcgggaatctagaccttagaacaacagcaatattcaaagattatctgaaagtccgagattctagattcccgcctgagcgggaatgacgaaaagtggcgggaatgacggttagcgttgcctcgccttagctcaaagagaacgattctctaaggtgctgaagcaccaagtgaatcggttccgtactatttgtactgtctgcggcttcgtcgccttgtcctgatttttgttaatccactatctcctgccgcaggggcgggttttgcatccgcccgttccgaaagaaaccgcgtgtgcgttttttgccgtctttataacccccggtttgcaatgccctccaataccctcccgagtaagtgttgtaaaaatgcaaatcttaaaaaatttaaataaccatatgttataaaacaaaaaatacccataatatctctatccgtccttcaaaatgcacatcgaattccacacaaaaacaggcagaagtttgttttttcagacaggaacatctatagtttcagacatgtaatcgccgagcccctcggcggtaaatgcaaagctaagcggcttggaaagcccggcctgcttaaatttcttaaccaaaaaaggaatacagcaatgaaaaaatccctgattgccctgactttggcagcccttcctgttgcagcaatggctgacgttaccctgtacggcaccatcaaaaccggcgta

SEQ ID NO: 26 - DNA, 537 bp, Neisseria meningitidis
gttttctgtttttgagggaatgacgggatgtaggttcgtaagaatgacgggatataggtttccgtgcggatggattcgtcattcccgcgcaggcgggaatctagaacgtggaatctaagaaaccgttttatccgataagttttccgtgcggacaagtttggattcccgcctgcgcgggaatgacgggattttaggtttctaattttggttttctgtttttgagggaatgacgggatgtaggttcgtaggaatgacgggatataggtttccgtgcggatggattcgtcattcccgcgcaggcgggaatccagaccttagaacaacagcaatattcaaagattatctgaaagtccgagattctagattcccgcctgagcgggaatgacgaaaagtggcgggaatgacggttagcgttgcctcgccttagctcaaagagaacgattctctaaggtgctgaagcactaagtgaatcggttccgtactatttgtactgtctgcggcttcgtcgccttgtcctgatttttgttaatccactat

SEQ ID NO: 27 - DNA, 1000 bp, Neisseria meningitidis
atacggccaatggcttcagaaagcgataagcctctggctgaaaaaccgatttcttgtgttctccccaccgcacccatagacgtaaaggtatagggattggtaatcatggtaaccacatcaccgcgacgcagcaaaatattttgtcgcggatttgcaactaaatcttccaaggcaacagttcgtactacattgccacgtgtcagctgcacattcgtatcctgcacatttgccgttgaaccacctaccgcagccaccgcatccaacacacgctcaccggctgccgtcagcggcatacgcacactattcccagcacgaatcaccgacacattcgccgcattattctgcaccaaacgcaccatcacttgtggctgattggccatttttttcaggcggcctttaataatttcctgaacctgaccaggcgttttaccgaccaccgaaatatcgccaacaaacggcacagaaaccgtaccacgtgccgtgaccaactgctctggcaacttagtttgatgcgcactacccgagcccatcgaagaaaggccaccaccaaacaatactgccggcggcgcttcccaaatcataatatccaatacatcaccaatatttagcgtaccagccgaagcataaccatcgccaaactgagtgaatgactgatttatctgagccttatataataactgagcaaccgtatgattcacatcaatcagctccacttcaggaatttgaacttcagattgttgccctaaagagacaattttttttgcgctggggcctgatgaaggaatcgcagagcatcctacaattaaacttccacacaataataatactgcgtgacgaatataaaatttcactttaaacacaagccaaatcctaatataattataaatggcctaattatagcacttaatcgaaataaatttatgagtacgtagagtataattagtattcttctttccaacttccttatacttatatatatatacttatagattctaaaatc

SEQ ID NO: 28 - DNA, 1000 bp, Neisseria meningitidis
gccaaagcattgggcgcggatgccgccgctgccgaacgcgccgcgcgtcttgccaaagccgacttggtaaccgaaatggtcggcgagttccccgaactgcaaggcacgatgggcaaatactatgcctgtttggacggcgaaaccgaagaaattgccgaagccgtcgagcagcactatcagccgcgttttgccggcgacaagctgcccgaaagcaaaattgccgccgccgtggcactggccgacaaactagaaaccttggtcggcatttggggcatcggtctgattccgaccggcgacaaagacccctacgccctgcgccgcgctgccttgggtattttgcgtatgctgatgcagtatggtttggacgtgaacgaactgattcagacggcattcgacagcttccccaaaggtttgctcaacgaaaaaacgccgtctgaaaccgccgactttatgcaggcgcgccttgccgtgttgctgcaaaacgattatccgcaagacatcgttgccgccgtactcgccaaacagccgcgccgtttggacgatttgaccgccaaactgcaggccgttgccgcgttcaaacaactgcccgaagccgccgcgctcgccgccgccaacaaacgcgtgcaaaacctgctgaaaaaagccgatgccgagttgggcgcggttaacgaaagcctgttgcaacaggacgaagaaaaagccctctttgccgccgcgcaaggcttgcagccgaaaatcgccgccgccgtcgccgaaggcaatttccaaaccgccttgtccgaactggcttccgtcaaaccgcaagtcgatgcattctttgacggcgtgatggtaatggcggaagatgccgccgtaaaacaaaaccgcctgaacctgctgaaccgcttggcagagcaaatgaacgcggtagccgacatcgcgcttttgggcgagtaaccgttgtacagtccaaatgccgtctgaagccttcagacggcatcgtgcctatcgggagaataaa

SEQ ID NO: 29 - DNA, 1000 bp, Neisseria meningitidis
gaacgaaccggattcccactttcgtgggaatgacgaatttcaggttactgtttttggttttctgtttttgtgaaaataatgggatttcagcttgtgggtatttaccggaaaaaacagaaaccgctccgccgtcattcccgcgcaggcgggaatctaggtctgtcggtgcggaaacttatcggataaaacggtttcttgagatttttcgtcctggattcccactttcgtgggaatgacgcgaacagaaaccgctccgccgtcattcccgcgcaggcgggaatctagacattcaatgctaaggcaatttatcgggaatgactgaaactcaaaaaactggattcccactttcgtgggaatgacgtggtgcaggtttccgtatggatggattcgtcattcccgcgcaggcgggaatctagaccttcaatactaaggcaatttatcggaaatgactgaaactcgaaaaactggattcccacttttgtgggaatgacgcgattagagtttcaaaatttattctaaatagctgaaactcaacacactggattcccgcctgcgcgggaatgacgaagtggaagttacccgaaacttaaaacaagcgaaaccgaacgaactggattcccactttcgtgggaatgacggaatgtaggttcgtgggaatgacggcggagcggtttctgctttttccaataaatgaccccaacttaaaatcccgtcattcccgcgcaggcgggaatctaggtctgtcggtgcggaaacttatcgggtaaaacggtttcttgagattttgcgtcctggattcccactttcgtgggaatgacggaatgtaggttcgtgggaatgacgggatataggtttccgtgcggacgcgttcggattcatgactgcgcgggaatgacgggattttggtgtattccctaaaaaaataaaaaagtatttgcaaatttgttaaaaataaataaaataataatccttatcattctttaattgaattggatttatt

SEQ ID NO: 30 - DNA, 1000 bp, Neisseria meningitidis
caaaggctacgacagtgcggaaaaccggcaacatctggaagaacatcagttgttggacggcattatgcgcaaagcctgccgcaaccgtccgctgtcggaaacgcaaaccaaacgcaaccggtatttgtcgaagacccgttatagtggattaaatttaaatcaggacaaggcgacgaagccgcagacagtacaaatagtacggcaaggcgaggcaacgccgtactggtttaaatttaatccactatatgtggtcgaacagagcttcggtacgctgcaccgtaaattccgctatgcgcgggcagcctatttcggactgattaaagtgagtgcgcaaagccatctgaaggcgatgtgtttgaacctgttgaaagccgccaacaagctaagtgcgcccgctgccgcctaaaaggagaccggatgcctgattatcgggtatccggggagggttaagggggtatttgggtaaaattaggaggtatttggggcgaaaatagacgaaaacctgtgtttgggtttcggctgtcgggagggaaaggaattttgcaaagatctcatcctgttattttcacaaaaacagaaaaccaaaaacagcaacctgaaattcgtcattcccgcgcaggcgggaatccagacccccaacgcggcaggaatctatcggaaataaccgaaaccggacgaacctagattcccgctttcgcgggaatgacggcagagtggtttcagttgctcccgataaatgccgccatctcaagtctcgtcattcccttaaaacagaaaaccgaaatcagaaacctaaaatttcgtcattcccataaaaaacagaaaaccaagtgagaataacaattcgttgtaaacaaataactatttgttaatttttattaatatatgtaaaatcccccccccccccccccgaaagcttaagaatataattgtaagcgtaacgattatttacgttatgttaccatatccgactacaatccaaattttggagattttaact

SEQ ID NO: 31 - DNA, 1000 bp, Neisseria meningitidis
ataatgcaggcgctgaagttgttaaacatcaaacacacatcgttgaagacgaaatgtctgatgaggccaaacaagtcattccaggcaatgcagatgtctctatttatgaaattatggaacgttgcgccctgaatgaagaagatgagattaaattaaaagaatacgtagagagtaagggtatgatttttatcagtactcctttctctcgtgcagctgctttacgattacaacgtatggatattccagcatataaaatcggctctggcgaatgtaataactacccattaattaaactggtggcctcttttggtaagcctattattctctctaccggcatgaattctattgaaagcatcaaaaagtcggtagaaattattcgagaagcaggggtaccttatgctttgcttcactgtaccaacatctacccaaccccttacgaagatgttcgattgggtggtatgaacgatttatctgaagcctttccagacgcaatcattggcctgtctgaccataccttagataactatgcttgcttaggagcagtagctttaggcggttcgattttagagcgtcactttactgaccgcatggatcgcccaggtccggatattgtatgctctatgaatccggatacttttaaagagctcaagcaaggcgctcatgctttaaaattggcacgcggcggcaaaaaagacacgattatcgcgggagaaaagccaactaaagatttcgcctttgcatctgtcgtagcagataaagacattaaaaaaggagaactgttgtccggagataacctatgggttaaacgcccaggcaatggagacttcagcgtcaacgaatatgaaacattatttggtaaggtcgctgcttgcaatattcgcaaaggtgctcaaatcaaaaaaactgatattgaataatgcttattaacttagttactttattaacagaggattggctattacatatagctaattctcattaatttttaagagatacaata

SEQ ID NO: 32 - DNA, 1000 bp, Neisseria meningitidis
atacctgcacttgagttgccgaccataaatttagcatgtttcaataagactaaaaaatattcaaatcgaatggaaggaaatgcaataaatttatcagattgatattttaataattcttgcagaatactttcagtgccagtgtcattattagggtagatgctaatgatattttggccacttaattctaatgctttgaaatattgggccgcatattgtggcattaaatgtgcttctgtagtcacggggtgaaacatagaaataccataattttcgtatggtaaaccgtaatattctttgacttcttctaaggatgggagggtggaagaggccataacatctaaatcgggggagccgatgatgtgaatatgctttcttttttctcccatttgcactaggcgagtgacagcttgttcatttgctaccaagtggatatgagaaagtttactaatagaatgacgaatggagtcatctactgtaccagatagttcaccaccttcgatatggcaaactaaacggctgcttaatgcacctacagctgcgcctgctagtgcttctaaacggtcgccgtgaatcatgaccatatcaggttcaatttcatcagatagacgagagataaacgtaatggtattgcctaaaacggcacccattggttcaccttggatttgatttgaaaacagatatgtatgttgatagttttctcgagttacttccttgtaggttctgccatatgttttcatcatatgcataccagttacaatcaaatgcaattcaaggtctgggtgattttcaatataggctaataaaggttttagcttgccgaagtcggctctggtacctgtaatgcaaagaattcttttcatgattttagaatctataagtatatatatataagtataaggaagttggaaagaagaatactaattatactctacgtactcataaatttatttcgattaagtgctataattaggccatttataattatattaggatttggctt

SEQ ID NO: 33 - DNA, 1000 bp, Neisseria meningitidis
tctttttcggactgaaaggacgcatcatcccgacatcgagcgcgtgttcgtccggcagccaaggcataggttatgcctacgaagccatcaaatacggtctgaccgatatgatgctggcgggcggaggcgaagaatttttcccgtccgaagtgtatgttttcgactcgctttatgccgccagccgccgcaacggcgaaccggaaaaaaccccgcgcccatacgacgcgaaccgcgacgggctggtcatcggcgaaggcgcggggattttcgtgctggaagaattggaacacgccaaacggcgcggtgcgataatttacgccgaactcgtcggctacggagccaacagcgatgcctaccatatttccacgccccgccccgacgcgcaaggcgcaatccttgcctttcagacggcattgcaacacgcagaccttgcgcccgaagacatcggctggattaatctgcacggcaccgggacgcaccacaacgacagtatggaaagccgcgccgttgcagcggttttcggcaacaatacgccctgcacgtccaccaagccgcaaaccggacacacgctgggcgcggcgggcgcaatcgaagccgcgttcgcgtggggcattgctgaccggaaaagcaatcccgaagggaaacttccgccccagctttgggacgggcagaacgatcccgaccttcccgccatcaacctgaccggcagcggcagccgctgggaaaccgaaaaacgcattgccgccagctcgtcgtttgccttcggaggaagcaactgcgttttactcatcggatgaaataagtttgtcaatcccaccgctatgctatacaatacgcgcctactcttgatgggtctgtagctcaggggttagagcaggggactcataatcccttggtcgtgggttcgagccccaccggacccaccaattcccaagcccggacgtatgtttgggcttttttgccgccctgtgaaaccaaaatgctttgagaaaccttgata

SEQ ID NO: 34 - DNA, 1000 bp, Neisseria meningitidis
tagaaaaatatttcgcccaatcattagccgccgtcgtgaatcagacttggcgcaacttggagattttgattgtcgatgacggctcgacagacggtacgcttgccattgccaaggattttcaaaagcgggacagccgtatcaaaatccttgcacaagctcaaaattccggcctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcatcgcgatgggcgcgtggctggaagttttgtcggaagaaaaggacggcaaccggctggcgcggcatcacaggcacggcaaaatttggaaaaagccgacccggcacgaagatattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgcgtggctggattttgcggcagacggcaggatgcggcggctgtttaccttgaggcaatacttcggcattttgcaccgattgctgaaaaaccgttgaaaaacgccgctttatccaacagacaaaaaacaggataaatt

SEQ ID NO: 35 - DNA, 806 bp, Neisseria meningitidis
gcgcacggctttttcttcatcggtttgagggtcggcaggataatcggggacggcaaagcctttagactgcaattctttaatcgcggcggtcagttgaggtacggatgcgctgatgttcggcagtttgattacgtttgcatcgggctgtttcaccagttcgcccaattcggcaagcgcgtcgggtacgcgctgcgcttcggtcagatattcggggaatgccgccaaaatacggccggacagggaaatgtcggcagttttgacatcaatatcggcgtggcgggcaaacgcctgcacaatcggcagcagcgattgggtcgccagcgcgggggcttcgtcggtatgggtataaacaatggtggatttttgagtcataggattattctcttgtaggttggttttttcttttggaacacattgcgcggggaatgtgcgcggctattatggcatattttggcggctttgttcgcgctttgttcgatcttggcgtgtttgaacgcggcagcgtgaaaggaagggggaaatggttttcccgcgtttggcggcggtgtcggaggtgctgtgcctgatgtgcggcggcatattttcggtgaaattgattttatagtggtttaaatttaaaccagtacagcgttgcctcgccttgtcgtactatctgtactgtctgcggcttcgttgccttgtcctgatttaaatttaaaccactataatattcggtaactgtcggaatatctgctaaaattccgcatttttccgcctcgggacactcggggcgtatgtttaatttgtcggaatggagttttagggat

SEQ ID NO: 36 - DNA, 1000 bp, Neisseria meningitidis
  misc_feature 840: n = A,T,C or G
gcccgacggcgaacagacacgtcgtgaaatcaaccgcttggacagtacggcggcgcaatacgacatgcttgcaggttatcttgaaagacttgccggaaaaaccgaccgttgggcgtgcgcctaccgccaaaatgccgtctgaacacccgattatccttttgaaagcgcgattatgccccatacccttcccgatatttcccaatgtatcagacaaaatttggaacaatatttcaaagacctgaacggtaccgaaccttgcggcgtgtacgatatggtcttgcatcaggtggaaaaaccgctgctggtgtgcgtgatggaacaatgcggcggcaaccagtccaaagcctccgtcatgttgggactgaaccgcaatactttgcgtaaaaaactgattcaacacggtttgctgtgaatatgtcggcaaccgtccgtatcttgggtattgacccgggcagtcgcgtaacgggtttcggtgtcatcgatgtcagggggcgcgatcatttttacgtcgcctccggctgcatcaaaacgcctgccgatgcgcctctggcagacaggattgccgtgattgtgcggcatatcggcgaagtcgttaccgtttacaagcctcaacaggcggcagtggaacaggtgttcgtcaacgtcaatccggcatcgacgctgatgctcggtcaggctaggggcgcggcattggcggcattggtcagccataagctgcccgtttcggaatacacggccttgcaggtcaaacaggcggtagtcggcaagggcaaggcggcaaaagaacaggtgcagcatatggtggtgcagatgctggggctttcgggaacgccgcaggantggcggcggacggtcttgccgtcgcgctgacccacgccttacgcaaccacgggcttgccgccaaactcaatccttcggggatgcaggtcaagcgcggcaggtttcaatagtttcagacggcatttgtattttgccgtctgaaaagaaaatgtgtatcgag

SEQ ID NO: 37 - DNA, 1000 bp, Neisseria meningitidis
ccgccaagcgtttccccctttgtcgggcttaacatttgctttgtacggcagactttttcccttcataacgccgcctttccgaaaagacgatggtaggcgcgacgtaattctcaacccttaaggtacggttggacgaaaagttttccttttcattccacctgccaacttttcggctacaccgagtggtctcgttaggtttgggcgaactacgcccttaaaaaaacggacattctttgcatgcccgtctctaaggtttcacggtaagtttacccttataaagagttgacttaccatacttatccctttaaaacgatataaagggcgacagctgtaatacaagtatgttgtacggcagacttcttctaccaaacaaaaagttccttttagagttactcgcttatagacaaatgaaggcttagccataggcttccggtaggcctatttcaacggctggttcacaggctacgctaaaacctacggtagaaccgcgttctggggtttcgcgcacagcggcgtctttggaaccagttgtgtccgaacacgcataaccgcccgctttaatggtggtggcgggttcacctgatgtagtttcagcgtgcgctttggtagtttgcgtagccgatgttgaggaggctcgacccgaaactacggttgccgacgcgccagccgcacatgatgctggtcgttagaggcctgtagcgggttccgcacttgcttccgcttccgtaactgaacttggttccgcgaccgctggttccaaactacaagccgatacggacgctgctttggggctgggactacggcaaacggtagataatgtcggtggcggactacgtcgcagtttcgcttaatgcgtttctgccggaggacggaaccgacgcagggctgcgttttcgggttgactggcaccaaatgctatcgcttaggccgtttcattttgcgtaactatggcagcaggagagatacgttgtgctgggcctttagccaatacttctcaact

SEQ ID NO: 38 - DNA, 1000 bp, Neisseria meningitidis
cacaaaaaccaagttatgacgggaataaggtacagcagccaaaccaaggcctcgccctgcgtcggatggtcggtatagccgaaaaatccgccgagcagcacgcccaacgggctgtcttcgtgcaaatattttgatgagtcgaacacaatgtcctgaagcgcgttccaaatgcctgcttcgtgcagcgcacgcagcgaaccggcaagcagaccagcggcaacgataatcagaaacgcccctgtccaacggaaaaacttcgccagattcaggcgcatcccaccctgataaatcaacgcgccaatcacggcggcagccaaaacccccgctaccgcaccggccggcatctgccacgtcgggctctgtttgaatacggcaagcaggaaaaaaacgctctccaaaccttcgcgcgccacggcaagaaacgccataccgaccaaggcccatccttgaccgctgccacggttcaaagccgcctgcacagaatcctgaagctgccgcttcatcgaacgggcggcttttttcatccataaaatcatataagtcagcatcgcgacagcaaccaaaccgataatgccgacgacgaactcctgctgcttctggggaatctcgcccgttgccgaatggattccgtaccccagccccaaacacatcaaagaagcaagaacaaccccgaaccagaccttaggcatcagtttggaatgtccggactgtttcagaaaaccggcaacgatgccgacgatgagcgcggcttcgataccctcgcgcaacataattaaaaaagcgaccagcataaacgcgaacgaacaaggatgatgaataatatattatcggaatattttcattgcttgtaaatacaaatgcaagttatttttatctgcagtaccgcgcggcggaaagttccgcagctgcagctgcgccctgtgttaaaatcccctctccacggctgccgcaacgccgcccgaaaccatctttcttattactgccggcaacattgtccatt

SEQ ID NO: 39 - DNA, 1000 bp, Moraxella catarrhalis
gctgatttgtgagcaagcgggcgcatcagggattaccttgcatttgcgagaagatcgtcgacatattcaagatgaagatgtttatgaattgattgggcaattgacaacacgcatgaatcttgagatggcagtcactgatgagatgctaaatattgccctaaaggtacgaccagcatgggtgtgtttagtaccagaaaaacgccaagagctgactacagaaggtgggcttgatatcgccaatttatcaaatattcaagcatttatacacagtcttcagcaggcggatattaaggtttctttattcatcgatccagatccgcatcaaattgatgctgcaattgctttgggtgctgatgcgattgagctgcatacgggagcttatgctcaagcgactttacaaaataatcaaaagcttgttgataaagagcttgaccgtattcaaaaagccgttgcaatggcacaaaaaaaatcatcattattgattaatgcaggtcatggtttgacgcgtgataatgttgcagcgattgcccaaattgatggtattcatgagctgaatatcgggcatgcattgatttcagatgcgatatttatggggcttgataatgcagtcaaggcaatgaaaatggcttttattcaagataaaacgaccaatcattgatgcgttagaaagaaaatcgtaaataatgatgactattgtgtaatattatgtatttttgttcaaaaaaaggttgtaaaaaaattcatttaccattaagctaagcccacaagccacaatgaatacctattggtttgactcattagtcactaagaatctgcaaaattttgtaacagattattggcaggtcttggatcgctatgctaaaataggtgcggtaatcttgaaaaaccaaccattccttggaggaatttatgaaaaagggatataaacgctcttgcggtcatcgcagccgttgcagctccagttgcagctccagttgctgctcaagctggtgtgacagtc

SEQ ID NO: 40 - DNA, 1000 bp, Moraxella catarrhalis
gatgctgttaaagtgggtattggtcctggttctatttgtacaacccgtattgttgcaggcattggcgtcccgcagataagtgccattgatagtgtggcaagtgcgttaaaagatcgcattcctttgattgccgatggcggtattcgtttttcgggtgatatcgccaaagccatcgcagcaggcgcttcatgtattatggtgggtagcttgttggcaggtaccgaagaagcacctggtgaggtggaattattccaaggtcgttattataaggcttatcgtggtatgggcagcttgggggcaatgtctggtcaaaatggctcatcggatcgttattttcaagatgccaaagatggtgttgaaaaactggttccagagggtatcgaaggccgtgttccttataaaggccctgtggcaggcatcatcggtcaattggcaggtggtctaagatcatccatgggttatacaggttgccagaccatcgaacagatgcgtaagaataccagctttgtcaaagtgacttccgcaggcatgaaggaatcgcatgtacacgatgtacagattaccaaagaagcacccaattatcgccaaaattaactctattaatagcaaatacaagcactcattagatagggtgggtgctttttagagcataaaaaataaactgacacatgacttattgtcatatttttaaaatgcttttaatttagatttttaatttagataatggctaaaaataacagaatattaatttaaagttttcaaaatcaagcgattagatgaaattatgaaaataaataacaataattctgatttattttaaccaataatatcaattatcatttacaagaaaaattttttttgataaaattcttacttgtaccttgctattttttcttatttatcatttttggcggtattttcgttgattttagtaagtagatgagcaagggataatttgacaaaaacaaatttgatttcaagcctcataatcggagttatt

SEQ ID NO: 41 - DNA, 1000 bp, Moraxella catarrhalis
aaaactggtgatgtcttcactgctattcatggtgaaccaatcaatgattggctaagtgccaccaagattattcaggcaaatccagaaaccatgcttgatgtgacagtcatgcgtcaaggtaagcaggttgatttaaaattaatgccccgtggtgtaaagacacaaaacggcgtagtcggtcaactgggtattcgcccccagattgatatcgatacgctcattcctgatgaatatcgtatgacgattcaatatgatgtcggtgaggcatttactcaagccatccgacgaacttatgatttatcaataatgaccttagatgcgatgggtaagatgattacaggattgattggcattgaaaatctatcaggtcccattgccattgccgatgtttctaagaccagttttgagttgggatttcaagaagtgttatcgacagccgcaatcatcagtttaagcttggcagtactgaatcttttacccattccagtgttagatggcgggcatttggtattttatacttatgaatggattatgggcaaatctatgaatgaagcggtgcagatggcagcatttaaagcgggtgcgttattgcttttttgtttcatgttacttgcaatcagtaacgatatcatgcgattttttggctaagttctgatttatcgtaccattaacaaaatttttggcttttttaagctgaaatacttgccaaatttaactttttggcttacctttacacaatataaatttgggtgtagaaaattttggatacatttttataccttatttttagaaattttaaaaattaagtttggatagacttatgcgtaattcatattttaaaggttttcaggtcagtgcaatgacaatggctgtcatgatggtaatgtcaactcatgcacaagcggcggattttatggcaaatgacattgccatcacaggactacagcgagtgaccattgaaagcttacaaagcgtgctgccgtttcgcttgggtcaagtg

SEQ ID NO: 42 - DNA, 1000 bp, Moraxella catarrhalis
acttggcgaaaataccatttatatcgattgtgatgttatacaggcagatggcggtacacgcacagccagtatcagtggtgctgcggtggcacttattgatgctttagaacacttgcagcgtcgtaaaaagcttacccaagatccgcttttgggcttggtggcagcggtttctgtgggtgttaatcaaggccgtgtattgcttgatttggattatgctgaagattcaacttgtgataccgatttaaatgtggtcatgacgcaggcaggtgggtttattgagattcaaggcacagcagaagaaaagccatttactcgtgctgaagctaatgcgatgcttgatttggcagagctgggaattgggcagattatcgaagcccaaaagcaagtattaggctggtgatatgctaatcgttgaagataatggcgtgatcatcacattaaatggacaagtaaaagacccattattttggtggtcgatgatattgctgctgctgggtgtcttggtggcaatcatttgtttgattgcacccgttttttatgcaatcggtgcgttggctttatttgcagttgtggtatttgtgtttaatattcaaaggcaaaaagccaaaacttgtcatatgttttcacaaggtcgcttgaagattacgtccaaacgctttgagattcataacaaatcactaaccttatcagcatcggcaacaatatctgctaaagataacaaaatgacaattgttgatcggggcattgaatatcattttacaggttttgctgatgaccgtgaaattaatatagccaaacaggtacttttgggaaagtcaatcaaaaccaatgcggtggcggtaacattggctaagtagttgttgtgatacagacaggttggatggtctttaactccacccacctaactttttctttgtttggatttaagagtatgttatgatgggcaggattttattttaagtcatcatttaatgcaatcagttgtccagagtagccgttc

SEQ ID NO: 43 - DNA, 1000 bp, Moraxella catarrhalis
gtgatcggcaacaccccaccattcaggagcaaccaaaattgcccgtgccttgcctgtcttggtggtatcatttggcagggcaatgtggctaagtagtggtgtgccatcaggtgcggtggtggtgagtgtacgattcgttattgtcataaaattatccttttgggttggatgatatcaatgaaataccctacggttgtatggaattttatccattgtaccacggtattggtctttttaaattaacaagcagcttctagcaagtcaaagtttttatgcctattttttcagattttaaggtacaataaagccaattgttaataatatggtattgtcatgatttatgatgaattgcgaccaaaattttgggaaaattatcccttagatgcgttaacagatgctgaatgggaagcattatgtgacggatgtggcgcgtgttgtttggtgaaatttcttgatgatgacaatgttaaattgaccgaatataccgatgttgcctgccagctattggattgctcaacaggattttgccaaaactatgccaagcgtcaaacgattgtgccagattgtattcgcttaacacctgatatgctgcctgatatgctgtggttgccacgccattgtgcttataagcggttgtatcttgggcaaaatctgccagcatggcacaggctcattaaacatagccaaaaccatggtgcaggatttgcgaaagtttcaactgctgggcgatgtgtgagtgagcttggtatgagtgatgaagacatagaaaggcgagtggtgaaatgggttaaaccttgacatgattgttgacatgattgacagacaataaaaattggcaaatttgataaaattggtgtatgtgtgtgattttatcaaaagcacttgaataaaaccgagtgatacgctaaattgtagcaaaccaatcaattcatcataattttaatgaacacgaggttaaattatactgtctatgtctgatgacaattcaagcacttggtcg

SEQ ID NO: 44 - DNA, 1000 bp, Moraxella catarrhalis
taacaaaggcaacccaacacgcagttattttgtgcaaggcggtcaagcggatgtcagtactcagctgcccagtgcaggtaaattcacctataatggtctttgggcaggctacctgacccagaaaaaagacaaaggttatagcaaagatgaggataccatcaagcaaaaaggtcttaaagattatatattgaccaaagactttatcccacaagatgacgatgacgatgacgatgacgatagtttgaccgcatctgatgattcacaagatgataatacacatggcgatgatgatttgattgcatctgatgattcacaagatgatgacgcagatggcgatgacgattcagatgatttgggtgatggtgcagatgatgacgccgcaggcaaagtgtatcatgcaggtaatattcgccctgaatttgaaaacaaatacttgcccattaatgagcctactcatgaaaaaacctttgccctagatggtaaaaataaggctaagtttgatgtaaactttgacaccaacagcctaactggtaaattaaacgatgagagaggtgatatcgtctttgatatcaaaaatggcaaaattgatggcacaggatttaccgccaaagccgatgtgccaaactatcgtgaagaagtgggtaacaaccaaggtggcggtttcttatacaacatcaaagatattgatgttaaggggcaattttttggcacaaatggcgaagagttggcaggacggttacatcatgacaaaggcgatggcatcactgacaccgccgaaaaagcaggggctgtctttggggctgttaaagataaataaagcccccctcatcatcgtttagtcgcttgaccgacagttgatgacgcccttggcaatgtcttaaaacagcactttgaaacagtgccttgggcgaattcttggataaatgcaccagatttgcctcgggctaatatcttgataaaacatcgccataaaatagaaaataaagtttaggattttttt

SEQ ID NO: 45 - DNA, 1000 bp, Moraxella catarrhalis
cagcttgtaccatttggtgaatatataccatttggtggtttgttggatattttaccagggcttgagggtgtcgctagcctaagccgtggcgatgataagcaaccaccgctcaaattgggcggcggcgtgggcgatacgattggtgcggcaatttgttatgaggtggcatatcctgagacgacgcgtaaaaatgcacttggcagtaattttttattaaccgtctcaaacgatgcttggtttggtacaacagcaggtcctttgcagcatttacaaatggtgcaaatgcgaagcttggagacggggcgatggtttgtgcgtgcaacaaacaacggagtgactgcattaattgaccatcaaggacggattatcaagcagataccgcagtttcagcgagatattttgcgaggtgatgtacccagttatgttggacacacgccttatatggtttgggggcattatcccatgttggggttttctttggtgctgatttttcttagtatcatggcaaagaaaatgaaaaataccaccgccaaacgagaaaaattttataccgctgatggtgtggtagaccgctgaattgtgccactttgggcgttagagcatgagcaagattaggcgttgggtgagctttggttgtattactcatcagcctacccgaaacctgccaaacatcaccgcccaaaacctaaacatacaatggctaaaaatatcagaaaataacttgctgtattgtaaattcttatgttatcatgtgataataattatcattagtaccaagatatccattactaaacttcatcccccatcttaacagttaccaagcggtgagcggattatccgattgacagcaagcttagcatgatggcatcggctgattgtctttttgccttgttgtgtgtttgtgggagttgattgtacttaccttagtggtggatgcttgggctgatttaattaaatttgatcaaagcggtcttcacaacacaccaaacgagatatcacc

SEQ ID NO: 46 - DNA, 1000 bp, Moraxella catarrhalis
agtttgccctgattttgagagccactgccatcatgaatttgttggcgtaaacaccactcgtattcttcttcggtttcccctttccatgcaaacacagggataccagcggccgccatggcagcggcggcgtggtcttgggtgctaaaaatattgcatgatgtccagcgaacttctgcacccaaggcaaccaaagtctcaatcagcaccgctgtttgaatggtcatgtggatacagcctaggattttagcacccttaagtggttgctggtcttgatagcgttttcttaaccccatcagggctggcatctcagcttctgccaaggcaatctcacggcgaccataatcggctaaacggatatcagcgactttataatcggtgaagttttgggtggtacttggattgattgaggtaggcatatctttattcctaagctattttaaagtatttttaacaataattttgatgaatttgagataattgatgctaaaaggttgaatgaccaaaccatcgctaacaatcaagaaaagacattttaagcataaaaagcaaatgtgtcttgatggcttattataacagttattatgataaatttgggtagaaagttaaatggatcgttgggtaagtttgttggctatccttaattaattataattttttaataatgcttttactttattttaaaaatagagtaaaaaatggttggctttgggtttttatctcactatggtagataaaattgatacaaaatggtttgtattatcacttgtatttgtattataattttacttatttttacaaactatacactaaaatcaaaaattaatcactttggttgggtggttttagcaagcaaatggttattttggtaaacaattaagttcttaaaaacgatacacgctcataaacagatggtttttggcatctgcaatttgatgcctgccttgtgattggttggggtgtatcggtgtatcaaagtgcaaaagccaacaggtggtcattg

SEQ ID NO: 47 - DNA, 1000 bp, Moraxella catarrhalis
ttgggggcggataaaaagtggtctttgcccaaaggggcatatgtgggagcgaacacccaaatctatggcaaacatcatcaaaatcacaaaaaatacaacgaccattggggcagactgggggcaaatttgggctttgctgatgccaaaaaagaccttagcattgagacctatggtgaaaaaagattttatgggcatgagcgttataccgacaccatcggcatacgcatgtcggttgattatagaatcaacccaaaatttcaaagcctaaacgccatagacatatcacgcctaaccaaccatcggacgcccagggctgacagtaataacactttatacagcacatcattgatttattacccaaatgccacacgctattatcttttgggggcagacttttatgatgaaaaagtgccacaagacccatctgacagctatgagcgtcgtggcatacgcacagcgtgggggcaagaatgggcgggtggtctttcaagccgtgcccaaatcagcatcaacaaacgccattaccaaggggcaaacctaaccagtggcggacaaattcgccatgataaacagatgcaagcgtctttatcgctttggcacagagacattcacaaatggggcatcacgccacggctgaccatcagtacaaacatcaataaaagcaatgacatcaaggcaaattatcacaaaaatcaaatgtttgttgagtttagtcgcattttttgatgggataagcacgccctacttttgtttttgtaaaaaaatgtgccatcatagacaatatcaagaaaaaatcaagaaaaaaagattacaaatttaatgataattgttattgtttatgttattatttatcaatgtaaatttgccgtattttgtccatcacaaacgcatttatcatcaatgcccagacaaatacgccaaatgcacattgtcaacatgccaaaataggcattaacagacttttttagataataccatcaacccatcagaggattatttt

SEQ ID NO: 48 - DNA, 1000 bp, Moraxella catarrhalis
aaagacattacacatcatcattcaaacgcccaaccatgtacctctgccccgtggtcgcacgccaacgctttttgatgcggtgcgttgggttcagatggcttgtcaatcatttggttttattaaaattcatacctttggtagtttggctttacctgatatgtcatttgattatcgaaacaatacgcagttgaccaaacatcaatttttagccatttgccaagcactcaatattaccgctcatacgaccatgcttggtattaaatcatcacataaagatactttacatccatttgaattgacattacccaaatacggccatgcctcaaattatgatgatgaattggtgcaaaacaatccattggcttattttcatcaactgtctgccgtctgccgatatttttatacccaaacggtttgtattgttggcggtgaaagctcagggaaaactaccttggtgcaaaaacttgccaattattatggtgccagcatcgcacctgaaatgggtcgattatacacacactcccatctcggcggtagcgaacttgcccttcaatacagcgactacgcatccattgccatcaatcacgccaacgctatcgaaaccgctcgtaccactgccagctctgctgttacactgattgatactgattttgcgacaacgcaagcattttgtgaaatttatgaagggcgaacgcatccgcttgtcgcagaatttgctaaacaaatgcgattggattttacgatttatttagataataatgttgcttgggtcgctgatggcatgcgtaggcttggtgatgatcatcaacgcagtttgttcgccaataaattgcttgagattttggcacgatatgatattagttatcatatcattaatgacaccgactaccacaaacgctatctacaagcattaagcttgatagacaatcatatttttaatcattttacaaaaattcatgacaattaattagggaaaatctgatgaaaattgatattttag

SEQ ID NO: 49 - DNA, 1000 bp, Moraxella catarrhalis
ggatgtggcatatctgcccatcgacccaatacacatcggtcgaggctatcaagatgtggtacgaattaatagccagtcaggtaagggcggtgctgcgtatatcttgcagcggcattttggttttaatttaccacgctggacacagattgattttgctcgtgtggtacaggcttatgcagaaagtatggcgcgtgaactaaaaactgatgagctgcttgaaatttttacccaagcgtatcttaagcaagataaattccgcctaagtgactataccatcagcaataaaggcgatgctgtcagcttccaaggccaagtagcgacacccaaagcggtgtttgaggtgattggtcaaggcaatggtgcgttatctgcgttcattgatggcttggtgaaatccacaggcagacagattcatgtcaccaattacgccgaacacgccatcgataacaaaacccatcaaaaaaccgatacggataaccaaaccgatgccgccgtgccgcttatatccagctgtcggtagaggggcagatttattcaggcatcgccacttgccatagcaccgtatccgccatgctaaaaggtgcattatccgctttggcacaggcgtggtaatctgacccaatcaaaatcctgcatgatggcaggattttattatttagtgggctgcccaacaatgatgatcatcagcatgtgagcaaatgactggcgtaaatgactgatgagtgtctatttaatgaaagatatcaatatataaaagttgactatagcgatgcaatacagtaaaatttgttacggctaaacataacgacggtccaagatggcggatatcgccatttaccaacctgataatcagtttgatagccattagcgatggcatcaagttgtgttgttgtattgtcatataaacggtaaatttggtttggtggatgccccatctgatttaccgtccccctaataagtgagggggggggagaccccagtcatttattaggagactaag

SEQ ID NO: 50 - DNA, 1000 bp, Moraxella catarrhalis
ccccaagctttccgtttgtgtgcctgctggtgtcgggcggtcataccatgctggtgcgtgccgatggtgtgggcgtgtatcagatattgggcgagtctatcgatgatgcggtgggtgaatgctttgataaaacggcaaaaatgctcaaactgccctatcctggtggcccaaatatcgaaaaattagccaaaaacggcaacccacacgcctatgagctgccaagacccatgcagcataaagggctggatttttcgttcagtggcatgaaaaccgccattcataatctcatcaaagacacaccaaacgcccaaagcgaccccgccacacgagcagacatcgccgcaagctttgagtatgcggtggtggatactttggtcaaaaaatgcaccaaagcactacagatgacaggcattcgccagctggtggtcgcagggggcgtctctgccaatcagatgctacgccgcaccctgaccgagacgctccgccaaatcgatgcgtcggtgtactatgccccgaccgagctatgcacggataatggtgcgatgatcgcctatgctggcttttgtcggctcagctgtggacagtcggatgacttggcggttcgctgtattccccgatgggatatgacgacgcttggcgtatcggctcatagatagccacatcaatcataccaaccaaatcgtacaaacggttgatacatgccaaaaataccatattgaaagtagggtttgggtattatttatgtaacttatatctaatttggtgttgatactttgataaagccttgctatactgtaacctaaatggatatgatagagatttttccatttatgccagcaaaagagatagatagatagatagatagatagaactctgtcttttatctgtccgctgatgctttctgcctgccaccgatgatatcatttatctgctttttaggcatcagttatttcaccgtgatgactgatgtgatgacttaaccaccaaaagagagtgctaa

SEQ ID NO: 51 - DNA, 1000 bp, Moraxella catarrhalis
gagtgaactttattgtaaaatatgattcattaaagtatcaaaatcatcaaacgcagcatcagggtttgctaaatcaattttttcaccataattatagccataacgcacagcaagcgtagttatgccagcggcttgccctgataaaatatcatttttggaatcaccaaccataatggcatcagtcggtgcgatgcccagtgattgacacaggtataataaaggcgttgggtcgggctttttgacgctgagcgtatcaccgccaatcacttggtcaaacagtgtcagccatccaaaatgtgataaaattttaggcaaataacgctcaggcttattggtacaaattgccaaataaaaccccgctgcttttaatcgttcaagcccttgtataacccctgcatagctttgcgtattttcaattgttttatgggcatattctgccaaaaataactcatgggcatggtgaatcatagtcgtatcatagatatgatgtgcttgcattgctcgctcaaccaattttagcgaaccattgcccacccagcttttgatgatatcaattggcataggcggtaagttaagcttggcatacatgccattgaccgccgccgccaaatcaggggcactatcgataagcgtaccatccaaatcaaatataatcagttttttgccagtcattgacagtgtttgcatgctttttccttattcttaaaattggcggctgtttggtattttttaaatcagtcaatttttaccatttgtcatataatgacaaagtacaaatttagcaatattttagtgcattttttggcgaagttttatgaaaactggtcattggttgcaaaactttacacagtacctataaaacttgcacagttaataagaaatattttgttactataggggcgtcatttggaacaagacagttatttgtaaatagttatttgcaaaagacggctaaaagacagaacagcgtttgtttcagtgattaactaggagaaaaaca

SEQ ID NO: 52 - DNA, 1000 bp, Moraxella catarrhalis
ttgatcggttttgccccactgtttcatgatttactcaaaacaggcggcttgatcgtgctggcaggtctgacccaaaaccaaacccaagcggtcatcgatgcctactcgccttatgttacgcttgatacgccattttgttatgcagatgcccaagactgccattggcaacgcctaagcggcatcaaacctaccaacccataagcgatatgccatgagccacaaacctaagccaacaccgctatatcaacaagttgagcagaccgccaagcgttattttgagacattgggcgatgctcatactcatgatgtctatgccacttttttggccgaatttgaaaaaccgctgctcatcgccgcactcaatcacacgcacggcaatcagtcaaaaaccgcccaaatccttggtatcaatcgtggcacattacgcaccaaaatgaaaacccatcacttactttagaccgccagttatcgccatggatatgggcaggtgtgctcgcctgccgtatgatggcgatgacaccccatttgccccatatctgcacgatttgacatgatttaacatgtgatatgatttaacatgtgacatgatttaacattgtttaatactgttgccatcattaccataatttagtaacgcatttgtaaaaatcattgccccctttttttatgtgtatcatatgaatagaatattatgattgtatctgattattgtatcagaatggtgatgcctacgagttgatttgggttaatcactctattatttgatatgttttgaaactaatctattgacttaaatcaccatatggttataatttagcataatggtaggctttttgtaaaaatcacatcgcaatattgttctactgttaccaccatgcttgaatgacgatccaaatcaccagattcattcaagtgatgtgtttgtatacgcaccatttaccctaattatttcaatcaaatgcctatgtcagcatgtatcatttttttaaggtaaaccacc

SEQ ID NO: 53 - DNA, 1001 bp, Moraxella catarrhalis
actattctgctttttgtttttcacgaatgcgaatgcccaactcacgcaactggcgattatcaacttcagcaggtgcttcggtcaatgggcaatctgccgtcttggtttttgggaaggcgatcacatcacggattgagctggcaccaaccatcagcataatcaggcgatctagaccaaatgccaaaccaccgtgcggcggtgcaccaaaacgcaatgcatccatcaaaaacttaaacttaagctctgcttcttctttagaaatacccaaggcatcaaataccgcctcttgcatgtcaaccgtattaatacgcagcgaaccgccaccaatttctgtgccatttagtaccatgtcataggcaatggatagggcggtttcgggactttgtttgagttcctcaaccgagccttttgggcgtgtaaaaggatgatgaactgatgtccacttaccatcatcagtttcctcaaacattggaaaatcaacgacccaaagcggtgcccattcacaggtaaataaatttaaatcagtaccgattttaacacgcaatgcacccatagcatcattgacgattttggctttatcggcaccaaagaaaatgatatcgccagtttgggcatcggtacgctcaatcagctcaatcaaaacctcatcggtcatatttttaatgatgggtgattgtaatcctgattctttttcaacgccattattgatattgcttgcgtcattgaccttaatatatgccaatccacgagcgccataaataccaacaaatttggtgtactcatcaatctgcttgcgactcatgttaccgccatttggaatgcgtaaggcaacaacacggcctttaggatcttgggcgggccctgaaaatactttaaattcaacatgttgcatgatgtcagcaacatcaataagttttaagggaatgcgtaaatcaggcttatctgaggcataatcacgcatggcatctgcgtaagtcatgcgggggaaggtatcaaactca

SEQ ID NO: 54 - DNA, 1001 bp, Moraxella catarrhalis
tggatcatattctttattaatggtactgtttaaacctgtattttaaagtttattgggtcatattttcaagctcatcccatcgctcaagcttcatcatcaaaagctcatcaatctctaccaatcgctcaccagccttcgttgctgccgccaaatcggtattaaaccatgaaccatcttcaatctttttggcaagctgtgcctgctcttgttcaagtgcagcaatttcattaggcaaatcttcaagttcacgctgctctttatagctgagtttgcgtttttgggcaacgcctgattgaggtggtttgatttggatgggttcagcgggttttgtcgccttaggtttattgtctgtggcgtgatgagcaagccatctttcatgctgttgtacatagtcttcataaccgccaacatattccaaaacgataccgtcgccgtacttatcagtatcaaatacccaagtttgggtaacaacattatccataaaagcacggtcatggctgatgagtaataccgtgcctttaaaattgaccacaaaatcttctaaaagctcaagtgttgccatatccaaatcattggtaggctcatcaagcaccaaaacattggcaggttttagcaataatttggccaataaaacgcgtgctttttcaccgcctgatagtgctttaacaggtgtgcgagcacgatttggcgtgaataaaaaatcttgcaaatagcttaaaatgtgcgtagtttttccaccaacatcgacatggtcagagccttctgaaacattatctgcgatagatttttcagggtctaggtcgtctttgagttggtcaaaaaaagcaatatttagattggtgccaagcttaactgaacctgactgaatcgctgaatcatccaaacccaaaatgcttttaattaaggttgttttaccaacgccatttttgccaatgataccaactttatcaccacgaacaagcagcgttgaaaaatccttaactaaggttttattgtcgtat

SEQ ID NO: 55 - DNA, 1000 bp, Moraxella catarrhalis
caacttgaaaatcagctcaatgctctgccacgcacagcaccgatgagcgagattatcggaatgataaataccaaagcacaagcggttaatgtgcaggtggtgagtgcatcagttcaagcaggtcgtgaacaggattattataccgaacgccctatcgcagtgagtgcgacaggggattatcatgctttgggtcgatggttacttgagttgtcagaggctaaccatttgctgacagtgcatgattttgatctgaaggctggtttgaaccatcagctgatgatgattgttcagatgaaaacttatcaagcgaacaaacgcccaaaaccagttgctcagcaggtgcctgatgttcaatgaatattatcggtggggcattttgggtgcttggatttgggttgggattggatgtgctgatagcaccagtcaagttgttgatgataagcttgcacatattacccatgaagagcgtatggcgatcagtgagcctgtgccgatacccttatctgtgccgatgatatatcagcaaggcaaagatccttttatcaatccttatagaaatgttgaggttcttgataccaatcatgccgctgatcagcaagatgagccaaaaaccgaatctaccaaagcttggcctatggcagacactatgccatctcagccatctgatactcatcagtctgccaaggctcaggcacaagtcttcaaaggcgatccgatagtcattgataccaaccgtgttcgagagcctttagaaagctatgagttatcaagcctacgctatcatggtcgtatttttgatgatgttagacttgtggcactcattatgagtcctgatggcatcgttcatcgtgtgagtactggacaatatcttggtaaaaatcacggaaaaattacccatattgacagtcgtacgatacatctgattgaagcggtcgctgatacacaaggtggctattatcgccgtgatgtaaacattcattttattcataagcaatgacac

SEQ ID NO: 56 - DNA, 1001 bp, Moraxella catarrhalis
ttcatgcaacaagcgaccatcttggccgatgataccatcctgctcacctaagaaaatcagtttatcagcttgcagggcaatggctgtggtcagtgctacatcttctgccaatagattaaaaatttcgcccgtaaccgaaaaacctgtcggtcctagtaggacaatatggtcattatccaaattatggcgaatggcatcgacatcaattgagcgtacctcacctgtcatctgataatccataccatctctgatgccgtaagggcgagcggtgacaaaattacccgaaatggcatcaatacgagatccgtacattggggagttagcaagccccatcgacagccgagcttcgatttgtagacgaattgagccgactgcctccaagatggcaggcatagattcatacggtgttacacgcacattctcatgtaggtttgatatcagcttgcgattttgtaaatttttttccacttgtgggcgtacaccatgcacaagcaccaatttgatgcccaagctgtgtagcagtgcaaaatcatgaatcagcgtactaaaattgtcacgagcgaccgcctcatcaccaaacataaccacaaaggttttgccacgatgggtgttaatgtacggggcagaattacgaaaccaatgcacaggtgtgagtgcaggagtgttctgataggtgctgacagaattcatgaatgctccaaagagtcaatggctggtaaaataagaatggcgaacaatatatggcgagagcgtctgatgttggtcaaatgtcccattaataactatcaagataccatcataccatagcaaagttttgggcagatgccaagcgaatttatcagcttgataaggttggcatatgataaaatctaccatcatcgtcgccagttttgagcatgtgtaagtagttaccataattaaacagtcaagaaattcacaccgtcaatcagctgtgctatgcttatgggcacataaaacttgaccaacacaggataaattta

SEQ ID NO: 57 - DNA, 1001 bp, Moraxella catarrhalis
ggcatacttttgccatgctttattttggcataactgctataagcccattgctactttttatcatttatccatatgtccaataatgtgctttatgtaatttaggcacactattaactcgtgccactgttaacattcagcataaaaatcttaacaatgaatcaaagcatcgtattggctgttaaatgataagcttatatttatttaaattcagactaaatgattgtaatatggacatatcaaggttgaaatcaaaaattttggagagttatgtacgataatgataaaaaattgaccaccatcgtaggggtgttgtatacggtgtcttatattgccatatggttggtcagtggctatattttatggggctggattggtgtgacaggatttactcgtgcgatactttggctgatcgcttggatgattgtgggtacgattgctgatagaattctgataccgattattttgaccgtcgtggttgggttattttctatcttttttgaaaaaaggcgataatttggttattttttcacaaaaaatcatgattttttttgtaaactatctaaaatatcaattatgttatattatgtgataaaagatgggcatgcttaagttttggattgcaaaaatcctaatatcatcactgaccaaagctgtgatgatatcaaaactttatcaaagttcttagggtattatcaagatatcataccaaatgaatacttacccaacttactataaaaatcaaatgatatgactgtgattttattatcatagatacaaaaatcaaaacgcatgagccaaaggtatgatgaatgaatacaaaatttcgcacacattatgacaatctaaatgtcgccagaaacgctgacattgcggtgatttggtgggataggggtcaagccagtgcgattaagctaaatttttatgtgggcaatcgctgactttattttatttgtgccagttggaacaattcgtggtctaatgtatttattttaaggagataa

SEQ ID NO: 58 - DNA, 1001 bp, Moraxella catarrhalis
tctggtctacatcccaaactatttacacaagaaacactaaagacagtggagcagatgacgctcaaaaaggcatcttatagtaatttgacagttaattttcgtcaagtgcttgtacaaaaatacaccatcgtgcaagaagtttgtaccaatttaagcacaatcattttggcacacactgtcaagcaatgcttcaggcaaattagctgctggtaaagatacttgggtcatcatgcaatcgcatcaacccttcttgctgcgttgaagcgataagtttgccatcttgccaaaattgaccatggtttagacccttggcgtggcttgtggtatcgctccacatgtcgtagagtagatattcggtcatatcaaaagggcgatggaaatgtatggaatggtcaatactagccatttgtagaccttgtgtcatcaggcttagcccatgactcattaaacctgtgctgaccaaataataatcagacacaaacgcaagtagtgcttgatgaatggcaactggctgctccccaatatcagcgatacgcacccaattggcttggcgtggacgctcaggcttgggtgtcacagggtctcgtggtgtgacggggcggatttcgacatgacgctgacgcataaatcttgctttgagtggttcgggaattttatgtaaataatccgctttgagttcttgctcggtttttaggctttcagggggtggataatcaggcatggtttcttggtaatcaagcccgccttccatgggtgaaaatgaggcaatcatcgaaaaaatgacctgttcattggtcgtatgattaccgtttttgtcggtggttggcacatattgcaccgcaatgacttctcgagctgataaactgcgtccatcacgtaagcggcgtacttgatagatgactggtagacgaatatcgccacctcgtaaaaaataaccatgtaggctatgacaaggtttatcaatcgttaatgtgttagcaccagcaagcagcgcttgggca

SEQ ID NO: 59 - DNA, 1001 bp, Moraxella catarrhalis
taaaatgaccttacaaaataaaattatatgttcaaaaatcgcttaagtattgaaaaaagctataaaaacttatctattaaagcataaaagatattaaagcataaaagacgagaaaagagcaagcgtcaatgatgatatttcatataaaaacttatgaaatttttcaattttttatcgattgattcagcttggctatcggtggtcaactttggctgccaagacatcgccggctttttgaaaaatcatcacaatggcaacaatgatgatggttgaaatccacttgacatataccatgttgcgatgctcaccatagttaatcgcaaggcttcccaagccaccaccgccaaccacacctgccattgcagaataaccaatcaaagacaccaaggtcaatgtgaccgcattaatcaaaatgggcaggctttcagcaaaatagtatttgctgacaacctgccaatgcgttgcacccatagatttggcagcttcggtcagtcctgtgggtacttctaataaagcattggcactcaagcgtgcaaaaaatggaattgctgccacactcaaagggacgatggcggctgttgtgccaagggttgttcccaccaaaaatcgtgtgactggcatgagaataatgagcaaaataataaaaggaacggagcgaccaatattaataataacatccaaaattacaaatacactgcgattttcaaggatacgccctttatcggttaaaaatgccaaaaaccctatcggtagcccaaccaaaacagcgatggcagtggcagcaagccccatatagatggtttcccaagtggattgggcaaccatctcccacattcttgggtgcatttcactgacaaattttgtgacgatttcattccacatagccgataatctcaatattgacccgatgggtggttaaaaattctattgcttgcatgaccgaggtgccttcaccgataagctcagcaatggtaaagccaaattttatatcacctgcataa

SEQ ID NO: 60 - DNA, 1001 bp, Moraxella catarrhalis
agtaaacaatggtaacaaatacagcagtgtcgcacagtcctcagtacgatgattctgaatttgaatatgcaggattttggatacgatttgtggcatgtcttgtcgataatttaattgttatgattataattgcaccgtattggttttataattatcagcaaatgatggccatgcctgctgaccaaataccgttttatagtgttggggatgccatcctttatagtgctggggatgctatcctaaacttagtgatggcggcggcggttgtttggttttgggtaaaaaaaggtgcaacaccaggtaaaatgctctttgggctgcaagtccgtgatgccaaaacagggcaatttatcagtgtgccaagggcattattgcgatattttagttatctgatttcatccgtgattctttgtttgggacttatttgggttggttttgataagaaaaaacaaggctggcatgataaaattgccaaaactgttgtggtaaaacgcattcgctgatgggtcgccagttaaacaataaaaccatcaaacgcaagcagggcgatgtgtttgagcagttggcggtagataagctaaaacaagcaggctatgaaattattttaaccaactttaccaccccatttgttggtgagattgatattatcgccagacagcctttggagcaatcgcaccgtttggtgcagccaagattttgtacggtatttgttgaagtgcgtagccgaacaagttctgtgtatggtacagcgcttgagagtgttacctcaaaaaagcaggcaaaaatctaccgaacagcagaacgatttttaatcaattatcccaaatatattgatgatgcataccgttttgatgtcatggtttttgatttggttgatggattgattgaacatgaatggataaaaaatgcgttttgattggctcaatggtcgtgaattaaaatcaatcaagcaatccgtagctttactataagatatatcccagtaatatggaaacatagca

SEQ ID NO: 61 - DNA, 1001 bp, Moraxella catarrhalis
cgtttagcttcatacgcagaccttgtgcaccttcgggcaaccgaagcatcacgccagcatcacgcatccgcacaaaacccatcatgccatcaatttcgctgctgatatgatatacccccaccaaagtaaaccgcttaaatcgtggaataacgcctgctgctgagggtgaggcttcaggcaaaaccaaggtaaccttatcccccaacttaagtcccatgtcagagacaatggactcacctaatataataccaaactcgccgatatgtaaatcatccaaattgcctgcggtcatatgctcatcaatgatagaaacttgcttttcgtaatcaggctcaatgccagaaaccacgattccagtcacctgaccttcagcggttaacataccttgtagttgaatataaggggcaactgcttgcacttctggattttgcattttgattttttcggcaagttcttgccaatttgtcaaaatttctgttgaggtaactgaagcttgaggcaccatgccaagaatgcgtgatttaatttcacggtcaaagccattcatgaccgacaaaaccgtgataagcactgcaaccccaagcgtaagcccaatggttgagataaaagaaataaaggaaataaagccatttttacgcttagctttggtatatctaagcccaataaataacgccaagggacgaaacataagctgtgttccaaacgacccaaccgtgctagtttagcacttttttggacaaataccaaacatcacataacaaatgaatcatcaggttggttttgttgcgcttgtgtatctgtatgataagtttcttgctaaaacagcttttttatgtcagaatacagaaaaggtatatacttatatttttaactttaaatagatctgcttttttataccgatgatttggcatgaagtttatcggtctgatatgctggatataagtttatcggcttgatataaattttaattaatcatcaaatttttaaggaatttatcatta

SEQ ID NO: 62 - DNA, 1001 bp, Moraxella catarrhalis
taaggataccagattttggcttgtcaatcgttgtgttaatcattgtaacggtttatagtgattgtcaattaataagggtaaaaaagtatttatcaagtaataatctttcttatatgtgaatataatgacaaatttatcacatttttacaaggattttttatcaagattaggatatgttccagcttaattattagtgatgagcgtgtgattatttggcatcgttaaatttatgagtgctaaaattgccaaatgattaaaattttgctaacatgatagcccctttggtaggctttatttggtattgatgagcaataataatataccgagttaaatggattaacttaacatacgccaaaaacttaacaacgaaaagtagatgattatgacagatacagtacaaaaagatacagcacagtcccccaaaaaagtttatctaaaagactacacgccgccagtatatgcagttaataaagtggatttggatatccgcttgtttgatgatcatgctgtcgttggtgccaaacttaaaatgacacgagcacacgcaggcgagcttcggcttcttgggcgagatttaaagcttaaaagcattcacctaaatggtcaggaattagagtcgcaggcgtatcatcttgataaggaaggcttaacaattttagatgcaccagatgtcgcagtgattgagacattggttgagatttcaccacaaaccaacacaacacttgaagggctatatcaagcaggaacaggtgatgataagatgtttgtgacacaatgcgaacctgagggttttcgcaaaatcacctttttccctgaccgccctgatgttttgacagaatacaccacacgcctagaagcaccaaagcattttaaaaccttgcttgccaatggtaatttggttgagtcaggagatgtggatgaaaatcgccattataccatttggcatgatcctaccaaaaaacccagctatctattcgccgctgtcattgccaatctagaag

SEQ ID NO: 63 - DNA, 1001 bp, Haemophilus influenzae
aaatcaagcgcctgtgcctgctggtgatggttgtggagacgaattatattcttggtttgaaccgccaaaaccaggcacttcagtgagcaaacctaaagttacaccgcctgagccgtttttgtgccaacagattttgaactcaccgaatcggagagaatggttagaatagcattgaggtaaatcaatatggatatcggcattgatcttttagcaatattgttttgtgttggttttgtcgcatcatttatcgatgcaattgctggcggtggtggattaatcaccattccagcgttactcatgacaggtatgccaccagcaatggcgttaggcaccaacaaattgcaagctatgggcggtgcattatccgcaagcctttatttcttgcgaaaaagagcggtcaatttacgcgatatttggtttattttgatttgggttttcttaggttctgccctaggtacattattaattcaatcaattgacgtggcgattttcaaaaaaatgcttccttttttgattttagccattggtctatattttttatttactcctaaattaggtgatgaagatcgaaaacaacgattaagttatctgttatttggtcttttagttagcccatttttaggtttttatgatggcttctttgggccagggactggctcaatcatgagtttagcctgtgttactttgctaggatttaatctcccgaaagcggcagcacatgcaaaagtgatgaacttcacttcgaaccttgcttcttttgcacttttcttattgggcggacaaattctttggaaagtgggtttcgtgatgatggctgggagcattttaggtgcaaatttaggtgccaaaatggtgatgacgaaaggtaaaaccttgattcgaccgatggttgttatcatgtcttttatgatgacggctaaaatggtttacgatcagggttggtttcatttttaattcggaaagcgcgcaaaagtgcggttaaaattaattacattttatta

SEQ ID NO: 64 - DNA, 1001 bp, Haemophilus influenzae
ttgaagtccccaatttacccaccacaattcctgcggcaacattggctaggtaacaagattcttcgaaagaacgtccatctgctaatgtggttgctaatacactaatgacagtgtcaccggctcccgtcacatcaaacacttcttttgcaacggttggcaaatgataaggctcttgatttgggcgtaataatgtcatgcctttttcagaacgcgtcaccaaaagtgcggttaattcaatatcagaaattaattttaaacctttcttaataatctcttcttctgtattacatttacctacaacggcttcaaattcagacatattgggtgtcaataatgtagccccacgataacgttcaaaatcagttccctttggatcgatcaacacaggcacattcgctttgcgtgcaatttgaatcattttctgaacatctttaagcgtgcctttgccgtaatcagaaagaatcaaagcaccgtaatttttcaccgcactttctaacttcgctaataaatccttgcaatctacattattgaaatcttcttcaaaatcaaggcggagcagctgttgatgacgagataaaatacgtaatttagtaatggttggatgggtttctaatgcaacaaaattacaatcaatcttttgtttttctaataagtgggaaagtgcagaacctgtctcatcttgtccaatcaatcccattaactgaacgggtacattgagtgaagcaatattcatcgccacatttgcagcaccgcccgcgcgttcttcattttcttgtacgcgaactactggcactggtgcttctggtgaaatacggttggttgcaccgaaccaataacgatcaagcatcacatcgcctaatacaagtacttttgcttgcttaaattctgctgaatattgagccattttaaaatctctctatttgaataaccaaaattgtggcgattttaccacaactcaaatttacgataaactacgcccctaacttacgtggaaagaacaa

SEQ ID NO: 65 - DNA, 1000 bp, Haemophilus influenzae
agcaataattatagctggaatattctttaaagatgaaagagatcgtataagacaaaaagaattttatattggagaattattagcaattattggttcgctaatattcgtaataaatagttcaaataatgatggaaatacagacttttttcttggggcaatatttctttttacagctatttttattcaatctgtacagaatttaattgtaaaaaaagtagccaaaaagataaatgctgttgtaataagtgcatcgacagcaacaatttcaggagtattatttttatgtttagcttttaatactaaacaaatatatttattacaagatgttggcattggaatgttgataggtttagtttgcgctggcttttatgggatgctaacagggatgttgatggctttttatattgttcaaaaacagggaatcactgtttttaacattttgcaattattaattcctctttcaactgcgataataggttacttaacattagatgaaagaataaatatctatcagggaattagcggtattattgtaattattggttgtgtattggcattaaaaagaaaaaacaaggagtgttgatatataaagtagatgatgttggtggaataggtatagttaaatatctggttcaattggttttattaagggcgttagcaattctccatttaagtttatgtttgaattagatattttgggaaaagatggaagaataaagctgttaaataatgctgaaacatatgaactataccaatactcaaataaaaataattctgctggaaatgattataaatctctaattctaacttgtagagaggataatgactatcaatcagaaagaatgattaaagccattaaaaatattattcattgtatgactaataatcatcaacctatttcaagtgctgaaacatctttagaaactattaaaattattcacggaataattaattctgttaaaataggtaatgatcctaacaatatataaggagaataagt

SEQ ID NO: 66 - DNA, 1001 bp, Haemophilis influenzae
taaatactccaaaataaatttcagataacgtggtctgtaagacaaaaaaataaaaaaaatgttcaataagaggagagcaaattatcttgtttaaaaggaaatcggagcagtacaaaaacggtcttacaagtagcaaattctataaatttatgttctaatacgcgcaattttctagtcaataaaaaggtcaaaaaatgagctggattaaccgaatttttagtaaaagtccttcttcttccactcgaaaagccaatgtgccagaaggcgtatggacaaaatgtactgcttgtgaacaagtactttatagtgaagaactcaaacgtaatctgtatgtttgcccgaaatgtggtcatcatatgcgtattgatgctcgtgagcgtttattaaatttattggacgaagattcaagccaagaaattgcggcagatttagaaccaaaagatattttaaaattcaaagatttaaagaaatataaagatcgtatcaatgcggcgcaaaaagaaacgggcgagaaagatgcgctaattactatgacaggtacactttataatatgccaatcgttgtggctgcatcgaactttgcttttatgggcggttcaatgggttctgtagttggtgcaaaatttgttaaagcggctgaaaaagcgatggaaatgaattgtccatttgtgtgtttctctgcgagtggtggtgctcgtatgcaggaagcattattctctttaatgcaaatggcaaaaactagtgccgtacttgctcaaatgcgtgaaaagggtgtgccatttatttcagtattaacggatccgactttaggcggcgtatcagccagttttgcgatgttaggggatttaaatattgccgagccaaaagccttaattggttttgcagggccacgcgttattgaacaaactgtgcgtgaaaaattgccagaaggtttccaacgtagtgagtttctacttgagaaaggggcaattgatatgatcgtgaaacgttcagaaatgcgt

SEQ ID NO: 67 - DNA, 1001 bp, Haemophilus influenzae
tcacttaattcaagcgcatcaatgttttctaaaacatcaacagaattgaccgcacttgtatctaaaatttcgccatttattaagactgcgcgtaatgccaaaacatgattagaggttttaccatattgcaatgagccttgcccagaggcatcggtgttaatcattccacctaaagtcgctcgattgctggtggacagttctggggcaaagaacaaaccatgtggttttaaaaattgattaagttgatcttttactacgcctgcttgtactcgaacccaacgttcttttacattgagttctaagatggctgtcatatgacgagaaagatccactattatattgttattgatggattgcccatttgtgccagtgcctccaccgcgaggcgtaaagctgattgattgatattcaggtaaatttgccaattttgttatccgcactatatcagcaaccgttttcggaaaaagaattgcttgtggaagttgttggtaaacgctgttatccgtagccagacttaatctatctgcatagtttgtcgcaatatccccctcaaaatgttggcattgaagatcatcaagataatcaagtacatattgttcaacttgaggaatgcgatttagatttggcaacatagtatttgacccatttaaacatatcagatggaggctttgataatatcctaaggctagaataatgtcgattaggaaagagagaggagaaagtaaaaagtctgtttaagaaagtgttattttggataaaaactaaacaaaaaattcaaaagaatttgatcttttcaatttttataggataataagcgcacttttgaacgttcctttggggtaaacataagcaaaggaattgaatttgtcaaaaggtaataaagtagggcaaattcaaaaccctagttaagtgactgtttataatgtagctttaattaaaagttcagtataaacaaggacactttttattactattcgatcactaaatagaggacatcaaaa

SEQ ID NO: 68 - DNA, 1001 bp, Haemophilus influenzae
tcgattgtatcctatataaattatagacgtaaaaaatcattaaataatgcaaacaccgttaagcttaataacagtgctgcgccaattcgataacagatgctttgcacccgctcagaaacaggttttcctttaacagcttccattgttaaaaaaactaaatgaccgccatctaatactggtaatggaaataaattcataatccctaaatttacactaatcaatgccataaaacttaaaaaatacaccaatccaatatttgctgatgcgccagcaccttttgcaatagaaattggcccacttaaattatttaatgacaaatcgccagtaagtaatttccctaatattttcaaggttaaaagggaaagctgtcctgttttttcaatgcctttttgtaaagattcaagaataccatattttaattcagtacggtattcatccgctaattttgttaaggctgggctaaccccaacaaaccatttgccattttgattacgcactggagttaggactttgtcaaatgtttctccattacgttcaactttaatagaaaaagattcgccttgttcgacctgttttataaaatcttgccaaggaagtgcggttaaattttcttttaaaattttatcaccgatttgtaaaccagctttctcagcgggagaattttgaacaactttagaaagcaccatttcaattttaggacgcataggcataatccctaatgcctcaaaagcactttctttttcaggatcgaatgtccaatttgtaagatttaaagtccgttgttgttcaatattagaattgaaaggagaaaggctaatctcaacattaggctcccccatttttgtggcaagtagcatattgatggtttcccaatcttgagtttcttcgccatcaattgtaagaatttgcgtattgggttcaatgtgggcttgtgctgcgattgagtttggtgttattgattcaatcactggtttaaccgttggcattccataaaggtaaat

SEQ ID NO: 69 - DNA, 1001 bp, Haemophilus influenzae
tttgataaatatccttaattaaatgatgggtttaatattttctctgcccaattaaattaggcagagaacgttgtttttgagttctgatgaagaaaaaagttcaatttattagaaagaacctccaatactaaattggaactgttcgacatcatcattttcatattttttaattggtttggcataagagaataccaatggcccaataggagattgccattggaatccgacacctgtagaggcgcgaatacggcttgatttgccataatcgggtaagctttttaatacattgttatctaacccactcttatccgatttccacttagtattccaaacacttgccgcatcaacaaatagggaggttcggactgtattttggcttttatcactcacaaacggtgttggtacaataagttctgcactcgcagttgtgattgcattaccaccaatcacatcagaacttatcttcttaaaagtaccattaccattaccatgttctgcataaattgcgttaggtccaatactaccataagcaaaaccacgtaatgaaccgatgccacccgctgtataagtttgatagaacggtaaacgcttgtttccaaaaccatttgcatatcctgcagatgcttttgcagatacaacccagaggtgatctctgtctaatgggtagaaaccctgtacgtctgcacttagtttgtagtatttgttatcagaacctggaatagtaactcgtccaccaagacttgctttaacccctttagttgggaaatagcctctattaaggctgttatagttccaaccaaaagaaaaatcaaagtcatttgttttaatgccattacctttaaatttcattgattgaatatataaattacggttatattctagagcaaagttactaattttattataggtatggcctaatcctacataataggagttattttcatttacagggaaacctaaagtaacattacttccataagtcgtacgcttatagttagagg

SEQ ID NO: 70 - DNA, 1001 bp, Haemophilus influenzae
ttagatttctcctaaatgagttttttatttagttaagtatggagaccaagctggaaatttaacttgaccatcacttcctggaaggctcgccttaaagcgaccatctgcggaaaccaattgtagcacctttcctaagccctgtgtagaactataaataatcataattccatttggagagaggcttgggctttcgcctagaaaagatgtactaagtacctctgaaacgcccgttgtgagatcttgtttaactacattattgttaccattaatcatcacaagtgtttttccatctgcactaatttgtgcgctaccgcgaccacccactgctgttgcactaccaccgcttgcatccattcgataaacttgtggcgaaccacttctatcggatgtaaataaaattgaatttccgtctggcgaccacgctggttcagtattattacccgcaccactcgtcaattgagtaggtgtaccgccatttgctcccataacgtaaatattcagaacaccatcacgagaagaagcaaaagctaaacgagaaccatctggcgaaaaggctggtgcgccattatgcccttgaaaagatgccactactttacgtgcgccagaatttaaatcctgtacaacaagttgtgattttttattttcaaacgatacataagccaaacgctggccgtctggagaccaagctggagacataattggttgggcactacgattgacgataaattgattatagccatcataatctgctacacgaacttcataaggttgcgaaccgccatttttttgcacaacataagcgatacgagttctaaaggcaccacggatcgcagttaatttttcaaaaacttcatcgctcacagtatgcgcgccatagcgtaaccatttatttgttactgtatagctattttgcattaatacagtccctggcgtacctgatgcaccaaccgtatcaattaattgataagtaatactataaccattacccgatggaaccactt

SEQ ID NO: 71 - DNA, 1001 bp, Haemophilus influenzae
ggcgataaccgagtttttggggtatttagtgccaaagaagacccacaaaacccaaaattatccagagaaaccttaattgatggcaagctaactacttttaaaagaactgatgcaaaaaccaatacaacagccgatacaacaaccaataaaacaaccaatgcaataaccgatgaaaaaaactttaagacggaagatatactaagttttggtgaagctgattatcttttaattgacaatcagcctgttccgcttttacctgaaaaaaatactgatgatttcataagtagtaggcatcatactgtaggaaataaacgctataaagtggaagcatgttgcaagaatctaagctatgtaaaatttggtatgtattatgaagacccacttaaagaagaagaaaaagaaaaagaaaaagaaaaagaccaagaaaaaaaagaaaaagaaaaacaaacgacgacaacatctatcgagacttattatcaattcttattaggtcaccgtactgccaaggccgacatacctgcaacgggaaacgtgaaatatcgcggtaattggtttggttatattggtgatgacacgacatcttactccactactggagataaaaatgctctcgccgagtttgatgtaaattttgccgataaaaagctaacaggcgaattaaaacgacacgataatggaaataccgtatttaaaattactgcagaccttcaaagtggtaagaatgacttcactggtacagcaaccgcaacaaattttgtaatagatggtaacaatagtcaaactggaaatacccaaattaatattaaaactgaagtaaatggggcattttatggacctaaggctacagaattaggcggttatttcacctataacggaaattctacagctaaaaattcctcaaccgtaccttcaccacccaattcaccaaatgcaagagctgcagttgtgtttggagctaaaaaacaacaagtagaaacaaccaagtaatggaatactaaaaa

SEQ ID NO: 72 - DNA, 1001 bp, Haemophilus influenzae
tagaattatattcttatacaaaattgataattgttcgcattatcattttttttttgtaataatgtcaacttataattttttaagttcatggataaaatatgaaaaatggcgtaaaacaactttttctcttatcattaataggcttatcattaacgaatgtagcttgggcagaagttgcacgtcctaaaaatgatacattgacaaatacgattcaaagtgcggaattaaaaacctcctctttttcctctatgcctaagaaagaaataccaaataggcatattatttctctttccaaaagccaattagcgcaccatccaaggcttgttttgcgtgggttaattcctgctttatatcaaaataacactcaggcagttcaactgttattaccactatataaacaatttcctcaacaagataatttcttactaacttgggcaaaggctattgaagctcgtgaacaaggtgatttaactcaatctattgcttattatcgtgaattattcgctcgagacgcatctttactacctttacgttattaattagctcaagctctattttttaactatgaaaatgaagctgccaaaattcaatttgaaaaattacgtacagaggtagatgatgaaaaatttttaggtgttattgatcagtatcttttaacactaaatcagcggaatcaatggatatggcaagtaggattaaattttttaaatgatgataatttgaataacgctccaaaaagtggcacaaaaattggtagttggaccgcttgggaaaaagaaagtgggcagggggtagggtattctttatcagtagaaaaaaaatggccatgggcagatcatttttttagtaaaactatgtttaatgggaatggaaaatattattgggataataaaaaatacaatgaggctactgtgcgtataggtggtggtttaggctatcaaactgcctcagttgaagtctcgttgtttccttttcaagaaaaacgctggtatgcaggcggt

SEQ ID NO: 73 - DNA, 1001 bp, Haemophilus influenzae
taataaattgctccataaagaggtttgtgccttataaataaggcaataaagattaatataaaccgtttattaaaatgccaaaggcttaataaacagcaaactttgttttcccaaaaaaagtaaaaaactcttccattatatatatatatatatataattaaagccctttttgaaaaatttcatatttttttgaattaattcgctgtaggttgggtttttgcccacatggagacatataaaaaagatttgtagggtgggcgtaagcccacgcggaacatcatcaaacaactgtaatgttgtattaggcacggtgggcttatgcctcgcctacggggaaatgaataaggataaatatgggcttagcccagtttatggatttaattatgttgaaatggggaaaacaatgtttaaaaaaacacttttattttttaccgcactattttttgccgcactttgtgcattttcagccaatgcagatgtgattatcactggcaccagagtgatttatcccgctgggcaaaaaaatgttatcgtgaagttagaaaacaatgatgattcggcagcattggtgcaagcctggattgataatggcaatccaaatgccgatccaaaatacaccaaaaccccttttgtgattaccccgcctgttgctcgagtggaagcgaaatcagggcaaagtttgcggattacgttcacaggcagcgagcctttacctgatgatcgcgaaagcctcttttattttaatttgttagatattccgccgaaacctgatgcggcatttctggcaaaacacggcagctttatgcaaattgccattcgctcacgtttgaagttgttttatcgccctgcgaaactctcgatggattctcgtgatgcaatgaaaaaagtagtgtttaaagccacacctgaaggggtgttggtggataatcaaaccccttattatatgaactacattggtttgttacatcaaaataaacctgcgaaaaatgtcaaaatggttg

SEQ ID NO: 74 - DNA, 1001 bp, Haemophilus influenzae
tagtagatttccgcacgggcaaaaatacaatggtgttatttaacctcactttgccaaatggcgagccagtgccaatggcatccaccgcacaagatagcgaaggggcatttgtgggcgatgtggtgcaaggtggtgtgcttttcgctaataaacttacccagccaaaaggcgagttaatcgtcaaatggggtgagcgagaaagcgaacaatgccgtttccaatatcaagttgatttggataacgcacaaatacaaagtcacgatattcaatgcaaaaccgcaaaataaataattgaagaggatttatgcaaaaaacacccaaaaaattaaccgcgcttttccatcaaaaatccactgctacttgtagtggagcaaattatagtggagcaaattatagtggctcaaaatgctttaggtttcatcgtctggctctgcttgcttgcgtggctctgcttgattgcattgtggcactgcctgcttatgcttacgatggcagagtgacctttcaaggggagattttaagtgatggcacttgtaaaattgaaacagacagccaaaatcgcacggttaccctgccaacagtgggaaaagctaatttaagccacgcagggcaaaccgccgcccctgtgcctttttccatcacgttaaaagaatgcaatgcagatgatgctatgaaagctaatctgctatttaaagggggagacaacacaacagggcaatcttatctttccaataaggcaggcaacggcaaagccaccaacgtgggcattcaaattgtcaaagccgatggcataggcacgcctatcaaggtggacggcaccgaagccaacagcgaaaaagcccccgacacaggtaaagcgcaaaacggcacagttattcaaccccgttttggctactttggctcgttattacgccacaggtgaagccaccgcaggcgacgttgaagccactgcaacttttgaagtgcagtataactaaaatatttattatccagtgaaaaaa

SEQ ID NO: 75 - DNA, 1001 bp, Haemophilus influenzae
  misc_feature 55: n = A,T,C or G
ttatccgctaacatttcatcagtaattccatgaactttaatcgcatcaggatcancggggcgatctggcttaatataaatatgayaattattacctgtgtaacgacgatttattaattcaactgcaccaatttcaataatgcagtgtccttcataatgcgcgccaagctgattcatacctgtagtttcagtatctaatacaatttggcgattgggattaatcatttgttcaacctatctctttccattaaaatacttgccattctacacaacaacctttttgttatgcckaaacagattgaaatttttactgatggatcttgcttaggtaatccaggggcgggcggaattggtgccgtattgcgttataaacaacatgaaaaaacactctccaaaggctatttccaaaccaccaataatcgaatggaattacgcgctgtcattgaagcattaaatacattaaaagaaccttgcttgatcacgctttatagtgatagccaatatatgaaaaatggcataaccaaatggatctttaactggaaaaaaaataattggaaagcaagttctggaaagcctgtaaaaaaccaagatttatggatagccttagatgaatccatccaacgtcataaaattaattggcaatgggtaaaaggccatgctggacacagagaaaatgaaatttgcgatgaattagcaaaaaaaggggcagaaaatccgacattggaagatatggggtacatagaagaataatacaactgatataacgtcatatttttcgatacctaaaaatatttaatacttaaacctaaaacagaataaaaaataatcaaattcatttaaaaaatgtgatctcgatcagatttcaagaaaattaaaattttggagtattgacatcaaaaattttttttgtaaagatgcagctcgtccgttttggcgattggacaattctattggagaaaagttcaatcatagatagtaaacaaccataaggaatacaaatta

SEQ ID NO: 76 - DNA, 1483 bp, Moraxella catarrhalis
  CDS (1)...(924)
  misc_feature 213, 218, 284, 289, 298, 304, 306, 437, 511, 590, 598,671, 736, 750, 1141, 1147, 1206, 1225, 1283, 1375, 1378,1387, 1440, 1454: n = A,T,C or G
tcagtgcttggttttttaagatatgtaccgctgtcagtcctgcatggasrvaugyhuargtyrvarusrvauhsgyttggcggcgtgtgcgtcttatatttcctatcattgcaggcttagtattuaaaacysaasrtyrsrtyrhscysargusrtatcgcagcatccaagccaatttaatcttggttcaccccaagatgccatyrargsrgnaaasnuuvahsrysmtrgacgcacagcggcaaaaactcgccaaacaaatcctaaaaaatcagctcasaagnarggnysuaaysgnuysasngnuatcagtgcagtcgacagtcttaaaacttgggcaatgccaccaaaatggsraavaassruysthrtraamtrrystrtctatcgcacaaattaaaacggttcatcatgaagatatcctaatcaaasraagnysthrvahshsguasuysgcacttgccaatccaagtggtatgcttgccattgtgcctcatatcggcaauaaasnrsrgymtuaavarhsgyacttgggagatgatgaatgcttggctcaatacctttggctcccctactthrtrgumtmtasnaatruasnthrhgysrrthratcatgtataagcccatcaaaaatgcggcggtagatcgctttgttttamttyrysrysasnaaaavaasarghvaucaggggcgtgaaagactaaatgccagccttgtacccacagatgctagtgngyargguarguasnaasruvarthrasaasrggtgttaaggcaatttttaaaacactcaaagcaggtggatttagtatcgyvaysaahysthruysaagygyhsratactgcccgaccatgtacctgatccatcaggtggtgagattgctccturashsvarasrsrgygyguaarttttttggtattaaaaccctaaccagtacgctggcgtcaaagcttgcthhgyysthruthrsrthruaasrysuaagcaaaaactggttgtgctcttgttggcttaagctgtattcggcgtgaaaaysthrgycysaauvagyusrcysargarggugatggcgatggttttgaaattttttgttatgaattaaatgatgaacaaasgyasgyhguhcystyrguuasnasgugnctttattcaaaaaataccaaaattgcaaccactgctttaaatggtgcgutyrsrysasnthrysaathrthraauasngyaaatggaacaaatgatttatccacattttttgcattatatgtggagctatmtgugnmttyrrhshuhstyrmttrsrtyrcgtcggttcaagcatacaccactattaaataatccttatttacttaatargarghyshsthrruuasnasnrtyruuasngaaaatgagctaaaaaaaatagccataaagcttcaagccatgtcaaagguasnguuysysaaysugnaamtsrysgatagttatgagassrtyrgu

SEQ ID NO: 77 - DNA, 1439 bp, Neisseria meningitidis
  CDS (1)...(894)
  misc_feature 57, 211, 297, 304, 361, 539, 589, 683, 685, 814, 895,903, 986, 1215, 1279, 1287, 1357, 1363, 1385, 1428: n = A,T,C or G
atgtttcgtttacaattcgggctgtttccccctttgcgaaccgccatgmthargugnhgyuhrruargthraamtcacatcctgttgaccgccctgctcaaatgcctctccctgctgccactthsuuthraauuyscysusruurutcctgtctgcacacgctgggaaaccggctcggacatctggcgttttacsrcysuhsthrugyasnargugyhsuaahtyrcttttaaaggaagaccgcgcgcgcatcgtcgccaatatgcgtcaggcauuysguasargaaargvaaaasnmtarggnaaggcatgaatcccgaccccaaaacagtcaaagccgtttttgcggaaacggymtasnrasrysthrvaysaavahaaguthrgcaaaaggcggtttggaacttgcccccgcgtttttcagaaaaccggaaaaysgygyuguuaaraahhargysrgugacatagaaacaatgttcaaagcggtacacggctgggaacatgtgcagasguthrmthysaavahsgytrguhsvagncaggctttggacaaacacgaagggctgctattcatcacgccgcacatcgnaauasyshsgugyuuhthrrhsggcagctacgatttgggcggacgctacatcagccagcagcttccgttcgysrtyrasugygyargtyrsrgngnurhccgctgaccgccatgtacaaaccgccgaaaatcaaagcgatagacaaaruthraamttyrysrrysysaaasysatcatgcaggcgggcagggttcgcggcaaaggaaaaaccgcgcctaccmtgnaagyargvaarggyysgyysthraarthragcatacaaggggtcaaacaaatcatcaaagccctgcgttcgggcgaasrgngyvaysgnysaauargsrgygugcaaccatcgtcctgcccgaccacgtcccctcccctcaagaaggcgggaathrvaurashsvarsrrgngugygygaaggcgtatgggtggatttcttcggcaaacctgcctataccatgacggugyvatrvaashhgyysraatyrthrmtthrctggcggcaaaattggcacacgtcaaaggcgtgaaaaccctgtttttcuaaaaysuaahsvaysgyvaysthruhhtgctgcgaacgcctgcctggcggacaaggtttcgatttgcacatccgccyscysguargurgygygngyhasuhsargcccgtccaaggggaattgaacggcgacaaagcccatgatgccgccgtgrvagngyguuasngyasysaahsasaaaavattcaaccgcaatgccgaatattggatacgccgttttccgacgcagtathasnargasnaagutyrtrargarghrthrgntyrctgtttatgtacaaccgctacaaaatgccguhmttyrasnargtyrysmtr

SEQ ID NO: 78 - DNA, 1514 bp, Haemophilus influenzae
  CDS (1)...(936)
  misc_feature 55, 63, 66, 354, 373, 434, 439, 604, 606, 759, 764, 822,827, 841, 1053, 1153, 1156, 1222, 1308, 1377, 1379, 1445,1502: n = A,T,C or G
atgaaaaacgaaaaactccctcaatttcaaccgcactttttagccccamtysasnguysurgnhgnrhshuaaraaatactggcttttttggctaggcgtggcaatttggcgaagtattttaystyrtruhtrugyvaaatrargsrutgtcttccctatcctattttgcgccatattggtcatggtttcggttggcysurtyrruarghsgyhsgyhgytrctgttttcacatttaaaagtgggtaaacgtcgagctgccattgcacgcuhsrhsuysvagyysargargaaaaaaargcgtaatcttgaactttgtttccctgatatgcctgaaaacgaacgtgagargasnuguucyshrasmtrguasnguargguacgattttgcaagaaaatcttcgttcagtaggcatggcaattatcgaathrugnguasnuargsrvagymtaaguactggcatggcttggttttggtcggattcacgtatcaaaaaatggtcgthrgymtaatrhtrsrassrargysystrsraaagttgaaggcttacattatctaaaagaaaatcaaaaagatggaattysvagugyuhstyruysguasngnysasgygttctcgtcggtgttcatttcttaacgctagaacttggcgcacgcatcvauvagyvahshuthruguugyaaargattggtttacatcatcctggcattggtgtttatcgtccaaatgataatgyuhshsrgygyvatyrargrasnasasncctttgcttgattggctacaaacacaaggccgtttacgctccaataaaruuastrugnthrgngyarguargsrasnysgatatgcttgatcgtaaagatttacgcggaatgatcaaagctttacgcasmtuasargysasuarggymtysaauargcacgaagaaaccatttggtatgcgcctgatcacgattacggcagaaaahsguguthrtrtyraarashsastyrgyargysaatgccgtttttgttcctttttttgcagtacctgacacttgcactactasnaavahvarhhaavarasthrcysthrthractggtagttattatttattgaaatcctcgcaaaacagcaaagtgattthrgysrtyrtyruuyssrsrgnasnsrysvaccatttgcgccattacgcaataaagatggttcaggctataccgtgagtrhaaruargasnysasgysrgytyrthrvasratttcagcgcctgttgattttacggatttacaagatgaaacggcgattsraarvaashthrasugnasguthraagctgcgcgaatgaatcaaatcgtagaaaaggaaatcatgaagggcataaaaaargmtasngnvaguysgumtysgytcacaatatatgtggctacatcgccgttttaaaacacgtccagatgaasrgntyrmttruhsargarghysthrargrasguaatacgcctagtttatacgattaaasnthrrsrutyras

SEQ ID NO: 79 - DNA, 1546 bp, Haemophilus influenzae
  CDS (1)...(957)
  misc_feature 57, 59, 61, 64, 154, 361, 374, 380, 383, 450, 452, 459,526, 601, 670, 685, 763, 831, 840, 910, 979, 982, 1079,1161, 1290, 1368, 1380, 1394, 1464, 1519: n = A,T,C or G
atgtcggataatcaacaaaatttacgtttgacggcgagagtgggctatmtsrasasngngnasnuarguthraaargvagytyrgaagcgcacttttcatggtcgtatttaaagcctcaatattgggggattguaahshsrtrsrtyruysrgntyrtrgytggcttggtattttctttttattgttgttagcatttgtgccttttcgttrugyhhuuuuaahvarhargctgcgcgataaattgacgggaaaattaggtatttggattgggcataaauargasysuthrgyysugytrgyhsysgcaaagaaacagcgtacgcgtgcacaaactaacttgcaatattgtttcaaysysgnargthrargaagnthrasnugntyrcyshcctcattggactgaacaacaacgtgagcaagtgattgataaaatgtttrhstrthrgugngnarggugnvaasysmthgcggttgtcgctcaggttatgtttggtattggtgagattgccatccgtaavavaaagnvamthgygyguaaargtcaaagaaacatttgcaaaaacgcagcgaatttatcggtcttgaacatsrysyshsugnysargsrguhgyuguhsatcgaacaggcaaaagctgaaggaaagaatattattcttatggtgccagugnaaysaagugyysasnumtvarcatggctgggcgattgatgcgtctggcattattttgcacactcaaggchsgytraaasaasrgyuhsthrgngyatgccaatgacttctatgtataatccacaccgtaatccattggtggatmtrmtthrsrmttyrasnrhsargasnruvaastggctttggacgattacacgccaacgtttcggcggaaaaatgcatgcatrutrthrthrarggnarghgygyysmthsaacgccaaaatggtattaaaccttttttaagtcatgttcgtaaaggcgaaarggnasngyysrhusrhsvaargysgyguatgggttattacttacccgatgaagattttggggcggaacaaagcgtamtgytyrtyrurasguashgyaagugnsrvatttgttgatttctttgggacttataaagcgacattaccagggttaaathvaashhgythrtyrysaathrurgyuasnaaaatggcaaaactttctaaagccgttgttattccaatgtttcctcgtysmtaaysusrysaavavarmthrargtataacgctgaaacgggcaaatatgaaatggaaattcatcctgcaatgtyrasnaaguthrgyystyrgumtguhsraamtaatttaagtgatgatcctgaacaatcagcccgagcaatgaacgaagaaasnusrasasrgugnsraaargaamtasnguguatagaatcttttgttacgccagcgccagagcaatatgtttggattttggusrhvathrraargugntyrvatrucaattattgcgtacaaggaaagatggcgaagatctttatgattaagnuuargthrargysasgyguasutyras

SEQ ID NO: 80 - DNA, 1685 bp, Moraxella catarrhalis
  CDS (1)...(1044)
  misc_feature 61, 67, 151, 157, 356, 439, 451, 454, 511, 513, 536,590, 592, 665, 686, 743, 747, 750, 841, 911, 914, 917,979, 997, 1050, 1073, 1138, 1228, 1297, 1363, 1454,1531, 1539, 1542, 1592, 1602, 1663, 1675, 1683: n = A,T,C or G
atgagttgccatcatcagcataagcagacacccaaacacgccatatccmtsrcyshshsgnhsysgnthrryshsaasrattaagcatatgccaagcttgacagatactcataaacaaagtagccaayshsmtrsruthrasthrhsysgnsrsrgngctgagccaaaatcgtttgaatgggcgtttttacatcccaaatattggaaguryssrhgutraahuhsrystyrtrggagtttggctggcttttgcgttgattttaccgctgatttttctaccggyvatruaahaauuruhurctgcgttggcagttttggatcggcaagcgtcttggcattttggtacatuargtrgnhtrgyysargugyuvahstacttagctaaaagccgagttcaagacactctaaccaacctgcagctttyruaayssrargvagnasthruthrasnugnuaccttcccaaatcaaccaaaatcaaaacacaaggccaccgcacggcaathrhrasngnryssryshsysaathraaarggngtatttattaatcaaggtattggtatttttgaaagtttatgtgcatggvahasngngygyhgusrucysaatrtttcgccctaatgtctttaaacgcacttttagcatttctggtttacaghargrasnvahysargthrhsrsrgyugncatttgattgatgcccaaaaacaaaataaagcggtgattttacttggthsuasaagnysgnasnysaavauugyggacatcgcacgacgcttgatttgggcggtcggttatgtacacagtttgyhsargthrthruasugygyargucysthrgnhtttgcggcggactgcgtgtatcgcccacaaaacaaccctttgcttgaahaaaaascysvatyrargrgnasnasnruugutggtttatctataatgcacgccgctgtatctttgatgagcaaatctcatrhtyrasnaaargargcyshasgugnsraatcgtgatatgaaaaaactcatcactcggctcaaacaaggtcggataasnargasmtysysuthrarguysgngyargatttggtattcacctgatcaagattttggtcttgagcatggcgtgatgtrtyrsrrasgnashgyuguhsgyvamtgcgaccttttttggtgtgcctgcagcaacgattaccgctcagcgtcgtaathrhhgyvaraaaathrthraagnargargcttattaagctgggtgataaagccaatcctcctgtcatcatcatgatguysugyasysaaasnrrvamtmtgatatgctcagacaaacgcccgattatatcgcaaaaggtcaccgtccaasmtuarggnthrrastyraaysgyhsargrcattatcacatcagcctaagcgctgtgttaaaaaattatcccagcgathstyrhssrusraavauysasntyrrsrasgacgaaaccgccgatgctgaacgcatcaatcgactgattgagcaaaatasguthraaasaaguargasnargugugnasnattcaaaaagatttaacccagtggatgtggtttcatcgccgctttaaagnysasuthrgntrmttrhhsargarghysactcaagccgatgacaccaattactatcaacattaatgthrgnaaasasthrasntyrtyrgnhs

SEQ ID NO: 81 - DNA, 1410 bp, Neisseria meningitidis
  CDS (1)...(876)
  misc_feature 66, 215, 300, 459, 613, 615, 682, 687, 744, 755, 766,844, 916, 927, 992, 1069, 1141, 1225, 1233, 1289, 1306: n = A,T,C or G
atgaaatttatattttttgtactgtatgttttgcagtttctgccgtttmtyshhhvautyrvaugnhurhgcgctgctgcacaaacttgccgacctgacgggtttgctcgcctaccttaauuhsysuaaasuthrgyuuaatyruttggtcaaaccccgccgccgtatcggcgaaatcaatttggcaaaatgcuvaysrargargarggyguasnuaayscystttcccgagtgggacggaaaaaagcgcgaaaccgtattgaagcagcathrgutrasgyysysargguthrvauysgnhsttcaaacatatggcgaaactgatgcttgaatacggcttatattggtachyshsmtaaysumtugutyrgyutyrtrtyrgcgcctgccgggcgtttgaaatcgctggtgcgttaccgcaataagcataaraagyarguyssruvaargtyrargasnyshstatttggacgacgcgctggcggcgggggaaaaagtcatcattctgtactyruasasaauaaaagyguysvautyrccgcacttcaccgcgttcgagatggcggtgtacgcgcttaatcaggatrhshthraahgumtaavatyraauasngnasgtaccgctgatcagtatgtattcccaccaaaaaaacaagatattggacvarusrmttyrsrhsgnysasnysuasgcacagattttgaaaggccgcaaccgctacgacaatgtcttccttatcaagnuysgyargasnargtyrasasnvahugggcgcaccgaaggcgtgcgcgccctcgtcaaacagttccgcaaaagcgyargthrgugyvaargaauvaysgnhargyssragcgcgccgtttctgtatctgcccgatcaggatttcggacgcaacgatsraarhutyrurasgnashgyargasnastcggtttttgtggattttttcggtattcagacggcaacgattaccggcsrvahvaashhgygnthraathrthrgyttgagccgcattgccgcgcttgcaaatgcaaaagtgatacccgccatcusrargaaaauaaasnaaysvaraacccgtccgcgaggcggacaatacggttacattgcatttctacccggctrvaargguaaasasnthrvathruhshtyrraatgggaatcctttccgagtgaagatgcgcaggccgacgcgcagcgcatgtrgusrhrsrguasaagnaaasaagnargmtaaccgttttatcgaggaaccgtgcgcgaacatcccgagcagtatttttasnarghgugurcysaaasnrsrsrhggctgcacaagcgtttcaaaacccgtccggaaggcagccccgatttttgycysthrsrvasrysrvaargysaaaarhactgatacgtaathrasthr

SEQ ID NO: 82 - DNA, 38 bp, Artificial Sequence
  PorA5′ Fwd primer
cccaagcttgccgtctgaatacatcccgtcattcctca

SEQ ID NO: 83 - DNA, 34 bp, Artificial Sequence
  PorA5′ Rev primer
cgatgctcgcgactccagagacctcgtgcgggcc

SEQ ID NO: 84 - DNA, 38 bp, Artificial Sequence
  PorA3′ Fwd primer
ggaagatctgattaaataggcgaaaataccagctacga

SEQ ID NO: 85 - DNA, 37 bp, Artificial Sequence
  PorA3′ Rev primer
gccgaattcttcagacggcgcagcaggaatttatcgg

SEQ ID NO: 86 - DNA, 41 bp, Artificial Sequence
  PoLa Rev1 primer
gaattgttatccgctcacaattccgggcaaacacccgatac

SEQ ID NO: 87 - DNA, 70 bp, Artificial Sequence
  PoLa Rev2 primer
gaattccatatgatcggcttccttttgtaaatttgataaaaacctaaaaacatcgaattgttatccgctc

SEQ ID NO: 88 - DNA, 30 bp, Artificial Sequence
  PorAlacO Fwd primer
aagctctgcaggaggtctgcgcttgaattg

SEQ ID NO: 89 - DNA, 28 bp, Artificial Sequence
  PorAlacO Rev primer
cttaaggcatatgggcttccttttgtaa

SEQ ID NO: 90 - DNA, 22 bp, Artificial Sequence
  PPA1 primer
gcggccgttgccgatgtcagcc

SEQ ID NO: 91 - DNA, 24 bp, Artificial Sequence
  PPA2 primer
ggcatagctgatgcgtggaactgc

SEQ ID NO: 92 - DNA, 33 bp, Artificial Sequence
  N-full-01 primer
gggaattccatatgaaaaaagcacttgccacac

SEQ ID NO: 93 - DNA, 31 bp, Artificial Sequence
  Nde-NspA-3 primer
ggaattccatatgtcagaatttgacgcgcac

SEQ ID NO: 94 - DNA, 30 bp, PNS1 primer
ccgcgaattcggaaccgaacacgccgttcg

SEQ ID NO: 95 - DNA, 27 bp, Artificial Sequence
  PNS1 primer
cgtctagacgtagcggtatccggctgc

SEQ ID NO: 96 - DNA, 38 bp, Artificial Sequence
  PromD15-51X primer
gggcgaattcgcggccgccgtcaacggcacacccgttg

SEQ ID NO: 97 - DNA, 28 bp, Artificial Sequence
  ProD15-52 primer
gctctagagcggaatgcggtttcagacg

SEQ ID NO: 98 - DNA, 47 bp, Artificial Sequence
  PNS4 primer
agctttatttaaatccttaattaacgcgtccggaaaatatgcttatc

SEQ ID NO: 99 - DNA, 33 bp, Artificial Sequence
  PNS5 primer
agctttgtttaaaccctgttccgctgcttcggc

SEQ ID NO: 100 - DNA, 43 bp, Artificial Sequence
  D15-S4 primer
gtccgcatttaaatccttaattaagcagccggacagggcgtgg

SEQ ID NO: 101 - DNA, 33 bp, Artificial Sequence
  D15-S5 primer
agctttgtttaaaggatcagggtgtggtcgggc

SEQ ID NO: 102 - DNA, 28 bp, Artificial Sequence
  DT88 primer
gaagagaaggtggaaatggcgttttggc

SEQ ID NO: 103 - DNA, 27 bp, Artificial Sequence
  DT89 primer
ccaaaacgccatttccaccttctcttc

SEQ ID NO: 104 - DNA, 25 bp, Artificial Sequence
  PorA3 primer
ccaaatcctcgctccccttaaagcc

SEQ ID NO: 105 - DNA, 24 bp, Artificial Sequence
  p1-2 primer
cgctgattttcgtcctgatgcggc

SEQ ID NO: 106 - DNA, 25 bp, Artificial Sequence
  p1-1 primer
ggtcaattgcgcctggatgttcctg

SEQ ID NO: 107 - DNA, 26 bp, Artificial Sequence
  porB1 primer
ggtagcggttgtaacttcagtaactt

SEQ ID NO: 108 - DNA, 25 bp, Artificial Sequence
  porB2 primer
gtcttcttggcctttgaagccgatt

SEQ ID NO: 109 - DNA, 25 bp, Artificial Sequence
  porB3 primer
ggagtcagtaccggcgatagatgct

SEQ ID NO: 110 - DNA, 37 bp, Artificial Sequence
  ProD15-51X primer
gggcgaattcgcggccgccgtcaacggcacaccgttg

SEQ ID NO: 111 - DNA, 43 bp, Artificial Sequence
  TnRD15-KpnI/XbaI + US primer
cgccggtacctctagagccgtctgaaccactcgtggacaaccc

SEQ ID NO: 112 - DNA, 29 bp, Artificial Sequence
  TnR03Cam (KpnI) primer
cgccggtaccgccgctaactataacggtc

SEQ ID NO: 113 - DNA, 31 bp, Artificial Sequence
  PorA-01 primer
cgccggtaccgaggtctgcgcttgaattgtg

SEQ ID NO: 114 - DNA, 33 bp, Artificial Sequence
  PorA02 primer
cgccggtacctctagacatcgggcaaacacccg

SEQ ID NO: 115 - DNA, 20 bp, Artificial Sequence
  Cam-05 primer
gtactgcgatgagtggcagg

SEQ ID NO: 116 - DNA, 31 bp, Artificial Sequence
  Hsf 01-Nde primer
ggaattccatatgatgaacaaaatataccgc

SEQ ID NO: 117 - DNA, 31 bp, Artificial Sequence
  Hsf 02-Nhe primer
gtagctagctagcttaccactgataaccgac

SEQ ID NO: 118 - DNA, 36 bp, Artificial Sequence
  GFP-mut-Asn primer
aactgcagaattaatatgaaaggagaagaacttttc

SEQ ID NO: 119 - DNA, 33 bp, Artificial Sequence
  GFP-Spe primer
gacatactagtttatttgtagagctcatccatg

SEQ ID NO: 120 - DNA, 30 bp, Artificial Sequence
  RP1 (SacII) primer
tccccgcgggccgtctgaatacatcccgtc

SEQ ID NO: 121 - DNA, 51 bp, Artificial Sequence
  RP2 primer
catatgggcttccttttgtaaatttgagggcaaacacccgatacgtcttca

SEQ ID NO: 122 - DNA, 48 bp, Artificial Sequence
  RP3 primer
agacgtatcgggtgtttgccctcaaatttacaaaaggaagcccatatg

SEQ ID NO: 123 - DNA, 33 bp, Artificial Sequence
  RP4 (ApaI) primer
gggtattccgggcccttcagacggcgcagcagg

SEQ ID NO: 124 - DNA, 28 bp, Artificial Sequence
  PNS1′ primer
ccgcgaattcgacgaagccgccctcgac

SEQ ID NO: 125 - DNA, 37 bp, Artificial Sequence
  BAD01-2 primer
ggcgcccgggctcgagcttatcgatggaaaacgcagc

SEQ ID NO: 126 - DNA, 47 bp, Artificial Sequence
  BAD02-2 primer
ggcgcccgggctcgagttcagacggcgcgcttatatagtggattaac

SEQ ID NO: 127 - DNA, 39 bp, Artificial Sequence
  BAD 15-2 primer
ggcgcccgggctcgagtctagacatcgggcaaacacccg

SEQ ID NO: 128 - DNA, 39 bp, Artificial Sequence
  BAD 03-2 primer
ggcgcccgggctcgagcactagtattaccctgttatccc

SEQ ID NO: 129 - DNA, 19 bp, Artificial Sequence
  BAD 25 primer
gagcgaagccgtcgaacgc

SEQ ID NO: 130 - DNA, 20 bp, Artificial Sequence
  BAD08 primer
cttaagcgtcggacatttcc

SEQ ID NO: 131 - DNA, 31 bp, Artificial Sequence
  PLA1 Amo5 primer
gccgtctgaatttaaaattgcgcgtttacag

SEQ ID NO: 132 - DNA, 38 bp, Artificial Sequence
  PLA1 Amo3 primer
gtagtctagattcagacggcgcaatttggtttccgcac

SEQ ID NO: 133 - DNA, 27 bp, Artificial Sequence
  CIRC1-Bg1 primer
cctagatctctccgccccccattgtcg

SEQ ID NO: 134 - DNA, 46 bp, Artificial Sequence
  CIRC1-XH-RBS/2 primer
ccgctcgagtacaaaaggaagccgatatgaatatacggaatatgcg

SEQ ID NO: 135 - DNA, 24 bp, Artificial Sequence
  CIRC2-XHO/2 primer
ccgctcgagatgaatatacggaat

SEQ ID NO: 136 - DNA, 38 bp, Artificial Sequence
  BAD20 primer
tcccccgggagatctcactagtattaccctgttatccc

SEQ ID NO: 137 - DNA, 32 bp, Artificial Sequence
  CM-PORA-3 primer
ccgctcgagataaaaacctaaaaacatcgggc

SEQ ID NO: 138 - DNA, 28 bp, Artificial Sequence
  CM-PORA-D15/3 primer
cggctcgagtgtcagttccttgtggtgc

SEQ ID NO: 139 - DNA, 45 bp, Artificial Sequence
  BAD16 primer
ggcctagctagccgtctgaagcgattagagtttcaaaatttattc

SEQ ID NO: 140 - DNA, 42 bp, Artificial Sequence
  BAD17 primer
ggccaagcttcagacggcgttcgaccgagtttgagcctttgc

SEQ ID NO: 141 - DNA, 39 bp, Artificial Sequence
  BAD18 primer
tcccccgggaagatctggacgaaaaatctcaagaaaccg

SEQ ID NO: 142 - DNA, 64 bp, Artificial Sequence
  BAD19 primer
ggaagatctccgctcgagcaaatttacaaaaggaagccgatatgcaacagcaacatttgttccg

SEQ ID NO: 143 - DNA, 36 bp, Artificial Sequence
  BAD21 primer
ggaagatctccgctcgagacatcgggcaaacacccg

SEQ ID NO: 144 - DNA, 36 bp, Artificial Sequence
  PQ-rec5-Nhe primer
ctagctagcgccgtctgaacgacgcgaagccaaagc

SEQ ID NO: 145 - DNA, 37 bp, Artificial Sequence
  PQ-rec3-Hin primer
gccaagcttttcagacggcacggtatcgtccgattcg

SEQ ID NO: 146 - DNA, 30 bp, Artificial Sequence
  CIRC1-PQ-Bg1 primer
ggaagatctaatggagtaatcctcttctta

SEQ ID NO: 147 - DNA, 50 bp, Artificial Sequence
  CIRC1-PQ-XHO primer
ccgctcgagtacaaaaggaagccgatatgattaccaaactgacaaaaatc

SEQ ID NO: 148 - DNA, 33 bp, Artificial Sequence
  CIRC2-PQ-X primer
ccgctcgagatgaataccaaactgacaaaaatc

SEQ ID NO: 149 - DNA, 40 bp, Artificial Sequence
  CM-PORA-3 primer
ccgctcgagataaaaacctaaaaacatcgggcaaacaccc

SEQ ID NO: 150 - DNA, 28 bp, Artificial Sequence
  CM-PORA-D153 primer
gggctcgagtgtcagttccttgtggtgc

SEQ ID NO: 151 - DNA, 32 bp, Artificial Sequence
  CIRC-Kan-Nco primer
catgccatggttagaaaaactcatcgagcatc

SEQ ID NO: 152 - DNA, 31 bp, Artificial Sequence
  CIRC-Kan-Xba primer
ctagtctagatcagaattggttaattggttg

SEQ ID NO: 153 - DNA, 43 bp, Artificial Sequence
  SAC/NCO/NEW5 primer
catgccatgggaggatgaacgatgaacatcaaaaagtttgcaa

SEQ ID NO: 154 - DNA, 33 bp, Artificial Sequence
  SAC/NCO/NEW3 primer
gatcccatggttatttgttaactgttaattgtc

SEQ ID NO: 155 - DNA, 72 bp, Artificial Sequence
  Kan-PorA-5 primer
gccgtctgaacccgtcattcccgcgcaggcgggaatccagtccgttcagtttcgggaaagccacgttgtgtc

SEQ ID NO: 156 - DNA, 69 bp, Artificial Sequence
  Kan-PorA-3 primer
ttcagacggcgcagcaggaatttatcggaaataactgaaaccgaacagactaggctgaggtctgcctcg